SEQUENCE LISTING (1175 sequences)

SEQ ID NO: 1 - DNA, 60 bp, Artificial Sequence
  Synthetic oligonucleotide
  misc_feature (19)..(26): n is a, c, g, or t
acgacgctcttccgatctnnnnnnnnttctcgcatgttttttttttttttttttttttgc

SEQ ID NO: 2 - DNA, 60 bp, Artificial Sequence
  Synthetic oligonucleotide
  misc_feature (19)..(26): n is a, c, g, or t
acgacgctcttccgatctnnnnnnnntcctaccagtttttttttttttttttttttttgc

SEQ ID NO: 3 - DNA, 60 bp, Artificial Sequence
  Synthetic oligonucleotide
  misc_feature (19)..(26): n is a, c, g, or t
acgacgctcttccgatctnnnnnnnngcgttggagcttttttttttttttttttttttgc

SEQ ID NO: 4 - DNA, 60 bp, Artificial Sequence
  Synthetic oligonucleotide
  misc_feature (19)..(26): n is a, c, g, or t
acgacgctcttccgatctnnnnnnnngatcttacgcttttttttttttttttttttttgc

SEQ ID NO: 5 - DNA, 60 bp, Artificial Sequence
  Synthetic oligonucleotide
  misc_feature (19)..(26): n is a, c, g, or t
acgacgctcttccgatctnnnnnnnnctgatggtcattttttttttttttttttttttgc

SEQ ID NO: 6 - DNA, 60 bp, Artificial Sequence
  Synthetic oligonucleotide
  misc_feature (19)..(26): n is a, c, g, or t
acgacgctcttccgatctnnnnnnnnccgagaatccttttttttttttttttttttttgc

SEQ ID NO: 7 - DNA, 60 bp, Artificial Sequence
  Synthetic oligonucleotide
  misc_feature (19)..(26): n is a, c, g, or t
acgacgctcttccgatctnnnnnnnngccgcaacgattttttttttttttttttttttgc

SEQ ID NO: 8 - DNA, 60 bp, Artificial Sequence
  Synthetic oligonucleotide
  misc_feature (19)..(26): n is a, c, g, or t
acgacgctcttccgatctnnnnnnnntgagtctggcttttttttttttttttttttttgc

SEQ ID NO: 9 - DNA, 60 bp, Artificial Sequence
  Synthetic oligonucleotide
  misc_feature (19)..(26): n is a, c, g, or t
acgacgctcttccgatctnnnnnnnntgcggacctattttttttttttttttttttttgc

SEQ ID NO: 10 - DNA, 60 bp, Artificial Sequence
  Synthetic oligonucleotide
  misc_feature (19)..(26): n is a, c, g, or t
acgacgctcttccgatctnnnnnnnnacctcgttgattttttttttttttttttttttgc

SEQ ID NO: 11 - DNA, 60 bp, Artificial Sequence
  Synthetic oligonucleotide
  misc_feature (19)..(26): n is a, c, g, or t
acgacgctcttccgatctnnnnnnnnacggaggcggttttttttttttttttttttttgc

SEQ ID NO: 12 - DNA, 60 bp, Artificial Sequence
  Synthetic oligonucleotide
  misc_feature (19)..(26): n is a, c, g, or t
acgacgctcttccgatctnnnnnnnntagatctactttttttttttttttttttttttgc

SEQ ID NO: 13 - DNA, 60 bp, Artificial Sequence
  Synthetic oligonucleotide
  misc_feature (19)..(26): n is a, c, g, or t
acgacgctcttccgatctnnnnnnnnaattaagactttttttttttttttttttttttgc

SEQ ID NO: 14 - DNA, 60 bp, Artificial Sequence
  Synthetic oligonucleotide
  misc_feature (19)..(26): n is a, c, g, or t
acgacgctcttccgatctnnnnnnnnccattgcgttttttttttttttttttttttttgc

SEQ ID NO: 15 - DNA, 60 bp, Artificial Sequence
  Synthetic oligonucleotide
  misc_feature (19)..(26): n is a, c, g, or t
acgacgctcttccgatctnnnnnnnnttattcattcttttttttttttttttttttttgc

SEQ ID NO: 16 - DNA, 60 bp, Artificial Sequence
  Synthetic oligonucleotide
  misc_feature (19)..(26): n is a, c, g, or t
acgacgctcttccgatctnnnnnnnnatctccgaacttttttttttttttttttttttgc

SEQ ID NO: 17 - DNA, 60 bp, Artificial Sequence
  Synthetic oligonucleotide
  misc_feature (19)..(26): n is a, c, g, or t
acgacgctcttccgatctnnnnnnnnttgacttcagttttttttttttttttttttttgc

SEQ ID NO: 18 - DNA, 60 bp, Artificial Sequence
  Synthetic oligonucleotide
  misc_feature (19)..(26): n is a, c, g, or t
acgacgctcttccgatctnnnnnnnnggcaggtattttttttttttttttttttttttgc

SEQ ID NO: 19 - DNA, 60 bp, Artificial Sequence
  Synthetic oligonucleotide
  misc_feature (19)..(26): n is a, c, g, or t
acgacgctcttccgatctnnnnnnnnagagctataattttttttttttttttttttttgc

SEQ ID NO: 20 - DNA, 60 bp, Artificial Sequence
  Synthetic oligonucleotide
  misc_feature (19)..(26): n is a, c, g, or t
acgacgctcttccgatctnnnnnnnnctaagagaagttttttttttttttttttttttgc

SEQ ID NO: 21 - DNA, 60 bp, Artificial Sequence
  Synthetic oligonucleotide
  misc_feature (19)..(26): n is a, c, g, or t
acgacgctcttccgatctnnnnnnnnactcaataggttttttttttttttttttttttgc

SEQ ID NO: 22 - DNA, 60 bp, Artificial Sequence
  Synthetic oligonucleotide
  misc_feature (19)..(26): n is a, c, g, or t
acgacgctcttccgatctnnnnnnnncttgcgccgcttttttttttttttttttttttgc

SEQ ID NO: 23 - DNA, 60 bp, Artificial Sequence
  Synthetic oligonucleotide
  misc_feature (19)..(26): n is a, c, g, or t
acgacgctcttccgatctnnnnnnnnaatcgtagcgttttttttttttttttttttttgc

SEQ ID NO: 24 - DNA, 60 bp, Artificial Sequence
  Synthetic oligonucleotide
  misc_feature (19)..(26): n is a, c, g, or t
acgacgctcttccgatctnnnnnnnnggtactgcctttttttttttttttttttttttgc

SEQ ID NO: 25 - DNA, 60 bp, Artificial Sequence
  Synthetic oligonucleotide
  misc_feature (19)..(26): n is a, c, g, or t
acgacgctcttccgatctnnnnnnnntagaattaacttttttttttttttttttttttgc

SEQ ID NO: 26 - DNA, 60 bp, Artificial Sequence
  Synthetic oligonucleotide
  misc_feature (19)..(26): n is a, c, g, or t
acgacgctcttccgatctnnnnnnnngccattctccttttttttttttttttttttttgc

SEQ ID NO: 27 - DNA, 60 bp, Artificial Sequence
  Synthetic oligonucleotide
  misc_feature (19)..(26): n is a, c, g, or t
acgacgctcttccgatctnnnnnnnntgccggcagattttttttttttttttttttttgc

SEQ ID NO: 28 - DNA, 60 bp, Artificial Sequence
  Synthetic oligonucleotide
  misc_feature (19)..(26): n is a, c, g, or t
acgacgctcttccgatctnnnnnnnnttaccgaggcttttttttttttttttttttttgc

SEQ ID NO: 29 - DNA, 60 bp, Artificial Sequence
  Synthetic oligonucleotide
  misc_feature (19)..(26): n is a, c, g, or t
acgacgctcttccgatctnnnnnnnnatcatattagttttttttttttttttttttttgc

SEQ ID NO: 30 - DNA, 60 bp, Artificial Sequence
  Synthetic oligonucleotide
  misc_feature (19)..(26): n is a, c, g, or t
acgacgctcttccgatctnnnnnnnntggtcagccattttttttttttttttttttttgc

SEQ ID NO: 31 - DNA, 60 bp, Artificial Sequence
  Synthetic oligonucleotide
  misc_feature (19)..(26): n is a, c, g, or t
acgacgctcttccgatctnnnnnnnnactatgcaatttttttttttttttttttttttgc

SEQ ID NO: 32 - DNA, 60 bp, Artificial Sequence
  Synthetic oligonucleotide
  misc_feature (19)..(26): n is a, c, g, or t
acgacgctcttccgatctnnnnnnnncgacgcgactttttttttttttttttttttttgc

SEQ ID NO: 33 - DNA, 60 bp, Artificial Sequence
  Synthetic oligonucleotide
  misc_feature (19)..(26): n is a, c, g, or t
acgacgctcttccgatctnnnnnnnngatacggaacttttttttttttttttttttttgc

SEQ ID NO: 34 - DNA, 60 bp, Artificial Sequence
  Synthetic oligonucleotide
  misc_feature (19)..(26): n is a, c, g, or t
acgacgctcttccgatctnnnnnnnnttatccggatttttttttttttttttttttttgc

SEQ ID NO: 35 - DNA, 60 bp, Artificial Sequence
  Synthetic oligonucleotide
  misc_feature (19)..(26): n is a, c, g, or t
acgacgctcttccgatctnnnnnnnntagagtaatattttttttttttttttttttttgc

SEQ ID NO: 36 - DNA, 60 bp, Artificial Sequence
  Synthetic oligonucleotide
  misc_feature (19)..(26): n is a, c, g, or t
acgacgctcttccgatctnnnnnnnngcaggtccgtttttttttttttttttttttttgc

SEQ ID NO: 37 - DNA, 60 bp, Artificial Sequence
  Synthetic oligonucleotide
  misc_feature (19)..(26): n is a, c, g, or t
acgacgctcttccgatctnnnnnnnntcggccttacttttttttttttttttttttttgc

SEQ ID NO: 38 - DNA, 60 bp, Artificial Sequence
  Synthetic oligonucleotide
  misc_feature (19)..(26): n is a, c, g, or t
acgacgctcttccgatctnnnnnnnnagaacgtctcttttttttttttttttttttttgc

SEQ ID NO: 39 - DNA, 60 bp, Artificial Sequence
  Synthetic oligonucleotide
  misc_feature (19)..(26): n is a, c, g, or t
acgacgctcttccgatctnnnnnnnnccagttccaattttttttttttttttttttttgc

SEQ ID NO: 40 - DNA, 60 bp, Artificial Sequence
  Synthetic oligonucleotide
  misc_feature (19)..(26): n is a, c, g, or t
acgacgctcttccgatctnnnnnnnnggcgttaaggttttttttttttttttttttttgc

SEQ ID NO: 41 - DNA, 60 bp, Artificial Sequence
  Synthetic oligonucleotide
  misc_feature (19)..(26): n is a, c, g, or t
acgacgctcttccgatctnnnnnnnnacttaaccttttttttttttttttttttttttgc

SEQ ID NO: 42 - DNA, 60 bp, Artificial Sequence
  Synthetic oligonucleotide
  misc_feature (19)..(26): n is a, c, g, or t
acgacgctcttccgatctnnnnnnnncaaccgctaattttttttttttttttttttttgc

SEQ ID NO: 43 - DNA, 60 bp, Artificial Sequence
  Synthetic oligonucleotide
  misc_feature (19)..(26): n is a, c, g, or t
acgacgctcttccgatctnnnnnnnngaccttgatattttttttttttttttttttttgc

SEQ ID NO: 44 - DNA, 60 bp, Artificial Sequence
  Synthetic oligonucleotide
  misc_feature (19)..(26): n is a, c, g, or t
acgacgctcttccgatctnnnnnnnntctgataccattttttttttttttttttttttgc

SEQ ID NO: 45 - DNA, 60 bp, Artificial Sequence
  Synthetic oligonucleotide
  misc_feature (19)..(26): n is a, c, g, or t
acgacgctcttccgatctnnnnnnnngaagatcgagttttttttttttttttttttttgc

SEQ ID NO: 46 - DNA, 60 bp, Artificial Sequence
  Synthetic oligonucleotide
  misc_feature (19)..(26): n is a, c, g, or t
acgacgctcttccgatctnnnnnnnnaggagcggtattttttttttttttttttttttgc

SEQ ID NO: 47 - DNA, 60 bp, Artificial Sequence
  Synthetic oligonucleotide
  misc_feature (19)..(26): n is a, c, g, or t
acgacgctcttccgatctnnnnnnnnaagaagctagttttttttttttttttttttttgc

SEQ ID NO: 48 - DNA, 60 bp, Artificial Sequence
  Synthetic oligonucleotide
  misc_feature (19)..(26): n is a, c, g, or t
acgacgctcttccgatctnnnnnnnntccggcctcgttttttttttttttttttttttgc

SEQ ID NO: 49 - DNA, 60 bp, Artificial Sequence
  Synthetic oligonucleotide
  misc_feature (19)..(26): n is a, c, g, or t
acgacgctcttccgatctnnnnnnnnagagaaggttttttttttttttttttttttttgc

SEQ ID NO: 50 - DNA, 60 bp, Artificial Sequence
  Synthetic oligonucleotide
  misc_feature (19)..(26): n is a, c, g, or t
acgacgctcttccgatctnnnnnnnncatactccgattttttttttttttttttttttgc

SEQ ID NO: 51 - DNA, 60 bp, Artificial Sequence
  Synthetic oligonucleotide
  misc_feature (19)..(26): n is a, c, g, or t
acgacgctcttccgatctnnnnnnnngctaacttgcttttttttttttttttttttttgc

SEQ ID NO: 52 - DNA, 60 bp, Artificial Sequence
  Synthetic oligonucleotide
  misc_feature (19)..(26): n is a, c, g, or t
acgacgctcttccgatctnnnnnnnnaatccatcttttttttttttttttttttttttgc

SEQ ID NO: 53 - DNA, 60 bp, Artificial Sequence
  Synthetic oligonucleotide
  misc_feature (19)..(26): n is a, c, g, or t
acgacgctcttccgatctnnnnnnnnggctgagctcttttttttttttttttttttttgc

SEQ ID NO: 54 - DNA, 60 bp, Artificial Sequence
  Synthetic oligonucleotide
  misc_feature (19)..(26): n is a, c, g, or t
acgacgctcttccgatctnnnnnnnnccgattcctgttttttttttttttttttttttgc

SEQ ID NO: 55 - DNA, 60 bp, Artificial Sequence
  Synthetic oligonucleotide
  misc_feature (19)..(26): n is a, c, g, or t
acgacgctcttccgatctnnnnnnnnaccgccaaccttttttttttttttttttttttgc

SEQ ID NO: 56 - DNA, 60 bp, Artificial Sequence
  Synthetic oligonucleotide
  misc_feature (19)..(26): n is a, c, g, or t
acgacgctcttccgatctnnnnnnnntggcctgaagttttttttttttttttttttttgc

SEQ ID NO: 57 - DNA, 60 bp, Artificial Sequence
  Synthetic oligonucleotide
  misc_feature (19)..(26): n is a, c, g, or t
acgacgctcttccgatctnnnnnnnnaacctcattcttttttttttttttttttttttgc

SEQ ID NO: 58 - DNA, 60 bp, Artificial Sequence
  Synthetic oligonucleotide
  misc_feature (19)..(26): n is a, c, g, or t
acgacgctcttccgatctnnnnnnnnataaggagcattttttttttttttttttttttgc

SEQ ID NO: 59 - DNA, 60 bp, Artificial Sequence
  Synthetic oligonucleotide
  misc_feature (19)..(26): n is a, c, g, or t
acgacgctcttccgatctnnnnnnnncgaacgccggttttttttttttttttttttttgc

SEQ ID NO: 60 - DNA, 60 bp, Artificial Sequence
  Synthetic oligonucleotide
  misc_feature (19)..(26): n is a, c, g, or t
acgacgctcttccgatctnnnnnnnnggtatgcttgttttttttttttttttttttttgc

SEQ ID NO: 61 - DNA, 60 bp, Artificial Sequence
  Synthetic oligonucleotide
  misc_feature (19)..(26): n is a, c, g, or t
acgacgctcttccgatctnnnnnnnnaacctgcgtattttttttttttttttttttttgc

SEQ ID NO: 62 - DNA, 60 bp, Artificial Sequence
  Synthetic oligonucleotide
  misc_feature (19)..(26): n is a, c, g, or t
acgacgctcttccgatctnnnnnnnnggcagacgccttttttttttttttttttttttgc

SEQ ID NO: 63 - DNA, 60 bp, Artificial Sequence
  Synthetic oligonucleotide
  misc_feature (19)..(26): n is a, c, g, or t
acgacgctcttccgatctnnnnnnnntagccgtcatttttttttttttttttttttttgc

SEQ ID NO: 64 - DNA, 60 bp, Artificial Sequence
  Synthetic oligonucleotide
  misc_feature (19)..(26): n is a, c, g, or t
acgacgctcttccgatctnnnnnnnncctggaagagttttttttttttttttttttttgc

SEQ ID NO: 65 - DNA, 60 bp, Artificial Sequence
  Synthetic oligonucleotide
  misc_feature (19)..(26): n is a, c, g, or t
acgacgctcttccgatctnnnnnnnnggaggttctattttttttttttttttttttttgc

SEQ ID NO: 66 - DNA, 60 bp, Artificial Sequence
  Synthetic oligonucleotide
  misc_feature (19)..(26): n is a, c, g, or t
acgacgctcttccgatctnnnnnnnnctagtagtctttttttttttttttttttttttgc

SEQ ID NO: 67 - DNA, 60 bp, Artificial Sequence
  Synthetic oligonucleotide
  misc_feature (19)..(26): n is a, c, g, or t
acgacgctcttccgatctnnnnnnnnatcatcaacgttttttttttttttttttttttgc

SEQ ID NO: 68 - DNA, 60 bp, Artificial Sequence
  Synthetic oligonucleotide
  misc_feature (19)..(26): n is a, c, g, or t
acgacgctcttccgatctnnnnnnnnacgcgagattttttttttttttttttttttttgc

SEQ ID NO: 69 - DNA, 60 bp, Artificial Sequence
  Synthetic oligonucleotide
  misc_feature (19)..(26): n is a, c, g, or t
acgacgctcttccgatctnnnnnnnngaagaggcatttttttttttttttttttttttgc

SEQ ID NO: 70 - DNA, 60 bp, Artificial Sequence
  Synthetic oligonucleotide
  misc_feature (19)..(26): n is a, c, g, or t
acgacgctcttccgatctnnnnnnnnggtatccgccttttttttttttttttttttttgc

SEQ ID NO: 71 - DNA, 60 bp, Artificial Sequence
  Synthetic oligonucleotide
  misc_feature (19)..(26): n is a, c, g, or t
acgacgctcttccgatctnnnnnnnnaactaggcgcttttttttttttttttttttttgc

SEQ ID NO: 72 - DNA, 60 bp, Artificial Sequence
  Synthetic oligonucleotide
  misc_feature (19)..(26): n is a, c, g, or t
acgacgctcttccgatctnnnnnnnntcgctaagcattttttttttttttttttttttgc

SEQ ID NO: 73 - DNA, 60 bp, Artificial Sequence
  Synthetic oligonucleotide
  misc_feature (19)..(26): n is a, c, g, or t
acgacgctcttccgatctnnnnnnnntatatactaattttttttttttttttttttttgc

SEQ ID NO: 74 - DNA, 60 bp, Artificial Sequence
  Synthetic oligonucleotide
  misc_feature (19)..(26): n is a, c, g, or t
acgacgctcttccgatctnnnnnnnnacttgctagattttttttttttttttttttttgc

SEQ ID NO: 75 - DNA, 60 bp, Artificial Sequence
  Synthetic oligonucleotide
  misc_feature (19)..(26): n is a, c, g, or t
acgacgctcttccgatctnnnnnnnnaaccattggattttttttttttttttttttttgc

SEQ ID NO: 76 - DNA, 60 bp, Artificial Sequence
  Synthetic oligonucleotide
  misc_feature (19)..(26): n is a, c, g, or t
acgacgctcttccgatctnnnnnnnntcgcggttggttttttttttttttttttttttgc

SEQ ID NO: 77 - DNA, 60 bp, Artificial Sequence
  Synthetic oligonucleotide
  misc_feature (19)..(26): n is a, c, g, or t
acgacgctcttccgatctnnnnnnnncgtagttaccttttttttttttttttttttttgc

SEQ ID NO: 78 - DNA, 60 bp, Artificial Sequence
  Synthetic oligonucleotide
  misc_feature (19)..(26): n is a, c, g, or t
acgacgctcttccgatctnnnnnnnntccaatcatcttttttttttttttttttttttgc

SEQ ID NO: 79 - DNA, 60 bp, Artificial Sequence
  Synthetic oligonucleotide
  misc_feature (19)..(26): n is a, c, g, or t
acgacgctcttccgatctnnnnnnnnaatcgataatttttttttttttttttttttttgc

SEQ ID NO: 80 - DNA, 60 bp, Artificial Sequence
  Synthetic oligonucleotide
  misc_feature (19)..(26): n is a, c, g, or t
acgacgctcttccgatctnnnnnnnnccattatctattttttttttttttttttttttgc

SEQ ID NO: 81 - DNA, 60 bp, Artificial Sequence
  Synthetic oligonucleotide
  misc_feature (19)..(26): n is a, c, g, or t
acgacgctcttccgatctnnnnnnnntcaacgtaagttttttttttttttttttttttgc

SEQ ID NO: 82 - DNA, 60 bp, Artificial Sequence
  Synthetic oligonucleotide
  misc_feature (19)..(26): n is a, c, g, or t
acgacgctcttccgatctnnnnnnnntctaatagtattttttttttttttttttttttgc

SEQ ID NO: 83 - DNA, 60 bp, Artificial Sequence
  Synthetic oligonucleotide
  misc_feature (19)..(26): n is a, c, g, or t
acgacgctcttccgatctnnnnnnnnaaccgctggtttttttttttttttttttttttgc

SEQ ID NO: 84 - DNA, 60 bp, Artificial Sequence
  Synthetic oligonucleotide
  misc_feature (19)..(26): n is a, c, g, or t
acgacgctcttccgatctnnnnnnnngatcgcttctttttttttttttttttttttttgc

SEQ ID NO: 85 - DNA, 60 bp, Artificial Sequence
  Synthetic oligonucleotide
  misc_feature (19)..(26): n is a, c, g, or t
acgacgctcttccgatctnnnnnnnnctaactagatttttttttttttttttttttttgc

SEQ ID NO: 86 - DNA, 60 bp, Artificial Sequence
  Synthetic oligonucleotide
  misc_feature (19)..(26): n is a, c, g, or t
acgacgctcttccgatctnnnnnnnngctggaacttttttttttttttttttttttttgc

SEQ ID NO: 87 - DNA, 60 bp, Artificial Sequence
  Synthetic oligonucleotide
  misc_feature (19)..(26): n is a, c, g, or t
acgacgctcttccgatctnnnnnnnnaggttagttcttttttttttttttttttttttgc

SEQ ID NO: 88 - DNA, 60 bp, Artificial Sequence
  Synthetic oligonucleotide
  misc_feature (19)..(26): n is a, c, g, or t
acgacgctcttccgatctnnnnnnnncattcgacggttttttttttttttttttttttgc

SEQ ID NO: 89 - DNA, 60 bp, Artificial Sequence
  Synthetic oligonucleotide
  misc_feature (19)..(26): n is a, c, g, or t
acgacgctcttccgatctnnnnnnnncattcaatcattttttttttttttttttttttgc

SEQ ID NO: 90 - DNA, 60 bp, Artificial Sequence
  Synthetic oligonucleotide
  misc_feature (19)..(26): n is a, c, g, or t
acgacgctcttccgatctnnnnnnnncggattagaattttttttttttttttttttttgc

SEQ ID NO: 91 - DNA, 60 bp, Artificial Sequence
  Synthetic oligonucleotide
  misc_feature (19)..(26): n is a, c, g, or t
acgacgctcttccgatctnnnnnnnnatcggctatcttttttttttttttttttttttgc

SEQ ID NO: 92 - DNA, 60 bp, Artificial Sequence
  Synthetic oligonucleotide
  misc_feature (19)..(26): n is a, c, g, or t
acgacgctcttccgatctnnnnnnnnccttgatcgtttttttttttttttttttttttgc

SEQ ID NO: 93 - DNA, 60 bp, Artificial Sequence
  Synthetic oligonucleotide
  misc_feature (19)..(26): n is a, c, g, or t
acgacgctcttccgatctnnnnnnnnacgaagtcaattttttttttttttttttttttgc

SEQ ID NO: 94 - DNA, 60 bp, Artificial Sequence
  Synthetic oligonucleotide
  misc_feature (19)..(26): n is a, c, g, or t
acgacgctcttccgatctnnnnnnnnttacctcgacttttttttttttttttttttttgc

SEQ ID NO: 95 - DNA, 60 bp, Artificial Sequence
  Synthetic oligonucleotide
  misc_feature (19)..(26): n is a, c, g, or t
acgacgctcttccgatctnnnnnnnnggaggatagcttttttttttttttttttttttgc

SEQ ID NO: 96 - DNA, 60 bp, Artificial Sequence
  Synthetic oligonucleotide
  misc_feature (19)..(26): n is a, c, g, or t
acgacgctcttccgatctnnnnnnnnggctctctatttttttttttttttttttttttgc

SEQ ID NO: 97 - DNA, 60 bp, Artificial Sequence
  Synthetic oligonucleotide
  misc_feature (19)..(26): n is a, c, g, or t
acgacgctcttccgatctnnnnnnnncggtcaagaattttttttttttttttttttttgc

SEQ ID NO: 98 - DNA, 60 bp, Artificial Sequence
  Synthetic oligonucleotide
  misc_feature (19)..(26): n is a, c, g, or t
acgacgctcttccgatctnnnnnnnncgctcctaacttttttttttttttttttttttgc

SEQ ID NO: 99 - DNA, 60 bp, Artificial Sequence
  Synthetic oligonucleotide
  misc_feature (19)..(26): n is a, c, g, or t
acgacgctcttccgatctnnnnnnnnatccatgactttttttttttttttttttttttgc

SEQ ID NO: 100 - DNA, 60 bp, Artificial Sequence
  Synthetic oligonucleotide
  misc_feature (19)..(26): n is a, c, g, or t
acgacgctcttccgatctnnnnnnnnaacctggtctttttttttttttttttttttttgc

SEQ ID NO: 101 - DNA, 60 bp, Artificial Sequence; identical to SEQ ID NO: 100
  Synthetic oligonucleotide
  misc_feature (19)..(26): n is a, c, g, or t

SEQ ID NO: 102 - DNA, 60 bp, Artificial Sequence
  Synthetic oligonucleotide
  misc_feature (19)..(26): n is a, c, g, or t
acgacgctcttccgatctnnnnnnnnggtaccggcattttttttttttttttttttttgc

SEQ ID NO: 103 - DNA, 60 bp, Artificial Sequence
  Synthetic oligonucleotide
  misc_feature (19)..(26): n is a, c, g, or t
acgacgctcttccgatctnnnnnnnnaagccagttattttttttttttttttttttttgc

SEQ ID NO: 104 - DNA, 60 bp, Artificial Sequence
  Synthetic oligonucleotide
  misc_feature (19)..(26): n is a, c, g, or t
acgacgctcttccgatctnnnnnnnntcttgccgacttttttttttttttttttttttgc

SEQ ID NO: 105 - DNA, 60 bp, Artificial Sequence
  Synthetic oligonucleotide
  misc_feature (19)..(26): n is a, c, g, or t
acgacgctcttccgatctnnnnnnnnaagaccgttgttttttttttttttttttttttgc

SEQ ID NO: 106 - DNA, 60 bp, Artificial Sequence
  Synthetic oligonucleotide
  misc_feature (19)..(26): n is a, c, g, or t
acgacgctcttccgatctnnnnnnnnaggttagcatttttttttttttttttttttttgc

SEQ ID NO: 107 - DNA, 60 bp, Artificial Sequence
  Synthetic oligonucleotide
  misc_feature (19)..(26): n is a, c, g, or t
acgacgctcttccgatctnnnnnnnnttcgcctccattttttttttttttttttttttgc

SEQ ID NO: 108 - DNA, 60 bp, Artificial Sequence
  Synthetic oligonucleotide
  misc_feature (19)..(26): n is a, c, g, or t
acgacgctcttccgatctnnnnnnnnagagccaaggttttttttttttttttttttttgc

SEQ ID NO: 109 - DNA, 60 bp, Artificial Sequence
  Synthetic oligonucleotide
  misc_feature (19)..(26): n is a, c, g, or t
acgacgctcttccgatctnnnnnnnnaataccatccttttttttttttttttttttttgc

SEQ ID NO: 110 - DNA, 60 bp, Artificial Sequence
  Synthetic oligonucleotide
  misc_feature (19)..(26): n is a, c, g, or t
acgacgctcttccgatctnnnnnnnnagctctcctcttttttttttttttttttttttgc

SEQ ID NO: 111 - DNA, 60 bp, Artificial Sequence
  Synthetic oligonucleotide
  misc_feature (19)..(26): n is a, c, g, or t
acgacgctcttccgatctnnnnnnnncttgattgccttttttttttttttttttttttgc

SEQ ID NO: 112 - DNA, 60 bp, Artificial Sequence
  Synthetic oligonucleotide
  misc_feature (19)..(26): n is a, c, g, or t
acgacgctcttccgatctnnnnnnnnagcttatccgttttttttttttttttttttttgc

SEQ ID NO: 113 - DNA, 60 bp, Artificial Sequence
  Synthetic oligonucleotide
  misc_feature (19)..(26): n is a, c, g, or t
acgacgctcttccgatctnnnnnnnnaagaatctgattttttttttttttttttttttgc

SEQ ID NO: 114 - DNA, 60 bp, Artificial Sequence
  Synthetic oligonucleotide
  misc_feature (19)..(26): n is a, c, g, or t
acgacgctcttccgatctnnnnnnnncatctctgcattttttttttttttttttttttgc

SEQ ID NO: 115 - DNA, 60 bp, Artificial Sequence
  Synthetic oligonucleotide
  misc_feature (19)..(26): n is a, c, g, or t
acgacgctcttccgatctnnnnnnnnacctggccaattttttttttttttttttttttgc

SEQ ID NO: 116 - DNA, 60 bp, Artificial Sequence
  Synthetic oligonucleotide
  misc_feature (19)..(26): n is a, c, g, or t
acgacgctcttccgatctnnnnnnnntaactggttattttttttttttttttttttttgc

SEQ ID NO: 117 - DNA, 60 bp, Artificial Sequence
  Synthetic oligonucleotide
  misc_feature (19)..(26): n is a, c, g, or t
acgacgctcttccgatctnnnnnnnnttgctaacggttttttttttttttttttttttgc

SEQ ID NO: 118 - DNA, 60 bp, Artificial Sequence
  Synthetic oligonucleotide
  misc_feature (19)..(26): n is a, c, g, or t
acgacgctcttccgatctnnnnnnnnactagagagtttttttttttttttttttttttgc

SEQ ID NO: 119 - DNA, 60 bp, Artificial Sequence
  Synthetic oligonucleotide
  misc_feature (19)..(26): n is a, c, g, or t
acgacgctcttccgatctnnnnnnnnaatgccgcttttttttttttttttttttttttgc

SEQ ID NO: 120 - DNA, 60 bp, Artificial Sequence
  Synthetic oligonucleotide
  misc_feature (19)..(26): n is a, c, g, or t
acgacgctcttccgatctnnnnnnnntatagacgcattttttttttttttttttttttgc

SEQ ID NO: 121 - DNA, 60 bp, Artificial Sequence
  Synthetic oligonucleotide
  misc_feature (19)..(26): n is a, c, g, or t
acgacgctcttccgatctnnnnnnnntcaatcgcatttttttttttttttttttttttgc

SEQ ID NO: 122 - DNA, 60 bp, Artificial Sequence
  Synthetic oligonucleotide
  misc_feature (19)..(26): n is a, c, g, or t
acgacgctcttccgatctnnnnnnnnttcttaataattttttttttttttttttttttgc

SEQ ID NO: 123 - DNA, 60 bp, Artificial Sequence
  Synthetic oligonucleotide
  misc_feature (19)..(26): n is a, c, g, or t
acgacgctcttccgatctnnnnnnnngtcctagaggttttttttttttttttttttttgc

SEQ ID NO: 124 - DNA, 60 bp, Artificial Sequence
  Synthetic oligonucleotide
  misc_feature (19)..(26): n is a, c, g, or t
acgacgctcttccgatctnnnnnnnnatattgatacttttttttttttttttttttttgc

SEQ ID NO: 125 - DNA, 60 bp, Artificial Sequence
  Synthetic oligonucleotide
  misc_feature (19)..(26): n is a, c, g, or t
acgacgctcttccgatctnnnnnnnnccgctgccagttttttttttttttttttttttgc

SEQ ID NO: 126 - DNA, 60 bp, Artificial Sequence
  Synthetic oligonucleotide
  misc_feature (19)..(26): n is a, c, g, or t
acgacgctcttccgatctnnnnnnnncctagtacgtttttttttttttttttttttttgc

SEQ ID NO: 127 - DNA, 60 bp, Artificial Sequence
  Synthetic oligonucleotide
  misc_feature (19)..(26): n is a, c, g, or t
acgacgctcttccgatctnnnnnnnncaattaccgtttttttttttttttttttttttgc

SEQ ID NO: 128 - DNA, 60 bp, Artificial Sequence
  Synthetic oligonucleotide
  misc_feature (19)..(26): n is a, c, g, or t
acgacgctcttccgatctnnnnnnnnggccgtagtcttttttttttttttttttttttgc

SEQ ID NO: 129 - DNA, 60 bp, Artificial Sequence
  Synthetic oligonucleotide
  misc_feature (19)..(26): n is a, c, g, or t
acgacgctcttccgatctnnnnnnnncgattacggcttttttttttttttttttttttgc

SEQ ID NO: 130 - DNA, 60 bp, Artificial Sequence
  Synthetic oligonucleotide
  misc_feature (19)..(26): n is a, c, g, or t
acgacgctcttccgatctnnnnnnnntaatgaacgattttttttttttttttttttttgc

SEQ ID NO: 131 - DNA, 60 bp, Artificial Sequence
  Synthetic oligonucleotide
  misc_feature (19)..(26): n is a, c, g, or t
acgacgctcttccgatctnnnnnnnnccgttccttattttttttttttttttttttttgc

SEQ ID NO: 132 - DNA, 60 bp, Artificial Sequence
  Synthetic oligonucleotide
  misc_feature (19)..(26): n is a, c, g, or t
acgacgctcttccgatctnnnnnnnnggtaccatatttttttttttttttttttttttgc

SEQ ID NO: 133 - DNA, 60 bp, Artificial Sequence
  Synthetic oligonucleotide
  misc_feature (19)..(26): n is a, c, g, or t
acgacgctcttccgatctnnnnnnnnccgattcgcattttttttttttttttttttttgc

SEQ ID NO: 134 - DNA, 60 bp, Artificial Sequence
  Synthetic oligonucleotide
  misc_feature (19)..(26): n is a, c, g, or t
acgacgctcttccgatctnnnnnnnnatggctctgcttttttttttttttttttttttgc

SEQ ID NO: 135 - DNA, 60 bp, Artificial Sequence
  Synthetic oligonucleotide
  misc_feature (19)..(26): n is a, c, g, or t
acgacgctcttccgatctnnnnnnnngtataatacgttttttttttttttttttttttgc

SEQ ID NO: 136 - DNA, 60 bp, Artificial Sequence
  Synthetic oligonucleotide
  misc_feature (19)..(26): n is a, c, g, or t
acgacgctcttccgatctnnnnnnnnatcagcaagtttttttttttttttttttttttgc

SEQ ID NO: 137 - DNA, 60 bp, Artificial Sequence
  Synthetic oligonucleotide
  misc_feature (19)..(26): n is a, c, g, or t
acgacgctcttccgatctnnnnnnnnggcgaactcgttttttttttttttttttttttgc

SEQ ID NO: 138 - DNA, 60 bp, Artificial Sequence
  Synthetic oligonucleotide
  misc_feature (19)..(26): n is a, c, g, or t
acgacgctcttccgatctnnnnnnnnttaattgaatttttttttttttttttttttttgc

SEQ ID NO: 139 - DNA, 60 bp, Artificial Sequence
  Synthetic oligonucleotide
  misc_feature (19)..(26): n is a, c, g, or t
acgacgctcttccgatctnnnnnnnnttaggaccggttttttttttttttttttttttgc

SEQ ID NO: 140 - DNA, 60 bp, Artificial Sequence
  Synthetic oligonucleotide
  misc_feature (19)..(26): n is a, c, g, or t
acgacgctcttccgatctnnnnnnnnaagtaagagcttttttttttttttttttttttgc

SEQ ID NO: 141 - DNA, 60 bp, Artificial Sequence
  Synthetic oligonucleotide
  misc_feature (19)..(26): n is a, c, g, or t
acgacgctcttccgatctnnnnnnnnccttggtccattttttttttttttttttttttgc

SEQ ID NO: 142 - DNA, 60 bp, Artificial Sequence
  Synthetic oligonucleotide
  misc_feature (19)..(26): n is a, c, g, or t
acgacgctcttccgatctnnnnnnnncatcagaatgttttttttttttttttttttttgc

SEQ ID NO: 143 - DNA, 60 bp, Artificial Sequence
  Synthetic oligonucleotide
  misc_feature (19)..(26): n is a, c, g, or t
acgacgctcttccgatctnnnnnnnnttatagcagattttttttttttttttttttttgc

SEQ ID NO: 144 - DNA, 60 bp, Artificial Sequence
  Synthetic oligonucleotide
  misc_feature (19)..(26): n is a, c, g, or t
acgacgctcttccgatctnnnnnnnnttacttggaattttttttttttttttttttttgc

SEQ ID NO: 145 - DNA, 60 bp, Artificial Sequence
  Synthetic oligonucleotide
  misc_feature (19)..(26): n is a, c, g, or t
acgacgctcttccgatctnnnnnnnngctcagccggttttttttttttttttttttttgc

SEQ ID NO: 146 - DNA, 60 bp, Artificial Sequence
  Synthetic oligonucleotide
  misc_feature (19)..(26): n is a, c, g, or t
acgacgctcttccgatctnnnnnnnnacgtccgcagttttttttttttttttttttttgc

SEQ ID NO: 147 - DNA, 60 bp, Artificial Sequence
  Synthetic oligonucleotide
  misc_feature (19)..(26): n is a, c, g, or t
acgacgctcttccgatctnnnnnnnnttgactgacgttttttttttttttttttttttgc

SEQ ID NO: 148 - DNA, 60 bp, Artificial Sequence
  Synthetic oligonucleotide
  misc_feature (19)..(26): n is a, c, g, or t
acgacgctcttccgatctnnnnnnnnttgcgaggcattttttttttttttttttttttgc

SEQ ID NO: 149 - DNA, 60 bp, Artificial Sequence
  Synthetic oligonucleotide
  misc_feature (19)..(26): n is a, c, g, or t
acgacgctcttccgatctnnnnnnnnttccaaccgcttttttttttttttttttttttgc

SEQ ID NO: 150 - DNA, 60 bp, Artificial Sequence
  Synthetic oligonucleotide
  misc_feature (19)..(26): n is a, c, g, or t
acgacgctcttccgatctnnnnnnnntaaccttcggttttttttttttttttttttttgc

SEQ ID NO: 151 - DNA, 60 bp, Artificial Sequence
  Synthetic oligonucleotide
  misc_feature (19)..(26): n is a, c, g, or t
acgacgctcttccgatctnnnnnnnntcaagccgatttttttttttttttttttttttgc

SEQ ID NO: 152 - DNA, 60 bp, Artificial Sequence
  Synthetic oligonucleotide
  misc_feature (19)..(26): n is a, c, g, or t
acgacgctcttccgatctnnnnnnnncttgcaacctttttttttttttttttttttttgc

SEQ ID NO: 153 - DNA, 60 bp, Artificial Sequence
  Synthetic oligonucleotide
  misc_feature (19)..(26): n is a, c, g, or t
acgacgctcttccgatctnnnnnnnnccatcgcgaattttttttttttttttttttttgc

SEQ ID NO: 154 - DNA, 60 bp, Artificial Sequence
  Synthetic oligonucleotide
  misc_feature (19)..(26): n is a, c, g, or t
acgacgctcttccgatctnnnnnnnntagacttcttttttttttttttttttttttttgc

SEQ ID NO: 155 - DNA, 60 bp, Artificial Sequence
  Synthetic oligonucleotide
  misc_feature (19)..(26): n is a, c, g, or t
acgacgctcttccgatctnnnnnnnngtccttaagattttttttttttttttttttttgc

SEQ ID NO: 156 - DNA, 60 bp, Artificial Sequence
  Synthetic oligonucleotide
  misc_feature (19)..(26): n is a, c, g, or t
acgacgctcttccgatctnnnnnnnnagtaacggtcttttttttttttttttttttttgc

SEQ ID NO: 157 - DNA, 60 bp, Artificial Sequence
  Synthetic oligonucleotide
  misc_feature (19)..(26): n is a, c, g, or t
acgacgctcttccgatctnnnnnnnngttcgtcagattttttttttttttttttttttgc

SEQ ID NO: 158 - DNA, 60 bp, Artificial Sequence
  Synthetic oligonucleotide
  misc_feature (19)..(26): n is a, c, g, or t
acgacgctcttccgatctnnnnnnnncgcctaatgcttttttttttttttttttttttgc

SEQ ID NO: 159 - DNA, 60 bp, Artificial Sequence
  Synthetic oligonucleotide
  misc_feature (19)..(26): n is a, c, g, or t
acgacgctcttccgatctnnnnnnnnaccggaattattttttttttttttttttttttgc

SEQ ID NO: 160 - DNA, 60 bp, Artificial Sequence
  Synthetic oligonucleotide
  misc_feature (19)..(26): n is a, c, g, or t
acgacgctcttccgatctnnnnnnnntaggccatagttttttttttttttttttttttgc

SEQ ID NO: 161 - DNA, 60 bp, Artificial Sequence
  Synthetic oligonucleotide
  misc_feature (19)..(26): n is a, c, g, or t
acgacgctcttccgatctnnnnnnnntaactcttagttttttttttttttttttttttgc

SEQ ID NO: 162 - DNA, 60 bp, Artificial Sequence
  Synthetic oligonucleotide
  misc_feature (19)..(26): n is a, c, g, or t
acgacgctcttccgatctnnnnnnnntatgagttaattttttttttttttttttttttgc

SEQ ID NO: 163 - DNA, 60 bp, Artificial Sequence
  Synthetic oligonucleotide
  misc_feature (19)..(26): n is a, c, g, or t
acgacgctcttccgatctnnnnnnnntatcatgatcttttttttttttttttttttttgc

SEQ ID NO: 164 - DNA, 60 bp, Artificial Sequence
  Synthetic oligonucleotide
  misc_feature (19)..(26): n is a, c, g, or t
acgacgctcttccgatctnnnnnnnngagcatatggttttttttttttttttttttttgc

SEQ ID NO: 165 - DNA, 60 bp, Artificial Sequence
  Synthetic oligonucleotide
  misc_feature (19)..(26): n is a, c, g, or t
acgacgctcttccgatctnnnnnnnntaacgatccattttttttttttttttttttttgc

SEQ ID NO: 166 - DNA, 60 bp, Artificial Sequence
  Synthetic oligonucleotide
  misc_feature (19)..(26): n is a, c, g, or t
acgacgctcttccgatctnnnnnnnncggcgtaactttttttttttttttttttttttgc

SEQ ID NO: 167 - DNA, 60 bp, Artificial Sequence
  Synthetic oligonucleotide
  misc_feature (19)..(26): n is a, c, g, or t
acgacgctcttccgatctnnnnnnnncgtcgcagccttttttttttttttttttttttgc

SEQ ID NO: 168 - DNA, 60 bp, Artificial Sequence
  Synthetic oligonucleotide
  misc_feature (19)..(26): n is a, c, g, or t
acgacgctcttccgatctnnnnnnnngtagctccatttttttttttttttttttttttgc

SEQ ID NO: 169 - DNA, 60 bp, Artificial Sequence
  Synthetic oligonucleotide
  misc_feature (19)..(26): n is a, c, g, or t
acgacgctcttccgatctnnnnnnnnttgccttggcttttttttttttttttttttttgc

SEQ ID NO: 170 - DNA, 60 bp, Artificial Sequence
  Synthetic oligonucleotide
  misc_feature (19)..(26): n is a, c, g, or t
acgacgctcttccgatctnnnnnnnntgctaattctttttttttttttttttttttttgc

SEQ ID NO: 171 - DNA, 60 bp, Artificial Sequence
  Synthetic oligonucleotide
  misc_feature (19)..(26): n is a, c, g, or t
acgacgctcttccgatctnnnnnnnngtcctacttgttttttttttttttttttttttgc

SEQ ID NO: 172 - DNA, 60 bp, Artificial Sequence
  Synthetic oligonucleotide
  misc_feature (19)..(26): n is a, c, g, or t
acgacgctcttccgatctnnnnnnnnggtaggttagttttttttttttttttttttttgc

SEQ ID NO: 173 - DNA, 60 bp, Artificial Sequence
  Synthetic oligonucleotide
  misc_feature (19)..(26): n is a, c, g, or t
acgacgctcttccgatctnnnnnnnngagcatcattttttttttttttttttttttttgc

SEQ ID NO: 174 - DNA, 60 bp, Artificial Sequence
  Synthetic oligonucleotide
  misc_feature (19)..(26): n is a, c, g, or t
acgacgctcttccgatctnnnnnnnnccgctccggcttttttttttttttttttttttgc

SEQ ID NO: 175 - DNA, 60 bp, Artificial Sequence
  Synthetic oligonucleotide
  misc_feature (19)..(26): n is a, c, g, or t
acgacgctcttccgatctnnnnnnnnttcttccggtttttttttttttttttttttttgc

SEQ ID NO: 176 - DNA, 60 bp, Artificial Sequence
  Synthetic oligonucleotide
  misc_feature (19)..(26): n is a, c, g, or t
acgacgctcttccgatctnnnnnnnnaggagagaacttttttttttttttttttttttgc

SEQ ID NO: 177 - DNA, 60 bp, Artificial Sequence
  Synthetic oligonucleotide
  misc_feature (19)..(26): n is a, c, g, or t
acgacgctcttccgatctnnnnnnnntaactcaattttttttttttttttttttttttgc

SEQ ID NO: 178 - DNA, 60 bp, Artificial Sequence
  Synthetic oligonucleotide
  misc_feature (19)..(26): n is a, c, g, or t
acgacgctcttccgatctnnnnnnnnactataggttttttttttttttttttttttttgc

SEQ ID NO: 179 - DNA, 60 bp, Artificial Sequence
  Synthetic oligonucleotide
  misc_feature (19)..(26): n is a, c, g, or t
acgacgctcttccgatctnnnnnnnncaagatgccgttttttttttttttttttttttgc

SEQ ID NO: 180 - DNA, 60 bp, Artificial Sequence
  Synthetic oligonucleotide
  misc_feature (19)..(26): n is a, c, g, or t
acgacgctcttccgatctnnnnnnnnaacgtctagtttttttttttttttttttttttgc

SEQ ID NO: 181 - DNA, 60 bp, Artificial Sequence
  Synthetic oligonucleotide
  misc_feature (19)..(26): n is a, c, g, or t
acgacgctcttccgatctnnnnnnnnaggtatactcttttttttttttttttttttttgc

SEQ ID NO: 182 - DNA, 60 bp, Artificial Sequence
  Synthetic oligonucleotide
  misc_feature (19)..(26): n is a, c, g, or t
acgacgctcttccgatctnnnnnnnnttcataggacttttttttttttttttttttttgc

SEQ ID NO: 183 - DNA, 60 bp, Artificial Sequence
  Synthetic oligonucleotide
  misc_feature (19)..(26): n is a, c, g, or t
acgacgctcttccgatctnnnnnnnnggaggcctccttttttttttttttttttttttgc

SEQ ID NO: 184 - DNA, 60 bp, Artificial Sequence
  Synthetic oligonucleotide
  misc_feature (19)..(26): n is a, c, g, or t
acgacgctcttccgatctnnnnnnnnttcaatataattttttttttttttttttttttgc

SEQ ID NO: 185 - DNA, 60 bp, Artificial Sequence
  Synthetic oligonucleotide
  misc_feature (19)..(26): n is a, c, g, or t
acgacgctcttccgatctnnnnnnnnacgtcatatattttttttttttttttttttttgc

SEQ ID NO: 186 - DNA, 60 bp, Artificial Sequence
  Synthetic oligonucleotide
  misc_feature (19)..(26): n is a, c, g, or t
acgacgctcttccgatctnnnnnnnnttgaccaggattttttttttttttttttttttgc

SEQ ID NO: 187 - DNA, 60 bp, Artificial Sequence
  Synthetic oligonucleotide
  misc_feature (19)..(26): n is a, c, g, or t
acgacgctcttccgatctnnnnnnnncggttgcgcgttttttttttttttttttttttgc

SEQ ID NO: 188 - DNA, 60 bp, Artificial Sequence
  Synthetic oligonucleotide
  misc_feature (19)..(26): n is a, c, g, or t
acgacgctcttccgatctnnnnnnnncaaggaggtcttttttttttttttttttttttgc

SEQ ID NO: 189 - DNA, 60 bp, Artificial Sequence
  Synthetic oligonucleotide
  misc_feature (19)..(26): n is a, c, g, or t
acgacgctcttccgatctnnnnnnnnttacgatgaattttttttttttttttttttttgc

SEQ ID NO: 190 - DNA, 60 bp, Artificial Sequence
  Synthetic oligonucleotide
  misc_feature (19)..(26): n is a, c, g, or t
acgacgctcttccgatctnnnnnnnnttgctggcatttttttttttttttttttttttgc

SEQ ID NO: 191 - DNA, 60 bp, Artificial Sequence
  Synthetic oligonucleotide
  misc_feature (19)..(26): n is a, c, g, or t
acgacgctcttccgatctnnnnnnnngaggcatcaattttttttttttttttttttttgc

SEQ ID NO: 192 - DNA, 60 bp, Artificial Sequence
  Synthetic oligonucleotide
  misc_feature (19)..(26): n is a, c, g, or t
acgacgctcttccgatctnnnnnnnnattcgaccaattttttttttttttttttttttgc

SEQ ID NO: 193 - DNA, 60 bp, Artificial Sequence
  Synthetic oligonucleotide
  misc_feature (19)..(26): n is a, c, g, or t
acgacgctcttccgatctnnnnnnnnccgcggctcattttttttttttttttttttttgc

SEQ ID NO: 194 - DNA, 60 bp, Artificial Sequence
  Synthetic oligonucleotide
  misc_feature (19)..(26): n is a, c, g, or t
acgacgctcttccgatctnnnnnnnnggctcctcgtttttttttttttttttttttttgc

SEQ ID NO: 195 - DNA, 60 bp, Artificial Sequence
  Synthetic oligonucleotide
  misc_feature (19)..(26): n is a, c, g, or t
acgacgctcttccgatctnnnnnnnngttacgcaagttttttttttttttttttttttgc

SEQ ID NO: 196 - DNA, 60 bp, Artificial Sequence
  Synthetic oligonucleotide
  misc_feature (19)..(26): n is a, c, g, or t
acgacgctcttccgatctnnnnnnnnagccggtaccttttttttttttttttttttttgc

SEQ ID NO: 197 - DNA, 60 bp, Artificial Sequence
  Synthetic oligonucleotide
  misc_feature (19)..(26): n is a, c, g, or t
acgacgctcttccgatctnnnnnnnnacctctatctttttttttttttttttttttttgc

SEQ ID NO: 198 - DNA, 60 bp, Artificial Sequence
  Synthetic oligonucleotide
  misc_feature (19)..(26): n is a, c, g, or t
acgacgctcttccgatctnnnnnnnnggactactacttttttttttttttttttttttgc

SEQ ID NO: 199 - DNA, 60 bp, Artificial Sequence
  Synthetic oligonucleotide
  misc_feature (19)..(26): n is a, c, g, or t
acgacgctcttccgatctnnnnnnnngtatcatcgattttttttttttttttttttttgc

SEQ ID NO: 200 - DNA, 60 bp, Artificial Sequence
  Synthetic oligonucleotide
  misc_feature (19)..(26): n is a, c, g, or t
acgacgctcttccgatctnnnnnnnnccgcgattatttttttttttttttttttttttgc

SEQ ID NO: 201 - DNA, 60 bp, Artificial Sequence
  Synthetic oligonucleotide
  misc_feature (19)..(26): n is a, c, g, or t
acgacgctcttccgatctnnnnnnnnattcaggtacttttttttttttttttttttttgc

SEQ ID NO: 202 - DNA, 60 bp, Artificial Sequence
  Synthetic oligonucleotide
  misc_feature (19)..(26): n is a, c, g, or t
acgacgctcttccgatctnnnnnnnnatggaattggttttttttttttttttttttttgc

SEQ ID NO: 203 - DNA, 60 bp, Artificial Sequence
  Synthetic oligonucleotide
  misc_feature (19)..(26): n is a, c, g, or t
acgacgctcttccgatctnnnnnnnngacgaagcgtttttttttttttttttttttttgc

SEQ ID NO: 204 - DNA, 60 bp, Artificial Sequence
  Synthetic oligonucleotide
  misc_feature (19)..(26): n is a, c, g, or t
acgacgctcttccgatctnnnnnnnncttgcagtagttttttttttttttttttttttgc

SEQ ID NO: 205 - DNA, 60 bp, Artificial Sequence
  Synthetic oligonucleotide
  misc_feature (19)..(26): n is a, c, g, or t
acgacgctcttccgatctnnnnnnnncttggtaatgttttttttttttttttttttttgc

SEQ ID NO: 206 - DNA, 60 bp, Artificial Sequence
  Synthetic oligonucleotide
  misc_feature (19)..(26): n is a, c, g, or t
acgacgctcttccgatctnnnnnnnncaagtcgaccttttttttttttttttttttttgc

SEQ ID NO: 207 - DNA, 60 bp, Artificial Sequence
  Synthetic oligonucleotide
  misc_feature (19)..(26): n is a, c, g, or t
acgacgctcttccgatctnnnnnnnntaacgaattgttttttttttttttttttttttgc

SEQ ID NO: 208 - DNA, 60 bp, Artificial Sequence
  Synthetic oligonucleotide
  misc_feature (19)..(26): n is a, c, g, or t
acgacgctcttccgatctnnnnnnnntgagaaccaattttttttttttttttttttttgc

SEQ ID NO: 209 - DNA, 60 bp, Artificial Sequence
  Synthetic oligonucleotide
  misc_feature (19)..(26): n is a, c, g, or t
acgacgctcttccgatctnnnnnnnnttattctgagttttttttttttttttttttttgc

SEQ ID NO: 210 - DNA, 60 bp, Artificial Sequence
  Synthetic oligonucleotide
  misc_feature (19)..(26): n is a, c, g, or t
acgacgctcttccgatctnnnnnnnnttattatggtttttttttttttttttttttttgc

SEQ ID NO: 211 - DNA, 60 bp, Artificial Sequence
  Synthetic oligonucleotide
  misc_feature (19)..(26): n is a, c, g, or t
acgacgctcttccgatctnnnnnnnnatatgagccattttttttttttttttttttttgc

SEQ ID NO: 212 - DNA, 60 bp, Artificial Sequence
  Synthetic oligonucleotide
  misc_feature (19)..(26): n is a, c, g, or t
acgacgctcttccgatctnnnnnnnncaaccagtacttttttttttttttttttttttgc

SEQ ID NO: 213 - DNA, 60 bp, Artificial Sequence
  Synthetic oligonucleotide
  misc_feature (19)..(26): n is a, c, g, or t
acgacgctcttccgatctnnnnnnnncatccgactattttttttttttttttttttttgc

SEQ ID NO: 214 - DNA, 60 bp, Artificial Sequence
  Synthetic oligonucleotide
  misc_feature (19)..(26): n is a, c, g, or t
acgacgctcttccgatctnnnnnnnnatcatggctgttttttttttttttttttttttgc

SEQ ID NO: 215 - DNA, 60 bp, Artificial Sequence
  Synthetic oligonucleotide
  misc_feature (19)..(26): n is a, c, g, or t
acgacgctcttccgatctnnnnnnnnccgcaagttcttttttttttttttttttttttgc

SEQ ID NO: 216 - DNA, 60 bp, Artificial Sequence
  Synthetic oligonucleotide
  misc_feature (19)..(26): n is a, c, g, or t
acgacgctcttccgatctnnnnnnnncttctcattgttttttttttttttttttttttgc

SEQ ID NO: 217 - DNA, 60 bp, Artificial Sequence
  Synthetic oligonucleotide
  misc_feature (19)..(26): n is a, c, g, or t
acgacgctcttccgatctnnnnnnnncaggaggagattttttttttttttttttttttgc

SEQ ID NO: 218 - DNA, 60 bp, Artificial Sequence
  Synthetic oligonucleotide
  misc_feature (19)..(26): n is a, c, g, or t
acgacgctcttccgatctnnnnnnnngatatcggcgttttttttttttttttttttttgc

SEQ ID NO: 219 - DNA, 60 bp, Artificial Sequence
  Synthetic oligonucleotide
  misc_feature (19)..(26): n is a, c, g, or t
acgacgctcttccgatctnnnnnnnnccagtcctctttttttttttttttttttttttgc

SEQ ID NO: 220 - DNA, 60 bp, Artificial Sequence
  Synthetic oligonucleotide
  misc_feature (19)..(26): n is a, c, g, or t
acgacgctcttccgatctnnnnnnnncatagttcggttttttttttttttttttttttgc

SEQ ID NO: 221 - DNA, 60 bp, Artificial Sequence
  Synthetic oligonucleotide
  misc_feature (19)..(26): n is a, c, g, or t
acgacgctcttccgatctnnnnnnnncgtaatgcagttttttttttttttttttttttgc

SEQ ID NO: 222 - DNA, 60 bp, Artificial Sequence
  Synthetic oligonucleotide
  misc_feature (19)..(26): n is a, c, g, or t
acgacgctcttccgatctnnnnnnnnccgttcggatttttttttttttttttttttttgc

SEQ ID NO: 223 - DNA, 60 bp, Artificial Sequence
  Synthetic oligonucleotide
  misc_feature (19)..(26): n is a, c, g, or t
acgacgctcttccgatctnnnnnnnnccataagtccttttttttttttttttttttttgc

SEQ ID NO: 224 - DNA, 60 bp, Artificial Sequence
  Synthetic oligonucleotide
  misc_feature (19)..(26): n is a, c, g, or t
acgacgctcttccgatctnnnnnnnnggcaatgagattttttttttttttttttttttgc

SEQ ID NO: 225 - DNA, 60 bp, Artificial Sequence
  Synthetic oligonucleotide
  misc_feature (19)..(26): n is a, c, g, or t
acgacgctcttccgatctnnnnnnnncggttatgccttttttttttttttttttttttgc

SEQ ID NO: 226 - DNA, 60 bp, Artificial Sequence
  Synthetic oligonucleotide
  misc_feature (19)..(26): n is a, c, g, or t
acgacgctcttccgatctnnnnnnnntggccggcctttttttttttttttttttttttgc

SEQ ID NO: 227 - DNA, 60 bp, Artificial Sequence
  Synthetic oligonucleotide
  misc_feature (19)..(26): n is a, c, g, or t
acgacgctcttccgatctnnnnnnnnagctgcaatattttttttttttttttttttttgc

SEQ ID NO: 228 - DNA, 60 bp, Artificial Sequence
  Synthetic oligonucleotide
  misc_feature (19)..(26): n is a, c, g, or t
acgacgctcttccgatctnnnnnnnntggccatgcattttttttttttttttttttttgc

SEQ ID NO: 229 - DNA, 60 bp, Artificial Sequence
  Synthetic oligonucleotide
  misc_feature (19)..(26): n is a, c, g, or t
acgacgctcttccgatctnnnnnnnntgacgctccgttttttttttttttttttttttgc

SEQ ID NO: 230 - DNA, 60 bp, Artificial Sequence
  Synthetic oligonucleotide
  misc_feature (19)..(26): n is a, c, g, or t
acgacgctcttccgatctnnnnnnnnaactgctgccttttttttttttttttttttttgc

SEQ ID NO: 231 - DNA, 60 bp, Artificial Sequence
  Synthetic oligonucleotide
  misc_feature (19)..(26): n is a, c, g, or t
acgacgctcttccgatctnnnnnnnntgcgcgatgcttttttttttttttttttttttgc

SEQ ID NO: 232 - DNA, 60 bp, Artificial Sequence
  Synthetic oligonucleotide
  misc_feature (19)..(26): n is a, c, g, or t
acgacgctcttccgatctnnnnnnnnattgagattgttttttttttttttttttttttgc

SEQ ID NO: 233 - DNA, 60 bp, Artificial Sequence
  Synthetic oligonucleotide
  misc_feature (19)..(26): n is a, c, g, or t
acgacgctcttccgatctnnnnnnnnttgatatattttttttttttttttttttttttgc

SEQ ID NO: 234 - DNA, 60 bp, Artificial Sequence
  Synthetic oligonucleotide
  misc_feature (19)..(26): n is a, c, g, or t
acgacgctcttccgatctnnnnnnnncggtaggaatttttttttttttttttttttttgc

SEQ ID NO: 235 - DNA, 60 bp, Artificial Sequence
  Synthetic oligonucleotide
  misc_feature (19)..(26): n is a, c, g, or t
acgacgctcttccgatctnnnnnnnnaccagcgcagttttttttttttttttttttttgc

SEQ ID NO: 236 - DNA, 60 bp, Artificial Sequence
  Synthetic oligonucleotide
  misc_feature (19)..(26): n is a, c, g, or t
acgacgctcttccgatctnnnnnnnncgaatgagctttttttttttttttttttttttgc

SEQ ID NO: 237 - DNA, 60 bp, Artificial Sequence
  Synthetic oligonucleotide
  misc_feature (19)..(26): n is a, c, g, or t
acgacgctcttccgatctnnnnnnnnagttcgagtattttttttttttttttttttttgc

SEQ ID NO: 238 - DNA, 60 bp, Artificial Sequence
  Synthetic oligonucleotide
  misc_feature (19)..(26): n is a, c, g, or t
acgacgctcttccgatctnnnnnnnnttggacgctgttttttttttttttttttttttgc

SEQ ID NO: 239 - DNA, 60 bp, Artificial Sequence
  Synthetic oligonucleotide
  misc_feature (19)..(26): n is a, c, g, or t
acgacgctcttccgatctnnnnnnnnatagactaggttttttttttttttttttttttgc

SEQ ID NO: 240 - DNA, 60 bp, Artificial Sequence
  Synthetic oligonucleotide
  misc_feature (19)..(26): n is a, c, g, or t
acgacgctcttccgatctnnnnnnnntatagtaagcttttttttttttttttttttttgc

SEQ ID NO: 241 - DNA, 60 bp, Artificial Sequence
  Synthetic oligonucleotide
  misc_feature (19)..(26): n is a, c, g, or t
acgacgctcttccgatctnnnnnnnncggtcgttaattttttttttttttttttttttgc

SEQ ID NO: 242 - DNA, 60 bp, Artificial Sequence
  Synthetic oligonucleotide
  misc_feature (19)..(26): n is a, c, g, or t
acgacgctcttccgatctnnnnnnnnatggcggatcttttttttttttttttttttttgc

SEQ ID NO: 243 - DNA, 60 bp, Artificial Sequence
  Synthetic oligonucleotide
  misc_feature (19)..(26): n is a, c, g, or t
acgacgctcttccgatctnnnnnnnnctctgatcagttttttttttttttttttttttgc

SEQ ID NO: 244 - DNA, 60 bp, Artificial Sequence
  Synthetic oligonucleotide
  misc_feature (19)..(26): n is a, c, g, or t
acgacgctcttccgatctnnnnnnnnggccagtccgttttttttttttttttttttttgc

SEQ ID NO: 245 - DNA, 60 bp, Artificial Sequence
  Synthetic oligonucleotide
  misc_feature (19)..(26): n is a, c, g, or t
acgacgctcttccgatctnnnnnnnncggaagatatttttttttttttttttttttttgc

SEQ ID NO: 246 - DNA, 60 bp, Artificial Sequence
  Synthetic oligonucleotide
  misc_feature (19)..(26): n is a, c, g, or t
acgacgctcttccgatctnnnnnnnntggctgatgattttttttttttttttttttttgc

SEQ ID NO: 247 - DNA, 60 bp, Artificial Sequence
  Synthetic oligonucleotide
  misc_feature (19)..(26): n is a, c, g, or t
acgacgctcttccgatctnnnnnnnngaaggttgccttttttttttttttttttttttgc

SEQ ID NO: 248 - DNA, 60 bp, Artificial Sequence
  Synthetic oligonucleotide
  misc_feature (19)..(26): n is a, c, g, or t
acgacgctcttccgatctnnnnnnnngttgaaggatttttttttttttttttttttttgc

SEQ ID NO: 249 - DNA, 60 bp, Artificial Sequence
  Synthetic oligonucleotide
  misc_feature (19)..(26): n is a, c, g, or t
acgacgctcttccgatctnnnnnnnnccattcgtaattttttttttttttttttttttgc

SEQ ID NO: 250 - DNA, 60 bp, Artificial Sequence
  Synthetic oligonucleotide
  misc_feature (19)..(26): n is a, c, g, or t
acgacgctcttccgatctnnnnnnnntgcgccagaattttttttttttttttttttttgc

SEQ ID NO: 251 - DNA, 60 bp, Artificial Sequence
  Synthetic oligonucleotide
  misc_feature (19)..(26): n is a, c, g, or t
acgacgctcttccgatctnnnnnnnncgaataattcttttttttttttttttttttttgc

SEQ ID NO: 252 - DNA, 60 bp, Artificial Sequence
  Synthetic oligonucleotide
  misc_feature (19)..(26): n is a, c, g, or t
acgacgctcttccgatctnnnnnnnngcgacgccttttttttttttttttttttttttgc

SEQ ID NO: 253 - DNA, 60 bp, Artificial Sequence
  Synthetic oligonucleotide
  misc_feature (19)..(26): n is a, c, g, or t
acgacgctcttccgatctnnnnnnnnatcaacgattttttttttttttttttttttttgc

SEQ ID NO: 254 - DNA, 60 bp, Artificial Sequence
  Synthetic oligonucleotide
  misc_feature (19)..(26): n is a, c, g, or t
acgacgctcttccgatctnnnnnnnngttctgaattttttttttttttttttttttttgc

SEQ ID NO: 255 - DNA, 60 bp, Artificial Sequence
  Synthetic oligonucleotide
  misc_feature (19)..(26): n is a, c, g, or t
acgacgctcttccgatctnnnnnnnngctaacctcattttttttttttttttttttttgc

SEQ ID NO: 256 - DNA, 60 bp, Artificial Sequence
  Synthetic oligonucleotide
  misc_feature (19)..(26): n is a, c, g, or t
acgacgctcttccgatctnnnnnnnncaagcaactgttttttttttttttttttttttgc

SEQ ID NO: 257 - DNA, 60 bp, Artificial Sequence
  Synthetic oligonucleotide
  misc_feature (19)..(26): n is a, c, g, or t
acgacgctcttccgatctnnnnnnnnggagcggccgttttttttttttttttttttttgc

SEQ ID NO: 258 - DNA, 60 bp, Artificial Sequence
  Synthetic oligonucleotide
  misc_feature (19)..(26): n is a, c, g, or t
acgacgctcttccgatctnnnnnnnncgcgtacgacttttttttttttttttttttttgc

SEQ ID NO: 259 - DNA, 60 bp, Artificial Sequence
  Synthetic oligonucleotide
  misc_feature (19)..(26): n is a, c, g, or t
acgacgctcttccgatctnnnnnnnncgatggcgccttttttttttttttttttttttgc

SEQ ID NO: 260 - DNA, 60 bp, Artificial Sequence
  Synthetic oligonucleotide
  misc_feature (19)..(26): n is a, c, g, or t
acgacgctcttccgatctnnnnnnnntggtattcatttttttttttttttttttttttgc

SEQ ID NO: 261 - DNA, 60 bp, Artificial Sequence
  Synthetic oligonucleotide
  misc_feature (19)..(26): n is a, c, g, or t
acgacgctcttccgatctnnnnnnnngataaggcaattttttttttttttttttttttgc

SEQ ID NO: 262 - DNA, 60 bp, Artificial Sequence
  Synthetic oligonucleotide
  misc_feature (19)..(26): n is a, c, g, or t
acgacgctcttccgatctnnnnnnnngccggtcgagttttttttttttttttttttttgc

SEQ ID NO: 263 - DNA, 60 bp, Artificial Sequence
  Synthetic oligonucleotide
  misc_feature (19)..(26): n is a, c, g, or t
acgacgctcttccgatctnnnnnnnntgcgccatctttttttttttttttttttttttgc

SEQ ID NO: 264 - DNA, 60 bp, Artificial Sequence
  Synthetic oligonucleotide
  misc_feature (19)..(26): n is a, c, g, or t
acgacgctcttccgatctnnnnnnnnaagtcttccgttttttttttttttttttttttgc

SEQ ID NO: 265 - DNA, 60 bp, Artificial Sequence
  Synthetic oligonucleotide
  misc_feature (19)..(26): n is a, c, g, or t
acgacgctcttccgatctnnnnnnnnagactcaagcttttttttttttttttttttttgc

SEQ ID NO: 266 - DNA, 60 bp, Artificial Sequence
  Synthetic oligonucleotide
  misc_feature (19)..(26): n is a, c, g, or t
acgacgctcttccgatctnnnnnnnngcaggcgacgttttttttttttttttttttttgc

SEQ ID NO: 267 - DNA, 60 bp, Artificial Sequence
  Synthetic oligonucleotide
  misc_feature (19)..(26): n is a, c, g, or t
acgacgctcttccgatctnnnnnnnnaatactcttcttttttttttttttttttttttgc

SEQ ID NO: 268 - DNA, 60 bp, Artificial Sequence
  Synthetic oligonucleotide
  misc_feature (19)..(26): n is a, c, g, or t
acgacgctcttccgatctnnnnnnnnccaactaaccttttttttttttttttttttttgc

SEQ ID NO: 269 - DNA, 60 bp, Artificial Sequence
  Synthetic oligonucleotide
  misc_feature (19)..(26): n is a, c, g, or t
acgacgctcttccgatctnnnnnnnntatcctcaatttttttttttttttttttttttgc

SEQ ID NO: 270 - DNA, 60 bp, Artificial Sequence
  Synthetic oligonucleotide
  misc_feature (19)..(26): n is a, c, g, or t
acgacgctcttccgatctnnnnnnnngccgtcgcgtttttttttttttttttttttttgc

SEQ ID NO: 271 - DNA, 60 bp, Artificial Sequence
  Synthetic oligonucleotide
  misc_feature (19)..(26): n is a, c, g, or t
acgacgctcttccgatctnnnnnnnnccgctgcttcttttttttttttttttttttttgc

SEQ ID NO: 272 - DNA, 60 bp, Artificial Sequence
  Synthetic oligonucleotide
  misc_feature (19)..(26): n is a, c, g, or t
acgacgctcttccgatctnnnnnnnntgaccgaatcttttttttttttttttttttttgc

SEQ ID NO: 273 - DNA, 60 bp, Artificial Sequence
  Synthetic oligonucleotide
  misc_feature (19)..(26): n is a, c, g, or t
acgacgctcttccgatctnnnnnnnngtctccagagttttttttttttttttttttttgc

SEQ ID NO: 274 - DNA, 60 bp, Artificial Sequence
  Synthetic oligonucleotide
  misc_feature (19)..(26): n is a, c, g, or t
acgacgctcttccgatctnnnnnnnnaatgctagtcttttttttttttttttttttttgc

SEQ ID NO: 275 - DNA, 60 bp, Artificial Sequence
  Synthetic oligonucleotide
  misc_feature (19)..(26): n is a, c, g, or t
acgacgctcttccgatctnnnnnnnngacgacctgcttttttttttttttttttttttgc

SEQ ID NO: 276 - DNA, 60 bp, Artificial Sequence
  Synthetic oligonucleotide
  misc_feature (19)..(26): n is a, c, g, or t
acgacgctcttccgatctnnnnnnnnagagccagccttttttttttttttttttttttgc

SEQ ID NO: 277 - DNA, 60 bp, Artificial Sequence
  Synthetic oligonucleotide
  misc_feature (19)..(26): n is a, c, g, or t
acgacgctcttccgatctnnnnnnnnccaggccgcattttttttttttttttttttttgc

SEQ ID NO: 278 - DNA, 60 bp, Artificial Sequence
  Synthetic oligonucleotide
  misc_feature (19)..(26): n is a, c, g, or t
acgacgctcttccgatctnnnnnnnncaggtatggattttttttttttttttttttttgc

SEQ ID NO: 279 - DNA, 60 bp, Artificial Sequence
  Synthetic oligonucleotide
  misc_feature (19)..(26): n is a, c, g, or t
acgacgctcttccgatctnnnnnnnnccggagttgcttttttttttttttttttttttgc

SEQ ID NO: 280 - DNA, 60 bp, Artificial Sequence
  Synthetic oligonucleotide
  misc_feature (19)..(26): n is a, c, g, or t
acgacgctcttccgatctnnnnnnnnttaattattgttttttttttttttttttttttgc

SEQ ID NO: 281 - DNA, 60 bp, Artificial Sequence
  Synthetic oligonucleotide
  misc_feature (19)..(26): n is a, c, g, or t
acgacgctcttccgatctnnnnnnnnaatcagctgcttttttttttttttttttttttgc

SEQ ID NO: 282 - DNA, 60 bp, Artificial Sequence
  Synthetic oligonucleotide
  misc_feature (19)..(26): n is a, c, g, or t
acgacgctcttccgatctnnnnnnnnccgttgacttttttttttttttttttttttttgc

SEQ ID NO: 283 - DNA, 60 bp, Artificial Sequence
  Synthetic oligonucleotide
  misc_feature (19)..(26): n is a, c, g, or t
acgacgctcttccgatctnnnnnnnngccaggatcattttttttttttttttttttttgc

SEQ ID NO: 284 - DNA, 60 bp, Artificial Sequence
  Synthetic oligonucleotide
  misc_feature (19)..(26): n is a, c, g, or t
acgacgctcttccgatctnnnnnnnncttcggcgcattttttttttttttttttttttgc

SEQ ID NO: 285 - DNA, 60 bp, Artificial Sequence
  Synthetic oligonucleotide
  misc_feature (19)..(26): n is a, c, g, or t
acgacgctcttccgatctnnnnnnnncaaggcattcttttttttttttttttttttttgc

SEQ ID NO: 286 - DNA, 60 bp, Artificial Sequence
  Synthetic oligonucleotide
  misc_feature (19)..(26): n is a, c, g, or t
acgacgctcttccgatctnnnnnnnnaagaatggaattttttttttttttttttttttgc

SEQ ID NO: 287 - DNA, 60 bp, Artificial Sequence
  Synthetic oligonucleotide
  misc_feature (19)..(26): n is a, c, g, or t
acgacgctcttccgatctnnnnnnnncggatgaaggttttttttttttttttttttttgc

SEQ ID NO: 288 - DNA, 60 bp, Artificial Sequence
  Synthetic oligonucleotide
  misc_feature (19)..(26): n is a, c, g, or t
acgacgctcttccgatctnnnnnnnntatcgtcggcttttttttttttttttttttttgc

SEQ ID NO: 289 - DNA, 60 bp, Artificial Sequence
  Synthetic oligonucleotide
  misc_feature (19)..(26): n is a, c, g, or t
acgacgctcttccgatctnnnnnnnngccgtatgctttttttttttttttttttttttgc

SEQ ID NO: 290 - DNA, 60 bp, Artificial Sequence
  Synthetic oligonucleotide
  misc_feature (19)..(26): n is a, c, g, or t
acgacgctcttccgatctnnnnnnnnctgaactggtttttttttttttttttttttttgc

SEQ ID NO: 291 - DNA, 60 bp, Artificial Sequence
  Synthetic oligonucleotide
  misc_feature (19)..(26): n is a, c, g, or t
acgacgctcttccgatctnnnnnnnncataaccagcttttttttttttttttttttttgc

SEQ ID NO: 292 - DNA, 60 bp, Artificial Sequence
  Synthetic oligonucleotide
  misc_feature (19)..(26): n is a, c, g, or t
acgacgctcttccgatctnnnnnnnnaagttgccatttttttttttttttttttttttgc

SEQ ID NO: 293 - DNA, 60 bp, Artificial Sequence
  Synthetic oligonucleotide
  misc_feature (19)..(26): n is a, c, g, or t
acgacgctcttccgatctnnnnnnnnaggccgctcgttttttttttttttttttttttgc

SEQ ID NO: 294 - DNA, 60 bp, Artificial Sequence
  Synthetic oligonucleotide
  misc_feature (19)..(26): n is a, c, g, or t
acgacgctcttccgatctnnnnnnnnaggtaataggttttttttttttttttttttttgc

SEQ ID NO: 295 - DNA, 60 bp, Artificial Sequence
  Synthetic oligonucleotide
  misc_feature (19)..(26): n is a, c, g, or t
acgacgctcttccgatctnnnnnnnngtactagtaattttttttttttttttttttttgc

SEQ ID NO: 296 - DNA, 60 bp, Artificial Sequence
  Synthetic oligonucleotide
  misc_feature (19)..(26): n is a, c, g, or t
acgacgctcttccgatctnnnnnnnngcgcggtagtttttttttttttttttttttttgc

SEQ ID NO: 297 - DNA, 60 bp, Artificial Sequence
  Synthetic oligonucleotide
  misc_feature (19)..(26): n is a, c, g, or t
acgacgctcttccgatctnnnnnnnnctggattagtttttttttttttttttttttttgc

SEQ ID NO: 298 - DNA, 60 bp, Artificial Sequence
  Synthetic oligonucleotide
  misc_feature (19)..(26): n is a, c, g, or t
acgacgctcttccgatctnnnnnnnnttggatccttttttttttttttttttttttttgc

SEQ ID NO: 299 - DNA, 60 bp, Artificial Sequence
  Synthetic oligonucleotide
  misc_feature (19)..(26): n is a, c, g, or t
acgacgctcttccgatctnnnnnnnnttggaatctcttttttttttttttttttttttgc

SEQ ID NO: 300 - DNA, 60 bp, Artificial Sequence
  Synthetic oligonucleotide
  misc_feature (19)..(26): n is a, c, g, or t
acgacgctcttccgatctnnnnnnnnacctggacgcttttttttttttttttttttttgc

SEQ ID NO: 301 - DNA, 60 bp, Artificial Sequence
  Synthetic oligonucleotide
  misc_feature (19)..(26): n is a, c, g, or t
acgacgctcttccgatctnnnnnnnncctgacgttcttttttttttttttttttttttgc

SEQ ID NO: 302 - DNA, 60 bp, Artificial Sequence
  Synthetic oligonucleotide
  misc_feature (19)..(26): n is a, c, g, or t
acgacgctcttccgatctnnnnnnnngcgttcagctttttttttttttttttttttttgc

SEQ ID NO: 303 - DNA, 60 bp, Artificial Sequence
  Synthetic oligonucleotide
  misc_feature (19)..(26): n is a, c, g, or t
acgacgctcttccgatctnnnnnnnnttagcaataattttttttttttttttttttttgc

SEQ ID NO: 304 - DNA, 60 bp, Artificial Sequence
  Synthetic oligonucleotide
  misc_feature (19)..(26): n is a, c, g, or t
acgacgctcttccgatctnnnnnnnnttgatgctatttttttttttttttttttttttgc

SEQ ID NO: 305 - DNA, 60 bp, Artificial Sequence
  Synthetic oligonucleotide
  misc_feature (19)..(26): n is a, c, g, or t
acgacgctcttccgatctnnnnnnnnctctgcggcattttttttttttttttttttttgc

SEQ ID NO: 306 - DNA, 60 bp, Artificial Sequence
  Synthetic oligonucleotide
  misc_feature (19)..(26): n is a, c, g, or t
acgacgctcttccgatctnnnnnnnnaataataccattttttttttttttttttttttgc

SEQ ID NO: 307 - DNA, 60 bp, Artificial Sequence
  Synthetic oligonucleotide
  misc_feature (19)..(26): n is a, c, g, or t
acgacgctcttccgatctnnnnnnnnacgccgttcattttttttttttttttttttttgc

SEQ ID NO: 308 - DNA, 60 bp, Artificial Sequence
  Synthetic oligonucleotide
  misc_feature (19)..(26): n is a, c, g, or t
acgacgctcttccgatctnnnnnnnnttcgcttacgttttttttttttttttttttttgc

SEQ ID NO: 309 - DNA, 60 bp, Artificial Sequence
  Synthetic oligonucleotide
  misc_feature (19)..(26): n is a, c, g, or t
acgacgctcttccgatctnnnnnnnntacggctacgttttttttttttttttttttttgc

SEQ ID NO: 310 - DNA, 60 bp, Artificial Sequence
  Synthetic oligonucleotide
  misc_feature (19)..(26): n is a, c, g, or t
acgacgctcttccgatctnnnnnnnnttcttatcgattttttttttttttttttttttgc

SEQ ID NO: 311 - DNA, 60 bp, Artificial Sequence
  Synthetic oligonucleotide
  misc_feature (19)..(26): n is a, c, g, or t
acgacgctcttccgatctnnnnnnnnttccatggcattttttttttttttttttttttgc

SEQ ID NO: 312 - DNA, 60 bp, Artificial Sequence
  Synthetic oligonucleotide
  misc_feature (19)..(26): n is a, c, g, or t
acgacgctcttccgatctnnnnnnnnaagtagtcagttttttttttttttttttttttgc

SEQ ID NO: 313 - DNA, 60 bp, Artificial Sequence
  Synthetic oligonucleotide
  misc_feature (19)..(26): n is a, c, g, or t
acgacgctcttccgatctnnnnnnnntcagctctaattttttttttttttttttttttgc

SEQ ID NO: 314 - DNA, 60 bp, Artificial Sequence
  Synthetic oligonucleotide
  misc_feature (19)..(26): n is a, c, g, or t
acgacgctcttccgatctnnnnnnnncgaatagatgttttttttttttttttttttttgc

SEQ ID NO: 315 - DNA, 60 bp, Artificial Sequence
  Synthetic oligonucleotide
  misc_feature (19)..(26): n is a, c, g, or t
acgacgctcttccgatctnnnnnnnncggagatccgttttttttttttttttttttttgc

SEQ ID NO: 316 - DNA, 60 bp, Artificial Sequence
  Synthetic oligonucleotide
  misc_feature (19)..(26): n is a, c, g, or t
acgacgctcttccgatctnnnnnnnnaccgcagaatttttttttttttttttttttttgc

SEQ ID NO: 317 - DNA, 60 bp, Artificial Sequence
  Synthetic oligonucleotide
  misc_feature (19)..(26): n is a, c, g, or t
acgacgctcttccgatctnnnnnnnntctcctataattttttttttttttttttttttgc

SEQ ID NO: 318 - DNA, 60 bp, Artificial Sequence
  Synthetic oligonucleotide
  misc_feature (19)..(26): n is a, c, g, or t
acgacgctcttccgatctnnnnnnnncaacctatatttttttttttttttttttttttgc

SEQ ID NO: 319 - DNA, 60 bp, Artificial Sequence
  Synthetic oligonucleotide
  misc_feature (19)..(26): n is a, c, g, or t
acgacgctcttccgatctnnnnnnnnagtcgagaagttttttttttttttttttttttgc

SEQ ID NO: 320 - DNA, 60 bp, Artificial Sequence
  Synthetic oligonucleotide
  misc_feature (19)..(26): n is a, c, g, or t
acgacgctcttccgatctnnnnnnnnaagacggccattttttttttttttttttttttgc

SEQ ID NO: 321 - DNA, 60 bp, Artificial Sequence
  Synthetic oligonucleotide
  misc_feature (19)..(26): n is a, c, g, or t
acgacgctcttccgatctnnnnnnnngccaacgccattttttttttttttttttttttgc

SEQ ID NO: 322 - DNA, 60 bp, Artificial Sequence
  Synthetic oligonucleotide
  misc_feature (19)..(26): n is a, c, g, or t
acgacgctcttccgatctnnnnnnnntctaccattattttttttttttttttttttttgc

SEQ ID NO: 323 - DNA, 60 bp, Artificial Sequence
  Synthetic oligonucleotide
  misc_feature (19)..(26): n is a, c, g, or t
acgacgctcttccgatctnnnnnnnncttgcggtctttttttttttttttttttttttgc

SEQ ID NO: 324 - DNA, 60 bp, Artificial Sequence
  Synthetic oligonucleotide
  misc_feature (19)..(26): n is a, c, g, or t
acgacgctcttccgatctnnnnnnnnttacgtatacttttttttttttttttttttttgc

SEQ ID NO: 325 - DNA, 60 bp, Artificial Sequence
  Synthetic oligonucleotide
  misc_feature (19)..(26): n is a, c, g, or t
acgacgctcttccgatctnnnnnnnncgattggttattttttttttttttttttttttgc

SEQ ID NO: 326 - DNA, 60 bp, Artificial Sequence
  Synthetic oligonucleotide
  misc_feature (19)..(26): n is a, c, g, or t
acgacgctcttccgatctnnnnnnnnacttaactagttttttttttttttttttttttgc

SEQ ID NO: 327 - DNA, 60 bp, Artificial Sequence
  Synthetic oligonucleotide
  misc_feature (19)..(26): n is a, c, g, or t
acgacgctcttccgatctnnnnnnnngcagaccggtttttttttttttttttttttttgc

SEQ ID NO: 328 - DNA, 60 bp, Artificial Sequence
  Synthetic oligonucleotide
  misc_feature (19)..(26): n is a, c, g, or t
acgacgctcttccgatctnnnnnnnntgagtccagattttttttttttttttttttttgc

SEQ ID NO: 329 - DNA, 60 bp, Artificial Sequence
  Synthetic oligonucleotide
  misc_feature (19)..(26): n is a, c, g, or t
acgacgctcttccgatctnnnnnnnntggagaattcttttttttttttttttttttttgc

SEQ ID NO: 330 - DNA, 60 bp, Artificial Sequence
  Synthetic oligonucleotide
  misc_feature (19)..(26): n is a, c, g, or t
acgacgctcttccgatctnnnnnnnnaccagccttattttttttttttttttttttttgc

SEQ ID NO: 331 - DNA, 60 bp, Artificial Sequence
  Synthetic oligonucleotide
  misc_feature (19)..(26): n is a, c, g, or t
acgacgctcttccgatctnnnnnnnnggcgagcttattttttttttttttttttttttgc

SEQ ID NO: 332 - DNA, 60 bp, Artificial Sequence
  Synthetic oligonucleotide
  misc_feature (19)..(26): n is a, c, g, or t
acgacgctcttccgatctnnnnnnnntcgaggagtattttttttttttttttttttttgc

SEQ ID NO: 333 - DNA, 60 bp, Artificial Sequence
  Synthetic oligonucleotide
  misc_feature (19)..(26): n is a, c, g, or t
acgacgctcttccgatctnnnnnnnnccttactcctttttttttttttttttttttttgc

SEQ ID NO: 334 - DNA, 60 bp, Artificial Sequence
  Synthetic oligonucleotide
  misc_feature (19)..(26): n is a, c, g, or t
acgacgctcttccgatctnnnnnnnntcagacgaacttttttttttttttttttttttgc

SEQ ID NO: 335 - DNA, 60 bp, Artificial Sequence
  Synthetic oligonucleotide
  misc_feature (19)..(26): n is a, c, g, or t
acgacgctcttccgatctnnnnnnnnccgtccagtattttttttttttttttttttttgc

SEQ ID NO: 336 - DNA, 60 bp, Artificial Sequence
  Synthetic oligonucleotide
  misc_feature (19)..(26): n is a, c, g, or t
acgacgctcttccgatctnnnnnnnngttccgctaattttttttttttttttttttttgc

SEQ ID NO: 337 - DNA, 60 bp, Artificial Sequence
  Synthetic oligonucleotide
  misc_feature (19)..(26): n is a, c, g, or t
acgacgctcttccgatctnnnnnnnncagattcgatttttttttttttttttttttttgc

SEQ ID NO: 338 - DNA, 60 bp, Artificial Sequence
  Synthetic oligonucleotide
  misc_feature (19)..(26): n is a, c, g, or t
acgacgctcttccgatctnnnnnnnntgcatataacttttttttttttttttttttttgc

SEQ ID NO: 339 - DNA, 60 bp, Artificial Sequence
  Synthetic oligonucleotide
  misc_feature (19)..(26): n is a, c, g, or t
acgacgctcttccgatctnnnnnnnntaggcagatattttttttttttttttttttttgc

SEQ ID NO: 340 - DNA, 60 bp, Artificial Sequence
  Synthetic oligonucleotide
  misc_feature (19)..(26): n is a, c, g, or t
acgacgctcttccgatctnnnnnnnntatgccgagtttttttttttttttttttttttgc

SEQ ID NO: 341 - DNA, 60 bp, Artificial Sequence
  Synthetic oligonucleotide
  misc_feature (19)..(26): n is a, c, g, or t
acgacgctcttccgatctnnnnnnnnatagtcgtagttttttttttttttttttttttgc

SEQ ID NO: 342 - DNA, 60 bp, Artificial Sequence
  Synthetic oligonucleotide
  misc_feature (19)..(26): n is a, c, g, or t
acgacgctcttccgatctnnnnnnnnggatgcagcattttttttttttttttttttttgc

SEQ ID NO: 343 - DNA, 60 bp, Artificial Sequence
  Synthetic oligonucleotide
  misc_feature (19)..(26): n is a, c, g, or t
acgacgctcttccgatctnnnnnnnnccgctatattttttttttttttttttttttttgc

SEQ ID NO: 344 - DNA, 60 bp, Artificial Sequence
  Synthetic oligonucleotide
  misc_feature (19)..(26): n is a, c, g, or t
acgacgctcttccgatctnnnnnnnnatcgagtcgcttttttttttttttttttttttgc

SEQ ID NO: 345 - DNA, 60 bp, Artificial Sequence
  Synthetic oligonucleotide
  misc_feature (19)..(26): n is a, c, g, or t
acgacgctcttccgatctnnnnnnnngcgacgcagattttttttttttttttttttttgc

SEQ ID NO: 346 - DNA, 60 bp, Artificial Sequence
  Synthetic oligonucleotide
  misc_feature (19)..(26): n is a, c, g, or t
acgacgctcttccgatctnnnnnnnnaatggtcgacttttttttttttttttttttttgc

SEQ ID NO: 347 - DNA, 60 bp, Artificial Sequence
  Synthetic oligonucleotide
  misc_feature (19)..(26): n is a, c, g, or t
acgacgctcttccgatctnnnnnnnntggaactagattttttttttttttttttttttgc

SEQ ID NO: 348 - DNA, 60 bp, Artificial Sequence
  Synthetic oligonucleotide
  misc_feature (19)..(26): n is a, c, g, or t
acgacgctcttccgatctnnnnnnnngtccaactcattttttttttttttttttttttgc

SEQ ID NO: 349 - DNA, 60 bp, Artificial Sequence
  Synthetic oligonucleotide
  misc_feature (19)..(26): n is a, c, g, or t
acgacgctcttccgatctnnnnnnnngttatggatcttttttttttttttttttttttgc

SEQ ID NO: 350 - DNA, 60 bp, Artificial Sequence
  Synthetic oligonucleotide
  misc_feature (19)..(26): n is a, c, g, or t
acgacgctcttccgatctnnnnnnnnttataagaacttttttttttttttttttttttgc

SEQ ID NO: 351 - DNA, 60 bp, Artificial Sequence
  Synthetic oligonucleotide
  misc_feature (19)..(26): n is a, c, g, or t
acgacgctcttccgatctnnnnnnnncaagcttcatttttttttttttttttttttttgc

SEQ ID NO: 352 - DNA, 60 bp, Artificial Sequence
  Synthetic oligonucleotide
  misc_feature (19)..(26): n is a, c, g, or t
acgacgctcttccgatctnnnnnnnnctgattaagattttttttttttttttttttttgc

SEQ ID NO: 353 - DNA, 60 bp, Artificial Sequence
  Synthetic oligonucleotide
  misc_feature (19)..(26): n is a, c, g, or t
acgacgctcttccgatctnnnnnnnntacttacttattttttttttttttttttttttgc

SEQ ID NO: 354 - DNA, 60 bp, Artificial Sequence
  Synthetic oligonucleotide
  misc_feature (19)..(26): n is a, c, g, or t
acgacgctcttccgatctnnnnnnnnggatctgcagttttttttttttttttttttttgc

SEQ ID NO: 355 - DNA, 60 bp, Artificial Sequence
  Synthetic oligonucleotide
  misc_feature (19)..(26): n is a, c, g, or t
acgacgctcttccgatctnnnnnnnnatgcaatatgttttttttttttttttttttttgc

SEQ ID NO: 356 - DNA, 60 bp, Artificial Sequence
  Synthetic oligonucleotide
  misc_feature (19)..(26): n is a, c, g, or t
acgacgctcttccgatctnnnnnnnnttcctagaccttttttttttttttttttttttgc

SEQ ID NO: 357 - DNA, 60 bp, Artificial Sequence
  Synthetic oligonucleotide
  misc_feature (19)..(26): n is a, c, g, or t
acgacgctcttccgatctnnnnnnnnactgccgatattttttttttttttttttttttgc

SEQ ID NO: 358 - DNA, 60 bp, Artificial Sequence
  Synthetic oligonucleotide
  misc_feature (19)..(26): n is a, c, g, or t
acgacgctcttccgatctnnnnnnnntccagaaggtttttttttttttttttttttttgc

SEQ ID NO: 359 - DNA, 60 bp, Artificial Sequence
  Synthetic oligonucleotide
  misc_feature (19)..(26): n is a, c, g, or t
acgacgctcttccgatctnnnnnnnnttcaagaccattttttttttttttttttttttgc

SEQ ID NO: 360 - DNA, 60 bp, Artificial Sequence
  Synthetic oligonucleotide
  misc_feature (19)..(26): n is a, c, g, or t
acgacgctcttccgatctnnnnnnnntattactcatttttttttttttttttttttttgc

SEQ ID NO: 361 - DNA, 60 bp, Artificial Sequence
  Synthetic oligonucleotide
  misc_feature (19)..(26): n is a, c, g, or t
acgacgctcttccgatctnnnnnnnnaactgatcttttttttttttttttttttttttgc

SEQ ID NO: 362 - DNA, 60 bp, Artificial Sequence
  Synthetic oligonucleotide
  misc_feature (19)..(26): n is a, c, g, or t
acgacgctcttccgatctnnnnnnnnccgcggaccgttttttttttttttttttttttgc

SEQ ID NO: 363 - DNA, 60 bp, Artificial Sequence
  Synthetic oligonucleotide
  misc_feature (19)..(26): n is a, c, g, or t
acgacgctcttccgatctnnnnnnnnaatacgcaggttttttttttttttttttttttgc

SEQ ID NO: 364 - DNA, 60 bp, Artificial Sequence
  Synthetic oligonucleotide
  misc_feature (19)..(26): n is a, c, g, or t
acgacgctcttccgatctnnnnnnnnggtcgcgtcattttttttttttttttttttttgc

SEQ ID NO: 365 - DNA, 60 bp, Artificial Sequence
  Synthetic oligonucleotide
  misc_feature (19)..(26): n is a, c, g, or t
acgacgctcttccgatctnnnnnnnnaattatcagcttttttttttttttttttttttgc

SEQ ID NO: 366 - DNA, 60 bp, Artificial Sequence
  Synthetic oligonucleotide
  misc_feature (19)..(26): n is a, c, g, or t
acgacgctcttccgatctnnnnnnnncagctatcgtttttttttttttttttttttttgc

SEQ ID NO: 367 - DNA, 60 bp, Artificial Sequence
  Synthetic oligonucleotide
  misc_feature (19)..(26): n is a, c, g, or t
acgacgctcttccgatctnnnnnnnnattgcgctgattttttttttttttttttttttgc

SEQ ID NO: 368 - DNA, 60 bp, Artificial Sequence
  Synthetic oligonucleotide
  misc_feature (19)..(26): n is a, c, g, or t
acgacgctcttccgatctnnnnnnnnttggtaggcgttttttttttttttttttttttgc

SEQ ID NO: 369 - DNA, 60 bp, Artificial Sequence
  Synthetic oligonucleotide
  misc_feature (19)..(26): n is a, c, g, or t
acgacgctcttccgatctnnnnnnnnagctaaggtattttttttttttttttttttttgc

SEQ ID NO: 370 - DNA, 60 bp, Artificial Sequence
  Synthetic oligonucleotide
  misc_feature (19)..(26): n is a, c, g, or t
acgacgctcttccgatctnnnnnnnntcgtagagaattttttttttttttttttttttgc

SEQ ID NO: 371 - DNA, 60 bp, Artificial Sequence
  Synthetic oligonucleotide
  misc_feature (19)..(26): n is a, c, g, or t
acgacgctcttccgatctnnnnnnnntgatggccttttttttttttttttttttttttgc

SEQ ID NO: 372 - DNA, 60 bp, Artificial Sequence
  Synthetic oligonucleotide
  misc_feature (19)..(26): n is a, c, g, or t
acgacgctcttccgatctnnnnnnnntggaagtaccttttttttttttttttttttttgc

SEQ ID NO: 373 - DNA, 60 bp, Artificial Sequence
  Synthetic oligonucleotide
  misc_feature (19)..(26): n is a, c, g, or t
acgacgctcttccgatctnnnnnnnnctccaaggatttttttttttttttttttttttgc

SEQ ID NO: 374 - DNA, 60 bp, Artificial Sequence
  Synthetic oligonucleotide
  misc_feature (19)..(26): n is a, c, g, or t
acgacgctcttccgatctnnnnnnnnagatatatcgttttttttttttttttttttttgc

SEQ ID NO: 375 - DNA, 60 bp, Artificial Sequence
  Synthetic oligonucleotide
  misc_feature (19)..(26): n is a, c, g, or t
acgacgctcttccgatctnnnnnnnncatgctggttttttttttttttttttttttttgc

SEQ ID NO: 376 - DNA, 60 bp, Artificial Sequence
  Synthetic oligonucleotide
  misc_feature (19)..(26): n is a, c, g, or t
acgacgctcttccgatctnnnnnnnntcctcgagtcttttttttttttttttttttttgc

SEQ ID NO: 377 - DNA, 60 bp, Artificial Sequence
  Synthetic oligonucleotide
  misc_feature (19)..(26): n is a, c, g, or t
acgacgctcttccgatctnnnnnnnngcaaggaatattttttttttttttttttttttgc

SEQ ID NO: 378 - DNA, 60 bp, Artificial Sequence
  Synthetic oligonucleotide
  misc_feature (19)..(26): n is a, c, g, or t
acgacgctcttccgatctnnnnnnnnggcatagcttttttttttttttttttttttttgc

SEQ ID NO: 379 - DNA, 60 bp, Artificial Sequence
  Synthetic oligonucleotide
  misc_feature (19)..(26): n is a, c, g, or t
acgacgctcttccgatctnnnnnnnnctacggtagcttttttttttttttttttttttgc

SEQ ID NO: 380 - DNA, 60 bp, Artificial Sequence
  Synthetic oligonucleotide
  misc_feature (19)..(26): n is a, c, g, or t
acgacgctcttccgatctnnnnnnnnagtaagcatattttttttttttttttttttttgc

SEQ ID NO: 381 - DNA, 60 bp, Artificial Sequence
  Synthetic oligonucleotide
  misc_feature (19)..(26): n is a, c, g, or t
acgacgctcttccgatctnnnnnnnncgcctcgaacttttttttttttttttttttttgc

SEQ ID NO: 382 - DNA, 60 bp, Artificial Sequence
  Synthetic oligonucleotide
  misc_feature (19)..(26): n is a, c, g, or t
acgacgctcttccgatctnnnnnnnnttaggatctattttttttttttttttttttttgc

SEQ ID NO: 383 - DNA, 60 bp, Artificial Sequence
  Synthetic oligonucleotide
  misc_feature (19)..(26): n is a, c, g, or t
acgacgctcttccgatctnnnnnnnnactactgaagttttttttttttttttttttttgc

SEQ ID NO: 384 - DNA, 60 bp, Artificial Sequence
  Synthetic oligonucleotide
  misc_feature (19)..(26): n is a, c, g, or t
acgacgctcttccgatctnnnnnnnnaatctggagtttttttttttttttttttttttgc

SEQ ID NO: 385 - DNA, 26 bp, Artificial Sequence
  Synthetic oligonucleotide
  misc_feature (1)..(1): 5′ Biosg
taaacgaacwwcgcagagtgaatggg

SEQ ID NO: 386 - DNA, 51 bp, Artificial Sequence
  Synthetic oligonucleotide
  misc_feature (1)..(1): 5′ Biosg
  misc_feature (34)..(42): n is a, c, g, or t
acactctttccctacacgacgctcttccgatctnnnnnnnnnkswtcttwk

SEQ ID NO: 387 - DNA, 51 bp, Artificial Sequence
  Synthetic oligonucleotide
  misc_feature (1)..(1): 5′ Biosg
  misc_feature (35)..(40): n is a, c, g, or t
gtctcgtgggctcggagatgtgtataagagacagnnnnnntaaacgaacww

SEQ ID NO: 388 - DNA, 51 bp, Artificial Sequence
  Synthetic oligonucleotide
  misc_feature (1)..(1): 5′ Biosg
  misc_feature (35)..(40): n is a, c, g, or t
gtctcgtgggctcggagatgtgtataagagacagnnnnnngaccccaaaat

SEQ ID NO: 389 - DNA, 51 bp, Artificial Sequence
  Synthetic oligonucleotide
  misc_feature (1)..(1): 5′ Biosg
  misc_feature (34)..(42): n is a, c, g, or t
acactctttccctacacgacgctcttccgatctnnnnnnnnntctggttac

SEQ ID NO: 390 - DNA, 51 bp, Artificial Sequence
  Synthetic oligonucleotide
  misc_feature (1)..(1): 5′ Biosg
  misc_feature (35)..(40): n is a, c, g, or t
gtctcgtgggctcggagatgtgtataagagacagnnnnnnttacaaacatt

SEQ ID NO: 391 - DNA, 51 bp, Artificial Sequence
  Synthetic oligonucleotide
  misc_feature (1)..(1): 5′ Biosg
  misc_feature (34)..(42): n is a, c, g, or t
acactctttccctacacgacgctcttccgatctnnnnnnnnngcgcgacat

SEQ ID NO: 392 - DNA, 51 bp, Artificial Sequence
  Synthetic oligonucleotide
  misc_feature (1)..(1): 5′ Biosg
  misc_feature (35)..(40): n is a, c, g, or t
gtctcgtgggctcggagatgtgtataagagacagnnnnnnagatttggacc

SEQ ID NO: 393 - DNA, 51 bp, Artificial Sequence
  Synthetic oligonucleotide
  misc_feature (1)..(1): 5′ Biosg
  misc_feature (34)..(42): n is a, c, g, or t
acactctttccctacacgacgctcttccgatctnnnnnnnnngagcggctg

SEQ ID NO: 394 - DNA, 33 bp, Artificial Sequence
  Synthetic oligonucleotide
acactctttccctacacgacgctcttccgatct

SEQ ID NO: 395 - DNA, 23 bp, Artificial Sequence
  Synthetic oligonucleotide
aagcagtggtatcaacgcagagt

SEQ ID NO: 396 - DNA, 33 bp, Artificial Sequence
  Synthetic oligonucleotide
aagcagtggtatcaacgcagagtgaatrgrgrg

SEQ ID NO: 397 - DNA, 47 bp, Artificial Sequence
  Synthetic oligonucleotide
  misc_feature (47)..(47): n is a, c, g, or t
aagcagtggtatcaacgcagagtttttttttttttttttttttttvn

SEQ ID NO: 398 - DNA, 36 bp, Artificial Sequence
  Synthetic oligonucleotide
  misc_feature (36)..(36): 3′ C6
ttagagggacaagtggcgttcagccacccgagattg

SEQ ID NO: 399 - DNA, 36 bp, Artificial Sequence
  Synthetic oligonucleotide
  misc_feature (36)..(36): 3′ C6
caatctcgggtggctgaacgccacttgtccctctaa

SEQ ID NO: 400 - DNA, 72 bp, Artificial Sequence
  Synthetic oligonucleotide
aatgatacggcgaccaccgagatctacacctccatcgagacactctttccctacacgacgctcttccgatct

SEQ ID NO: 401 - DNA, 72 bp, Artificial Sequence
  Synthetic oligonucleotide
aatgatacggcgaccaccgagatctacacttggtagtcgacactctttccctacacgacgctcttccgatct

SEQ ID NO: 402 - DNA, 72 bp, Artificial Sequence
  Synthetic oligonucleotide
aatgatacggcgaccaccgagatctacacggccgtcaacacactctttccctacacgacgctcttccgatct

SEQ ID NO: 403 - DNA, 72 bp, Artificial Sequence
  Synthetic oligonucleotide
aatgatacggcgaccaccgagatctacaccctagacgagacactctttccctacacgacgctcttccgatct

SEQ ID NO: 404 - DNA, 72 bp, Artificial Sequence
  Synthetic oligonucleotide
aatgatacggcgaccaccgagatctacactcgttagagcacactctttccctacacgacgctcttccgatct

SEQ ID NO: 405 - DNA, 72 bp, Artificial Sequence
  Synthetic oligonucleotide
aatgatacggcgaccaccgagatctacaccgttctatcaacactctttccctacacgacgctcttccgatct

SEQ ID NO: 406 - DNA, 72 bp, Artificial Sequence
  Synthetic oligonucleotide
aatgatacggcgaccaccgagatctacaccggaatctaaacactctttccctacacgacgctcttccgatct

SEQ ID NO: 407 - DNA, 72 bp, Artificial Sequence
  Synthetic oligonucleotide
aatgatacggcgaccaccgagatctacacatgactgatcacactctttccctacacgacgctcttccgatct

SEQ ID NO: 408 - DNA, 72 bp, Artificial Sequence
  Synthetic oligonucleotide
aatgatacggcgaccaccgagatctacactcaatatcgaacactctttccctacacgacgctcttccgatct

SEQ ID NO: 409 - DNA, 72 bp, Artificial Sequence
  Synthetic oligonucleotide
aatgatacggcgaccaccgagatctacacgtagacctggacactctttccctacacgacgctcttccgatct

SEQ ID NO: 410 - DNA, 72 bp, Artificial Sequence
  Synthetic oligonucleotide
aatgatacggcgaccaccgagatctacacttatgaccaaacactctttccctacacgacgctcttccgatct

SEQ ID NO: 411 - DNA, 72 bp, Artificial Sequence
  Synthetic oligonucleotide
aatgatacggcgaccaccgagatctacacttggtccgttacactctttccctacacgacgctcttccgatct

SEQ ID NO: 412 - DNA, 72 bp, Artificial Sequence
  Synthetic oligonucleotide
aatgatacggcgaccaccgagatctacacggtacgttaaacactctttccctacacgacgctcttccgatct

SEQ ID NO: 413 - DNA, 72 bp, Artificial Sequence
  Synthetic oligonucleotide
aatgatacggcgaccaccgagatctacaccaatgagtccacactctttccctacacgacgctcttccgatct

SEQ ID NO: 414 - DNA, 72 bp, Artificial Sequence
  Synthetic oligonucleotide
aatgatacggcgaccaccgagatctacacgatgcagttcacactctttccctacacgacgctcttccgatct

SEQ ID NO: 415 - DNA, 72 bp, Artificial Sequence
  Synthetic oligonucleotide
aatgatacggcgaccaccgagatctacacccatcgttccacactctttccctacacgacgctcttccgatct

SEQ ID NO: 416 - DNA, 72 bp, Artificial Sequence
  Synthetic oligonucleotide
aatgatacggcgaccaccgagatctacacttgagagagtacactctttccctacacgacgctcttccgatct

SEQ ID NO: 417 - DNA, 72 bp, Artificial Sequence
  Synthetic oligonucleotide
aatgatacggcgaccaccgagatctacacactgagcgacacactctttccctacacgacgctcttccgatct

SEQ ID NO: 418 - DNA, 72 bp, Artificial Sequence
  Synthetic oligonucleotide
aatgatacggcgaccaccgagatctacactgaggaatcaacactctttccctacacgacgctcttccgatct

SEQ ID NO: 419 - DNA, 72 bp, Artificial Sequence
  Synthetic oligonucleotide
aatgatacggcgaccaccgagatctacaccctccgacggacactctttccctacacgacgctcttccgatct

SEQ ID NO: 420 - DNA, 72 bp, Artificial Sequence
  Synthetic oligonucleotide
aatgatacggcgaccaccgagatctacaccattgacgctacactctttccctacacgacgctcttccgatct

SEQ ID NO: 421 - DNA, 72 bp, Artificial Sequence
  Synthetic oligonucleotide
aatgatacggcgaccaccgagatctacactcgtccttcgacactctttccctacacgacgctcttccgatct

SEQ ID NO: 422 - DNA, 72 bp, Artificial Sequence
  Synthetic oligonucleotide
aatgatacggcgaccaccgagatctacactgatactcaaacactctttccctacacgacgctcttccgatct

SEQ ID NO: 423 - DNA, 72 bp, Artificial Sequence
  Synthetic oligonucleotide
aatgatacggcgaccaccgagatctacacttctacctcaacactctttccctacacgacgctcttccgatct

SEQ ID NO: 424 - DNA, 72 bp, Artificial Sequence
  Synthetic oligonucleotide
aatgatacggcgaccaccgagatctacactcgtcggaacacactctttccctacacgacgctcttccgatct

SEQ ID NO: 425 - DNA, 72 bp, Artificial Sequence
  Synthetic oligonucleotide
aatgatacggcgaccaccgagatctacacatcgagatgaacactctttccctacacgacgctcttccgatct

SEQ ID NO: 426 - DNA, 72 bp, Artificial Sequence
  Synthetic oligonucleotide
aatgatacggcgaccaccgagatctacactagactagtcacactctttccctacacgacgctcttccgatct

SEQ ID NO: 427 - DNA, 72 bp, Artificial Sequence
  Synthetic oligonucleotide
aatgatacggcgaccaccgagatctacacgtcgaagcagacactctttccctacacgacgctcttccgatct

SEQ ID NO: 428 - DNA, 72 bp, Artificial Sequence
  Synthetic oligonucleotide
aatgatacggcgaccaccgagatctacacaggcgctaggacactctttccctacacgacgctcttccgatct

SEQ ID NO: 429 - DNA, 72 bp, Artificial Sequence
  Synthetic oligonucleotide
aatgatacggcgaccaccgagatctacacagatgcaactacactctttccctacacgacgctcttccgatct

SEQ ID NO: 430 - DNA, 72 bp, Artificial Sequence
  Synthetic oligonucleotide
aatgatacggcgaccaccgagatctacacaagcctacgaacactctttccctacacgacgctcttccgatct

SEQ ID NO: 431 - DNA, 72 bp, Artificial Sequence
  Synthetic oligonucleotide
aatgatacggcgaccaccgagatctacacgtaggcaattacactctttccctacacgacgctcttccgatct

SEQ ID NO: 432 - DNA, 72 bp, Artificial Sequence
  Synthetic oligonucleotide
aatgatacggcgaccaccgagatctacactgccagttgcacactctttccctacacgacgctcttccgatct

SEQ ID NO: 433 - DNA, 72 bp, Artificial Sequence
  Synthetic oligonucleotide
aatgatacggcgaccaccgagatctacaccttaggtatcacactctttccctacacgacgctcttccgatct

SEQ ID NO: 434 - DNA, 72 bp, Artificial Sequence
  Synthetic oligonucleotide
aatgatacggcgaccaccgagatctacacgagacctaccacactctttccctacacgacgctcttccgatct

SEQ ID NO: 435 - DNA, 72 bp, Artificial Sequence
  Synthetic oligonucleotide
aatgatacggcgaccaccgagatctacacattgaccgagacactctttccctacacgacgctcttccgatct

SEQ ID NO: 436 - DNA, 72 bp, Artificial Sequence
  Synthetic oligonucleotide
aatgatacggcgaccaccgagatctacacggaggcggcgacactctttccctacacgacgctcttccgatct

SEQ ID NO: 437 - DNA, 72 bp, Artificial Sequence
  Synthetic oligonucleotide
aatgatacggcgaccaccgagatctacacccagtacttgacactctttccctacacgacgctcttccgatct

SEQ ID NO: 438 - DNA, 72 bp, Artificial Sequence
  Synthetic oligonucleotide
aatgatacggcgaccaccgagatctacacggtctcgccgacactctttccctacacgacgctcttccgatct

SEQ ID NO: 439 - DNA, 72 bp, Artificial Sequence
  Synthetic oligonucleotide
aatgatacggcgaccaccgagatctacacggcggaggtcacactctttccctacacgacgctcttccgatct

SEQ ID NO: 440 - DNA, 72 bp, Artificial Sequence
  Synthetic oligonucleotide
aatgatacggcgaccaccgagatctacactagttctagaacactctttccctacacgacgctcttccgatct

SEQ ID NO: 441 - DNA, 72 bp, Artificial Sequence
  Synthetic oligonucleotide
aatgatacggcgaccaccgagatctacacttggagttagacactctttccctacacgacgctcttccgatct

SEQ ID NO: 442 - DNA, 72 bp, Artificial Sequence
  Synthetic oligonucleotide
aatgatacggcgaccaccgagatctacacagatcttggtacactctttccctacacgacgctcttccgatct

SEQ ID NO: 443 - DNA, 72 bp, Artificial Sequence
  Synthetic oligonucleotide
aatgatacggcgaccaccgagatctacacgtaatgatcgacactctttccctacacgacgctcttccgatct

SEQ ID NO: 444 - DNA, 72 bp, Artificial Sequence
  Synthetic oligonucleotide
aatgatacggcgaccaccgagatctacaccagagaggtcacactctttccctacacgacgctcttccgatct

SEQ ID NO: 445 - DNA, 72 bp, Artificial Sequence
  Synthetic oligonucleotide
aatgatacggcgaccaccgagatctacacttaattagccacactctttccctacacgacgctcttccgatct

SEQ ID NO: 446 - DNA, 72 bp, Artificial Sequence
  Synthetic oligonucleotide
aatgatacggcgaccaccgagatctacacctctaactcgacactctttccctacacgacgctcttccgatct

SEQ ID NO: 447 - DNA, 72 bp, Artificial Sequence
  Synthetic oligonucleotide
aatgatacggcgaccaccgagatctacactacgatcatcacactctttccctacacgacgctcttccgatct

SEQ ID NO: 448 - DNA, 72 bp, Artificial Sequence
  Synthetic oligonucleotide
aatgatacggcgaccaccgagatctacacaggcgagagcacactctttccctacacgacgctcttccgatct

SEQ ID NO: 449 - DNA, 72 bp, Artificial Sequence
  Synthetic oligonucleotide
aatgatacggcgaccaccgagatctacactcaagatagtacactctttccctacacgacgctcttccgatct

SEQ ID NO: 450 - DNA, 72 bp, Artificial Sequence
  Synthetic oligonucleotide
aatgatacggcgaccaccgagatctacactaattgacctacactctttccctacacgacgctcttccgatct

SEQ ID NO: 451 - DNA, 72 bp, Artificial Sequence
  Synthetic oligonucleotide
aatgatacggcgaccaccgagatctacaccagccggcttacactctttccctacacgacgctcttccgatct

SEQ ID NO: 452 - DNA, 72 bp, Artificial Sequence
  Synthetic oligonucleotide
aatgatacggcgaccaccgagatctacacagaaccggagacactctttccctacacgacgctcttccgatct

SEQ ID NO: 453 - DNA, 72 bp, Artificial Sequence
  Synthetic oligonucleotide
aatgatacggcgaccaccgagatctacacgagatgcatgacactctttccctacacgacgctcttccgatct

SEQ ID NO: 454 - DNA, 72 bp, Artificial Sequence
  Synthetic oligonucleotide
aatgatacggcgaccaccgagatctacacgattaccggaacactctttccctacacgacgctcttccgatct

SEQ ID NO: 455 - DNA, 72 bp, Artificial Sequence
  Synthetic oligonucleotide
aatgatacggcgaccaccgagatctacactcgtaacggtacactctttccctacacgacgctcttccgatct

SEQ ID NO: 456 - DNA, 72 bp, Artificial Sequence
  Synthetic oligonucleotide
aatgatacggcgaccaccgagatctacactggcgacggaacactctttccctacacgacgctcttccgatct

SEQ ID NO: 457 - DNA, 72 bp, Artificial Sequence
  Synthetic oligonucleotide
aatgatacggcgaccaccgagatctacacagtcatagccacactctttccctacacgacgctcttccgatct

SEQ ID NO: 458 - DNA, 72 bp, Artificial Sequence
  Synthetic oligonucleotide
aatgatacggcgaccaccgagatctacacgtcaagtccaacactctttccctacacgacgctcttccgatct

SEQ ID NO: 459 - DNA, 72 bp, Artificial Sequence
  Synthetic oligonucleotide
aatgatacggcgaccaccgagatctacacattcggaagtacactctttccctacacgacgctcttccgatct

SEQ ID NO: 460 - DNA, 72 bp, Artificial Sequence
  Synthetic oligonucleotide
aatgatacggcgaccaccgagatctacacgtcggtagttacactctttccctacacgacgctcttccgatct

SEQ ID NO: 461 - DNA, 72 bp, Artificial Sequence
  Synthetic oligonucleotide
aatgatacggcgaccaccgagatctacacaggacggacgacactctttccctacacgacgctcttccgatct

SEQ ID NO: 462 - DNA, 72 bp, Artificial Sequence
  Synthetic oligonucleotide
aatgatacggcgaccaccgagatctacacctcctggaccacactctttccctacacgacgctcttccgatct

SEQ ID NO: 463 - DNA, 72 bp, Artificial Sequence
  Synthetic oligonucleotide
aatgatacggcgaccaccgagatctacactagcctcgttacactctttccctacacgacgctcttccgatct

SEQ ID NO: 464 - DNA, 72 bp, Artificial Sequence
  Synthetic oligonucleotide
aatgatacggcgaccaccgagatctacacggttgaacgtacactctttccctacacgacgctcttccgatct

SEQ ID NO: 465 - DNA, 72 bp, Artificial Sequence
  Synthetic oligonucleotide
aatgatacggcgaccaccgagatctacacaggtcctcgtacactctttccctacacgacgctcttccgatct

SEQ ID NO: 466 - DNA, 72 bp, Artificial Sequence
  Synthetic oligonucleotide
aatgatacggcgaccaccgagatctacacggaagttataacactctttccctacacgacgctcttccgatct

SEQ ID NO: 467 - DNA, 72 bp, Artificial Sequence
  Synthetic oligonucleotide
aatgatacggcgaccaccgagatctacactggtaatcctacactctttccctacacgacgctcttccgatct

SEQ ID NO: 468 - DNA, 72 bp, Artificial Sequence
  Synthetic oligonucleotide
aatgatacggcgaccaccgagatctacacaagctaggttacactctttccctacacgacgctcttccgatct

SEQ ID NO: 469 - DNA, 72 bp, Artificial Sequence
  Synthetic oligonucleotide
aatgatacggcgaccaccgagatctacactccgcggactacactctttccctacacgacgctcttccgatct

SEQ ID NO: 470 - DNA, 72 bp, Artificial Sequence
  Synthetic oligonucleotide
aatgatacggcgaccaccgagatctacactgcggatagtacactctttccctacacgacgctcttccgatct

SEQ ID NO: 471 - DNA, 72 bp, Artificial Sequence
  Synthetic oligonucleotide
aatgatacggcgaccaccgagatctacactggcagctcgacactctttccctacacgacgctcttccgatct

SEQ ID NO: 472 - DNA, 72 bp, Artificial Sequence
  Synthetic oligonucleotide
aatgatacggcgaccaccgagatctacactgctacggtcacactctttccctacacgacgctcttccgatct

SEQ ID NO: 473 - DNA, 72 bp, Artificial Sequence
  Synthetic oligonucleotide
aatgatacggcgaccaccgagatctacacgcgcaatgacacactctttccctacacgacgctcttccgatct

SEQ ID NO: 474 - DNA, 72 bp, Artificial Sequence
  Synthetic oligonucleotide
aatgatacggcgaccaccgagatctacaccttaatcttgacactctttccctacacgacgctcttccgatct

SEQ ID NO: 475 - DNA, 72 bp, Artificial Sequence
  Synthetic oligonucleotide
aatgatacggcgaccaccgagatctacacggagttgcgtacactctttccctacacgacgctcttccgatct

SEQ ID NO: 476 - DNA, 72 bp, Artificial Sequence
  Synthetic oligonucleotide
aatgatacggcgaccaccgagatctacacactcgtatcaacactctttccctacacgacgctcttccgatct

SEQ ID NO: 477 - DNA, 72 bp, Artificial Sequence
  Synthetic oligonucleotide
aatgatacggcgaccaccgagatctacacggtaataatgacactctttccctacacgacgctcttccgatct

SEQ ID NO: 478 - DNA, 72 bp, Artificial Sequence
  Synthetic oligonucleotide
aatgatacggcgaccaccgagatctacactccttatagaacactctttccctacacgacgctcttccgatct

SEQ ID NO: 479 - DNA, 72 bp, Artificial Sequence
  Synthetic oligonucleotide
aatgatacggcgaccaccgagatctacacccgactccaaacactctttccctacacgacgctcttccgatct

SEQ ID NO: 480 - DNA, 72 bp, Artificial Sequence
  Synthetic oligonucleotide
aatgatacggcgaccaccgagatctacacgccaagcttgacactctttccctacacgacgctcttccgatct

SEQ ID NO: 481 - DNA, 72 bp, Artificial Sequence
  Synthetic oligonucleotide
aatgatacggcgaccaccgagatctacaccatatcctatacactctttccctacacgacgctcttccgatct

SEQ ID NO: 482 - DNA, 72 bp, Artificial Sequence
  Synthetic oligonucleotide
aatgatacggcgaccaccgagatctacacacctacgccaacactctttccctacacgacgctcttccgatct

SEQ ID NO: 483 - DNA, 72 bp, Artificial Sequence
  Synthetic oligonucleotide
aatgatacggcgaccaccgagatctacacggaattcagtacactctttccctacacgacgctcttccgatct

SEQ ID NO: 484 - DNA, 72 bp, Artificial Sequence
  Synthetic oligonucleotide
aatgatacggcgaccaccgagatctacactggcgtagaaacactctttccctacacgacgctcttccgatct

SEQ ID NO: 485 - DNA, 72 bp, Artificial Sequence
  Synthetic oligonucleotide
aatgatacggcgaccaccgagatctacacattgcggccaacactctttccctacacgacgctcttccgatct

SEQ ID NO: 486 - DNA, 72 bp, Artificial Sequence
  Synthetic oligonucleotide
aatgatacggcgaccaccgagatctacacttcagcttggacactctttccctacacgacgctcttccgatct

SEQ ID NO: 487 - DNA, 72 bp, Artificial Sequence
  Synthetic oligonucleotide
aatgatacggcgaccaccgagatctacacccatctggcaacactctttccctacacgacgctcttccgatct

SEQ ID NO: 488 - DNA, 72 bp, Artificial Sequence
  Synthetic oligonucleotide
aatgatacggcgaccaccgagatctacaccttataagttacactctttccctacacgacgctcttccgatct

SEQ ID NO: 489 - DNA, 72 bp, Artificial Sequence
  Synthetic oligonucleotide
aatgatacggcgaccaccgagatctacacgattagatgaacactctttccctacacgacgctcttccgatct

SEQ ID NO: 490 - DNA, 72 bp, Artificial Sequence
  Synthetic oligonucleotide
aatgatacggcgaccaccgagatctacactataggatctacactctttccctacacgacgctcttccgatct

SEQ ID NO: 491 - DNA, 72 bp, Artificial Sequence
  Synthetic oligonucleotide
aatgatacggcgaccaccgagatctacacagcttataggacactctttccctacacgacgctcttccgatct

SEQ ID NO: 492 - DNA, 72 bp, Artificial Sequence
  Synthetic oligonucleotide
aatgatacggcgaccaccgagatctacacgtctgcaatcacactctttccctacacgacgctcttccgatct

SEQ ID NO: 493 - DNA, 72 bp, Artificial Sequence
  Synthetic oligonucleotide
aatgatacggcgaccaccgagatctacaccgcctcttatacactctttccctacacgacgctcttccgatct

SEQ ID NO: 494 - DNA, 72 bp, Artificial Sequence
  Synthetic oligonucleotide
aatgatacggcgaccaccgagatctacacgttggatcttacactctttccctacacgacgctcttccgatct

SEQ ID NO: 495 - DNA, 72 bp, Artificial Sequence
  Synthetic oligonucleotide
aatgatacggcgaccaccgagatctacacgcgattgcagacactctttccctacacgacgctcttccgatct

SEQ ID NO: 496 - DNA, 10 bp, Artificial Sequence
  Synthetic oligonucleotide
ctcgatggag

SEQ ID NO: 497 - DNA, 10 bp, Artificial Sequence
  Synthetic oligonucleotide
cgactaccaa

SEQ ID NO: 498 - DNA, 10 bp, Artificial Sequence
  Synthetic oligonucleotide
gttgacggcc

SEQ ID NO: 499 - DNA, 10 bp, Artificial Sequence
  Synthetic oligonucleotide
ctcgtctagg

SEQ ID NO: 500 - DNA, 10 bp, Artificial Sequence
  Synthetic oligonucleotide
gctctaacga

SEQ ID NO: 501 - DNA, 10 bp, Artificial Sequence
  Synthetic oligonucleotide
tgatagaacg

SEQ ID NO: 502 - DNA, 10 bp, Artificial Sequence
  Synthetic oligonucleotide
ttagattccg

SEQ ID NO: 503 - DNA, 10 bp, Artificial Sequence
  Synthetic oligonucleotide
gatcagtcat

SEQ ID NO: 504 - DNA, 10 bp, Artificial Sequence
  Synthetic oligonucleotide
tcgatattga

SEQ ID NO: 505 - DNA, 10 bp, Artificial Sequence
  Synthetic oligonucleotide
ccaggtctac

SEQ ID NO: 506 - DNA, 10 bp, Artificial Sequence
  Synthetic oligonucleotide
ttggtcataa

SEQ ID NO: 507 - DNA, 10 bp, Artificial Sequence
  Synthetic oligonucleotide
aacggaccaa

SEQ ID NO: 508 - DNA, 10 bp, Artificial Sequence
  Synthetic oligonucleotide
ttaacgtacc

SEQ ID NO: 509 - DNA, 10 bp, Artificial Sequence
  Synthetic oligonucleotide
ggactcattg

SEQ ID NO: 510 - DNA, 10 bp, Artificial Sequence
  Synthetic oligonucleotide
gaactgcatc

SEQ ID NO: 511 - DNA, 10 bp, Artificial Sequence
  Synthetic oligonucleotide
ggaacgatgg

SEQ ID NO: 512 - DNA, 10 bp, Artificial Sequence
  Synthetic oligonucleotide
actctctcaa

SEQ ID NO: 513 - DNA, 10 bp, Artificial Sequence
  Synthetic oligonucleotide
gtcgctcagt

SEQ ID NO: 514 - DNA, 10 bp, Artificial Sequence
  Synthetic oligonucleotide
tgattcctca

SEQ ID NO: 515 - DNA, 10 bp, Artificial Sequence
  Synthetic oligonucleotide
ccgtcggagg

SEQ ID NO: 516 - DNA, 10 bp, Artificial Sequence
  Synthetic oligonucleotide
agcgtcaatg

SEQ ID NO: 517 - DNA, 10 bp, Artificial Sequence
  Synthetic oligonucleotide
cgaaggacga

SEQ ID NO: 518 - DNA, 10 bp, Artificial Sequence
  Synthetic oligonucleotide
ttgagtatca

SEQ ID NO: 519 - DNA, 10 bp, Artificial Sequence
  Synthetic oligonucleotide
tgaggtagaa

SEQ ID NO: 520 - DNA, 10 bp, Artificial Sequence
  Synthetic oligonucleotide
gttccgacga

SEQ ID NO: 521 - DNA, 10 bp, Artificial Sequence
  Synthetic oligonucleotide
tcatctcgat

SEQ ID NO: 522 - DNA, 10 bp, Artificial Sequence
  Synthetic oligonucleotide
gactagtcta

SEQ ID NO: 523 - DNA, 10 bp, Artificial Sequence
  Synthetic oligonucleotide
ctgcttcgac

SEQ ID NO: 524 - DNA, 10 bp, Artificial Sequence
  Synthetic oligonucleotide
cctagcgcct

SEQ ID NO: 525 - DNA, 10 bp, Artificial Sequence
  Synthetic oligonucleotide
agttgcatct

SEQ ID NO: 526 - DNA, 10 bp, Artificial Sequence
  Synthetic oligonucleotide
tcgtaggctt

SEQ ID NO: 527 - DNA, 10 bp, Artificial Sequence
  Synthetic oligonucleotide
aattgcctac

SEQ ID NO: 528 - DNA, 10 bp, Artificial Sequence
  Synthetic oligonucleotide
gcaactggca

SEQ ID NO: 529 - DNA, 10 bp, Artificial Sequence
  Synthetic oligonucleotide
gatacctaag

SEQ ID NO: 530 - DNA, 10 bp, Artificial Sequence
  Synthetic oligonucleotide
ggtaggtctc

SEQ ID NO: 531 - DNA, 10 bp, Artificial Sequence
  Synthetic oligonucleotide
ctcggtcaat

SEQ ID NO: 532 - DNA, 10 bp, Artificial Sequence
  Synthetic oligonucleotide
cgccgcctcc

SEQ ID NO: 533 - DNA, 10 bp, Artificial Sequence
  Synthetic oligonucleotide
caagtactgg

SEQ ID NO: 534 - DNA, 10 bp, Artificial Sequence
  Synthetic oligonucleotide
cggcgagacc

SEQ ID NO: 535 - DNA, 10 bp, Artificial Sequence
  Synthetic oligonucleotide
gacctccgcc

SEQ ID NO: 536 - DNA, 10 bp, Artificial Sequence
  Synthetic oligonucleotide
tctagaacta

SEQ ID NO: 537 - DNA, 10 bp, Artificial Sequence
  Synthetic oligonucleotide
ctaactccaa

SEQ ID NO: 538 - DNA, 10 bp, Artificial Sequence
  Synthetic oligonucleotide
accaagatct

SEQ ID NO: 539 - DNA, 10 bp, Artificial Sequence
  Synthetic oligonucleotide
cgatcattac

SEQ ID NO: 540 - DNA, 10 bp, Artificial Sequence
  Synthetic oligonucleotide
gacctctctg

SEQ ID NO: 541 - DNA, 10 bp, Artificial Sequence
  Synthetic oligonucleotide
ggctaattaa

SEQ ID NO: 542 - DNA, 10 bp, Artificial Sequence
  Synthetic oligonucleotide
cgagttagag

SEQ ID NO: 543 - DNA, 10 bp, Artificial Sequence
  Synthetic oligonucleotide
gatgatcgta

SEQ ID NO: 544 - DNA, 10 bp, Artificial Sequence
  Synthetic oligonucleotide
gctctcgcct

SEQ ID NO: 545 - DNA, 10 bp, Artificial Sequence
  Synthetic oligonucleotide
actatcttga

SEQ ID NO: 546 - DNA, 10 bp, Artificial Sequence
  Synthetic oligonucleotide
aggtcaatta

SEQ ID NO: 547 - DNA, 10 bp, Artificial Sequence
  Synthetic oligonucleotide
aagccggctg

SEQ ID NO: 548 - DNA, 10 bp, Artificial Sequence
  Synthetic oligonucleotide
ctccggttct

SEQ ID NO: 549 - DNA, 10 bp, Artificial Sequence
  Synthetic oligonucleotide
catgcatctc

SEQ ID NO: 550 - DNA, 10 bp, Artificial Sequence
  Synthetic oligonucleotide
tccggtaatc

SEQ ID NO: 551 - DNA, 10 bp, Artificial Sequence
  Synthetic oligonucleotide
accgttacga

SEQ ID NO: 552 - DNA, 10 bp, Artificial Sequence
  Synthetic oligonucleotide
tccgtcgcca

SEQ ID NO: 553 - DNA, 10 bp, Artificial Sequence
  Synthetic oligonucleotide
ggctatgact

SEQ ID NO: 554 - DNA, 10 bp, Artificial Sequence
  Synthetic oligonucleotide
tggacttgac

SEQ ID NO: 555 - DNA, 10 bp, Artificial Sequence
  Synthetic oligonucleotide
acttccgaat

SEQ ID NO: 556 - DNA, 10 bp, Artificial Sequence
  Synthetic oligonucleotide
aactaccgac

SEQ ID NO: 557 - DNA, 10 bp, Artificial Sequence
  Synthetic oligonucleotide
cgtccgtcct

SEQ ID NO: 558 - DNA, 10 bp, Artificial Sequence
  Synthetic oligonucleotide
ggtccaggag

SEQ ID NO: 559 - DNA, 10 bp, Artificial Sequence
  Synthetic oligonucleotide
aacgaggcta

SEQ ID NO: 560 - DNA, 10 bp, Artificial Sequence
  Synthetic oligonucleotide
acgttcaacc

SEQ ID NO: 561 - DNA, 10 bp, Artificial Sequence
  Synthetic oligonucleotide
acgaggacct

SEQ ID NO: 562 - DNA, 10 bp, Artificial Sequence
  Synthetic oligonucleotide
tataacttcc

SEQ ID NO: 563 - DNA, 10 bp, Artificial Sequence
  Synthetic oligonucleotide
aggattacca

SEQ ID NO: 564 - DNA, 10 bp, Artificial Sequence
  Synthetic oligonucleotide
aacctagctt

SEQ ID NO: 565 - DNA, 10 bp, Artificial Sequence
  Synthetic oligonucleotide
agtccgcgga

SEQ ID NO: 566 - DNA, 10 bp, Artificial Sequence
  Synthetic oligonucleotide
actatccgca

SEQ ID NO: 567 - DNA, 10 bp, Artificial Sequence
  Synthetic oligonucleotide
cgagctgcca

SEQ ID NO: 568 - DNA, 10 bp, Artificial Sequence
  Synthetic oligonucleotide
gaccgtagca

SEQ ID NO: 569 - DNA, 10 bp, Artificial Sequence
  Synthetic oligonucleotide
gtcattgcgc

SEQ ID NO: 570 - DNA, 10 bp, Artificial Sequence
  Synthetic oligonucleotide
caagattaag

SEQ ID NO: 571 - DNA, 10 bp, Artificial Sequence
  Synthetic oligonucleotide
acgcaactcc

SEQ ID NO: 572 - DNA, 10 bp, Artificial Sequence
  Synthetic oligonucleotide
tgatacgagt

SEQ ID NO: 573 - DNA, 10 bp, Artificial Sequence
  Synthetic oligonucleotide
cattattacc

SEQ ID NO: 574 - DNA, 10 bp, Artificial Sequence
  Synthetic oligonucleotide
tctataagga

SEQ ID NO: 575 - DNA, 10 bp, Artificial Sequence
  Synthetic oligonucleotide
ttggagtcgg

SEQ ID NO: 576 - DNA, 10 bp, Artificial Sequence
  Synthetic oligonucleotide
caagcttggc

SEQ ID NO: 577 - DNA, 10 bp, Artificial Sequence
  Synthetic oligonucleotide
ataggatatg

SEQ ID NO: 578 - DNA, 10 bp, Artificial Sequence
  Synthetic oligonucleotide
tggcgtaggt

SEQ ID NO: 579 - DNA, 10 bp, Artificial Sequence
  Synthetic oligonucleotide
actgaattcc

SEQ ID NO: 580 - DNA, 10 bp, Artificial Sequence
  Synthetic oligonucleotide
ttctacgcca

SEQ ID NO: 581 - DNA, 10 bp, Artificial Sequence
  Synthetic oligonucleotide
tggccgcaat

SEQ ID NO: 582 - DNA, 10 bp, Artificial Sequence
  Synthetic oligonucleotide
ccaagctgaa

SEQ ID NO: 583 - DNA, 10 bp, Artificial Sequence
  Synthetic oligonucleotide
tgccagatgg

SEQ ID NO: 584 - DNA, 10 bp, Artificial Sequence
  Synthetic oligonucleotide
aacttataag

SEQ ID NO: 585 - DNA, 10 bp, Artificial Sequence
  Synthetic oligonucleotide
tcatctaatc

SEQ ID NO: 586 - DNA, 10 bp, Artificial Sequence
  Synthetic oligonucleotide
agatcctata

SEQ ID NO: 587 - DNA, 10 bp, Artificial Sequence
  Synthetic oligonucleotide
cctataagct

SEQ ID NO: 588 - DNA, 10 bp, Artificial Sequence
  Synthetic oligonucleotide
gattgcagac

SEQ ID NO: 589 - DNA, 10 bp, Artificial Sequence
  Synthetic oligonucleotide
ataagaggcg

SEQ ID NO: 590 - DNA, 10 bp, Artificial Sequence
  Synthetic oligonucleotide
aagatccaac

SEQ ID NO: 591 - DNA, 10 bp, Artificial Sequence
  Synthetic oligonucleotide
ctgcaatcgc

SEQ ID NO: 592 - DNA, 49 bp, Artificial Sequence
  Synthetic oligonucleotide
caagcagaagacggcatacgagatccgaatccgagtctcgtgggctcgg

SEQ ID NO: 593 - DNA, 49 bp, Artificial Sequence
  Synthetic oligonucleotide
caagcagaagacggcatacgagatataagccggagtctcgtgggctcgg

SEQ ID NO: 594 - DNA, 49 bp, Artificial Sequence
  Synthetic oligonucleotide
caagcagaagacggcatacgagatccggcggcgagtctcgtgggctcgg

SEQ ID NO: 595 - DNA, 49 bp, Artificial Sequence
  Synthetic oligonucleotide
caagcagaagacggcatacgagatggcttgccaagtctcgtgggctcgg

SEQ ID NO: 596 - DNA, 49 bp, Artificial Sequence
  Synthetic oligonucleotide
caagcagaagacggcatacgagatccgctagctggtctcgtgggctcgg

SEQ ID NO: 597 - DNA, 49 bp, Artificial Sequence
  Synthetic oligonucleotide
caagcagaagacggcatacgagatcttatcctacgtctcgtgggctcgg

SEQ ID NO: 598 - DNA, 49 bp, Artificial Sequence
  Synthetic oligonucleotide
caagcagaagacggcatacgagattgagctacttgtctcgtgggctcgg

SEQ ID NO: 599 - DNA, 49 bp, Artificial Sequence
  Synthetic oligonucleotide
caagcagaagacggcatacgagattcaggacttagtctcgtgggctcgg

SEQ ID NO: 600 - DNA, 49 bp, Artificial Sequence
  Synthetic oligonucleotide
caagcagaagacggcatacgagatccgcagccgcgtctcgtgggctcgg

SEQ ID NO: 601 - DNA, 49 bp, Artificial Sequence
  Synthetic oligonucleotide
caagcagaagacggcatacgagattgcgcctggtgtctcgtgggctcgg

SEQ ID NO: 602 - DNA, 49 bp, Artificial Sequence
  Synthetic oligonucleotide
caagcagaagacggcatacgagataatcatacgggtctcgtgggctcgg

SEQ ID NO: 603 - DNA, 49 bp, Artificial Sequence
  Synthetic oligonucleotide
caagcagaagacggcatacgagatcgccaatcaagtctcgtgggctcgg

SEQ ID NO: 604 - DNA, 49 bp, Artificial Sequence
  Synthetic oligonucleotide
caagcagaagacggcatacgagatcaaggcttaggtctcgtgggctcgg

SEQ ID NO: 605 - DNA, 49 bp, Artificial Sequence
  Synthetic oligonucleotide
caagcagaagacggcatacgagatgcgctcgacggtctcgtgggctcgg

SEQ ID NO: 606 - DNA, 49 bp, Artificial Sequence
  Synthetic oligonucleotide
caagcagaagacggcatacgagattccagcaatagtctcgtgggctcgg

SEQ ID NO: 607 - DNA, 49 bp, Artificial Sequence
  Synthetic oligonucleotide
caagcagaagacggcatacgagatcatgagaactgtctcgtgggctcgg

SEQ ID NO: 608 - DNA, 49 bp, Artificial Sequence
  Synthetic oligonucleotide
caagcagaagacggcatacgagataacgtaatctgtctcgtgggctcgg

SEQ ID NO: 609 - DNA, 49 bp, Artificial Sequence
  Synthetic oligonucleotide
caagcagaagacggcatacgagatattctcctctgtctcgtgggctcgg

SEQ ID NO: 610 - DNA, 49 bp, Artificial Sequence
  Synthetic oligonucleotide
caagcagaagacggcatacgagattctgcgcgttgtctcgtgggctcgg

SEQ ID NO: 611 - DNA, 49 bp, Artificial Sequence
  Synthetic oligonucleotide
caagcagaagacggcatacgagatgctcatatgcgtctcgtgggctcgg

SEQ ID NO: 612 - DNA, 49 bp, Artificial Sequence
  Synthetic oligonucleotide
caagcagaagacggcatacgagatagcggtaacggtctcgtgggctcgg

SEQ ID NO: 613 - DNA, 49 bp, Artificial Sequence
  Synthetic oligonucleotide
caagcagaagacggcatacgagataatgaatagtgtctcgtgggctcgg

SEQ ID NO: 614 - DNA, 49 bp, Artificial Sequence
  Synthetic oligonucleotide
caagcagaagacggcatacgagatccgtatctgggtctcgtgggctcgg

SEQ ID NO: 615 - DNA, 49 bp, Artificial Sequence
  Synthetic oligonucleotide
caagcagaagacggcatacgagatccttagtctggtctcgtgggctcgg

SEQ ID NO: 616 - DNA, 49 bp, Artificial Sequence
  Synthetic oligonucleotide
caagcagaagacggcatacgagatacctagttaggtctcgtgggctcgg

SEQ ID NO: 617 - DNA, 49 bp, Artificial Sequence
  Synthetic oligonucleotide
caagcagaagacggcatacgagatataggagtacgtctcgtgggctcgg

SEQ ID NO: 618 - DNA, 49 bp, Artificial Sequence
  Synthetic oligonucleotide
caagcagaagacggcatacgagatctacgacgaggtctcgtgggctcgg

SEQ ID NO: 619 - DNA, 49 bp, Artificial Sequence
  Synthetic oligonucleotide
caagcagaagacggcatacgagatagtcgagttcgtctcgtgggctcgg

SEQ ID NO: 620 - DNA, 49 bp, Artificial Sequence
  Synthetic oligonucleotide
caagcagaagacggcatacgagattggtccagtcgtctcgtgggctcgg

SEQ ID NO: 621 - DNA, 49 bp, Artificial Sequence
  Synthetic oligonucleotide
caagcagaagacggcatacgagatatctaagcaagtctcgtgggctcgg

SEQ ID NO: 622 - DNA, 49 bp, Artificial Sequence
  Synthetic oligonucleotide
caagcagaagacggcatacgagatcgaattcgttgtctcgtgggctcgg

SEQ ID NO: 623 - DNA, 49 bp, Artificial Sequence
  Synthetic oligonucleotide
caagcagaagacggcatacgagatcagcgatagagtctcgtgggctcgg

SEQ ID NO: 624 - DNA, 49 bp, Artificial Sequence
  Synthetic oligonucleotide
caagcagaagacggcatacgagatggtcgctatggtctcgtgggctcgg

SEQ ID NO: 625 - DNA, 49 bp, Artificial Sequence
  Synthetic oligonucleotide
caagcagaagacggcatacgagatatccgttagcgtctcgtgggctcgg

SEQ ID NO: 626 - DNA, 49 bp, Artificial Sequence
  Synthetic oligonucleotide
caagcagaagacggcatacgagattcgcaattaggtctcgtgggctcgg

SEQ ID NO: 627 - DNA, 49 bp, Artificial Sequence
  Synthetic oligonucleotide
caagcagaagacggcatacgagatggctggctaggtctcgtgggctcgg

SEQ ID NO: 628 - DNA, 49 bp, Artificial Sequence
  Synthetic oligonucleotide
caagcagaagacggcatacgagatacggtcttgcgtctcgtgggctcgg

SEQ ID NO: 629 - DNA, 49 bp, Artificial Sequence
  Synthetic oligonucleotide
caagcagaagacggcatacgagatgctccattcggtctcgtgggctcgg

SEQ ID NO: 630 - DNA, 49 bp, Artificial Sequence
  Synthetic oligonucleotide
caagcagaagacggcatacgagatacgataagcggtctcgtgggctcgg

SEQ ID NO: 631 - DNA, 49 bp, Artificial Sequence
  Synthetic oligonucleotide
caagcagaagacggcatacgagataccatagcgcgtctcgtgggctcgg

SEQ ID NO: 632 - DNA, 49 bp, Artificial Sequence
  Synthetic oligonucleotide
caagcagaagacggcatacgagatctcttagcgggtctcgtgggctcgg

SEQ ID NO: 633 - DNA, 49 bp, Artificial Sequence
  Synthetic oligonucleotide
caagcagaagacggcatacgagattgattcaactgtctcgtgggctcgg

SEQ ID NO: 634 - DNA, 49 bp, Artificial Sequence
  Synthetic oligonucleotide
caagcagaagacggcatacgagattatggccgcggtctcgtgggctcgg

SEQ ID NO: 635 - DNA, 49 bp, Artificial Sequence
  Synthetic oligonucleotide
caagcagaagacggcatacgagatagaggtcgcagtctcgtgggctcgg

SEQ ID NO: 636 - DNA, 49 bp, Artificial Sequence
  Synthetic oligonucleotide
caagcagaagacggcatacgagataggagattgagtctcgtgggctcgg

SEQ ID NO: 637 - DNA, 49 bp, Artificial Sequence
  Synthetic oligonucleotide
caagcagaagacggcatacgagatggctatataggtctcgtgggctcgg

SEQ ID NO: 638 - DNA, 49 bp, Artificial Sequence
  Synthetic oligonucleotide
caagcagaagacggcatacgagattcgcgtacttgtctcgtgggctcgg

SEQ ID NO: 639 - DNA, 49 bp, Artificial Sequence
  Synthetic oligonucleotide
caagcagaagacggcatacgagataataataatggtctcgtgggctcgg

SEQ ID NO: 640 - DNA, 49 bp, Artificial Sequence
  Synthetic oligonucleotide
caagcagaagacggcatacgagatttcgttccatgtctcgtgggctcgg

SEQ ID NO: 641 - DNA, 49 bp, Artificial Sequence
  Synthetic oligonucleotide
caagcagaagacggcatacgagattacctaatcagtctcgtgggctcgg

SEQ ID NO: 642 - DNA, 49 bp, Artificial Sequence
  Synthetic oligonucleotide
caagcagaagacggcatacgagataagtaatattgtctcgtgggctcgg

SEQ ID NO: 643 - DNA, 49 bp, Artificial Sequence
  Synthetic oligonucleotide
caagcagaagacggcatacgagatagctaagaatgtctcgtgggctcgg

SEQ ID NO: 644 - DNA, 49 bp, Artificial Sequence
  Synthetic oligonucleotide
caagcagaagacggcatacgagatgtcgaggtatgtctcgtgggctcgg

SEQ ID NO: 645 - DNA, 49 bp, Artificial Sequence
  Synthetic oligonucleotide
caagcagaagacggcatacgagatttattagtaggtctcgtgggctcgg

SEQ ID NO: 646 - DNA, 49 bp, Artificial Sequence
  Synthetic oligonucleotide
caagcagaagacggcatacgagattgcgaagatcgtctcgtgggctcgg

SEQ ID NO: 647 - DNA, 49 bp, Artificial Sequence
  Synthetic oligonucleotide
caagcagaagacggcatacgagataactacggctgtctcgtgggctcgg

SEQ ID NO: 648 - DNA, 49 bp, Artificial Sequence
  Synthetic oligonucleotide
caagcagaagacggcatacgagataacggaacgcgtctcgtgggctcgg

SEQ ID NO: 649 - DNA, 49 bp, Artificial Sequence
  Synthetic oligonucleotide
caagcagaagacggcatacgagatgatgctacgagtctcgtgggctcgg

SEQ ID NO: 650 - DNA, 49 bp, Artificial Sequence
  Synthetic oligonucleotide
caagcagaagacggcatacgagatatctgccaatgtctcgtgggctcgg

SEQ ID NO: 651 - DNA, 49 bp, Artificial Sequence
  Synthetic oligonucleotide
caagcagaagacggcatacgagatatcgtatcaagtctcgtgggctcgg

SEQ ID NO: 652 - DNA, 49 bp, Artificial Sequence
  Synthetic oligonucleotide
caagcagaagacggcatacgagataacgcctctagtctcgtgggctcgg

SEQ ID NO: 653 - DNA, 49 bp, Artificial Sequence
  Synthetic oligonucleotide
caagcagaagacggcatacgagatacggcaaccagtctcgtgggctcgg

SEQ ID NO: 654 - DNA, 49 bp, Artificial Sequence
  Synthetic oligonucleotide
caagcagaagacggcatacgagatcaggctaagagtctcgtgggctcgg

SEQ ID NO: 655 - DNA, 49 bp, Artificial Sequence
  Synthetic oligonucleotide
caagcagaagacggcatacgagatcgcaatatcagtctcgtgggctcgg

SEQ ID NO: 656 - DNA, 49 bp, Artificial Sequence
  Synthetic oligonucleotide
caagcagaagacggcatacgagatttcgataaccgtctcgtgggctcgg

SEQ ID NO: 657 - DNA, 49 bp, Artificial Sequence
  Synthetic oligonucleotide
caagcagaagacggcatacgagataacctcaagagtctcgtgggctcgg

SEQ ID NO: 658 - DNA, 49 bp, Artificial Sequence
  Synthetic oligonucleotide
caagcagaagacggcatacgagatcaggcgccatgtctcgtgggctcgg

SEQ ID NO: 659 - DNA, 49 bp, Artificial Sequence
  Synthetic oligonucleotide
caagcagaagacggcatacgagataactattatagtctcgtgggctcgg

SEQ ID NO: 660 - DNA, 49 bp, Artificial Sequence
  Synthetic oligonucleotide
caagcagaagacggcatacgagataagttacctagtctcgtgggctcgg

SEQ ID NO: 661 - DNA, 49 bp, Artificial Sequence
  Synthetic oligonucleotide
caagcagaagacggcatacgagatcggcagaggagtctcgtgggctcgg

SEQ ID NO: 662 - DNA, 49 bp, Artificial Sequence
  Synthetic oligonucleotide
caagcagaagacggcatacgagatgcctcaataagtctcgtgggctcgg

SEQ ID NO: 663 - DNA, 49 bp, Artificial Sequence
  Synthetic oligonucleotide
caagcagaagacggcatacgagatttaacgccgtgtctcgtgggctcgg

SEQ ID NO: 664 - DNA, 49 bp, Artificial Sequence
  Synthetic oligonucleotide
caagcagaagacggcatacgagatcatacgatgcgtctcgtgggctcgg

SEQ ID NO: 665 - DNA, 49 bp, Artificial Sequence
  Synthetic oligonucleotide
caagcagaagacggcatacgagataagctgacctgtctcgtgggctcgg

SEQ ID NO: 666 - DNA, 49 bp, Artificial Sequence
  Synthetic oligonucleotide
caagcagaagacggcatacgagatgagtccttatgtctcgtgggctcgg

SEQ ID NO: 667 - DNA, 49 bp, Artificial Sequence
  Synthetic oligonucleotide
caagcagaagacggcatacgagatcctacggcaagtctcgtgggctcgg

SEQ ID NO: 668 - DNA, 49 bp, Artificial Sequence
  Synthetic oligonucleotide
caagcagaagacggcatacgagataatattcgaagtctcgtgggctcgg

SEQ ID NO: 669 - DNA, 49 bp, Artificial Sequence
  Synthetic oligonucleotide
caagcagaagacggcatacgagatttcaagaatcgtctcgtgggctcgg

SEQ ID NO: 670 - DNA, 49 bp, Artificial Sequence
  Synthetic oligonucleotide
caagcagaagacggcatacgagatatgctcgcaagtctcgtgggctcgg

SEQ ID NO: 671 - DNA, 49 bp, Artificial Sequence
  Synthetic oligonucleotide
caagcagaagacggcatacgagatggagtaagccgtctcgtgggctcgg

SEQ ID NO: 672 - DNA, 49 bp, Artificial Sequence
  Synthetic oligonucleotide
caagcagaagacggcatacgagatttatcgtattgtctcgtgggctcgg

SEQ ID NO: 673 - DNA, 49 bp, Artificial Sequence
  Synthetic oligonucleotide
caagcagaagacggcatacgagataagtctaatagtctcgtgggctcgg

SEQ ID NO: 674 - DNA, 49 bp, Artificial Sequence
  Synthetic oligonucleotide
caagcagaagacggcatacgagatcggcttactagtctcgtgggctcgg

SEQ ID NO: 675 - DNA, 49 bp, Artificial Sequence
  Synthetic oligonucleotide
caagcagaagacggcatacgagatgatatggtctgtctcgtgggctcgg

SEQ ID NO: 676 - DNA, 49 bp, Artificial Sequence
  Synthetic oligonucleotide
caagcagaagacggcatacgagattagtcgtccagtctcgtgggctcgg

SEQ ID NO: 677 - DNA, 49 bp, Artificial Sequence
  Synthetic oligonucleotide
caagcagaagacggcatacgagattagctgctacgtctcgtgggctcgg

SEQ ID NO: 678 - DNA, 49 bp, Artificial Sequence
  Synthetic oligonucleotide
caagcagaagacggcatacgagatctcttcaagcgtctcgtgggctcgg

SEQ ID NO: 679 - DNA, 49 bp, Artificial Sequence
  Synthetic oligonucleotide
caagcagaagacggcatacgagatatgaacgcgcgtctcgtgggctcgg

SEQ ID NO: 680 - DNA, 49 bp, Artificial Sequence
  Synthetic oligonucleotide
caagcagaagacggcatacgagatgtcgacggaagtctcgtgggctcgg

SEQ ID NO: 681 - DNA, 49 bp, Artificial Sequence
  Synthetic oligonucleotide
caagcagaagacggcatacgagatactaattgaggtctcgtgggctcgg

SEQ ID NO: 682 - DNA, 49 bp, Artificial Sequence
  Synthetic oligonucleotide
caagcagaagacggcatacgagatcttgcataatgtctcgtgggctcgg

SEQ ID NO: 683 - DNA, 49 bp, Artificial Sequence
  Synthetic oligonucleotide
caagcagaagacggcatacgagattccttaccaagtctcgtgggctcgg

SEQ ID NO: 684 - DNA, 49 bp, Artificial Sequence
  Synthetic oligonucleotide
caagcagaagacggcatacgagattgcagcctacgtctcgtgggctcgg

SEQ ID NO: 685 - DNA, 49 bp, Artificial Sequence
  Synthetic oligonucleotide
caagcagaagacggcatacgagatggagctgagggtctcgtgggctcgg

SEQ ID NO: 686 - DNA, 49 bp, Artificial Sequence
  Synthetic oligonucleotide
caagcagaagacggcatacgagatgcagcggactgtctcgtgggctcgg

SEQ ID NO: 687 - DNA, 49 bp, Artificial Sequence
  Synthetic oligonucleotide
caagcagaagacggcatacgagatcatcgcgctcgtctcgtgggctcgg

SEQ ID NO: 688 - DNA, 10 bp, Artificial Sequence
  Synthetic oligonucleotide
ccgaatccga

SEQ ID NO: 689 - DNA, 10 bp, Artificial Sequence
  Synthetic oligonucleotide
ataagccgga

SEQ ID NO: 690 - DNA, 10 bp, Artificial Sequence
  Synthetic oligonucleotide
ccggcggcga

SEQ ID NO: 691 - DNA, 10 bp, Artificial Sequence
  Synthetic oligonucleotide
ggcttgccaa

SEQ ID NO: 692 - DNA, 10 bp, Artificial Sequence
  Synthetic oligonucleotide
ccgctagctg

SEQ ID NO: 693 - DNA, 10 bp, Artificial Sequence
  Synthetic oligonucleotide
cttatcctac

SEQ ID NO: 694 - DNA, 10 bp, Artificial Sequence
  Synthetic oligonucleotide
tgagctactt

SEQ ID NO: 695 - DNA, 10 bp, Artificial Sequence
  Synthetic oligonucleotide
tcaggactta

SEQ ID NO: 696 - DNA, 10 bp, Artificial Sequence
  Synthetic oligonucleotide
ccgcagccgc

SEQ ID NO: 697 - DNA, 10 bp, Artificial Sequence
  Synthetic oligonucleotide
tgcgcctggt

SEQ ID NO: 698 - DNA, 10 bp, Artificial Sequence
  Synthetic oligonucleotide
aatcatacgg

SEQ ID NO: 699 - DNA, 10 bp, Artificial Sequence
  Synthetic oligonucleotide
cgccaatcaa

SEQ ID NO: 700 - DNA, 10 bp, Artificial Sequence
  Synthetic oligonucleotide
caaggcttag

SEQ ID NO: 701 - DNA, 10 bp, Artificial Sequence
  Synthetic oligonucleotide
gcgctcgacg

SEQ ID NO: 702 - DNA, 10 bp, Artificial Sequence
  Synthetic oligonucleotide
tccagcaata

SEQ ID NO: 703 - DNA, 10 bp, Artificial Sequence
  Synthetic oligonucleotide
catgagaact

SEQ ID NO: 704 - DNA, 10 bp, Artificial Sequence
  Synthetic oligonucleotide
aacgtaatct

SEQ ID NO: 705 - DNA, 10 bp, Artificial Sequence
  Synthetic oligonucleotide
attctcctct

SEQ ID NO: 706 - DNA, 10 bp, Artificial Sequence
  Synthetic oligonucleotide
tctgcgcgtt

SEQ ID NO: 707 - DNA, 10 bp, Artificial Sequence
  Synthetic oligonucleotide
gctcatatgc

SEQ ID NO: 708 - DNA, 10 bp, Artificial Sequence
  Synthetic oligonucleotide
agcggtaacg

SEQ ID NO: 709 - DNA, 10 bp, Artificial Sequence
  Synthetic oligonucleotide
aatgaatagt

SEQ ID NO: 710 - DNA, 10 bp, Artificial Sequence
  Synthetic oligonucleotide
ccgtatctgg

SEQ ID NO: 711 - DNA, 10 bp, Artificial Sequence
  Synthetic oligonucleotide
ccttagtctg

SEQ ID NO: 712 - DNA, 10 bp, Artificial Sequence
  Synthetic oligonucleotide
acctagttag

SEQ ID NO: 713 - DNA, 10 bp, Artificial Sequence
  Synthetic oligonucleotide
ataggagtac

SEQ ID NO: 714 - DNA, 10 bp, Artificial Sequence
  Synthetic oligonucleotide
ctacgacgag

SEQ ID NO: 715 - DNA, 10 bp, Artificial Sequence
  Synthetic oligonucleotide
agtcgagttc

SEQ ID NO: 716 - DNA, 10 bp, Artificial Sequence
  Synthetic oligonucleotide
tggtccagtc

SEQ ID NO: 717 - DNA, 10 bp, Artificial Sequence
  Synthetic oligonucleotide
atctaagcaa

SEQ ID NO: 718 - DNA, 10 bp, Artificial Sequence
  Synthetic oligonucleotide
cgaattcgtt

SEQ ID NO: 719 - DNA, 10 bp, Artificial Sequence
  Synthetic oligonucleotide
cagcgataga

SEQ ID NO: 720 - DNA, 10 bp, Artificial Sequence
  Synthetic oligonucleotide
ggtcgctatg

SEQ ID NO: 721 - DNA, 10 bp, Artificial Sequence
  Synthetic oligonucleotide
atccgttagc

SEQ ID NO: 722 - DNA, 10 bp, Artificial Sequence
  Synthetic oligonucleotide
tcgcaattag

SEQ ID NO: 723 - DNA, 10 bp, Artificial Sequence
  Synthetic oligonucleotide
ggctggctag

SEQ ID NO: 724 - DNA, 10 bp, Artificial Sequence
  Synthetic oligonucleotide
acggtcttgc

SEQ ID NO: 725 - DNA, 10 bp, Artificial Sequence
  Synthetic oligonucleotide
gctccattcg

SEQ ID NO: 726 - DNA, 10 bp, Artificial Sequence
  Synthetic oligonucleotide
acgataagcg

SEQ ID NO: 727 - DNA, 10 bp, Artificial Sequence
  Synthetic oligonucleotide
accatagcgc

SEQ ID NO: 728 - DNA, 10 bp, Artificial Sequence
  Synthetic oligonucleotide
ctcttagcgg

SEQ ID NO: 729 - DNA, 10 bp, Artificial Sequence
  Synthetic oligonucleotide
tgattcaact

SEQ ID NO: 730 - DNA, 10 bp, Artificial Sequence
  Synthetic oligonucleotide
tatggccgcg

SEQ ID NO: 731 - DNA, 10 bp, Artificial Sequence
  Synthetic oligonucleotide
agaggtcgca

SEQ ID NO: 732 - DNA, 10 bp, Artificial Sequence
  Synthetic oligonucleotide
aggagattga

SEQ ID NO: 733 - DNA, 10 bp, Artificial Sequence
  Synthetic oligonucleotide
ggctatatag

SEQ ID NO: 734 - DNA, 10 bp, Artificial Sequence
  Synthetic oligonucleotide
tcgcgtactt

SEQ ID NO: 735 - DNA, 10 bp, Artificial Sequence
  Synthetic oligonucleotide
aataataatg

SEQ ID NO: 736 - DNA, 10 bp, Artificial Sequence
  Synthetic oligonucleotide
ttcgttccat

SEQ ID NO: 737 - DNA, 10 bp, Artificial Sequence
  Synthetic oligonucleotide
tacctaatca

SEQ ID NO: 738 - DNA, 10 bp, Artificial Sequence
  Synthetic oligonucleotide
aagtaatatt

SEQ ID NO: 739 - DNA, 10 bp, Artificial Sequence
  Synthetic oligonucleotide
agctaagaat

SEQ ID NO: 740 - DNA, 10 bp, Artificial Sequence
  Synthetic oligonucleotide
gtcgaggtat

SEQ ID NO: 741 - DNA, 10 bp, Artificial Sequence
  Synthetic oligonucleotide
ttattagtag

SEQ ID NO: 742 - DNA, 10 bp, Artificial Sequence
  Synthetic oligonucleotide
tgcgaagatc

SEQ ID NO: 743 - DNA, 10 bp, Artificial Sequence
  Synthetic oligonucleotide
aactacggct

SEQ ID NO: 744 - DNA, 10 bp, Artificial Sequence
  Synthetic oligonucleotide
aacggaacgc

SEQ ID NO: 745 - DNA, 10 bp, Artificial Sequence
  Synthetic oligonucleotide
gatgctacga

SEQ ID NO: 746 - DNA, 10 bp, Artificial Sequence
  Synthetic oligonucleotide
atctgccaat

SEQ ID NO: 747 - DNA, 10 bp, Artificial Sequence
  Synthetic oligonucleotide
atcgtatcaa

SEQ ID NO: 748 - DNA, 10 bp, Artificial Sequence
  Synthetic oligonucleotide
aacgcctcta

SEQ ID NO: 749 - DNA, 10 bp, Artificial Sequence
  Synthetic oligonucleotide
acggcaacca

SEQ ID NO: 750 - DNA, 10 bp, Artificial Sequence
  Synthetic oligonucleotide
caggctaaga

SEQ ID NO: 751 - DNA, 10 bp, Artificial Sequence
  Synthetic oligonucleotide
cgcaatatca

SEQ ID NO: 752 - DNA, 10 bp, Artificial Sequence
  Synthetic oligonucleotide
ttcgataacc

SEQ ID NO: 753 - DNA, 10 bp, Artificial Sequence
  Synthetic oligonucleotide
aacctcaaga

SEQ ID NO: 754 - DNA, 10 bp, Artificial Sequence
  Synthetic oligonucleotide
caggcgccat

SEQ ID NO: 755 - DNA, 10 bp, Artificial Sequence
  Synthetic oligonucleotide
aactattata

SEQ ID NO: 756 - DNA, 10 bp, Artificial Sequence
  Synthetic oligonucleotide
aagttaccta

SEQ ID NO: 757 - DNA, 10 bp, Artificial Sequence
  Synthetic oligonucleotide
cggcagagga

SEQ ID NO: 758 - DNA, 10 bp, Artificial Sequence
  Synthetic oligonucleotide
gcctcaataa

SEQ ID NO: 759 - DNA, 10 bp, Artificial Sequence
  Synthetic oligonucleotide
ttaacgccgt

SEQ ID NO: 760 - DNA, 10 bp, Artificial Sequence
  Synthetic oligonucleotide
catacgatgc

SEQ ID NO: 761 - DNA, 10 bp, Artificial Sequence
  Synthetic oligonucleotide
aagctgacct

SEQ ID NO: 762 - DNA, 10 bp, Artificial Sequence
  Synthetic oligonucleotide
gagtccttat

SEQ ID NO: 763 - DNA, 10 bp, Artificial Sequence
  Synthetic oligonucleotide
cctacggcaa

SEQ ID NO: 764 - DNA, 10 bp, Artificial Sequence
  Synthetic oligonucleotide
aatattcgaa

SEQ ID NO: 765 - DNA, 10 bp, Artificial Sequence
  Synthetic oligonucleotide
ttcaagaatc

SEQ ID NO: 766 - DNA, 10 bp, Artificial Sequence
  Synthetic oligonucleotide
atgctcgcaa

SEQ ID NO: 767 - DNA, 10 bp, Artificial Sequence
  Synthetic oligonucleotide
ggagtaagcc

SEQ ID NO: 768 - DNA, 10 bp, Artificial Sequence
  Synthetic oligonucleotide
ttatcgtatt

SEQ ID NO: 769 - DNA, 10 bp, Artificial Sequence
  Synthetic oligonucleotide
aagtctaata

SEQ ID NO: 770 - DNA, 10 bp, Artificial Sequence
  Synthetic oligonucleotide
cggcttacta

SEQ ID NO: 771 - DNA, 10 bp, Artificial Sequence
  Synthetic oligonucleotide
gatatggtct

SEQ ID NO: 772 - DNA, 10 bp, Artificial Sequence
  Synthetic oligonucleotide
tagtcgtcca

SEQ ID NO: 773 - DNA, 10 bp, Artificial Sequence
  Synthetic oligonucleotide
tagctgctac

SEQ ID NO: 774 - DNA, 10 bp, Artificial Sequence
  Synthetic oligonucleotide
ctcttcaagc

SEQ ID NO: 775 - DNA, 10 bp, Artificial Sequence
  Synthetic oligonucleotide
atgaacgcgc

SEQ ID NO: 776 - DNA, 10 bp, Artificial Sequence
  Synthetic oligonucleotide
gtcgacggaa

SEQ ID NO: 777 - DNA, 10 bp, Artificial Sequence
  Synthetic oligonucleotide
actaattgag

SEQ ID NO: 778 - DNA, 10 bp, Artificial Sequence
  Synthetic oligonucleotide
cttgcataat

SEQ ID NO: 779 - DNA, 10 bp, Artificial Sequence
  Synthetic oligonucleotide
tccttaccaa

SEQ ID NO: 780 - DNA, 10 bp, Artificial Sequence
  Synthetic oligonucleotide
tgcagcctac

SEQ ID NO: 781 - DNA, 10 bp, Artificial Sequence
  Synthetic oligonucleotide
ggagctgagg

SEQ ID NO: 782 - DNA, 10 bp, Artificial Sequence
  Synthetic oligonucleotide
gcagcggact

SEQ ID NO: 783 - DNA, 10 bp, Artificial Sequence
  Synthetic oligonucleotide
catcgcgctc

SEQ ID NO: 784 - DNA, 61 bp, Artificial Sequence
  Synthetic oligonucleotide
ccatctcatccctgcgtgtctccgactcagctccatcgaggatacgacgctcttccgatct

SEQ ID NO: 785 - DNA, 61 bp, Artificial Sequence
  Synthetic oligonucleotide
ccatctcatccctgcgtgtctccgactcagttggtagtcggatacgacgctcttccgatct

SEQ ID NO: 786 - DNA, 61 bp, Artificial Sequence
  Synthetic oligonucleotide
ccatctcatccctgcgtgtctccgactcagggccgtcaacgatacgacgctcttccgatct

SEQ ID NO: 787 - DNA, 61 bp, Artificial Sequence
  Synthetic oligonucleotide
ccatctcatccctgcgtgtctccgactcagcctagacgaggatacgacgctcttccgatct

SEQ ID NO: 788 - DNA, 61 bp, Artificial Sequence
  Synthetic oligonucleotide
ccatctcatccctgcgtgtctccgactcagtcgttagagcgatacgacgctcttccgatct

SEQ ID NO: 789 - DNA, 61 bp, Artificial Sequence
  Synthetic oligonucleotide
ccatctcatccctgcgtgtctccgactcagcgttctatcagatacgacgctcttccgatct

SEQ ID NO: 790 - DNA, 61 bp, Artificial Sequence
  Synthetic oligonucleotide
ccatctcatccctgcgtgtctccgactcagcggaatctaagatacgacgctcttccgatct

SEQ ID NO: 791 - DNA, 61 bp, Artificial Sequence
  Synthetic oligonucleotide
ccatctcatccctgcgtgtctccgactcagatgactgatcgatacgacgctcttccgatct

SEQ ID NO: 792 - DNA, 61 bp, Artificial Sequence
  Synthetic oligonucleotide
ccatctcatccctgcgtgtctccgactcagtcaatatcgagatacgacgctcttccgatct

SEQ ID NO: 793 - DNA, 61 bp, Artificial Sequence
  Synthetic oligonucleotide
ccatctcatccctgcgtgtctccgactcaggtagacctgggatacgacgctcttccgatct

SEQ ID NO: 794 - DNA, 61 bp, Artificial Sequence
  Synthetic oligonucleotide
ccatctcatccctgcgtgtctccgactcagttatgaccaagatacgacgctcttccgatct

SEQ ID NO: 795 - DNA, 61 bp, Artificial Sequence
  Synthetic oligonucleotide
ccatctcatccctgcgtgtctccgactcagttggtccgttgatacgacgctcttccgatct

SEQ ID NO: 796 - DNA, 61 bp, Artificial Sequence
  Synthetic oligonucleotide
ccatctcatccctgcgtgtctccgactcagggtacgttaagatacgacgctcttccgatct

SEQ ID NO: 797 - DNA, 61 bp, Artificial Sequence
  Synthetic oligonucleotide
ccatctcatccctgcgtgtctccgactcagcaatgagtccgatacgacgctcttccgatct

SEQ ID NO: 798 - DNA, 61 bp, Artificial Sequence
  Synthetic oligonucleotide
ccatctcatccctgcgtgtctccgactcaggatgcagttcgatacgacgctcttccgatct

SEQ ID NO: 799 - DNA, 61 bp, Artificial Sequence
  Synthetic oligonucleotide
ccatctcatccctgcgtgtctccgactcagccatcgttccgatacgacgctcttccgatct

SEQ ID NO: 800 - DNA, 61 bp, Artificial Sequence
  Synthetic oligonucleotide
ccatctcatccctgcgtgtctccgactcagttgagagagtgatacgacgctcttccgatct

SEQ ID NO: 801 - DNA, 61 bp, Artificial Sequence
  Synthetic oligonucleotide
ccatctcatccctgcgtgtctccgactcagactgagcgacgatacgacgctcttccgatct

SEQ ID NO: 802 - DNA, 61 bp, Artificial Sequence
  Synthetic oligonucleotide
ccatctcatccctgcgtgtctccgactcagtgaggaatcagatacgacgctcttccgatct

SEQ ID NO: 803 - DNA, 61 bp, Artificial Sequence
  Synthetic oligonucleotide
ccatctcatccctgcgtgtctccgactcagcctccgacgggatacgacgctcttccgatct

SEQ ID NO: 804 - DNA, 61 bp, Artificial Sequence
  Synthetic oligonucleotide
ccatctcatccctgcgtgtctccgactcagcattgacgctgatacgacgctcttccgatct

SEQ ID NO: 805 - DNA, 61 bp, Artificial Sequence
  Synthetic oligonucleotide
ccatctcatccctgcgtgtctccgactcagtcgtccttcggatacgacgctcttccgatct

SEQ ID NO: 806 - DNA, 61 bp, Artificial Sequence
  Synthetic oligonucleotide
ccatctcatccctgcgtgtctccgactcagtgatactcaagatacgacgctcttccgatct

SEQ ID NO: 807 - DNA, 61 bp, Artificial Sequence
  Synthetic oligonucleotide
ccatctcatccctgcgtgtctccgactcagttctacctcagatacgacgctcttccgatct

SEQ ID NO: 808 - DNA, 61 bp, Artificial Sequence
  Synthetic oligonucleotide
ccatctcatccctgcgtgtctccgactcagtcgtcggaacgatacgacgctcttccgatct

SEQ ID NO: 809 - DNA, 61 bp, Artificial Sequence
  Synthetic oligonucleotide
ccatctcatccctgcgtgtctccgactcagatcgagatgagatacgacgctcttccgatct

SEQ ID NO: 810 - DNA, 61 bp, Artificial Sequence
  Synthetic oligonucleotide
ccatctcatccctgcgtgtctccgactcagtagactagtcgatacgacgctcttccgatct

SEQ ID NO: 811 - DNA, 61 bp, Artificial Sequence
  Synthetic oligonucleotide
ccatctcatccctgcgtgtctccgactcaggtcgaagcaggatacgacgctcttccgatct

SEQ ID NO: 812 - DNA, 61 bp, Artificial Sequence
  Synthetic oligonucleotide
ccatctcatccctgcgtgtctccgactcagaggcgctagggatacgacgctcttccgatct

SEQ ID NO: 813 - DNA, 61 bp, Artificial Sequence
  Synthetic oligonucleotide
ccatctcatccctgcgtgtctccgactcagagatgcaactgatacgacgctcttccgatct

SEQ ID NO: 814 - DNA, 61 bp, Artificial Sequence
  Synthetic oligonucleotide
ccatctcatccctgcgtgtctccgactcagaagcctacgagatacgacgctcttccgatct

SEQ ID NO: 815 - DNA, 61 bp, Artificial Sequence
  Synthetic oligonucleotide
ccatctcatccctgcgtgtctccgactcaggtaggcaattgatacgacgctcttccgatct

SEQ ID NO: 816 - DNA, 61 bp, Artificial Sequence
  Synthetic oligonucleotide
ccatctcatccctgcgtgtctccgactcagtgccagttgcgatacgacgctcttccgatct

SEQ ID NO: 817 - DNA, 61 bp, Artificial Sequence
  Synthetic oligonucleotide
ccatctcatccctgcgtgtctccgactcagcttaggtatcgatacgacgctcttccgatct

SEQ ID NO: 818 - DNA, 61 bp, Artificial Sequence
  Synthetic oligonucleotide
ccatctcatccctgcgtgtctccgactcaggagacctaccgatacgacgctcttccgatct

SEQ ID NO: 819 - DNA, 61 bp, Artificial Sequence
  Synthetic oligonucleotide
ccatctcatccctgcgtgtctccgactcagattgaccgaggatacgacgctcttccgatct

SEQ ID NO: 820 - DNA, 61 bp, Artificial Sequence
  Synthetic oligonucleotide
ccatctcatccctgcgtgtctccgactcagggaggcggcggatacgacgctcttccgatct

SEQ ID NO: 821 - DNA, 61 bp, Artificial Sequence
  Synthetic oligonucleotide
ccatctcatccctgcgtgtctccgactcagccagtacttggatacgacgctcttccgatct

SEQ ID NO: 822 - DNA, 61 bp, Artificial Sequence
  Synthetic oligonucleotide
ccatctcatccctgcgtgtctccgactcagggtctcgccggatacgacgctcttccgatct

SEQ ID NO: 823 - DNA, 61 bp, Artificial Sequence
  Synthetic oligonucleotide
ccatctcatccctgcgtgtctccgactcagggcggaggtcgatacgacgctcttccgatct

SEQ ID NO: 824 - DNA, 61 bp, Artificial Sequence
  Synthetic oligonucleotide
ccatctcatccctgcgtgtctccgactcagtagttctagagatacgacgctcttccgatct

SEQ ID NO: 825 - DNA, 61 bp, Artificial Sequence
  Synthetic oligonucleotide
ccatctcatccctgcgtgtctccgactcagttggagttaggatacgacgctcttccgatct

SEQ ID NO: 826 - DNA, 61 bp, Artificial Sequence
  Synthetic oligonucleotide
ccatctcatccctgcgtgtctccgactcagagatcttggtgatacgacgctcttccgatct

SEQ ID NO: 827 - DNA, 61 bp, Artificial Sequence
  Synthetic oligonucleotide
ccatctcatccctgcgtgtctccgactcaggtaatgatcggatacgacgctcttccgatct

SEQ ID NO: 828 - DNA, 61 bp, Artificial Sequence
  Synthetic oligonucleotide
ccatctcatccctgcgtgtctccgactcagcagagaggtcgatacgacgctcttccgatct

SEQ ID NO: 829 - DNA, 61 bp, Artificial Sequence
  Synthetic oligonucleotide
ccatctcatccctgcgtgtctccgactcagttaattagccgatacgacgctcttccgatct

SEQ ID NO: 830 - DNA, 61 bp, Artificial Sequence
  Synthetic oligonucleotide
ccatctcatccctgcgtgtctccgactcagctctaactcggatacgacgctcttccgatct

SEQ ID NO: 831 - DNA, 61 bp, Artificial Sequence
  Synthetic oligonucleotide
ccatctcatccctgcgtgtctccgactcagtacgatcatcgatacgacgctcttccgatct

SEQ ID NO: 832 - DNA, 61 bp, Artificial Sequence
  Synthetic oligonucleotide
ccatctcatccctgcgtgtctccgactcagaggcgagagcgatacgacgctcttccgatct

SEQ ID NO: 833 - DNA, 61 bp, Artificial Sequence
  Synthetic oligonucleotide
ccatctcatccctgcgtgtctccgactcagtcaagatagtgatacgacgctcttccgatct

SEQ ID NO: 834 - DNA, 61 bp, Artificial Sequence
  Synthetic oligonucleotide
ccatctcatccctgcgtgtctccgactcagtaattgacctgatacgacgctcttccgatct

SEQ ID NO: 835 - DNA, 61 bp, Artificial Sequence
  Synthetic oligonucleotide
ccatctcatccctgcgtgtctccgactcagcagccggcttgatacgacgctcttccgatct

SEQ ID NO: 836 - DNA, 61 bp, Artificial Sequence
  Synthetic oligonucleotide
ccatctcatccctgcgtgtctccgactcagagaaccggaggatacgacgctcttccgatct

SEQ ID NO: 837 - DNA, 61 bp, Artificial Sequence
  Synthetic oligonucleotide
ccatctcatccctgcgtgtctccgactcaggagatgcatggatacgacgctcttccgatct

SEQ ID NO: 838 - DNA, 61 bp, Artificial Sequence
  Synthetic oligonucleotide
ccatctcatccctgcgtgtctccgactcaggattaccggagatacgacgctcttccgatct

SEQ ID NO: 839 - DNA, 61 bp, Artificial Sequence
  Synthetic oligonucleotide
ccatctcatccctgcgtgtctccgactcagtcgtaacggtgatacgacgctcttccgatct

SEQ ID NO: 840 - DNA, 61 bp, Artificial Sequence
  Synthetic oligonucleotide
ccatctcatccctgcgtgtctccgactcagtggcgacggagatacgacgctcttccgatct

SEQ ID NO: 841 - DNA, 61 bp, Artificial Sequence
  Synthetic oligonucleotide
ccatctcatccctgcgtgtctccgactcagagtcatagccgatacgacgctcttccgatct

SEQ ID NO: 842 - DNA, 61 bp, Artificial Sequence
  Synthetic oligonucleotide
ccatctcatccctgcgtgtctccgactcaggtcaagtccagatacgacgctcttccgatct

SEQ ID NO: 843 - DNA, 61 bp, Artificial Sequence
  Synthetic oligonucleotide
ccatctcatccctgcgtgtctccgactcagattcggaagtgatacgacgctcttccgatct

SEQ ID NO: 844 - DNA, 61 bp, Artificial Sequence
  Synthetic oligonucleotide
ccatctcatccctgcgtgtctccgactcaggtcggtagttgatacgacgctcttccgatct

SEQ ID NO: 845 - DNA, 61 bp, Artificial Sequence
  Synthetic oligonucleotide
ccatctcatccctgcgtgtctccgactcagaggacggacggatacgacgctcttccgatct

SEQ ID NO: 846 - DNA, 61 bp, Artificial Sequence
  Synthetic oligonucleotide
ccatctcatccctgcgtgtctccgactcagctcctggaccgatacgacgctcttccgatct

SEQ ID NO: 847 - DNA, 61 bp, Artificial Sequence
  Synthetic oligonucleotide
ccatctcatccctgcgtgtctccgactcagtagcctcgttgatacgacgctcttccgatct

SEQ ID NO: 848 - DNA, 61 bp, Artificial Sequence
  Synthetic oligonucleotide
ccatctcatccctgcgtgtctccgactcagggttgaacgtgatacgacgctcttccgatct

SEQ ID NO: 849 - DNA, 61 bp, Artificial Sequence
  Synthetic oligonucleotide
ccatctcatccctgcgtgtctccgactcagaggtcctcgtgatacgacgctcttccgatct

SEQ ID NO: 850 - DNA, 61 bp, Artificial Sequence
  Synthetic oligonucleotide
ccatctcatccctgcgtgtctccgactcagggaagttatagatacgacgctcttccgatct

SEQ ID NO: 851 - DNA, 61 bp, Artificial Sequence
  Synthetic oligonucleotide
ccatctcatccctgcgtgtctccgactcagtggtaatcctgatacgacgctcttccgatct

SEQ ID NO: 852 - DNA, 61 bp, Artificial Sequence
  Synthetic oligonucleotide
ccatctcatccctgcgtgtctccgactcagaagctaggttgatacgacgctcttccgatct

SEQ ID NO: 853 - DNA, 61 bp, Artificial Sequence
  Synthetic oligonucleotide
ccatctcatccctgcgtgtctccgactcagtccgcggactgatacgacgctcttccgatct

SEQ ID NO: 854 - DNA, 61 bp, Artificial Sequence
  Synthetic oligonucleotide
ccatctcatccctgcgtgtctccgactcagtgcggatagtgatacgacgctcttccgatct

SEQ ID NO: 855 - DNA, 61 bp, Artificial Sequence
  Synthetic oligonucleotide
ccatctcatccctgcgtgtctccgactcagtggcagctcggatacgacgctcttccgatct

SEQ ID NO: 856 - DNA, 61 bp, Artificial Sequence
  Synthetic oligonucleotide
ccatctcatccctgcgtgtctccgactcagtgctacggtcgatacgacgctcttccgatct

SEQ ID NO: 857 - DNA, 61 bp, Artificial Sequence
  Synthetic oligonucleotide
ccatctcatccctgcgtgtctccgactcaggcgcaatgacgatacgacgctcttccgatct

SEQ ID NO: 858 - DNA, 61 bp, Artificial Sequence
  Synthetic oligonucleotide
ccatctcatccctgcgtgtctccgactcagcttaatcttggatacgacgctcttccgatct

SEQ ID NO: 859 - DNA, 61 bp, Artificial Sequence
  Synthetic oligonucleotide
ccatctcatccctgcgtgtctccgactcagggagttgcgtgatacgacgctcttccgatct

SEQ ID NO: 860 - DNA, 61 bp, Artificial Sequence
  Synthetic oligonucleotide
ccatctcatccctgcgtgtctccgactcagactcgtatcagatacgacgctcttccgatct

SEQ ID NO: 861 - DNA, 61 bp, Artificial Sequence
  Synthetic oligonucleotide
ccatctcatccctgcgtgtctccgactcagggtaataatggatacgacgctcttccgatct

SEQ ID NO: 862 - DNA, 61 bp, Artificial Sequence
  Synthetic oligonucleotide
ccatctcatccctgcgtgtctccgactcagtccttatagagatacgacgctcttccgatct

SEQ ID NO: 863 - DNA, 61 bp, Artificial Sequence
  Synthetic oligonucleotide
ccatctcatccctgcgtgtctccgactcagccgactccaagatacgacgctcttccgatct

SEQ ID NO: 864 - DNA, 61 bp, Artificial Sequence
  Synthetic oligonucleotide
ccatctcatccctgcgtgtctccgactcaggccaagcttggatacgacgctcttccgatct

SEQ ID NO: 865 - DNA, 61 bp, Artificial Sequence
  Synthetic oligonucleotide
ccatctcatccctgcgtgtctccgactcagcatatcctatgatacgacgctcttccgatct

SEQ ID NO: 866 - DNA, 61 bp, Artificial Sequence
  Synthetic oligonucleotide
ccatctcatccctgcgtgtctccgactcagacctacgccagatacgacgctcttccgatct

SEQ ID NO: 867 - DNA, 61 bp, Artificial Sequence
  Synthetic oligonucleotide
ccatctcatccctgcgtgtctccgactcagggaattcagtgatacgacgctcttccgatct

SEQ ID NO: 868 - DNA, 61 bp, Artificial Sequence
  Synthetic oligonucleotide
ccatctcatccctgcgtgtctccgactcagtggcgtagaagatacgacgctcttccgatct

SEQ ID NO: 869 - DNA, 61 bp, Artificial Sequence
  Synthetic oligonucleotide
ccatctcatccctgcgtgtctccgactcagattgcggccagatacgacgctcttccgatct

SEQ ID NO: 870 - DNA, 61 bp, Artificial Sequence
  Synthetic oligonucleotide
ccatctcatccctgcgtgtctccgactcagttcagcttgggatacgacgctcttccgatct

SEQ ID NO: 871 - DNA, 61 bp, Artificial Sequence
  Synthetic oligonucleotide
ccatctcatccctgcgtgtctccgactcagccatctggcagatacgacgctcttccgatct

SEQ ID NO: 872 - DNA, 61 bp, Artificial Sequence
  Synthetic oligonucleotide
ccatctcatccctgcgtgtctccgactcagcttataagttgatacgacgctcttccgatct

SEQ ID NO: 873 - DNA, 61 bp, Artificial Sequence
  Synthetic oligonucleotide
ccatctcatccctgcgtgtctccgactcaggattagatgagatacgacgctcttccgatct

SEQ ID NO: 874 - DNA, 61 bp, Artificial Sequence
  Synthetic oligonucleotide
ccatctcatccctgcgtgtctccgactcagtataggatctgatacgacgctcttccgatct

SEQ ID NO: 875 - DNA, 61 bp, Artificial Sequence
  Synthetic oligonucleotide
ccatctcatccctgcgtgtctccgactcagagcttatagggatacgacgctcttccgatct

SEQ ID NO: 876 - DNA, 61 bp, Artificial Sequence
  Synthetic oligonucleotide
ccatctcatccctgcgtgtctccgactcaggtctgcaatcgatacgacgctcttccgatct

SEQ ID NO: 877 - DNA, 61 bp, Artificial Sequence
  Synthetic oligonucleotide
ccatctcatccctgcgtgtctccgactcagcgcctcttatgatacgacgctcttccgatct

SEQ ID NO: 878 - DNA, 61 bp, Artificial Sequence
  Synthetic oligonucleotide
ccatctcatccctgcgtgtctccgactcaggttggatcttgatacgacgctcttccgatct

SEQ ID NO: 879 - DNA, 61 bp, Artificial Sequence
  Synthetic oligonucleotide
ccatctcatccctgcgtgtctccgactcaggcgattgcaggatacgacgctcttccgatct

SEQ ID NO: 880 - DNA, 30 bp, Artificial Sequence
  Synthetic oligonucleotide
ccatctcatccctgcgtgtctccgactcag

SEQ ID NO: 881 - DNA, 10 bp, Artificial Sequence
  Synthetic oligonucleotide
ctccatcgag

SEQ ID NO: 882 - DNA, 10 bp, Artificial Sequence
  Synthetic oligonucleotide
ttggtagtcg

SEQ ID NO: 883 - DNA, 10 bp, Artificial Sequence
  Synthetic oligonucleotide
ggccgtcaac

SEQ ID NO: 884 - DNA, 10 bp, Artificial Sequence
  Synthetic oligonucleotide
cctagacgag

SEQ ID NO: 885 - DNA, 10 bp, Artificial Sequence
  Synthetic oligonucleotide
tcgttagagc

SEQ ID NO: 886 - DNA, 10 bp, Artificial Sequence
  Synthetic oligonucleotide
cgttctatca

SEQ ID NO: 887 - DNA, 10 bp, Artificial Sequence
  Synthetic oligonucleotide
cggaatctaa

SEQ ID NO: 888 - DNA, 10 bp, Artificial Sequence
  Synthetic oligonucleotide
atgactgatc

SEQ ID NO: 889 - DNA, 10 bp, Artificial Sequence
  Synthetic oligonucleotide
tcaatatcga

SEQ ID NO: 890 - DNA, 10 bp, Artificial Sequence
  Synthetic oligonucleotide
gtagacctgg

SEQ ID NO: 891 - DNA, 10 bp, Artificial Sequence
  Synthetic oligonucleotide
ttatgaccaa

SEQ ID NO: 892 - DNA, 10 bp, Artificial Sequence
  Synthetic oligonucleotide
ttggtccgtt

SEQ ID NO: 893 - DNA, 10 bp, Artificial Sequence
  Synthetic oligonucleotide
ggtacgttaa

SEQ ID NO: 894 - DNA, 10 bp, Artificial Sequence
  Synthetic oligonucleotide
caatgagtcc

SEQ ID NO: 895 - DNA, 10 bp, Artificial Sequence
  Synthetic oligonucleotide
gatgcagttc

SEQ ID NO: 896 - DNA, 10 bp, Artificial Sequence
  Synthetic oligonucleotide
ccatcgttcc

SEQ ID NO: 897 - DNA, 10 bp, Artificial Sequence
  Synthetic oligonucleotide
ttgagagagt

SEQ ID NO: 898 - DNA, 10 bp, Artificial Sequence
  Synthetic oligonucleotide
actgagcgac

SEQ ID NO: 899 - DNA, 10 bp, Artificial Sequence
  Synthetic oligonucleotide
tgaggaatca

SEQ ID NO: 900 - DNA, 10 bp, Artificial Sequence
  Synthetic oligonucleotide
cctccgacgg

SEQ ID NO: 901 - DNA, 10 bp, Artificial Sequence
  Synthetic oligonucleotide
cattgacgct

SEQ ID NO: 902 - DNA, 10 bp, Artificial Sequence
  Synthetic oligonucleotide
tcgtccttcg

SEQ ID NO: 903 - DNA, 10 bp, Artificial Sequence
  Synthetic oligonucleotide
tgatactcaa

SEQ ID NO: 904 - DNA, 10 bp, Artificial Sequence
  Synthetic oligonucleotide
ttctacctca

SEQ ID NO: 905 - DNA, 10 bp, Artificial Sequence
  Synthetic oligonucleotide
tcgtcggaac

SEQ ID NO: 906 - DNA, 10 bp, Artificial Sequence
  Synthetic oligonucleotide
atcgagatga

SEQ ID NO: 907 - DNA, 10 bp, Artificial Sequence
  Synthetic oligonucleotide
tagactagtc

SEQ ID NO: 908 - DNA, 10 bp, Artificial Sequence
  Synthetic oligonucleotide
gtcgaagcag

SEQ ID NO: 909 - DNA, 10 bp, Artificial Sequence
  Synthetic oligonucleotide
aggcgctagg

SEQ ID NO: 910 - DNA, 10 bp, Artificial Sequence
  Synthetic oligonucleotide
agatgcaact

SEQ ID NO: 911 - DNA, 10 bp, Artificial Sequence
  Synthetic oligonucleotide
aagcctacga

SEQ ID NO: 912 - DNA, 10 bp, Artificial Sequence
  Synthetic oligonucleotide
gtaggcaatt

SEQ ID NO: 913 - DNA, 10 bp, Artificial Sequence
  Synthetic oligonucleotide
tgccagttgc

SEQ ID NO: 914 - DNA, 10 bp, Artificial Sequence
  Synthetic oligonucleotide
cttaggtatc

SEQ ID NO: 915 - DNA, 10 bp, Artificial Sequence
  Synthetic oligonucleotide
gagacctacc

SEQ ID NO: 916 - DNA, 10 bp, Artificial Sequence
  Synthetic oligonucleotide
attgaccgag

SEQ ID NO: 917 - DNA, 10 bp, Artificial Sequence
  Synthetic oligonucleotide
ggaggcggcg

SEQ ID NO: 918 - DNA, 10 bp, Artificial Sequence
  Synthetic oligonucleotide
ccagtacttg

SEQ ID NO: 919 - DNA, 10 bp, Artificial Sequence
  Synthetic oligonucleotide
ggtctcgccg

SEQ ID NO: 920 - DNA, 10 bp, Artificial Sequence
  Synthetic oligonucleotide
ggcggaggtc

SEQ ID NO: 921 - DNA, 10 bp, Artificial Sequence
  Synthetic oligonucleotide
tagttctaga

SEQ ID NO: 922 - DNA, 10 bp, Artificial Sequence
  Synthetic oligonucleotide
ttggagttag

SEQ ID NO: 923 - DNA, 10 bp, Artificial Sequence
  Synthetic oligonucleotide
agatcttggt

SEQ ID NO: 924 - DNA, 10 bp, Artificial Sequence
  Synthetic oligonucleotide
gtaatgatcg

SEQ ID NO: 925 - DNA, 10 bp, Artificial Sequence
  Synthetic oligonucleotide
cagagaggtc

SEQ ID NO: 926 - DNA, 10 bp, Artificial Sequence
  Synthetic oligonucleotide
ttaattagcc

SEQ ID NO: 927 - DNA, 10 bp, Artificial Sequence
  Synthetic oligonucleotide
ctctaactcg

SEQ ID NO: 928 - DNA, 10 bp, Artificial Sequence
  Synthetic oligonucleotide
tacgatcatc

SEQ ID NO: 929 - DNA, 10 bp, Artificial Sequence
  Synthetic oligonucleotide
aggcgagagc

SEQ ID NO: 930 - DNA, 10 bp, Artificial Sequence
  Synthetic oligonucleotide
tcaagatagt

SEQ ID NO: 931 - DNA, 10 bp, Artificial Sequence
  Synthetic oligonucleotide
taattgacct

SEQ ID NO: 932 - DNA, 10 bp, Artificial Sequence
  Synthetic oligonucleotide
cagccggctt

SEQ ID NO: 933 - DNA, 10 bp, Artificial Sequence
  Synthetic oligonucleotide
agaaccggag

SEQ ID NO: 934 - DNA, 10 bp, Artificial Sequence
  Synthetic oligonucleotide
gagatgcatg

SEQ ID NO: 935 - DNA, 10 bp, Artificial Sequence
  Synthetic oligonucleotide
gattaccgga

SEQ ID NO: 936 - DNA, 10 bp, Artificial Sequence
  Synthetic oligonucleotide
tcgtaacggt

SEQ ID NO: 937 - DNA, 10 bp, Artificial Sequence
  Synthetic oligonucleotide
tggcgacgga

SEQ ID NO: 938 - DNA, 10 bp, Artificial Sequence
  Synthetic oligonucleotide
agtcatagcc

SEQ ID NO: 939 - DNA, 10 bp, Artificial Sequence
  Synthetic oligonucleotide
gtcaagtcca

SEQ ID NO: 940 - DNA, 10 bp, Artificial Sequence
  Synthetic oligonucleotide
attcggaagt

SEQ ID NO: 941 - DNA, 10 bp, Artificial Sequence
  Synthetic oligonucleotide
gtcggtagtt

SEQ ID NO: 942 - DNA, 10 bp, Artificial Sequence
  Synthetic oligonucleotide
aggacggacg

SEQ ID NO: 943 - DNA, 10 bp, Artificial Sequence
  Synthetic oligonucleotide
ctcctggacc

SEQ ID NO: 944 - DNA, 10 bp, Artificial Sequence
  Synthetic oligonucleotide
tagcctcgtt

SEQ ID NO: 945 - DNA, 10 bp, Artificial Sequence
  Synthetic oligonucleotide
ggttgaacgt

SEQ ID NO: 946 - DNA, 10 bp, Artificial Sequence
  Synthetic oligonucleotide
aggtcctcgt

SEQ ID NO: 947 - DNA, 10 bp, Artificial Sequence
  Synthetic oligonucleotide
ggaagttata

SEQ ID NO: 948 - DNA, 10 bp, Artificial Sequence
  Synthetic oligonucleotide
tggtaatcct

SEQ ID NO: 949 - DNA, 10 bp, Artificial Sequence
  Synthetic oligonucleotide
aagctaggtt

SEQ ID NO: 950 - DNA, 10 bp, Artificial Sequence
  Synthetic oligonucleotide
tccgcggact

SEQ ID NO: 951 - DNA, 10 bp, Artificial Sequence
  Synthetic oligonucleotide
tgcggatagt

SEQ ID NO: 952 - DNA, 10 bp, Artificial Sequence
  Synthetic oligonucleotide
tggcagctcg

SEQ ID NO: 953 - DNA, 10 bp, Artificial Sequence
  Synthetic oligonucleotide
tgctacggtc

SEQ ID NO: 954 - DNA, 10 bp, Artificial Sequence
  Synthetic oligonucleotide
gcgcaatgac

SEQ ID NO: 955 - DNA, 10 bp, Artificial Sequence
  Synthetic oligonucleotide
cttaatcttg

SEQ ID NO: 956 - DNA, 10 bp, Artificial Sequence
  Synthetic oligonucleotide
ggagttgcgt

SEQ ID NO: 957 - DNA, 10 bp, Artificial Sequence
  Synthetic oligonucleotide
actcgtatca

SEQ ID NO: 958 - DNA, 10 bp, Artificial Sequence
  Synthetic oligonucleotide
ggtaataatg

SEQ ID NO: 959 - DNA, 10 bp, Artificial Sequence
  Synthetic oligonucleotide
tccttataga

SEQ ID NO: 960 - DNA, 10 bp, Artificial Sequence
  Synthetic oligonucleotide
ccgactccaa

SEQ ID NO: 961 - DNA, 10 bp, Artificial Sequence
  Synthetic oligonucleotide
gccaagcttg

SEQ ID NO: 962 - DNA, 10 bp, Artificial Sequence
  Synthetic oligonucleotide
catatcctat

SEQ ID NO: 963 - DNA, 10 bp, Artificial Sequence
  Synthetic oligonucleotide
acctacgcca

SEQ ID NO: 964 - DNA, 10 bp, Artificial Sequence
  Synthetic oligonucleotide
ggaattcagt

SEQ ID NO: 965 - DNA, 10 bp, Artificial Sequence
  Synthetic oligonucleotide
tggcgtagaa

SEQ ID NO: 966 - DNA, 10 bp, Artificial Sequence
  Synthetic oligonucleotide
attgcggcca

SEQ ID NO: 967 - DNA, 10 bp, Artificial Sequence
  Synthetic oligonucleotide
ttcagcttgg

SEQ ID NO: 968 - DNA, 10 bp, Artificial Sequence
  Synthetic oligonucleotide
ccatctggca

SEQ ID NO: 969 - DNA, 10 bp, Artificial Sequence
  Synthetic oligonucleotide
cttataagtt

SEQ ID NO: 970 - DNA, 10 bp, Artificial Sequence
  Synthetic oligonucleotide
gattagatga

SEQ ID NO: 971 - DNA, 10 bp, Artificial Sequence
  Synthetic oligonucleotide
tataggatct

SEQ ID NO: 972 - DNA, 10 bp, Artificial Sequence
  Synthetic oligonucleotide
agcttatagg

SEQ ID NO: 973 - DNA, 10 bp, Artificial Sequence
  Synthetic oligonucleotide
gtctgcaatc

SEQ ID NO: 974 - DNA, 10 bp, Artificial Sequence
  Synthetic oligonucleotide
cgcctcttat

SEQ ID NO: 975 - DNA, 10 bp, Artificial Sequence
  Synthetic oligonucleotide
gttggatctt

SEQ ID NO: 976 - DNA, 10 bp, Artificial Sequence
  Synthetic oligonucleotide
gcgattgcag

SEQ ID NO: 977 - DNA, 21 bp, Artificial Sequence
  Synthetic oligonucleotide
gatacgacgctcttccgatct

SEQ ID NO: 978 - DNA, 48 bp, Artificial Sequence
  Synthetic oligonucleotide
cctctctatgggcagtcggtgatccgaatccgagtctcgtgggctcgg

SEQ ID NO: 979 - DNA, 48 bp, Artificial Sequence
  Synthetic oligonucleotide
cctctctatgggcagtcggtgatataagccggagtctcgtgggctcgg

SEQ ID NO: 980 - DNA, 48 bp, Artificial Sequence
  Synthetic oligonucleotide
cctctctatgggcagtcggtgatccggcggcgagtctcgtgggctcgg

SEQ ID NO: 981 - DNA, 48 bp, Artificial Sequence
  Synthetic oligonucleotide
cctctctatgggcagtcggtgatggcttgccaagtctcgtgggctcgg

SEQ ID NO: 982 - DNA, 48 bp, Artificial Sequence
  Synthetic oligonucleotide
cctctctatgggcagtcggtgatccgctagctggtctcgtgggctcgg

SEQ ID NO: 983 - DNA, 48 bp, Artificial Sequence
  Synthetic oligonucleotide
cctctctatgggcagtcggtgatcttatcctacgtctcgtgggctcgg

SEQ ID NO: 984 - DNA, 48 bp, Artificial Sequence
  Synthetic oligonucleotide
cctctctatgggcagtcggtgattgagctacttgtctcgtgggctcgg

SEQ ID NO: 985 - DNA, 48 bp, Artificial Sequence
  Synthetic oligonucleotide
cctctctatgggcagtcggtgattcaggacttagtctcgtgggctcgg

SEQ ID NO: 986 - DNA, 48 bp, Artificial Sequence
  Synthetic oligonucleotide
cctctctatgggcagtcggtgatccgcagccgcgtctcgtgggctcgg

SEQ ID NO: 987 - DNA, 48 bp, Artificial Sequence
  Synthetic oligonucleotide
cctctctatgggcagtcggtgattgcgcctggtgtctcgtgggctcgg

SEQ ID NO: 988 - DNA, 48 bp, Artificial Sequence
  Synthetic oligonucleotide
cctctctatgggcagtcggtgataatcatacgggtctcgtgggctcgg

SEQ ID NO: 989 - DNA, 48 bp, Artificial Sequence
  Synthetic oligonucleotide
cctctctatgggcagtcggtgatcgccaatcaagtctcgtgggctcgg

SEQ ID NO: 990 - DNA, 48 bp, Artificial Sequence
  Synthetic oligonucleotide
cctctctatgggcagtcggtgatcaaggcttaggtctcgtgggctcgg

SEQ ID NO: 991 - DNA, 48 bp, Artificial Sequence
  Synthetic oligonucleotide
cctctctatgggcagtcggtgatgcgctcgacggtctcgtgggctcgg

SEQ ID NO: 992 - DNA, 48 bp, Artificial Sequence
  Synthetic oligonucleotide
cctctctatgggcagtcggtgattccagcaatagtctcgtgggctcgg

SEQ ID NO: 993 - DNA, 48 bp, Artificial Sequence
  Synthetic oligonucleotide
cctctctatgggcagtcggtgatcatgagaactgtctcgtgggctcgg

SEQ ID NO: 994 - DNA, 48 bp, Artificial Sequence
  Synthetic oligonucleotide
cctctctatgggcagtcggtgataacgtaatctgtctcgtgggctcgg

SEQ ID NO: 995 - DNA, 48 bp, Artificial Sequence
  Synthetic oligonucleotide
cctctctatgggcagtcggtgatattctcctctgtctcgtgggctcgg

SEQ ID NO: 996 - DNA, 48 bp, Artificial Sequence
  Synthetic oligonucleotide
cctctctatgggcagtcggtgattctgcgcgttgtctcgtgggctcgg

SEQ ID NO: 997 - DNA, 48 bp, Artificial Sequence
  Synthetic oligonucleotide
cctctctatgggcagtcggtgatgctcatatgcgtctcgtgggctcgg

SEQ ID NO: 998 - DNA, 48 bp, Artificial Sequence
  Synthetic oligonucleotide
cctctctatgggcagtcggtgatagcggtaacggtctcgtgggctcgg

SEQ ID NO: 999 - DNA, 48 bp, Artificial Sequence
  Synthetic oligonucleotide
cctctctatgggcagtcggtgataatgaatagtgtctcgtgggctcgg

SEQ ID NO: 1000 - DNA, 48 bp, Artificial Sequence
  Synthetic oligonucleotide
cctctctatgggcagtcggtgatccgtatctgggtctcgtgggctcgg

SEQ ID NO: 1001 - DNA, 48 bp, Artificial Sequence
  Synthetic oligonucleotide
cctctctatgggcagtcggtgatccttagtctggtctcgtgggctcgg

SEQ ID NO: 1002 - DNA, 48 bp, Artificial Sequence
  Synthetic oligonucleotide
cctctctatgggcagtcggtgatacctagttaggtctcgtgggctcgg

SEQ ID NO: 1003 - DNA, 48 bp, Artificial Sequence
  Synthetic oligonucleotide
cctctctatgggcagtcggtgatataggagtacgtctcgtgggctcgg

SEQ ID NO: 1004 - DNA, 48 bp, Artificial Sequence
  Synthetic oligonucleotide
cctctctatgggcagtcggtgatctacgacgaggtctcgtgggctcgg

SEQ ID NO: 1005 - DNA, 48 bp, Artificial Sequence
  Synthetic oligonucleotide
cctctctatgggcagtcggtgatagtcgagttcgtctcgtgggctcgg

SEQ ID NO: 1006 - DNA, 48 bp, Artificial Sequence
  Synthetic oligonucleotide
cctctctatgggcagtcggtgattggtccagtcgtctcgtgggctcgg

SEQ ID NO: 1007 - DNA, 48 bp, Artificial Sequence
  Synthetic oligonucleotide
cctctctatgggcagtcggtgatatctaagcaagtctcgtgggctcgg

SEQ ID NO: 1008 - DNA, 48 bp, Artificial Sequence
  Synthetic oligonucleotide
cctctctatgggcagtcggtgatcgaattcgttgtctcgtgggctcgg

SEQ ID NO: 1009 - DNA, 48 bp, Artificial Sequence
  Synthetic oligonucleotide
cctctctatgggcagtcggtgatcagcgatagagtctcgtgggctcgg

SEQ ID NO: 1010 - DNA, 48 bp, Artificial Sequence
  Synthetic oligonucleotide
cctctctatgggcagtcggtgatggtcgctatggtctcgtgggctcgg

SEQ ID NO: 1011 - DNA, 48 bp, Artificial Sequence
  Synthetic oligonucleotide
cctctctatgggcagtcggtgatatccgttagcgtctcgtgggctcgg

SEQ ID NO: 1012 - DNA, 46 bp, Artificial Sequence
  Synthetic oligonucleotide
cctctctatgggcagtcggtgattcgcaattaggtctcgtgggctc

SEQ ID NO: 1013 - DNA, 48 bp, Artificial Sequence
  Synthetic oligonucleotide
cctctctatgggcagtcggtgatggctggctaggtctcgtgggctcgg

SEQ ID NO: 1014 - DNA, 48 bp, Artificial Sequence
  Synthetic oligonucleotide
cctctctatgggcagtcggtgatacggtcttgcgtctcgtgggctcgg

SEQ ID NO: 1015 - DNA, 48 bp, Artificial Sequence
  Synthetic oligonucleotide
cctctctatgggcagtcggtgatgctccattcggtctcgtgggctcgg

SEQ ID NO: 1016 - DNA, 48 bp, Artificial Sequence
  Synthetic oligonucleotide
cctctctatgggcagtcggtgatacgataagcggtctcgtgggctcgg

SEQ ID NO: 1017 - DNA, 48 bp, Artificial Sequence
  Synthetic oligonucleotide
cctctctatgggcagtcggtgataccatagcgcgtctcgtgggctcgg

SEQ ID NO: 1018 - DNA, 48 bp, Artificial Sequence
  Synthetic oligonucleotide
cctctctatgggcagtcggtgatctcttagcgggtctcgtgggctcgg

SEQ ID NO: 1019 - DNA, 48 bp, Artificial Sequence
  Synthetic oligonucleotide
cctctctatgggcagtcggtgattgattcaactgtctcgtgggctcgg

SEQ ID NO: 1020 - DNA, 48 bp, Artificial Sequence
  Synthetic oligonucleotide
cctctctatgggcagtcggtgattatggccgcggtctcgtgggctcgg

SEQ ID NO: 1021 - DNA, 48 bp, Artificial Sequence
  Synthetic oligonucleotide
cctctctatgggcagtcggtgatagaggtcgcagtctcgtgggctcgg

SEQ ID NO: 1022 - DNA, 48 bp, Artificial Sequence
  Synthetic oligonucleotide
cctctctatgggcagtcggtgataggagattgagtctcgtgggctcgg

SEQ ID NO: 1023 - DNA, 48 bp, Artificial Sequence
  Synthetic oligonucleotide
cctctctatgggcagtcggtgatggctatataggtctcgtgggctcgg

SEQ ID NO: 1024 - DNA, 48 bp, Artificial Sequence
  Synthetic oligonucleotide
cctctctatgggcagtcggtgattcgcgtacttgtctcgtgggctcgg

SEQ ID NO: 1025 - DNA, 48 bp, Artificial Sequence
  Synthetic oligonucleotide
cctctctatgggcagtcggtgataataataatggtctcgtgggctcgg

SEQ ID NO: 1026 - DNA, 48 bp, Artificial Sequence
  Synthetic oligonucleotide
cctctctatgggcagtcggtgatttcgttccatgtctcgtgggctcgg

SEQ ID NO: 1027 - DNA, 48 bp, Artificial Sequence
  Synthetic oligonucleotide
cctctctatgggcagtcggtgattacctaatcagtctcgtgggctcgg

SEQ ID NO: 1028 - DNA, 48 bp, Artificial Sequence
  Synthetic oligonucleotide
cctctctatgggcagtcggtgataagtaatattgtctcgtgggctcgg

SEQ ID NO: 1029 - DNA, 48 bp, Artificial Sequence
  Synthetic oligonucleotide
cctctctatgggcagtcggtgatagctaagaatgtctcgtgggctcgg

SEQ ID NO: 1030 - DNA, 48 bp, Artificial Sequence
  Synthetic oligonucleotide
cctctctatgggcagtcggtgatgtcgaggtatgtctcgtgggctcgg

SEQ ID NO: 1031 - DNA, 48 bp, Artificial Sequence
  Synthetic oligonucleotide
cctctctatgggcagtcggtgatttattagtaggtctcgtgggctcgg

SEQ ID NO: 1032 - DNA, 48 bp, Artificial Sequence
  Synthetic oligonucleotide
cctctctatgggcagtcggtgattgcgaagatcgtctcgtgggctcgg

SEQ ID NO: 1033 - DNA, 48 bp, Artificial Sequence
  Synthetic oligonucleotide
cctctctatgggcagtcggtgataactacggctgtctcgtgggctcgg

SEQ ID NO: 1034 - DNA, 48 bp, Artificial Sequence
  Synthetic oligonucleotide
cctctctatgggcagtcggtgataacggaacgcgtctcgtgggctcgg

SEQ ID NO: 1035 - DNA, 48 bp, Artificial Sequence
  Synthetic oligonucleotide
cctctctatgggcagtcggtgatgatgctacgagtctcgtgggctcgg

SEQ ID NO: 1036 - DNA, 48 bp, Artificial Sequence
  Synthetic oligonucleotide
cctctctatgggcagtcggtgatatctgccaatgtctcgtgggctcgg

SEQ ID NO: 1037 - DNA, 48 bp, Artificial Sequence
  Synthetic oligonucleotide
cctctctatgggcagtcggtgatatcgtatcaagtctcgtgggctcgg

SEQ ID NO: 1038 - DNA, 48 bp, Artificial Sequence
  Synthetic oligonucleotide
cctctctatgggcagtcggtgataacgcctctagtctcgtgggctcgg

SEQ ID NO: 1039 - DNA, 48 bp, Artificial Sequence
  Synthetic oligonucleotide
cctctctatgggcagtcggtgatacggcaaccagtctcgtgggctcgg

SEQ ID NO: 1040 - DNA, 48 bp, Artificial Sequence
  Synthetic oligonucleotide
cctctctatgggcagtcggtgatcaggctaagagtctcgtgggctcgg

SEQ ID NO: 1041 - DNA, 48 bp, Artificial Sequence
  Synthetic oligonucleotide
cctctctatgggcagtcggtgatcgcaatatcagtctcgtgggctcgg

SEQ ID NO: 1042 - DNA, 48 bp, Artificial Sequence
  Synthetic oligonucleotide
cctctctatgggcagtcggtgatttcgataaccgtctcgtgggctcgg

SEQ ID NO: 1043 - DNA, 48 bp, Artificial Sequence
  Synthetic oligonucleotide
cctctctatgggcagtcggtgataacctcaagagtctcgtgggctcgg

SEQ ID NO: 1044 - DNA, 48 bp, Artificial Sequence
  Synthetic oligonucleotide
cctctctatgggcagtcggtgatcaggcgccatgtctcgtgggctcgg

SEQ ID NO: 1045 - DNA, 48 bp, Artificial Sequence
  Synthetic oligonucleotide
cctctctatgggcagtcggtgataactattatagtctcgtgggctcgg

SEQ ID NO: 1046 - DNA, 48 bp, Artificial Sequence
  Synthetic oligonucleotide
cctctctatgggcagtcggtgataagttacctagtctcgtgggctcgg

SEQ ID NO: 1047 - DNA, 48 bp, Artificial Sequence
  Synthetic oligonucleotide
cctctctatgggcagtcggtgatcggcagaggagtctcgtgggctcgg

SEQ ID NO: 1048 - DNA, 48 bp, Artificial Sequence
  Synthetic oligonucleotide
cctctctatgggcagtcggtgatgcctcaataagtctcgtgggctcgg

SEQ ID NO: 1049 - DNA, 48 bp, Artificial Sequence
  Synthetic oligonucleotide
cctctctatgggcagtcggtgatttaacgccgtgtctcgtgggctcgg

SEQ ID NO: 1050 - DNA, 48 bp, Artificial Sequence
  Synthetic oligonucleotide
cctctctatgggcagtcggtgatcatacgatgcgtctcgtgggctcgg

SEQ ID NO: 1051 - DNA, 48 bp, Artificial Sequence
  Synthetic oligonucleotide
cctctctatgggcagtcggtgataagctgacctgtctcgtgggctcgg

SEQ ID NO: 1052 - DNA, 48 bp, Artificial Sequence
  Synthetic oligonucleotide
cctctctatgggcagtcggtgatgagtccttatgtctcgtgggctcgg

SEQ ID NO: 1053 - DNA, 48 bp, Artificial Sequence
  Synthetic oligonucleotide
cctctctatgggcagtcggtgatcctacggcaagtctcgtgggctcgg

SEQ ID NO: 1054 - DNA, 48 bp, Artificial Sequence
  Synthetic oligonucleotide
cctctctatgggcagtcggtgataatattcgaagtctcgtgggctcgg

SEQ ID NO: 1055 - DNA, 48 bp, Artificial Sequence
  Synthetic oligonucleotide
cctctctatgggcagtcggtgatttcaagaatcgtctcgtgggctcgg

SEQ ID NO: 1056 - DNA, 48 bp, Artificial Sequence
  Synthetic oligonucleotide
cctctctatgggcagtcggtgatatgctcgcaagtctcgtgggctcgg

SEQ ID NO: 1057 - DNA, 48 bp, Artificial Sequence
  Synthetic oligonucleotide
cctctctatgggcagtcggtgatggagtaagccgtctcgtgggctcgg

SEQ ID NO: 1058 - DNA, 48 bp, Artificial Sequence
  Synthetic oligonucleotide
cctctctatgggcagtcggtgatttatcgtattgtctcgtgggctcgg

SEQ ID NO: 1059 - DNA, 48 bp, Artificial Sequence
  Synthetic oligonucleotide
cctctctatgggcagtcggtgataagtctaatagtctcgtgggctcgg

SEQ ID NO: 1060 - DNA, 48 bp, Artificial Sequence
  Synthetic oligonucleotide
cctctctatgggcagtcggtgatcggcttactagtctcgtgggctcgg

SEQ ID NO: 1061 - DNA, 48 bp, Artificial Sequence
  Synthetic oligonucleotide
cctctctatgggcagtcggtgatgatatggtctgtctcgtgggctcgg

SEQ ID NO: 1062 - DNA, 48 bp, Artificial Sequence
  Synthetic oligonucleotide
cctctctatgggcagtcggtgattagtcgtccagtctcgtgggctcgg

SEQ ID NO: 1063 - DNA, 48 bp, Artificial Sequence
  Synthetic oligonucleotide
cctctctatgggcagtcggtgattagctgctacgtctcgtgggctcgg

SEQ ID NO: 1064 - DNA, 48 bp, Artificial Sequence
  Synthetic oligonucleotide
cctctctatgggcagtcggtgatctcttcaagcgtctcgtgggctcgg

SEQ ID NO: 1065 - DNA, 48 bp, Artificial Sequence
  Synthetic oligonucleotide
cctctctatgggcagtcggtgatatgaacgcgcgtctcgtgggctcgg

SEQ ID NO: 1066 - DNA, 48 bp, Artificial Sequence
  Synthetic oligonucleotide
cctctctatgggcagtcggtgatgtcgacggaagtctcgtgggctcgg

SEQ ID NO: 1067 - DNA, 48 bp, Artificial Sequence
  Synthetic oligonucleotide
cctctctatgggcagtcggtgatactaattgaggtctcgtgggctcgg

SEQ ID NO: 1068 - DNA, 48 bp, Artificial Sequence
  Synthetic oligonucleotide
cctctctatgggcagtcggtgatcttgcataatgtctcgtgggctcgg

SEQ ID NO: 1069 - DNA, 48 bp, Artificial Sequence
  Synthetic oligonucleotide
cctctctatgggcagtcggtgattccttaccaagtctcgtgggctcgg

SEQ ID NO: 1070 - DNA, 48 bp, Artificial Sequence
  Synthetic oligonucleotide
cctctctatgggcagtcggtgattgcagcctacgtctcgtgggctcgg

SEQ ID NO: 1071 - DNA, 48 bp, Artificial Sequence
  Synthetic oligonucleotide
cctctctatgggcagtcggtgatggagctgagggtctcgtgggctcgg

SEQ ID NO: 1072 - DNA, 48 bp, Artificial Sequence
  Synthetic oligonucleotide
cctctctatgggcagtcggtgatgcagcggactgtctcgtgggctcgg

SEQ ID NO: 1073 - DNA, 48 bp, Artificial Sequence
  Synthetic oligonucleotide
cctctctatgggcagtcggtgatcatcgcgctcgtctcgtgggctcgg

SEQ ID NO: 1074 - DNA, 23 bp, Artificial Sequence
  Synthetic oligonucleotide
cctctctatgggcagtcggtgat

SEQ ID NO: 1075 - DNA, 10 bp, Artificial Sequence
  Synthetic oligonucleotide
ccgaatccga

SEQ ID NO: 1076 - DNA, 10 bp, Artificial Sequence
  Synthetic oligonucleotide
ataagccgga

SEQ ID NO: 1077 - DNA, 10 bp, Artificial Sequence
  Synthetic oligonucleotide
ccggcggcga

SEQ ID NO: 1078 - DNA, 10 bp, Artificial Sequence
  Synthetic oligonucleotide
ggcttgccaa

SEQ ID NO: 1079 - DNA, 10 bp, Artificial Sequence
  Synthetic oligonucleotide
ccgctagctg

SEQ ID NO: 1080 - DNA, 10 bp, Artificial Sequence
  Synthetic oligonucleotide
cttatcctac

SEQ ID NO: 1081 - DNA, 10 bp, Artificial Sequence
  Synthetic oligonucleotide
tgagctactt

SEQ ID NO: 1082 - DNA, 10 bp, Artificial Sequence
  Synthetic oligonucleotide
tcaggactta

SEQ ID NO: 1083 - DNA, 10 bp, Artificial Sequence
  Synthetic oligonucleotide
ccgcagccgc

SEQ ID NO: 1084 - DNA, 10 bp, Artificial Sequence
  Synthetic oligonucleotide
tgcgcctggt

SEQ ID NO: 1085 - DNA, 10 bp, Artificial Sequence
  Synthetic oligonucleotide
aatcatacgg

SEQ ID NO: 1086 - DNA, 10 bp, Artificial Sequence
  Synthetic oligonucleotide
cgccaatcaa

SEQ ID NO: 1087 - DNA, 10 bp, Artificial Sequence
  Synthetic oligonucleotide
caaggcttag

SEQ ID NO: 1088 - DNA, 10 bp, Artificial Sequence
  Synthetic oligonucleotide
gcgctcgacg

SEQ ID NO: 1089 - DNA, 10 bp, Artificial Sequence
  Synthetic oligonucleotide
tccagcaata

SEQ ID NO: 1090 - DNA, 10 bp, Artificial Sequence
  Synthetic oligonucleotide
catgagaact

SEQ ID NO: 1091 - DNA, 10 bp, Artificial Sequence
  Synthetic oligonucleotide
aacgtaatct

SEQ ID NO: 1092 - DNA, 10 bp, Artificial Sequence
  Synthetic oligonucleotide
attctcctct

SEQ ID NO: 1093 - DNA, 10 bp, Artificial Sequence
  Synthetic oligonucleotide
tctgcgcgtt

SEQ ID NO: 1094 - DNA, 10 bp, Artificial Sequence
  Synthetic oligonucleotide
gctcatatgc

SEQ ID NO: 1095 - DNA, 10 bp, Artificial Sequence
  Synthetic oligonucleotide
agcggtaacg

SEQ ID NO: 1096 - DNA, 10 bp, Artificial Sequence
  Synthetic oligonucleotide
aatgaatagt

SEQ ID NO: 1097 - DNA, 10 bp, Artificial Sequence
  Synthetic oligonucleotide
ccgtatctgg

SEQ ID NO: 1098 - DNA, 10 bp, Artificial Sequence
  Synthetic oligonucleotide
ccttagtctg

SEQ ID NO: 1099 - DNA, 10 bp, Artificial Sequence
  Synthetic oligonucleotide
acctagttag

SEQ ID NO: 1100 - DNA, 10 bp, Artificial Sequence
  Synthetic oligonucleotide
ataggagtac

SEQ ID NO: 1101 - DNA, 10 bp, Artificial Sequence
  Synthetic oligonucleotide
ctacgacgag

SEQ ID NO: 1102 - DNA, 10 bp, Artificial Sequence
  Synthetic oligonucleotide
agtcgagttc

SEQ ID NO: 1103 - DNA, 10 bp, Artificial Sequence
  Synthetic oligonucleotide
tggtccagtc

SEQ ID NO: 1104 - DNA, 10 bp, Artificial Sequence
  Synthetic oligonucleotide
atctaagcaa

SEQ ID NO: 1105 - DNA, 10 bp, Artificial Sequence
  Synthetic oligonucleotide
cgaattcgtt

SEQ ID NO: 1106 - DNA, 10 bp, Artificial Sequence
  Synthetic oligonucleotide
cagcgataga

SEQ ID NO: 1107 - DNA, 10 bp, Artificial Sequence
  Synthetic oligonucleotide
ggtcgctatg

SEQ ID NO: 1108 - DNA, 10 bp, Artificial Sequence
  Synthetic oligonucleotide
atccgttagc

SEQ ID NO: 1109 - DNA, 10 bp, Artificial Sequence
  Synthetic oligonucleotide
tcgcaattag

SEQ ID NO: 1110 - DNA, 10 bp, Artificial Sequence
  Synthetic oligonucleotide
ggctggctag

SEQ ID NO: 1111 - DNA, 10 bp, Artificial Sequence
  Synthetic oligonucleotide
acggtcttgc

SEQ ID NO: 1112 - DNA, 10 bp, Artificial Sequence
  Synthetic oligonucleotide
gctccattcg

SEQ ID NO: 1113 - DNA, 10 bp, Artificial Sequence
  Synthetic oligonucleotide
acgataagcg

SEQ ID NO: 1114 - DNA, 10 bp, Artificial Sequence
  Synthetic oligonucleotide
accatagcgc

SEQ ID NO: 1115 - DNA, 10 bp, Artificial Sequence
  Synthetic oligonucleotide
ctcttagcgg

SEQ ID NO: 1116 - DNA, 10 bp, Artificial Sequence
  Synthetic oligonucleotide
tgattcaact

SEQ ID NO: 1117 - DNA, 10 bp, Artificial Sequence
  Synthetic oligonucleotide
tatggccgcg

SEQ ID NO: 1118 - DNA, 10 bp, Artificial Sequence
  Synthetic oligonucleotide
agaggtcgca

SEQ ID NO: 1119 - DNA, 10 bp, Artificial Sequence
  Synthetic oligonucleotide
aggagattga

SEQ ID NO: 1120 - DNA, 10 bp, Artificial Sequence
  Synthetic oligonucleotide
ggctatatag

SEQ ID NO: 1121 - DNA, 10 bp, Artificial Sequence
  Synthetic oligonucleotide
tcgcgtactt

SEQ ID NO: 1122 - DNA, 10 bp, Artificial Sequence
  Synthetic oligonucleotide
aataataatg

SEQ ID NO: 1123 - DNA, 10 bp, Artificial Sequence
  Synthetic oligonucleotide
ttcgttccat

SEQ ID NO: 1124 - DNA, 10 bp, Artificial Sequence
  Synthetic oligonucleotide
tacctaatca

SEQ ID NO: 1125 - DNA, 10 bp, Artificial Sequence
  Synthetic oligonucleotide
aagtaatatt

SEQ ID NO: 1126 - DNA, 10 bp, Artificial Sequence
  Synthetic oligonucleotide
agctaagaat

SEQ ID NO: 1127 - DNA, 10 bp, Artificial Sequence
  Synthetic oligonucleotide
gtcgaggtat

SEQ ID NO: 1128 - DNA, 10 bp, Artificial Sequence
  Synthetic oligonucleotide
ttattagtag

SEQ ID NO: 1129 - DNA, 10 bp, Artificial Sequence
  Synthetic oligonucleotide
tgcgaagatc

SEQ ID NO: 1130 - DNA, 10 bp, Artificial Sequence
  Synthetic oligonucleotide
aactacggct

SEQ ID NO: 1131 - DNA, 10 bp, Artificial Sequence
  Synthetic oligonucleotide
aacggaacgc

SEQ ID NO: 1132 - DNA, 10 bp, Artificial Sequence
  Synthetic oligonucleotide
gatgctacga

SEQ ID NO: 1133 - DNA, 10 bp, Artificial Sequence
  Synthetic oligonucleotide
atctgccaat

SEQ ID NO: 1134 - DNA, 10 bp, Artificial Sequence
  Synthetic oligonucleotide
atcgtatcaa

SEQ ID NO: 1135 - DNA, 10 bp, Artificial Sequence
  Synthetic oligonucleotide
aacgcctcta

SEQ ID NO: 1136 - DNA, 10 bp, Artificial Sequence
  Synthetic oligonucleotide
acggcaacca

SEQ ID NO: 1137 - DNA, 10 bp, Artificial Sequence
  Synthetic oligonucleotide
caggctaaga

SEQ ID NO: 1138 - DNA, 10 bp, Artificial Sequence
  Synthetic oligonucleotide
cgcaatatca

SEQ ID NO: 1139 - DNA, 10 bp, Artificial Sequence
  Synthetic oligonucleotide
ttcgataacc

SEQ ID NO: 1140 - DNA, 10 bp, Artificial Sequence
  Synthetic oligonucleotide
aacctcaaga

SEQ ID NO: 1141 - DNA, 10 bp, Artificial Sequence
  Synthetic oligonucleotide
caggcgccat

SEQ ID NO: 1142 - DNA, 10 bp, Artificial Sequence
  Synthetic oligonucleotide
aactattata

SEQ ID NO: 1143 - DNA, 10 bp, Artificial Sequence
  Synthetic oligonucleotide
aagttaccta

SEQ ID NO: 1144 - DNA, 10 bp, Artificial Sequence
  Synthetic oligonucleotide
cggcagagga

SEQ ID NO: 1145 - DNA, 10 bp, Artificial Sequence
  Synthetic oligonucleotide
gcctcaataa

SEQ ID NO: 1146 - DNA, 10 bp, Artificial Sequence
  Synthetic oligonucleotide
ttaacgccgt

SEQ ID NO: 1147 - DNA, 10 bp, Artificial Sequence
  Synthetic oligonucleotide
catacgatgc

SEQ ID NO: 1148 - DNA, 10 bp, Artificial Sequence
  Synthetic oligonucleotide
aagctgacct

SEQ ID NO: 1149 - DNA, 10 bp, Artificial Sequence
  Synthetic oligonucleotide
gagtccttat

SEQ ID NO: 1150 - DNA, 10 bp, Artificial Sequence
  Synthetic oligonucleotide
cctacggcaa

SEQ ID NO: 1151 - DNA, 10 bp, Artificial Sequence
  Synthetic oligonucleotide
aatattcgaa

SEQ ID NO: 1152 - DNA, 10 bp, Artificial Sequence
  Synthetic oligonucleotide
ttcaagaatc

SEQ ID NO: 1153 - DNA, 10 bp, Artificial Sequence
  Synthetic oligonucleotide
atgctcgcaa

SEQ ID NO: 1154 - DNA, 10 bp, Artificial Sequence
  Synthetic oligonucleotide
ggagtaagcc

SEQ ID NO: 1155 - DNA, 10 bp, Artificial Sequence
  Synthetic oligonucleotide
ttatcgtatt

SEQ ID NO: 1156 - DNA, 10 bp, Artificial Sequence
  Synthetic oligonucleotide
aagtctaata

SEQ ID NO: 1157 - DNA, 10 bp, Artificial Sequence
  Synthetic oligonucleotide
cggcttacta

SEQ ID NO: 1158 - DNA, 10 bp, Artificial Sequence
  Synthetic oligonucleotide
gatatggtct

SEQ ID NO: 1159 - DNA, 10 bp, Artificial Sequence
  Synthetic oligonucleotide
tagtcgtcca

SEQ ID NO: 1160 - DNA, 10 bp, Artificial Sequence
  Synthetic oligonucleotide
tagctgctac

SEQ ID NO: 1161 - DNA, 10 bp, Artificial Sequence
  Synthetic oligonucleotide
ctcttcaagc

SEQ ID NO: 1162 - DNA, 10 bp, Artificial Sequence
  Synthetic oligonucleotide
atgaacgcgc

SEQ ID NO: 1163 - DNA, 10 bp, Artificial Sequence
  Synthetic oligonucleotide
gtcgacggaa

SEQ ID NO: 1164 - DNA, 10 bp, Artificial Sequence
  Synthetic oligonucleotide
actaattgag

SEQ ID NO: 1165 - DNA, 10 bp, Artificial Sequence
  Synthetic oligonucleotide
cttgcataat

SEQ ID NO: 1166 - DNA, 10 bp, Artificial Sequence
  Synthetic oligonucleotide
tccttaccaa

SEQ ID NO: 1167 - DNA, 10 bp, Artificial Sequence
  Synthetic oligonucleotide
tgcagcctac

SEQ ID NO: 1168 - DNA, 10 bp, Artificial Sequence
  Synthetic oligonucleotide
ggagctgagg

SEQ ID NO: 1169 - DNA, 10 bp, Artificial Sequence
  Synthetic oligonucleotide
gcagcggact

SEQ ID NO: 1170 - DNA, 10 bp, Artificial Sequence
  Synthetic oligonucleotide
catcgcgctc

SEQ ID NO: 1171 - DNA, 15 bp, Artificial Sequence
  Synthetic oligonucleotide
gtctcgtgggctcgg

SEQ ID NO: 1172 - DNA, 36 bp, Artificial Sequence; identical to SEQ ID NO: 398
  Synthetic oligonucleotide
  misc_feature (36)..(36): 3′ C6

SEQ ID NO: 1173 - DNA, 36 bp, Artificial Sequence; identical to SEQ ID NO: 399
  Synthetic oligonucleotide
  misc_feature (36)..(36): 3′ C6

SEQ ID NO: 1174 - DNA, 12 bp, Artificial Sequence
  Synthetic oligonucleotide
huaaacgaacww

SEQ ID NO: 1175 - DNA, 10 bp, Artificial Sequence
  Synthetic oligonucleotide
  misc_feature (1)..(1): n is a, c, g, or t
nkswtcttwk

(19) United States
(12) Patent Application Publication
(10) Pub. No.: US 2023/0374592 A1
(43) Pub. Date: Nov. 23, 2023

(54) MASSIVELY PARALLELED MULTI-PATIENT ASSAY FOR PATHOGENIC INFECTION DIAGNOSIS AND HOST PHYSIOLOGY SURVEILLANCE USING NUCLEIC ACID SEQUENCING

(71) Applicant: The USA, as represented by the Secretary, Dept. of Health and Human Services, Bethesda, MD (US)

(72) Inventors: Oswaldo Alonso Lozoya, Cary, NC (US); Brian Nicholas Papas, Cary, NC (US)

(21) Appl. No.: 18/031,165

(22) PCT Filed: Nov. 19, 2021
(86) PCT No.: PCT/US2021/059996

§ 371 Date: Apr. 11, 2023

Related U.S. Application Data
(60) Provisional application No. 63/116,031, filed on Nov. 19, 2020.

(51) Int. Cl. C12Q 1/6883; C12Q 1/6806

(52) U.S. Cl. CPC C12Q 1/6883; C12Q 1/6806; C12Q 2600/106; C12Q 2600/118; C12Q 2600/158

(57) ABSTRACT

The invention generally relates to detecting the presence of a pathogen in a sample, specifically severe acute respiratory syndrome coronavirus 2 (SARS-CoV-2), and physiological effects on the host with prognostic value, by methods that can simultaneously detect the pathogen and a host's transcriptional response to infection by the pathogen.

### CROSS REFERENCE TO RELATED APPLICATIONS

This application claims the benefit of U.S. Provisional Application No. 63/116,031, filed Nov. 19, 2020, which is incorporated by reference herein in its entirety.

### GOVERNMENT LICENSE RIGHTS

This invention was made with Government support. The Government has certain rights in the invention.

### SEQUENCE LISTING

A computer readable form of the Sequence Listing is filed with this application by electronic submission and is incorporated into this application by reference in its entirety. The Sequence Listing is contained in the ASCII text file created on Nov. 19, 2021, having the file name “20-1516-WO_Sequence-Listing_ST25.txt” and is 275 kb in size.

### BACKGROUND OF THE INVENTION

### Field of the Invention

This disclosure generally relates to detecting the presence of a pathogen in a sample, specifically severe acute respiratory syndrome coronavirus 2 (SARS-CoV-2), and methods that simultaneously detect the pathogen and a host's transcriptional response to infection by the pathogen.

### Description of Related Art

Infectious disease outbreaks like Coronavirus Disease 2019 (COVID-19) can overwhelm healthcare systems when screening tools are lacking or scarce. Without available vaccines or proven disease management drugs against SARS-CoV-2 infection, healthcare systems must rely on screening to identify infected patients and manage them effectively. The current pandemic-level demand for clinical-grade COVID-19 diagnostics, along with technical limitations of qPCR-based fluorometric tests for SARS-CoV-2, are contributing to bottlenecks in COVID-19 diagnosis that play against the welfare of COVID-19 patients, who could otherwise be managed earlier in the course of infection and treated accordingly. Slow diagnostic times also increase the occupational hazard among healthcare workers for SARS-CoV-2 transmission, who are faced with the real threat of contracting COVID-19 from undiagnosed patients while waiting for test results.

In practice, diagnostic-level sensitivity with PCR-based assays is only guaranteed for single-target reactions, which effectively discourages the implementation of multiplexed (color) qPCR fluorometry for SARS-CoV-2 detection in clinical-grade tests. In the face of an ongoing COVID-19 pandemic and with single-plex qPCR fluorometry inadequate for high throughput clinical diagnostics, the demand for testing exceeds the capacity, leading to limited availability, long queue times, backlogs in COVID-19 diagnoses, and delayed access to specialized treatment for COVID-19 patients.

There is a need for an easily scalable and massively paralleled multiplexed screening method using next generation sequencing (NGS) with sample-specific barcoded indexes, that detects both SARS-COV-2 viral gRNA content and the host's transcriptional response to infection simultaneously, and matching existing SOPs for PCR-based sample processing routines of CLIA-certified facilities. The methods disclosed herein would provide the capability for testing tens of thousands of patient samples in a large bolus, and allow accurate and fast-turnaround SARS-CoV-2 testing capacity at population scale, permitting massive scale monitoring of at-risk individuals with minimal processing delay.

### SUMMARY OF THE INVENTION

It is against the above background that the present invention provides certain advantages over the prior art.

Although this invention as disclosed herein is not limited to specific advantages or functionalities (such for example, detection of severe acute respiratory syndrome coronavirus using next generation sequencing), the invention provides a scalable and massively paralleled screening for infectious pathogens using nucleic acid sequencing. In this approach, biological samples collected from donor are used to assemble agnostic libraries of nucleic acids, each one artificially appended with a prescribed, distinct, and donor-specific barcode, which capture underlying gene expression information from the donor and any infectious pathogens present in the biological sample. Then, to enhance detection of pathogen infection status, donor libraries are subjected to selective enrichment of pathogen-derived nucleic acids via targeted amplification anchored to interspersed, repetitive, evolutionarily conserved and/or genetically functional consensus sequences found across nucleic acids originating from one or many infectious pathogens. Next, nucleic acid libraries from many donors, each flagged with donor-specific barcodes and carrying copies of donor and/or any underlying pathogen-derived gene expression templates, are sequenced in a bolus. After, the collective of sequences read are assigned back to their respective donors based on their synthetic barcodes and bioinformatically aligned to reference host and pathogen genomes. Finally, using machine-learning methods, donors are parsed by their detected infection status and classified under prognostic, evolving or concomitant pathology groups based on sequences read from their respective specimens.

Also disclosed herein are methods for detection of both pathogen RNA and the donor host's transcriptional response to the pathogen infection simultaneously.

The disclosure provides a method for detecting a plurality of nucleic acids in a sample from a subject, comprising:
(a) obtaining the sample from the subject and extracting nucleic acid from the sample to generate a nucleic acid sample;
(b) preparing a library of nucleic acid sequences from the nucleic acid sample; wherein the library of nucleic acid sequences is prepared using:
   (i) an anchored oligonucleotide comprising:
      (1) a 3′ splint
      (2) a unique molecule identifier (UMI)
      (3) a sample-specific barcode; and
      (4) an oligo-dT;
   (ii) a pathogen-specific oligonucleotide primer comprising:
      (1) an extended 3′ end cDNA splint
      (2) a minimal 3′ end cDNA splint
      (3) a 3′ end cDNA UMI; and
      (4) a pathogen specific consensus sequence;
   (iii) a 3′ indexed adapter oligonucleotide comprising:
      (1) a 3′ adapter;
      (2) a 3′ barcode; and
      (3) a 3′ coupling sequence; and
   (iv) a 5′ indexed adapter oligonucleotide comprising:
      (1) a 5′ adapter;
      (2) a 5′ barcode; and
      (3) a 5′ coupling sequence; and
(c) detecting the plurality of nucleic acids by sequencing the library of nucleic acid sequences to generate a plurality of nucleic acid reads.

In one aspect of the methods disclosed herein, the method further comprises preparing the library using:
(v) a pathogen specific template switching oligonucleotide comprising:
   (1) a pathogen specific consensus sequence; and
   (2) a template switching motif
(vi) a generic template switching oligonucleotide comprising:
   (1) a generic tailing motif; and
   (2) a template switching motif; and
(vii) a universal cDNA coupler forward primer oligonucleotide comprising:
   (1) an extended 3′ end cDNA splint; and
   (2) a minimal 3′ end cDNA splint.

In one aspect of the methods disclosed herein, the method further comprises preparing the library using:
(viii) a pathogen specific enrichment coupler reverse primer oligonucleotide comprising:
   (1) a minimal 5′ end cDNA splint;
   (2) an extended 5′ end cDNA splint;
   (3) a 5′ end cDNA UMI; and
   (4) a pathogenic specific consensus sequence; and
(ix) a generic cDNA coupler reverse primer oligonucleotide comprising:
   (1) a generic tailing motif; and
   (2) a template switching motif.

In one aspect of the methods disclosed herein, the method further comprises preparing the library using:
(x) a rDNA blocking duplex oligonucleotide.

In one aspect of the methods disclosed herein, the nucleic acid sample comprises RNA, DNA, or both RNA and DNA.

In one aspect of the methods disclosed herein, the sample is a clinical sample.

In one aspect of the methods disclosed herein, the sample is selected from the group consisting of a nasopharyngeal swab, an oropharyngeal swab, a buccal swab, whole saliva sample, cell-free saliva sample, blood plasma, blood serum, whole blood, sputum, stool, urine, cerebral spinal fluid, synovial fluid, peritoneal fluid, pleural fluid, pericardial fluid, and bone marrow.

In one aspect of the methods disclosed herein, the sample comprises nucleic acid from a plurality of organisms.

In one aspect of the methods disclosed herein, the sample comprises nucleic acid from both the subject and the pathogen.

In one aspect of the methods disclosed herein, the pathogen specific consensus sequence comprises a sequence from a conserved region from the pathogen's genome.

In one aspect of the methods disclosed herein, the pathogen specific consensus sequence comprises a transcription-regulatory sequence motif.

In one aspect of the methods disclosed herein, the pathogen is selected from: Acinetobacter baumannii, Actinomyces gerencseriae, Actinomyces israelii, Alphavirus species (e.g., Chikungunya virus, Eastern equine encephalitis virus, Venezuelan equine encephalitis virus, and Western equine encephalitis virus), Anaplasma species, Ancylostoma duodenale, Angiostrongylus cantonensis, Angiostrongylus costaricensis, Arcanobacterium haemolyticum, Ascaris lumbricoides, Aspergillus species, Astroviridae species, Babesia species, Bacillus anthracis, Bacillus cereus, Bacteroides species, Balantidium coli, Bartonella bacilliformis, Bartonella henselae, Bartonella, Batrachochytrium dendrabatidis, Baylisascaris species, Blastocystis species, Blastomyces dermatitidis, Bordetella pertussis, Borrelia afzelii, Borrelia burgdorferi, Borrelia garinii, Brucella species, Burkholderia mallei, Burkholderia pseudomallei, Burkholderia species, Caliciviridae species, Campylobacter species, Candida albicans, Capillaria aerophila, Capillaria philippinensis, Chlamydia trachomatis, Chlamydophila pneumoniae, Clonorchis sinensis, Clostridium botulinum, Clostridium difficile, Clostridium perfringens, Clostridium tetani, Coccidioides immitis, Coccidioides posadasii, Colorado tick fever virus (CTFV), Corynebacterium diphtheria, Crimean-Congo hemorrhagic fever virus, Cryptococcus neoformans, Cryptosporidium species, Cyclospora cayetanensis, Cytomegalovirus, Dengue viruses (DEN-1, DEN-2, DEN-3 and DEN-4), Dientamoeba fragilis, Dracunculus medinensis, Ebolavirus (EBOV), Entamoeba histolytica, Enterobius vermicularis, Enterococcus species, Epstein-Barr virus (EBV), Escherichia coli, Fasciola gigantica, Fasciola hepatica, Fasciolopsis buski, Flavivirus species, Geotrichum candidum, Giardia lamblia, Haemophilus ducreyi, Haemophilus influenza, Hantaviridae family, Helicobacter pylori, Hepatitis A virus, Hepatitis B virus, Hepatitis C virus, Hepatitis D Virus, Hepatitis E virus, Herpes simplex virus 1 (HSV-1), Herpes simplex virus 2 (HSV-2), Histoplasma capsulatum, HIV (Human immunodeficiency virus), Human herpesvirus 6 (HHV-6), Human herpesvirus 7 (HHV-7), Human papillomavirus (HPV), Junin virus, Klebsiella granulomatis, Lassa virus, Legionella pneumophila, Leishmania species, Leptospira species, Listeria monocytogenes, Machupo virus, Measles morbillivirus, Metagonimus yokagawai, Middle East respiratory syndrome coronavirus (MERS), Monkeypox virus, Mumps orthorubulavirus, Mycobacterium leprae, Mycobacterium lepromatosis, Mycobacterium tuberculosis, Mycobacterium ulcerans, Mycoplasma genitalium, Mycoplasma pneumoniae, Necator americanus, Neisseria gonorrhea, Neisseria meningitides, Norovirus, Orthomyxoviridae species, Parvovirus B19, Piedraia hortae, Plasmodium species, Pneumocystis jirovecii, Poliovirus, Propionibacterium propionicus, Rabies virus, Rhinovirus, Rickettsia akari, Rickettsia rickettsia, Rickettsia species, Rickettsia typhi, Rift Valley fever virus, Rotavirus, Rubella virus, Sable virus, Salmonella species, Sarcoptes scabiei, Schistosoma species, Severe acute respiratory syndrome coronavirus 2 (SARS-CoV-2), Shigella species, Sin Nombre virus, Sporothrix schenckii, Staphylococcus aureus, Staphylococcus species, Streptococcus agalactiae, Streptococcus pneumoniae, Streptococcus pyogenes, Taenia solium, Toxoplasma gondii, Trichinella spiralis, Trichomonas vaginalis, Trichuris trichiura, Trypanosoma brucei, Trypanosoma cruzi, Varicella zoster virus (VZV), Variola major, Variola minor, Venezuelan equine encephalitis virus, Vibrio cholera, Vibrio vulnificus, West Nile virus, Yellow fever virus, Yersinia enterocolitica, Yersinia pestis, Yersinia pseudotuberculosis, Zeaspora fungus, and Zika virus.

In one aspect of the methods disclosed herein, the pathogen is SARS-CoV-2.

In one aspect of the methods disclosed herein, the subject is a vertebrate, a mammal, a mouse, a primate, a simian, or a human.

In one aspect of the methods disclosed herein, the subject is a human.

In one aspect of the methods disclosed herein, a plurality of samples are obtained, each corresponding to a plurality of subjects, and a plurality of nucleic acid libraries are prepared simultaneously and then sequenced simultaneously.

In one aspect of the methods disclosed herein, the method is performed in a single-pot, closed tube chemistry. In some methods, the method is performed in a single-pot, open tube chemistry. In some methods, the method is performed in a split-pot, multi-tube chemistry using PCR pre-amplification.

In one aspect of the methods disclosed herein, the method is performed in a split-pot, multi-tube chemistry using MDA pre-amplification.

In one aspect of the methods disclosed herein, the method further comprises determining an infection status of the subject based on the plurality of nucleic acid reads from the subject's library.

The disclosure also provides a method for screening for a pathogen in a plurality of samples using next generation sequencing (NGS), the method comprising:
(a) obtaining the plurality of samples from a plurality of subjects and preparing an agnostic nucleic acid library from each sample in the plurality of samples, wherein each agnostic nucleic acid library comprises a sample specific barcode;
(b) selectively enriching each agnostic nucleic acid library for a plurality of pathogen specific consensus sequences from the pathogen to generate a plurality of enriched, barcoded nucleic acid libraries, wherein selective enrichment comprises targeted amplification of the plurality of conserved sequences in the pathogen; and
(c) sequencing the plurality of enriched, barcoded nucleic acid libraries at the same time using NGS to detect the presence of one or more of the plurality of conserved sequences in the pathogen.

In one aspect of the methods disclosed herein, the method disclosed herein further comprises determining an infection status of the subject based on the subject's library.

In one aspect of the methods disclosed herein, the method comprises using one or more of the following oligonucleotides:
(i) an anchored oligonucleotide comprising:
   (1) a 3′ splint
   (2) a unique molecule identifier (UMI)
   (3) a sample-specific barcode; and
   (4) an oligo-dT;
(ii) a pathogen-specific oligonucleotide primer comprising:
   (1) an extended 3′ end cDNA splint
   (2) a minimal 3′ end cDNA splint
   (3) a 3′ end cDNA UMI; and
   (4) a pathogen specific consensus sequence;
(iii) a 3′ indexed adapter oligonucleotide comprising:
   (1) a 3′ adapter;
   (2) a 3′ barcode; and
   (3) a 3′ coupling sequence;
(iv) a 5′ indexed adapter oligonucleotide comprising:
   (1) a 5′ adapter;
   (2) a 5′ barcode; and
   (3) a 5′ coupling sequence;
(v) a pathogen specific template switching oligonucleotide comprising:
   (1) a pathogen specific consensus sequence; and
   (2) a template switching motif;
(vi) a generic template switching oligonucleotide comprising:
   (1) a generic tailing motif; and
   (2) a template switching motif;
(vii) a universal cDNA coupler forward primer oligonucleotide comprising:
   (1) an extended 3′ end cDNA splint; and
   (2) a minimal 3′ end cDNA splint;
(viii) a pathogen specific enrichment coupler reverse primer oligonucleotide comprising:
   (1) a minimal 5′ end cDNA splint;
   (2) an extended 5′ end cDNA splint;
   (3) a 5′ end cDNA UMI; and
   (4) a pathogenic specific consensus sequence;
(ix) a generic cDNA coupler reverse primer oligonucleotide comprising:
   (1) a generic tailing motif; and
   (2) a template switching motif; or
(x) a rDNA blocking duplex oligonucleotide.

In one aspect of the methods disclosed herein, the nucleic acid sample comprises RNA, DNA, or both RNA and DNA.

In one aspect of the methods disclosed herein, the sample is a clinical sample.

In one aspect of the methods disclosed herein, the sample is selected from the group consisting of a nasopharyngeal swab, an oropharyngeal swab, a buccal swab, whole saliva sample, cell-free saliva sample, blood plasma, blood serum, whole blood, sputum, stool, urine, cerebral spinal fluid, synovial fluid, peritoneal fluid, pleural fluid, pericardial fluid, and bone marrow.

In one aspect of the methods disclosed herein, the sample comprises nucleic acid from a plurality of organisms.

In one aspect of the methods disclosed herein, the sample comprises nucleic acid from both the subject and the pathogen.

In one aspect of the methods disclosed herein, the pathogen specific consensus sequence comprises a sequence from a conserved region from the pathogen's genome.

In one aspect of the methods disclosed herein, the pathogen specific consensus sequence comprises a transcription-regulatory sequence motif.

In one aspect of the methods disclosed herein, the pathogen is selected from: Acinetobacter baumannii, Actinomyces gerencseriae, Actinomyces israelii, Alphavirus species (e.g., Chikungunya virus, Eastern equine encephalitis virus, Venezuelan equine encephalitis virus, and Western equine encephalitis virus), Anaplasma species, Ancylostoma duodenale, Angiostrongylus cantonensis, Angiostrongylus costaricensis, Arcanobacterium haemolyticum, Ascaris lumbricoides, Aspergillus species, Astroviridae species, Babesia species, Bacillus anthracis, Bacillus cereus, Bacteroides species, Balantidium coli, Bartonella bacilliformis, Bartonella henselae, Bartonella, Batrachochytrium dendrabatidis, Baylisascaris species, Blastocystis species, Blastomyces dermatitidis, Bordetella pertussis, Borrelia afzelii, Borrelia burgdorferi, Borrelia garinii, Brucella species, Burkholderia mallei, Burkholderia pseudomallei, Burkholderia species, Caliciviridae species, Campylobacter species, Candida albicans, Capillaria aerophila, Capillaria philippinensis, Chlamydia trachomatis, Chlamydophila pneumoniae, Clonorchis sinensis, Clostridium botulinum, Clostridium difficile, Clostridium perfringens, Clostridium tetani, Coccidioides immitis, Coccidioides posadasii, Colorado tick fever virus (CTFV), Corynebacterium diphtheria, Crimean-Congo hemorrhagic fever virus, Cryptococcus neoformans, Cryptosporidium species, Cyclospora cayetanensis, Cytomegalovirus, Dengue viruses (DEN-1, DEN-2, DEN-3 and DEN-4), Dientamoeba fragilis, Dracunculus medinensis, Ebolavirus (EBOV), Entamoeba histolytica, Enterobius vermicularis, Enterococcus species, Epstein-Barr virus (EBV), Escherichia coli, Fasciola gigantica, Fasciola hepatica, Fasciolopsis buski, Flavivirus species, Geotrichum candidum, Giardia lamblia, Haemophilus ducreyi, Haemophilus influenza, Hantaviridae family, Helicobacter pylori, Hepatitis A virus, Hepatitis B virus, Hepatitis C virus, Hepatitis D Virus, Hepatitis E virus, Herpes simplex virus 1 (HSV-1), Herpes simplex virus 2 (HSV-2), Histoplasma capsulatum, HIV (Human immunodeficiency virus), Human herpesvirus 6 (HHV-6), Human herpesvirus 7 (HHV-7), Human papillomavirus (HPV), Junin virus, Klebsiella granulomatis, Lassa virus, Legionella pneumophila, Leishmania species, Leptospira species, Listeria monocytogenes, Machupo virus, Measles morbillivirus, Metagonimus yokagawai, Middle East respiratory syndrome coronavirus (MERS), Monkeypox virus, Mumps orthorubulavirus, Mycobacterium leprae, Mycobacterium lepromatosis, Mycobacterium tuberculosis, Mycobacterium ulcerans, Mycoplasma genitalium, Mycoplasma pneumoniae, Necator americanus, Neisseria gonorrhea, Neisseria meningitides, Norovirus, Orthomyxoviridae species, Parvovirus B19, Piedraia hortae, Plasmodium species, Pneumocystis jirovecii, Poliovirus, Propionibacterium propionicus, Rabies virus, Rhinovirus, Rickettsia akari, Rickettsia rickettsia, Rickettsia species, Rickettsia typhi, Rift Valley fever virus, Rotavirus, Rubella virus, Sabia\acute{ } virus, Salmonella species, Sarcoptes scabiei, Schistosoma species, Severe acute respiratory syndrome coronavirus 2 (SARS-CoV-2), Shigella species, Sin Nombre virus, Sporothrix schenckii, Staphylococcus aureus, Staphylococcus species, Streptococcus agalactiae, Streptococcus pneumoniae, Streptococcus pyogenes, Taenia solium, Toxoplasma gondii, Trichinella spiralis, Trichomonas vaginalis, Trichuris trichiura, Trypanosoma brucei, Trypanosoma cruzi, Varicella zoster virus (VZV), Variola major, Variola minor, Venezuelan equine encephalitis virus, Vibrio cholera, Vibrio vulnificus, West Nile virus, Yellow fever virus, Yersinia enterocolitica, Yersinia pestis, Yersinia pseudotuberculosis, Zeaspora fungus, and Zika virus.

In one aspect of the methods disclosed herein, the pathogen is SARS-CoV-2.

In one aspect of the methods disclosed herein, the subject is a vertebrate, a mammal, a mouse, a primate, a simian, or a human.

In one aspect of the methods disclosed herein, the subject is a human.

In one aspect of the methods disclosed herein, a plurality of samples are obtained, each corresponding to a plurality of subjects, and a plurality of nucleic acid libraries are prepared simultaneously and then sequenced simultaneously.

In one aspect of the methods disclosed herein, the method is performed in a single-pot, closed tube chemistry. In some methods, the method is performed in a single-pot, open tube chemistry.

In one aspect of the methods disclosed herein, the method is performed in a split-pot, multi-tube chemistry using PCR pre-amplification.

In one aspect of the methods disclosed herein, the method is performed in a split-pot, multi-tube chemistry using MDA pre-amplification.

In one aspect of the methods disclosed herein, the method further comprises determining an infection status of the subject based on the plurality of nucleic acid reads from the subject's library.

The disclosure also provides a method of diagnosing SARS-CoV-2 (COVID-19) infection in a subject comprising:
(a) obtaining a sample from a subject suspected of suffering from SARS-CoV-2;
(b) measuring the expression of one or more genes selected from the genes listed in Tables 14 and/or 16;
(c) comparing the measured expression levels of the one or more genes selected from Tables 14 and/or 16 to the expression levels of the same one or more genes measured in a sample from an individual not suffering from SARS-CoV-2; and
(d) detecting a difference in the expression levels of the one or more genes selected from Tables 14 and/or 16 in the subject suspected of suffering from SARS-CoV-2.

The disclosure also provides a method of diagnosing SARS-CoV-2 (COVID-19) in a subject comprising:
(a) obtaining a sample from a subject suspected of suffering from SARS-CoV-2;
(b) measuring the expression of one or more genes selected from the genes listed in Tables 14 and/or 16; and
(c) comparing the measured expression levels of the one or more genes selected from Tables 14 and/or 16 to a reference value, wherein a diagnosis of SARS-CoV-2 is made if the measured gene expression differs from the reference value.

The disclosure also provides a method of detecting SARS-CoV-2 (COVID-19) in a subject comprising:
(a) obtaining a sample from the subject;
(b) measuring the expression of one or more genes selected from the genes listed in Tables 14 and/or 16; and
(c) comparing the measured expression levels of the one or more genes to the expression levels of the same genes in one or more samples taken from one or more individuals without SARS-CoV-2, wherein SARS-CoV-2 is detected if the measured gene expression level in the sample taken from the subject differs from the gene expression level measured in the sample taken from the one or more individuals without SARS-CoV-2.

The disclosure also provides a method of treating SARS-CoV-2 (COVID-19) comprising:
(a) obtaining a sample from a subject suspected of having SARS-CoV-2;
(b) measuring the expression of one or more genes selected from the genes listed in Tables 14 and/16;
(c) determining a difference between the expression of the one or more genes in the sample and the expression of the one or more genes in one or more reference samples; and
(d) altering the treatment of the subject based on the difference.

The disclosure also provides a method of diagnosing and/or treating SARS-CoV-2 (COVID-19) in a subject comprising:
(a) obtaining a sample from a subject suspected of suffering from SARS-CoV-2;
(b) measuring the expression of one or more genes selected from the genes listed in Tables 14 and/16; and
(c) comparing the measured expression levels of the one or more genes selected from Tables 14 and/or 16 to a reference value; wherein a diagnosis of SARS-CoV-2 is made if the measured gene expression differs from the reference value; and
(d) altering the treatment of the subject based on the difference.

The disclosure also provides a method of screening patients for SARS-CoV-2 (COVID-19) comprising:
(a) obtaining a sample from the subject;
(b) measuring the expression of one or more genes selected from the genes listed in Tables 14 and/or 16;
(c) comparing the measured expression of the one or more genes to the expression of the same genes in a reference sample; and
(d) classifying the subject as having a low-risk, intermediate-risk, or high-risk of developing severe COVID-19.

In one aspect of the methods disclosed herein, the expression level of the one or more genes is measured by detecting RNA in the sample. In some embodiments, the expression level of the one or more genes is measured by PCR, qPCR, RT-PCR, qRT-PCR, hybridization, or sequencing.

In one aspect of the methods disclosed herein, the expression level of the one or more genes is determined by normalizing the expression to one or more housekeeping genes.

In one aspect of the methods disclosed herein, the sample is selected from the group consisting of a nasopharyngeal swab, an oropharyngeal swab, a buccal swab, whole saliva sample, cell-free saliva sample, blood plasma, blood serum, whole blood, sputum, stool, urine, cerebral spinal fluid, synovial fluid, peritoneal fluid, pleural fluid, pericardial fluid, and bone marrow.

In one aspect of the methods disclosed herein, the one or more genes comprises or consists of ARFIP2, ARMC10, ATG4C, BBX, CAMKK2, CNKSR3, DNAJC22, EFNB1, FLJ42627, HOXB7, INE2, INTS13, KDM4B, MAFF, MEAK7, NME8, NWD1, PPA2, PRKN, RBM27, SAA2, SGSM2, SYCP2, TNFAIP8L3, TNFRSF9, TNRC6A, and ZNF292.

In one aspect of the methods disclosed herein, the one or more genes comprises or consists of AHI1, ANXA4, ATXN1, BRAT1, CAMTA1, CCDC32, CD84, CES3, CLDN16, CLUAP1, DDHD1, ECE1, EYA4, FAM111B, FAM169A, GNAL, KLHL5, LRCH1, MAN1B1-DT, MCTS1, NM_014933, NR_027180, NRARP, OXTR, PKHD1, PNPLA6, PRDM16, PROCR, RBFOX3, RBM5, RDM1P5, RINL, RNF41, SCPEP1, SNAP29, TRIP10, TTC39A, ZBTB16, ZDHHC3, and ZNF445.

In one aspect of the methods disclosed herein, the method has an accuracy of at least about 90%, 91%, 92%, 93%, 94%, 95%, 96%, 97%, 98%, or 99%.

The disclosure also provides a kit for detecting SARS-CoV-2 (COVID-19) in a subject, wherein the kit comprises reagents useful, sufficient, and/or necessary for determining the level of one or more genes in Tables 14 and/or 16. In certain embodiments, the reagents comprise oligonucleotide probes specifically hybridizing under high stringency to mRNA or cDNA of one or more genes in Tables 14 and/or 16.

In one aspect of the kits disclosed herein, the one or more genes comprises or consists of ARFIP2, ARMC10, ATG4C, BBX, CAMKK2, CNKSR3, DNAJC22, EFNB1, FLJ42627, HOXB7, INE2, INTS13, KDM4B, MAFF, MEAK7, NME8, NWD1, PPA2, PRKN, RBM27, SAA2, SGSM2, SYCP2, TNFAIP8L3, TNFRSF9, TNRC6A, and ZNF292.

In one aspect of the kits disclosed herein, the one or more genes comprises or consists of AHI1, ANXA4, ATXN1, BRAT1, CAMTA1, CCDC32, CD84, CES3, CLDN16, CLUAP1, DDHD1, ECE1, EYA4, FAM111B, FAM169A, GNAL, KLHL5, LRCH1, MAN1B1-DT, MCTS1, NM_014933, NR_027180, NRARP, OXTR, PKHD1, PNPLA6, PRDM16, PROCR, RBFOX3, RBM5, RDM1P5, RINL, RNF41, SCPEP1, SNAP29, TRIP10, TTC39A, ZBTB16, ZDHHC3, and ZNF445.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1A-1C show a graphical summary of genetic and sequencing performance features of SARS-CoV-2 viral transcripts (modified from Kim et al. 2020 [DOI: 10.1016/j.cell.2020.04.011]). (FIG. 1A) Genomic, structural, and transcriptional features of annotated SARS-CoV-2 gRNA (GenBank: NC_045512.2); red triangles represent loci with consensus TRS motifs, both at the 5′ cap gRNA leader sequence (TRS-L) or flanking CDS of sgRNAs encoding structural proteins within the gRNA body (TRS-B); also depicted are the sgRNA transcripts of SARS-CoV-2 along with their expected CDS lengths flanked by interspersed TRS-B loci, as well as the relative location of the CDC-compliant N2-F primer used in diagnostic qPCR-based screening for SARS-CoV-2 infection. (FIG. 1B) Estimated lengths of poly(A) tails for sgRNA transcripts and host mRNA from infected Vero cells (inoculation MOI=0.05, total RNA extraction at 4th passage post-inoculation) by nanopore-based direct RNA sequencing; yeast ENO2 mRNA was used as a spike-in quality control template. (FIG. 1C). High-throughput short-read sequencing performance of unbiased single-shot poly(A) RNA-seq libraries from SARS-CoV-2 infected Vero cells, depicting information splits between viral and host transcriptomes, fraction of reads at splice junctions of expected canonical leader-to-body sgRNA fusions, and their apportionment among SARS-CoV-2 sgRNA species based on alignment of read sequences downstream of the TRS-B motif.

FIG. 2 shows a schematic exemplifying four distinctly barcoded reverse transcription tailing primers of a 384-well RT Anch-dT Plex Set at equimolar concentrations into a single well of a 96-well plate. Repeating for every well in the plate, and making sure all barcoded primers are distinct between wells (i.e., each of the barcoded reverse transcription tailing primers are used only once, into a single 4-plex well mix).

FIG. 3 shows a schematic for combinatorial dual-indexing 96-plex adapter sets.

FIG. 4 shows a breakdown of sequential biochemistry reactions involved in synthesis of LeaSH RNA-seq libraries from a plurality of nucleic acids in an individual specimen.

FIG. 5 shows a generic architecture of synthesized reads, their building elements, and their parsing through a bioinformatics flowchart after sequencing for decoding into specimen-specific gene expression interpretation thereof (i.e., a generic pipeline).

FIG. 6 shows quality assurance of accuracy, fidelity, dynamic range, representation rates, and assignment of bioinformatic and agnostically decoded barcodes in a hyperplexed NGS library enriched for poly(A)⁺-tailed RNA from a stable human cell line using 384 reverse transcription barcodes in tandem with a subset of 96 indexed adapter combinations out of a 9,216-plex total catalog of simultaneously assembled Illumina-based 3′×5′ combinatorial dual indices.

FIG. 7 shows a designed architecture of reads in SARS-CoV-2 LeaSH RNA-seq libraries for Illumina-based sequencing, and confirmatory quality assessment of appropriate 3′ read assembly based on preponderant representation of targeted structural regulatory motifs via unsupervised k-mer enrichment analysis (FASTQC software).

FIG. 8 shows a designed architecture of reads in SARS-CoV-2 LeaSH RNA-seq libraries for Illumina-based sequencing, and confirmatory quality assessment of appropriate 5′ read assembly based on preponderant representation of consensus transcription regulatory sequences via unsupervised k-mer enrichment analysis (FASTQC software).

FIG. 9 shows a frequency distribution analysis of identified transcripts in LeaSH RNA-seq libraries synthesized from a pool of reference lysates containing human and SARS-CoV-2 RNA molecules, and their statistical enrichment for expression profiles with respect COVID-19 NGS expression data in the extant scientific literature (Enrichr online analysis software). Reference lysates were sourced by the U.S. Centers for Disease Control and Prevention and obtained through the Biodefense and Emerging Infections Research Resources Repository [Cat. No. NR-52285, NR-52286, NR-52287, NR-52350, NR-52358, and NR-52388].

FIGS. 10A-10D show diagnostic interpretation of 1,620 confirmatory rRT-qPCR assay on remnants samples tested initially at CLIA-certified facilities and later re-processed at NIEHS. FIG. 10A shows “ground-truth” expectations, or Reported Dx, based on scores obtained from CLIA-certified facilities. FIG. 10B shows observed scores, or Test Dx, based on repeated processing and retesting at NIEHS of remnant samples. FIG. 10C shows the distribution of Ct values (i.e., the observed number of PCR cycles to fluorescence-based relative quantification threshold) for amplicon targets N1, N2, and RP via CDC EUA rRT-qPCR repeated assays of remnant samples at NIEHS, apportioned by their combined Reported Dx vs. Test Dx score classification. FIG. 10D shows the observed confirmation probability of SARS-CoV-2 positive diagnosis by rRT-qPCR testing at NIEHS, among remnant samples with reported SARS-CoV-2 positive status based on initial testing at CLIA-certified facilities, and relative to observed Ct values for N1, N2, or RP targets alone in CDC EUA rRT-qPCR retests at NIEHS (Kaplan-Meier Estimator, right-censored for assays with confirmed SARS-CoV-2 positivity per rRT-qPCR retest at NIEHS).

FIGS. 11A-11D show electroporetic profiles illustrating performance of enzymatic polishing by duplex-specific nuclease (DSN) normalization on targeted RNA-derived sequencing libraries ladden with short-length artifact templates. FIG. 11A shows an original amplicon-targeting Illumina sequencing library size-selected by 0.75×-SPRI with adapterized bleed-through ˜100-bp fusion PCR primer-dimers before DSN normalization.

FIG. 11B shows the Illumina sequencing library from FIG. 11A after DSN treatment, 18-cycle PCR re-amplification, and customary 1×-SPRI library clean-up. FIG. 11C shows an original motif-enriched Ion Torrent sequencing library size-selected by 0.75×-SPRI with adapterized bleed-through ˜100-bp fusion PCR primer-dimers before DSN normalization. FIG. 11D shows the Ion Torrent sequencing library from FIG. 11C after DSN treatment, 18-cycle PCR re-amplification, and customary 1×-SPRI library clean-up.

FIGS. 12A-12D show diagnostic performance of IonSwab assays on the UTEP-ReproCell reference panel of SARS-CoV-2 positive samples tested initially at CLIA-certified facilities and later re-processed at NIEHS. FIG. 12A shows the observed confirmation probability of SARS-CoV-2 positive diagnosis at NIEHS by sequencing of IonSwab libraries before or after DSN normalization in Ion Chips with different net read output capacities, and relative to observed Ct values for N1, N2, or RP targets alone in CDC EUA rRT-qPCR retests at NIEHS (Kaplan-Meier Estimator, right-censored for assays with confirmed SARS-CoV-2 positivity per sequencing method at NIEHS). FIG. 12B shows fitted regressions between SARS-CoV-2 transcript counts by sequencing of IonSwab libraries vs. observed Ct values for N1 or N2 targets alone in CDC EUA rRT-qPCR retests at NIEHS. FIG. 12C shows the total transcripts extracted from IonSwab libraries sequenced in Ion 540 chips before and after DSN library normalization, colored by captured target class. FIG. 12D shows the split by captured target class of transcripts extracted from IonSwab libraries sequenced in Ion 540 chips before and after DSN library normalization.

FIGS. 13A-13H show diagnostic performance of IonPrimed assays on the UTEP-ReproCell reference panel of SARS-CoV-2 positive samples tested initially at CLIA-certified facilities and later re-processed at NIEHS. FIG. 13A shows the observed confirmation probability of SARS-CoV-2 positive diagnosis at NIEHS by sequencing of IonPrimed libraries after DSN normalization in single Ion 540 Chips each, relative to observed Ct values for N1, N2, or RP targets alone in CDC EUA rRT-qPCR retests at NIEHS (Kaplan-Meier Estimator, right-censored for assays with confirmed SARS-CoV-2 positivity per sequencing method at NIEHS). FIG. 13B shows the total transcripts extracted from IonPrimed libraries, colored by captured target class. FIG. 13C shows the rate of raw read sequencing throughput from IonPrimed libraries that was retained past filtering stages against UMI tagging in terms of total or SARS-CoV-2 transcripts. FIG. 13D shows fitted regressions between SARS-CoV-2 transcript counts by sequencing of IonPrimed libraries vs. observed Ct values for N1 or N2 targets alone in CDC EUA rRT-qPCR retests at NIEHS. FIG. 13E shows genomic alignments across the SARS-CoV-2 genome for transcripts detected by IonRTMix libra sequencing. FIG. 13F shows unsupervised clustering of samples from the UTEP-ReproCell reference panel based on transcriptional data from IonRTMix sequencing (left panel: two-dimensional dendrogram heatmaps where columns are genes driving clustering, rows are individual samples; right panel: depiction of left panel clustering in 2D latent space; right inset: quantile density overlay onto 2D latent space map highlighting location of SARS-CoV-2 enriched samples). FIG. 13G shows the correspondence analysis between transcriptional groupings of samples and latent classification clusters of candidate biomarkers identified by IonRTMix sequencing. FIG. 13B shows statistically significant gene-enriched sets in the extant literature with respect to biomarkers correlated with SARS-CoV-2 expression identified by IonSwab sequencing.

FIGS. 14A-14B show diagnostic performance of IonLeaSH assays on the UTEP-ReproCell reference panel of SARS-CoV-2 positive samples tested initially at CLIA-certified facilities and later re-processed at NIEHS. FIG. 14A shows the observed confirmation probability of SARS-CoV-2 positive diagnosis at NIEHS by sequencing of IonLeaSH libraries after DSN normalization in pairs of Ion Chips with different net read output capacities, and relative to observed Ct values for N1, N2, or RP targets alone in CDC EUA rRT-qPCR retests at NIEHS (Kaplan-Meier Estimator, right-censored for assays with confirmed SARS-CoV-2 positivity per sequencing method at NIEHS). FIG. 14B shows the total transcripts extracted from IonLeaSH libraries per individual sequencing run, colored by captured target class. FIG. 14C shows the total SARS-CoV-2 transcripts detected by compiling data from duplicate IonLeaSH sequencing runs, relative to the total number of transcripts retained after filtering for UMI tagging at different sequencing throughputs.

FIGS. 15A-15D show diagnostic performance for COVID-19 presentation of IonLeaSH assays on clinically relevant 161 specimens from 111 healthy and diseased donors. FIG. 15A shows “ground-truth” expectations or Reported Dx (top panel) based on original reported scores vs. observed scores or Test Dx (bottom panel) based on repeated processing and retesting at NIEHS from two independent RNA extraction rounds. FIG. 15B shows observed Ct values among SARS-CoV-2 positive specimens per rRT-qPCR retests at NIEHS from two independent RNA extraction rounds for N1, N2, or RP targets. FIG. 15C shows unsupervised clustering in 2D latent space of clinically relevant samples based on transcriptional data from IonLeaSH sequencing, with back-coloring illustrating their major groupins (top-left panel), donor reported status (top-right panel), detected SARS-CoV-2 viral loads by IonLeaSH (bottom-left panel) and their reported history of mechanical ventilation treatment (bottom right); circling highlights major groupings predominant with samples from COVID-19 symptomatic donors that required mechanical ventilation treatment. FIG. 15D shows the correspondence analysis between transcriptional major groupings of samples and latent classification clusters of agnostically identified candidate biomarkers based on IonLeaSH sequencing data, highlighting major groupings predominant with samples from COVID-19 symptomatic donors that required mechanical ventilation treatment along with their corresponding biomarker candidates.

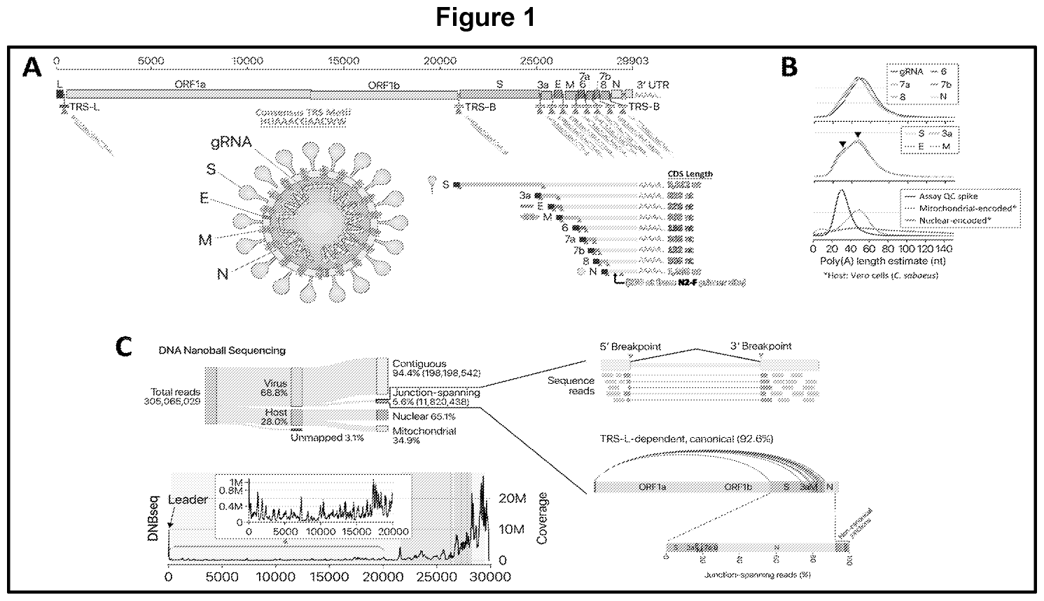

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION OF THE DISCLOSURE

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

Before describing the present invention in detail, a number of terms will be defined. Unless otherwise required by context, singular terms shall include pluralities and plural terms shall include the singular. For example, the singular forms “a,” “an,” and “the” include plural referents unless the context clearly dictates otherwise.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

The terms “comprises” and variations thereof do not have a limiting meaning where these terms appear in the description and claims.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “about” is used to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. Ranges and amounts can be expressed as “about” a particular value or range. About can also include the exact amount. Typically, the term “about” includes an amount that would be expected to be within experimental error. The term “about” includes values that are within 10% less to 10% greater of the value provided.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As utilized in accordance with the present disclosure, unless otherwise indicated, all technical and scientific terms shall be understood to have the same meaning as commonly understood by one of ordinary skill in the art.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, CA).

As used herein, the terms “polynucleotide,” “nucleotide,” “oligonucleotide,” and “nucleic acid” can be used interchangeably to refer to nucleic acid comprising deoxyribonucleic acid (DNA), ribonucleic acid (RNA), derivatives thereof, or combinations thereof, in either single-stranded or double-stranded embodiments depending on context as understood by the skilled worker. DNA can have one or more bases selected from the group consisting of adenine (symbol “A”), thymine (symbol “T”), cytosine (symbol “C”), or guanine (symbol “G”), and a ribonucleic acid can have one or more bases selected from the group consisting of adenine (symbol “A”), uracil (symbol “U”), cytosine (symbol “C”), or guanine (symbol “G”). Nucleic acids can also have the following IUPAC symbols:

TABLE 1
Nucleic acid IUPAC symbols.

| | | Bases | | | | Complementary |
|---|---|---|---|---|---|---|
| Description | Symbol | represented | | | | base |
| Weak | W | A | | | T | W |
| Strong | S | | C | G | | S |
| Amino | M | A | C | | | K |
| Keto | K | | | G | T | M |
| Purine | R | A | | G | | Y |
| Pyrimidine | Y | | C | | T | R |
| Not A | B | | C | G | T | V |
| Not C | D | A | | G | T | H |
| Not G | H | A | C | | T | D |
| Not T | V | A | C | G | | B |
| Any one base | N | A | C | G | T | N |

As used herein, the term “sample” generally refers to a biological sample from a subject from which nucleic acid (i.e., DNA, RNA, or both DNA and RNA) can be extracted or isolated. In certain embodiments, the sample may be a fluid sample, such as a blood sample, urine sample, or saliva sample. In some embodiments, the sample may be a tissue sample, such as a biopsy, core biopsy, needle aspirate, or fine needle aspirate. The sample may be a cell-free sample. A cell-free sample may include extracellular polynucleotides. In certain embodiments, the sample can be a nasopharyngeal swab, an oropharyngeal swab, a buccal swab, whole saliva sample, cell-free saliva sample, blood plasma, blood serum, whole blood, sputum, stool, urine, cerebral spinal fluid, synovial fluid, peritoneal fluid, pleural fluid, pericardial fluid, and bone marrow. In some embodiments, the sample is a clinical sample or clinical specimen.

As used herein, “pathogen” refers to any organism that can produce disease. A pathogen can refer to an infectious organism, for example such as a virus, bacterium, protozoan, prion, viroid, or fungus. A pathogen can include, but is not limited to, any of: Acinetobacter baumannii, Actinomyces gerencseriae, Actinomyces israelii, Alphavirus species (e.g., Chikungunya virus, Eastern equine encephalitis virus, Venezuelan equine encephalitis virus, and Western equine encephalitis virus), Anaplasma species, Ancylostoma duodenale, Angiostrongylus cantonensis, Angiostrongylus costaricensis, Arcanobacterium haemolyticum, Ascaris lumbricoides, Aspergillus species, Astroviridae species, Babesia species, Bacillus anthracis, Bacillus cereus, Bacteroides species, Balantidium coli, Bartonella bacilliformis, Bartonella henselae, Bartonella, Batrachochytrium dendrabatidis, Baylisascaris species, Blastocystis species, Blastomyces dermatitidis, Bordetella pertussis, Borrelia afzelii, Borrelia burgdorferi, Borrelia garinii, Brucella species, Burkholderia mallei, Burkholderia pseudomallei, Burkholderia species, Caliciviridae species, Campylobacter species, Candida albicans, Capillaria aerophila, Capillaria philippinensis, Chlamydia trachomatis, Chlamydophila pneumoniae, Clonorchis sinensis, Clostridium botulinum, Clostridium difficile, Clostridium perfringens, Clostridium tetani, Coccidioides immitis, Coccidioides posadasii, Colorado tick fever virus (CTFV), Corynebacterium diphtheria, Crimean-Congo hemorrhagic fever virus, Cryptococcus neoformans, Cryptosporidium species, Cyclospora cayetanensis, Cytomegalovirus, Dengue viruses (DEN-1, DEN-2, DEN-3 and DEN-4), Dientamoeba fragilis, Dracunculus medinensis, Ebolavirus (EBOV), Entamoeba histolytica, Enterobius vermicularis, Enterococcus species, Epstein-Barr virus (EBV), Escherichia coli, Fasciola gigantica, Fasciola hepatica, Fasciolopsis buski, Flavivirus species, Geotrichum candidum, Giardia lamblia, Haemophilus ducreyi, Haemophilus influenza, Hantaviridae family, Helicobacter pylori, Hepatitis A virus, Hepatitis B virus, Hepatitis C virus, Hepatitis D Virus, Hepatitis E virus, Herpes simplex virus 1 (HSV-1), Herpes simplex virus 2 (HSV-2), Histoplasma capsulatum, HIV (Human immunodeficiency virus), Human herpesvirus 6 (HHV-6), Human herpesvirus 7 (HHV-7), Human papillomavirus (HPV), Junin virus, Klebsiella granulomatis, Lassa virus, Legionella pneumophila, Leishmania species, Leptospira species, Listeria monocytogenes, Machupo virus, Measles morbillivirus, Metagonimus yokagawai, Middle East respiratory syndrome coronavirus (MERS), Monkeypox virus, Mumps orthorubulavirus, Mycobacterium leprae, Mycobacterium lepromatosis, Mycobacterium tuberculosis, Mycobacterium ulcerans, Mycoplasma genitalium, Mycoplasma pneumoniae, Necator americanus, Neisseria gonorrhea, Neisseria meningitides, Norovirus, Orthomyxoviridae species, Parvovirus B19, Piedraia hortae, Plasmodium species, Pneumocystis jirovecii, Poliovirus, Propionibacterium propionicus, Rabies virus, Rhinovirus, Rickettsia akari, Rickettsia rickettsia, Rickettsia species, Rickettsia typhi, Rift Valley fever virus, Rotavirus, Rubella virus, Sabia virus, Salmonella species, Sarcoptes scabiei, Schistosoma species, Severe acute respiratory syndrome coronavirus 2 (SARS-CoV-2), Shigella species, Sin Nombre virus, Sporothrix schenckii, Staphylococcus aureus, Staphylococcus species, Streptococcus agalactiae, Streptococcus pneumoniae, Streptococcus pyogenes, Taenia solium, Toxoplasma gondii, Trichinella spiralis, Trichomonas vaginalis, Trichuris trichiura, Trypanosoma brucei, Trypanosoma cruzi, Varicella zoster virus (VZV), Variola major, Variola minor, Venezuelan equine encephalitis virus, Vibrio cholera, Vibrio vulnificus, West Nile virus, Yellow fever virus, Yersinia enterocolitica, Yersinia pestis, Yersinia pseudotuberculosis, Zeaspora fungus, or Zika virus. In certain embodiments, the pathogen is SARS-CoV-2.

The term “subject” is intended to include human and non-human animals, particularly mammals. In some embodiments, the subject is a vertebrate, a mammal, a mouse, a primate, a simian, or a human. For example, a subject can be any mammal, including, but not limited to dogs, cats, horses, goats, sheep, cattle, pigs, mink, rats, or mice. In certain embodiments, the subject is human.

The phrase “measuring the expression of” or “determining the expression of,” as used herein, refers to determining or quantifying RNA or proteins expressed by the one or more gene or genes. The term “RNA” includes mRNA transcripts, and/or specific spliced variants of mRNA. The term “RNA product of the gene,” as used herein, refers to RNA transcripts transcribed from the gene and/or specific spliced variants. In some embodiments, mRNA is converted to cDNA before the gene expression levels are measured. With respect to proteins, gene expression refers to proteins translated from the RNA transcripts transcribed from the gene. The term “protein product of the gene” refers to proteins translated from RNA products of the gene. A number of methods can be used to detect or quantify the level of RNA products of the gene or genes within a sample, including microarrays, Real-Time PCR (RT-PCR; including quantitative RT-PCR), nuclease protection assays, RNA-sequencing (RNA-seq), and Northern blot analyses. A person skilled in the art will appreciate that a number of detection agents can be used to determine gene expression. For example, to detect RNA products of the biomarkers, probes, primers, complementary nucleotide sequences, or nucleotide sequences that hybridize to the RNA products can be used. In another example, to detect cDNA products of the biomarkers, probes, primers, complementary nucleotide sequences, or nucleotide sequences that hybridize to the cDNA products can be used. To detect protein products of the biomarkers, ligands or antibodies that specifically bind to the protein products can be used

In certain embodiments, gene expression can be analyzed using, e.g., direct DNA expression in microarray, Sanger sequencing analysis, Northern blot, the NANOSTRING® technology, serial analysis of gene expression (SAGE), RNA-seq, tissue microarray, or protein expression with immunohistochemistry or western blot technique. PCR generally involves the mixing of a nucleic acid sample, two or more primers that are designed to recognize the template DNA, a DNA polymerase, which may be a thermostable DNA polymerase such as Taq or Pfu, and deoxyribose nucleoside triphosphates (dNTP's). Reverse transcription PCR, quantitative reverse transcription PCR, and quantitative real time reverse transcription PCR are other specific examples of PCR. In real-time PCR analysis, additional reagents, methods, optical detection systems, and devices known in the art are used that allow a measurement of the magnitude of fluorescence in proportion to concentration of amplified DNA. In such analyses, incorporation of fluorescent dye into the amplified strands may be detected or measured.

The terms “next generation sequencing” and “NGS” are used interchangeably. Next-generation sequencing is based on the ability to sequence, in parallel, millions of nucleic acid fragments (DNA or RNA), and NGS technology has resulted in a dramatic increase in speed and content of sequencing at a fraction of the cost. NGS refers to sequencing methods that allow for massively parallel sequencing of clonally amplified molecules and of single nucleic acid molecules (DNA or RNA). Non-limiting examples of NGS include sequencing-by-synthesis using reversible dye terminators, and sequencing-by-ligation. Described briefly, first a nucleic acid library is prepared from a sample by fragmentation, purification and amplification of the nucleic acid in the sample. Individual fragments are then physically isolated by attachment to solid surfaces. The sequence of each of these nucleic acid fragments is read simultaneously by such techniques as sequencing by synthesis. The resulting sequence data (“reads”) are computationally aligned against a reference genome. Examples of NGS platforms include, but are not limited to: Illumine (Solexa) sequencing, Roche 454 sequencing, Ion Torrent: Proton/PGM sequencing, and SOLID sequencing.

The term “read” as used herein refers to a nucleic acid sequence from a portion of a nucleic acid sample. Typically, though not necessarily, a read represents a short sequence of contiguous base pairs in the nucleic acid sample. The read may be represented symbolically by the base pair sequence in “A”, “T”, “C”, and “G” of the sample portion, together with a probabilistic estimate of the correctness of the base (quality score). It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 20 bp) that can be used to identify a larger sequence or region. In certain embodiments, reads are aligned and mapped to a reference genome, a chromosome or a genomic region or a gene of the host and/or pathogen in the sample. A “reference genome” can refers to any particular known genome sequence, whether partial or complete, of any organism or virus which may be used to reference identified sequences from a subject.

In some embodiments, the methods disclosed herein comprise using one or more oligonucleotides comprising a barcode. As used herein, a “barcode” refers to a unique nucleic acid sequence such that each barcode is readily distinguishable from the other barcodes. Barcodes, which can also be referred to as an “index sequence”, “index”, or “tag” can be useful in downstream error correction, identification, or sequencing. Barcodes may be any length of nucleotide, but are preferably less than 30 nucleotides in length. For example, a barcode can be about 2 nucleotides, about 3 nucleotides, about 4 nucleotides, about 5 nucleotides, about 6 nucleotides, about 7 nucleotides, about 8 nucleotides, about 9 nucleotides, about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, or about 20 nucleotides. Detecting barcodes and determining the nucleic acid sequence of a barcode or plurality of barcodes allows large numbers of libraries to be pooled and sequenced simultaneously during a single run on a NGS instrument. Sample multiplexing exponentially increases the number of samples analyzed in a single run, without drastically increasing cost or time. Oligonucleotides containing a single barcode of the same sequence can be appended to a plurality of nucleic acids from an individual specimen. A collection of read sequences representing a plurality of nucleic acids from multiple specimens can then be parsed into data subsets originating from specific contributing specimens by their distinct barcode sequences present in sequenced reads.

In some embodiments, the methods disclosed here comprise using a single DNA duplex matching homologous rRNA sequences from ITS genomic loci in mammalian species. In some embodiments, the single DNA duplex has 3′ hexanediol-modified strands to block DNA polymerase processivity. In certain embodiments, the DNA duplex comprises:

Sequence 1
(SEQ ID NO: 1172)
TTAGAGGGACAAGTGGCGTTCAGCCACCCGAGATTG/3C6/
Complement
(SEQ ID NO: 1173)
CAATCTCGGGTGGCTGAACGCCACTTGTCCCTCTAA/3C6/

In some embodiments, the methods disclosed herein comprise using one or more oligonucleotides comprising a “unique molecular identifier” or “UMI.” Each unique molecular index (UMI) is an oligonucleotide sequence that can be used to identify an individual molecule or nucleic acid fragment present in the sample, or any of its amplified clonal copies thereafter, from within a plurality of derivative read sequences. To catalog the diversity of nucleic acids molecules in a sample while suppressing sequencing inaccuracy due to various sources of errors in NGS including, but not limited to, sample defects, PCR during library preparation, enrichment, clustering, and sequencing. UMI refers to a region of an oligonucleotide that includes a set of random “N” bases, wherein each “N” base is selected from any one of an “A” base, a “G” base, a “T” base, and a “C” base. A UMI can be any suitable nucleotide length. For example, about 2 “N” bases, about 3 “N” bases, about 4 N″ bases, about 5 “N” bases, about 6 “N” bases, about 7 “N” bases, about 8 “N” bases, about 9 “N” bases, about 10 “N” bases, about 12 “N” bases, about 14 “N” bases, about 16 “N” bases, about 18 “N” bases, about 20 “N” bases, or about 20 “N” bases. UMI sequence length can be determined based on the number of samples or targets to be screened and/or sequenced. For example, a longer UMI can facilitate a larger number of random base combinations and a greater number of unique identifiers. In an example, the UMI can be an 8N UMI. UMIs are similar to barcodes, which are commonly used to distinguish reads of one sample from reads of other samples, but UMIs are instead used to distinguish one source of DNA from another when many DNA molecules are sequenced together. Because there may be many more DNA molecules in a sample than samples in a sequencing run, there are typically many more distinct UMIs than distinct barcodes in a sequencing run. See also, International Publication No.: WO 2016/176091.

In some embodiments, the methods disclosed herein comprise using one or more oligonucleotides comprising “adaptors”, “adaptor regions” or “adapters.” Adapters generally refer to any linear oligonucleotide which can be ligated to a nucleic acid molecule of the disclosure. In some embodiments, the adapter is substantially non-complementary to the 3′ end or the 5′ end of any target sequence present in the sample. In some embodiments, suitable adapter lengths are in the range of about 10-100 nucleotides, about 12-60 nucleotides, or about 15-50 nucleotides in length. Generally, the adapter can include any combination of nucleotides and/or nucleic acids. In certain embodiments, the adapter can include a sequence that is substantially identical, or substantially complementary, to at least a portion of a primer. In next generation sequencing, adapters can be attached (by ligation or PCR) to the nucleic acid fragments of each sample library. Adapters can include platform-specific sequences for fragment recognition by the sequencing instrument (for example, the P5 and P7 sequences with Illumina platforms; see for example, U.S. Patent Application Publication No.: 20180023119). Each NGS instrument provider uses a specific set of adapter sequences for this purpose. Adapters can also comprise sample indexes. Sample indexes enable multiple samples to be sequenced together (i.e., multiplexed) on the same instrument flow cell or chip. Each sample index, typically 8-10 bases, is specific to a given sample library and is used for de-multiplexing during data analysis to assign individual sequence reads to the correct sample. In certain embodiments, adapters may contain single or dual sample indexes depending on the number of libraries combined and the level of accuracy desired.

As used herein, a “pathogen specific consensus sequence” refers a conserved region in a pathogen's genome that is well-conserved across a plurality of sequences belonging to the same pathogen species, and that can used to identify and/or confirm the presence of the pathogen in a sample. Conserved regions can be identified by locating a region within the genome of a pathogen that is a repeated sequence or represents, for example, a DNA binding motif, a DNA binding domain, or a DNA binding site, such as transcription regulatory motif. The consensus sequence can be about 4 to 30 nucleobase pairs long, but can be up to about 200 nucleotides in length. Conserved regions also can be determined by aligning sequences of the same or related genes from closely related species. In some embodiments, closely related species preferably are from the same genus. In some embodiments, alignment of sequences from two different species in a genus is adequate. Typically, DNA regions that exhibit at least about 50% sequence identity can be useful as conserved regions. In certain embodiments, conserved regions can exhibit at least 50% sequence identity, at least 60%, at least 70%, at least 80%, or at least 90% amino acid sequence identity. In some embodiments, a conserved region exhibits at least 91%, at least 92%, at least 93%, at least 94%, at least 95%, at least 96%, at least 97%, at least 98%, or at least 99% nucleic acid sequence identity. Multiple Sequence Analysis (MSA) is commonly used for aligning a set of sequences. Exemplary applications for MSA can include BLAST, BAlibase, T-Coffee, MAFFT, MUSCLE, Kalign, ClustalW2, or ClustalX2. In certain embodiments, a pathogen specific consensus sequence can be a DNA binding motif, such as a transcription regulatory sequence motif. In certain embodiments, a pathogen specific consensus sequence can be a transcription regulatory sequence motif from a virus of the Riboviria realm. In certain embodiments, a pathogen specific consensus sequence can be a transcription regulatory sequence motif from a virus of the Orthornavirae kingdom. In certain embodiments, a pathogen specific consensus sequence can be a transcription regulatory sequence motif from a virus of the Pisuviricota phylum. In certain embodiments, a pathogen specific consensus sequence can be a transcription regulatory sequence motif from a virus of the Pisoniviricetes class. In certain embodiments, a pathogen specific consensus sequence can be a transcription regulatory sequence motif from a virus of the Nidovirales order. In certain embodiments, a pathogen specific consensus sequence can be a transcription regulatory sequence motif from a virus of the Cornidovirineae suborder. In certain embodiments, a pathogen specific consensus sequence can be a transcription regulatory sequence motif from a virus of the Coronaviridae family. In certain embodiments, a pathogen specific consensus sequence can be a transcription regulatory sequence motif from a virus of the Orthocoronavirinae subfamily. In certain embodiments, a pathogen specific consensus sequence can be a transcription regulatory sequence motif from a virus of the Betacoronavirus genus. In certain embodiments, a pathogen specific consensus sequence can be a transcription regulatory sequence motif from a virus of the Sarbecovirus subgenus. In certain embodiments, a pathogen specific consensus sequence can be a transcription regulatory sequence motif from SARS-CoV-2. In an embodiment, the transcription regulatory sequence motif from SARS-CoV-2 is 5′-HUAAACGAACWW-3′ (SEQ ID NO:1174) or any of its possible reverse complementary sequences thereof.

In certain embodiments, a pathogen specific consensus sequence can be a structural regulatory sequence motif from a virus of the Riboviria realm. In certain embodiments, a pathogen specific consensus sequence can be a structural regulatory sequence motif from a virus of the Orthornavirae kingdom. In certain embodiments, a pathogen specific consensus sequence can be a structural regulatory sequence motif from a virus of the Pisuviricota phylum. In certain embodiments, a pathogen specific consensus sequence can be a structural regulatory sequence motif from a virus of the Pisoniviricetes class. In certain embodiments, a pathogen specific consensus sequence can be a structural regulatory sequence motif from a virus of the Nidovirales order. In certain embodiments, a pathogen specific consensus sequence can be a structural regulatory sequence motif from a virus of the Cornidovirineae suborder. In certain embodiments, a pathogen specific consensus sequence can be a structural regulatory sequence motif from a virus of the Coronaviridae family. In certain embodiments, a pathogen specific consensus sequence can be a structural regulatory sequence motif from a virus of the Orthocoronavirinae subfamily. In certain embodiments, a pathogen specific consensus sequence can be a structural regulatory sequence motif from a virus of the Betacoronavirus genus. In certain embodiments, a pathogen specific consensus sequence can be a structural regulatory sequence motif from a virus of the Sarbecovirus subgenus. In certain embodiments, a pathogen specific consensus sequence can be a structural regulatory sequence motif from SARS-CoV-2. In an embodiment, the structural regulatory sequence motif from SARS-CoV-2 is 5′-NKSWTCTTWK-3′ (SEQ ID NO:1175) or any of its possible reverse complementary sequences thereof.

In certain embodiments, an enrichment step can be beneficial to the methods disclosed herein. Enrichment can help in sequencing, detection, and analysis of targeted sequences of interest (a targeted sequence refers to selective and non-random amplification of two or more target sequences within a sample using at least one target-specific primer). Current techniques for targeted enrichment can be categorized according to the nature of their core reaction principle. In some embodiments, enrichment can be performed using: (1) ‘Hybrid capture’: wherein nucleic acid strands derived from the input sample are hybridized specifically to pre-prepared DNA fragments complementary to the targeted regions of interest, either in solution or on a solid support, so that one can physically capture and isolate the sequences of interest; (2) ‘Selective circularization’: also called molecular inversion probes (MIPs), gap-fill padlock probes and selector probes, wherein single-stranded DNA circles that include target region sequences are formed (by gap-filling and ligation chemistries) in a highly specific manner, creating structures with common DNA elements that are then used for selective amplification of the targeted regions of interest; or (3) PCR amplification: wherein polymerase chain reaction (PCR) is directed toward the targeted regions of interest by conducting multiple long-range PCRs in parallel, a limited number of standard multiplex PCRs or highly multiplexed PCR methods that amplify very large numbers of short fragments. In some embodiments, enrichment can be used after an initial round of reverse transcription (e.g., cDNA production). In certain embodiments, enrichment can be used after an initial round of reverse transcription and cDNA amplification for at least 5, 10, 15, 20, 25, 30, 40 or more cycles. In some embodiments, enrichment is employed after cDNA amplification. In some embodiments, amplified cDNA can be subjected to a clean-up step before the enrichment step using a column, gel extraction, or beads in order to remove unincorporated primers, unincorporated nucleotides, very short or very long nucleic acid fragments and enzymes. In some embodiments, enrichment is followed by a clean-up step before library preparation.

TABLE 2
mRNA_RT_Primers_3′_Anch-dT_384

| | | | SEQ |
|---|---|---|---|
| | | | ID |
| Well | Name | Sequence | NO. |
| A1 | Anch-dT.1 | ACGACGCTCTTCCGATCTNNNNNNNNTTCTCGCATGTTTTTTTTTTTTTTTTTTTTTTGC | 1 |
| A2 | Anch-dT.2 | ACGACGCTCTTCCGATCTNNNNNNNNTCCTACCAGTTTTTTTTTTTTTTTTTTTTTTTGC | 2 |
| A3 | Anch-dT.3 | ACGACGCTCTTCCGATCTNNNNNNNNGCGTTGGAGCTTTTTTTTTTTTTTTTTTTTTTGC | 3 |
| A4 | Anch-dT.4 | ACGACGCTCTTCCGATCTNNNNNNNNGATCTTACGCTTTTTTTTTTTTTTTTTTTTTTGC | 4 |
| A5 | Anch-dT.5 | ACGACGCTCTTCCGATCTNNNNNNNNCTGATGGTCATTTTTTTTTTTTTTTTTTTTTTGC | 5 |
| A6 | Anch-dT.6 | ACGACGCTCTTCCGATCTNNNNNNNNCCGAGAATCCTTTTTTTTTTTTTTTTTTTTTTGC | 6 |
| A7 | Anch-dT.7 | ACGACGCTCTTCCGATCTNNNNNNNNGCCGCAACGATTTTTTTTTTTTTTTTTTTTTTGC | 7 |
| A8 | Anch-dT.8 | ACGACGCTCTTCCGATCTNNNNNNNNTGAGTCTGGCTTTTTTTTTTTTTTTTTTTTTTGC | 8 |
| A9 | Anch-dT.9 | ACGACGCTCTTCCGATCTNNNNNNNNTGCGGACCTATTTTTTTTTTTTTTTTTTTTTTGC | 9 |
| A10 | Anch-dT.10 | ACGACGCTCTTCCGATCTNNNNNNNNACCTCGTTGATTTTTTTTTTTTTTTTTTTTTTGC | 10 |
| A11 | Anch-dT.11 | ACGACGCTCTTCCGATCTNNNNNNNNACGGAGGCGGTTTTTTTTTTTTTTTTTTTTTTGC | 11 |
| A12 | Anch-dT.12 | ACGACGCTCTTCCGATCTNNNNNNNNTAGATCTACTTTTTTTTTTTTTTTTTTTTTTTGC | 12 |
| A13 | Anch-dT.13 | ACGACGCTCTTCCGATCTNNNNNNNNAATTAAGACTTTTTTTTTTTTTTTTTTTTTTTGC | 13 |
| A14 | Anch-dT.14 | ACGACGCTCTTCCGATCTNNNNNNNNCCATTGCGTTTTTTTTTTTTTTTTTTTTTTTTGC | 14 |
| A15 | Anch-dT.15 | ACGACGCTCTTCCGATCTNNNNNNNNTTATTCATTCTTTTTTTTTTTTTTTTTTTTTTGC | 15 |
| A16 | Anch-dT.16 | ACGACGCTCTTCCGATCTNNNNNNNNATCTCCGAACTTTTTTTTTTTTTTTTTTTTTTGC | 16 |
| A17 | Anch-dT.17 | ACGACGCTCTTCCGATCTNNNNNNNNTTGACTTCAGTTTTTTTTTTTTTTTTTTTTTTGC | 17 |
| A18 | Anch-dT.18 | ACGACGCTCTTCCGATCTNNNNNNNNGGCAGGTATTTTTTTTTTTTTTTTTTTTTTTTGC | 18 |
| A19 | Anch-dT.19 | ACGACGCTCTTCCGATCTNNNNNNNNAGAGCTATAATTTTTTTTTTTTTTTTTTTTTTGC | 19 |
| A20 | Anch-dT.20 | ACGACGCTCTTCCGATCTNNNNNNNNCTAAGAGAAGTTTTTTTTTTTTTTTTTTTTTTGC | 20 |
| A21 | Anch-dT.21 | ACGACGCTCTTCCGATCTNNNNNNNNACTCAATAGGTTTTTTTTTTTTTTTTTTTTTTGC | 21 |
| A22 | Anch-dT.22 | ACGACGCTCTTCCGATCTNNNNNNNNCTTGCGCCGCTTTTTTTTTTTTTTTTTTTTTTGC | 22 |
| A23 | Anch-dT.23 | ACGACGCTCTTCCGATCTNNNNNNNNAATCGTAGCGTTTTTTTTTTTTTTTTTTTTTTGC | 23 |
| A24 | Anch-dT.24 | ACGACGCTCTTCCGATCTNNNNNNNNGGTACTGCCTTTTTTTTTTTTTTTTTTTTTTTGC | 24 |
| B1 | Anch-dT.25 | ACGACGCTCTTCCGATCTNNNNNNNNTAGAATTAACTTTTTTTTTTTTTTTTTTTTTTGC | 25 |
| B2 | Anch-dT.26 | ACGACGCTCTTCCGATCTNNNNNNNNGCCATTCTCCTTTTTTTTTTTTTTTTTTTTTTGC | 26 |
| B3 | Anch-dT.27 | ACGACGCTCTTCCGATCTNNNNNNNNTGCCGGCAGATTTTTTTTTTTTTTTTTTTTTTGC | 27 |
| B4 | Anch-dT.28 | ACGACGCTCTTCCGATCTNNNNNNNNTTACCGAGGCTTTTTTTTTTTTTTTTTTTTTTGC | 28 |
| B5 | Anch-dT.29 | ACGACGCTCTTCCGATCTNNNNNNNNATCATATTAGTTTTTTTTTTTTTTTTTTTTTTGC | 29 |
| B6 | Anch-dT.30 | ACGACGCTCTTCCGATCTNNNNNNNNTGGTCAGCCATTTTTTTTTTTTTTTTTTTTTTGC | 30 |
| B7 | Anch-dT.31 | ACGACGCTCTTCCGATCTNNNNNNNNACTATGCAATTTTTTTTTTTTTTTTTTTTTTTGC | 31 |
| B8 | Anch-dT.32 | ACGACGCTCTTCCGATCTNNNNNNNNCGACGCGACTTTTTTTTTTTTTTTTTTTTTTTGC | 32 |
| B9 | Anch-dT.33 | ACGACGCTCTTCCGATCTNNNNNNNNGATACGGAACTTTTTTTTTTTTTTTTTTTTTTGC | 33 |
| B10 | Anch-dT.34 | ACGACGCTCTTCCGATCTNNNNNNNNTTATCCGGATTTTTTTTTTTTTTTTTTTTTTTGC | 34 |
| B11 | Anch-dT.35 | ACGACGCTCTTCCGATCTNNNNNNNNTAGAGTAATATTTTTTTTTTTTTTTTTTTTTTGC | 35 |
| B12 | Anch-dT.36 | ACGACGCTCTTCCGATCTNNNNNNNNGCAGGTCCGTTTTTTTTTTTTTTTTTTTTTTTGC | 36 |
| B13 | Anch-dT.37 | ACGACGCTCTTCCGATCTNNNNNNNNTCGGCCTTACTTTTTTTTTTTTTTTTTTTTTTGC | 37 |
| B14 | Anch-dT.38 | ACGACGCTCTTCCGATCTNNNNNNNNAGAACGTCTCTTTTTTTTTTTTTTTTTTTTTTGC | 38 |
| B15 | Anch-dT.39 | ACGACGCTCTTCCGATCTNNNNNNNNCCAGTTCCAATTTTTTTTTTTTTTTTTTTTTTGC | 39 |
| B16 | Anch-dT.40 | ACGACGCTCTTCCGATCTNNNNNNNNGGCGTTAAGGTTTTTTTTTTTTTTTTTTTTTTGC | 40 |
| B17 | Anch-dT.41 | ACGACGCTCTTCCGATCTNNNNNNNNACTTAACCTTTTTTTTTTTTTTTTTTTTTTTTGC | 41 |
| B18 | Anch-dT.42 | ACGACGCTCTTCCGATCTNNNNNNNNCAACCGCTAATTTTTTTTTTTTTTTTTTTTTTGC | 42 |
| B19 | Anch-dT.43 | ACGACGCTCTTCCGATCTNNNNNNNNGACCTTGATATTTTTTTTTTTTTTTTTTTTTTGC | 43 |
| B2C | Anch-dT.44 | ACGACGCTCTTCCGATCTNNNNNNNNTCTGATACCATTTTTTTTTTTTTTTTTTTTTTGC | 44 |
| B21 | Anch-dT.45 | ACGACGCTCTTCCGATCTNNNNNNNNGAAGATCGAGTTTTTTTTTTTTTTTTTTTTTTGC | 45 |
| B22 | Anch-dT.46 | ACGACGCTCTTCCGATCTNNNNNNNNAGGAGCGGTATTTTTTTTTTTTTTTTTTTTTTGC | 46 |
| B23 | Anch-dT.47 | ACGACGCTCTTCCGATCTNNNNNNNNAAGAAGCTAGTTTTTTTTTTTTTTTTTTTTTTGC | 47 |
| B24 | Anch-dT.48 | ACGACGCTCTTCCGATCTNNNNNNNNTCCGGCCTCGTTTTTTTTTTTTTTTTTTTTTTGC | 48 |
| C1 | Anch-dT.49 | ACGACGCTCTTCCGATCTNNNNNNNNAGAGAAGGTTTTTTTTTTTTTTTTTTTTTTTTGC | 49 |
| C2 | Anch-dT.50 | ACGACGCTCTTCCGATCTNNNNNNNNCATACTCCGATTTTTTTTTTTTTTTTTTTTTTGC | 50 |
| C3 | Anch-dT.51 | ACGACGCTCTTCCGATCTNNNNNNNNGCTAACTTGCTTTTTTTTTTTTTTTTTTTTTTGC | 51 |
| C4 | Anch-dT.52 | ACGACGCTCTTCCGATCTNNNNNNNNAATCCATCTTTTTTTTTTTTTTTTTTTTTTTTGC | 52 |
| C5 | Anch-dT.53 | ACGACGCTCTTCCGATCTNNNNNNNNGGCTGAGCTCTTTTTTTTTTTTTTTTTTTTTTGC | 53 |
| C6 | Anch-dT.54 | ACGACGCTCTTCCGATCTNNNNNNNNCCGATTCCTGTTTTTTTTTTTTTTTTTTTTTTGC | 54 |
| C7 | Anch-dT.55 | ACGACGCTCTTCCGATCTNNNNNNNNACCGCCAACCTTTTTTTTTTTTTTTTTTTTTTGC | 55 |
| C8 | Anch-dT.56 | ACGACGCTCTTCCGATCTNNNNNNNNTGGCCTGAAGTTTTTTTTTTTTTTTTTTTTTTGC | 56 |
| C9 | Anch-dT.57 | ACGACGCTCTTCCGATCTNNNNNNNNAACCTCATTCTTTTTTTTTTTTTTTTTTTTTTGC | 57 |
| C10 | Anch-dT.58 | ACGACGCTCTTCCGATCTNNNNNNNNATAAGGAGCATTTTTTTTTTTTTTTTTTTTTTGC | 58 |
| C11 | Anch-dT.59 | ACGACGCTCTTCCGATCTNNNNNNNNCGAACGCCGGTTTTTTTTTTTTTTTTTTTTTTGC | 59 |
| C12 | Anch-dT.60 | ACGACGCTCTTCCGATCTNNNNNNNNGGTATGCTTGTTTTTTTTTTTTTTTTTTTTTTGC | 60 |
| C13 | Anch-dT.61 | ACGACGCTCTTCCGATCTNNNNNNNNAACCTGCGTATTTTTTTTTTTTTTTTTTTTTTGC | 61 |
| C14 | Anch-dT.62 | ACGACGCTCTTCCGATCTNNNNNNNNGGCAGACGCCTTTTTTTTTTTTTTTTTTTTTTGC | 62 |
| C15 | Anch-dT.63 | ACGACGCTCTTCCGATCTNNNNNNNNTAGCCGTCATTTTTTTTTTTTTTTTTTTTTTTGC | 63 |
| C16 | Anch-dT.64 | ACGACGCTCTTCCGATCTNNNNNNNNCCTGGAAGAGTTTTTTTTTTTTTTTTTTTTTTGC | 64 |
| C17 | Anch-dT.65 | ACGACGCTCTTCCGATCTNNNNNNNNGGAGGTTCTATTTTTTTTTTTTTTTTTTTTTTGC | 65 |
| C18 | Anch-dT.66 | ACGACGCTCTTCCGATCTNNNNNNNNCTAGTAGTCTTTTTTTTTTTTTTTTTTTTTTTGC | 66 |
| C19 | Anch-dT.67 | ACGACGCTCTTCCGATCTNNNNNNNNATCATCAACGTTTTTTTTTTTTTTTTTTTTTTGC | 67 |
| C20 | Anch-dT.68 | ACGACGCTCTTCCGATCTNNNNNNNNACGCGAGATTTTTTTTTTTTTTTTTTTTTTTTGC | 68 |
| C21 | Anch-dT.69 | ACGACGCTCTTCCGATCTNNNNNNNNGAAGAGGCATTTTTTTTTTTTTTTTTTTTTTTGC | 69 |
| C22 | Anch-dT.70 | ACGACGCTCTTCCGATCTNNNNNNNNGGTATCCGCCTTTTTTTTTTTTTTTTTTTTTTGC | 70 |
| C23 | Anch-dT.71 | ACGACGCTCTTCCGATCTNNNNNNNNAACTAGGCGCTTTTTTTTTTTTTTTTTTTTTTGC | 71 |
| C24 | Anch-dT.72 | ACGACGCTCTTCCGATCTNNNNNNNNTCGCTAAGCATTTTTTTTTTTTTTTTTTTTTTGC | 72 |
| D1 | Anch-dT.73 | ACGACGCTCTTCCGATCTNNNNNNNNTATATACTAATTTTTTTTTTTTTTTTTTTTTTGC | 73 |
| D2 | Anch-dT.74 | ACGACGCTCTTCCGATCTNNNNNNNNACTTGCTAGATTTTTTTTTTTTTTTTTTTTTTGC | 74 |
| D3 | Anch-dT.75 | ACGACGCTCTTCCGATCTNNNNNNNNAACCATTGGATTTTTTTTTTTTTTTTTTTTTTGC | 75 |
| D4 | Anch-dT.76 | ACGACGCTCTTCCGATCTNNNNNNNNTCGCGGTTGGTTTTTTTTTTTTTTTTTTTTTTGC | 76 |
| D5 | Anch-dT.77 | ACGACGCTCTTCCGATCTNNNNNNNNCGTAGTTACCTTTTTTTTTTTTTTTTTTTTTTGC | 77 |
| D6 | Anch-dT.78 | ACGACGCTCTTCCGATCTNNNNNNNNTCCAATCATCTTTTTTTTTTTTTTTTTTTTTTGC | 78 |
| D7 | Anch-dT.79 | ACGACGCTCTTCCGATCTNNNNNNNNAATCGATAATTTTTTTTTTTTTTTTTTTTTTTGC | 79 |
| D8 | Anch-dT.80 | ACGACGCTCTTCCGATCTNNNNNNNNCCATTATCTATTTTTTTTTTTTTTTTTTTTTTGC | 80 |
| D9 | Anch-dT.81 | ACGACGCTCTTCCGATCTNNNNNNNNTCAACGTAAGTTTTTTTTTTTTTTTTTTTTTTGC | 81 |
| D10 | Anch-dT.82 | ACGACGCTCTTCCGATCTNNNNNNNNTCTAATAGTATTTTTTTTTTTTTTTTTTTTTTGC | 82 |
| D11 | Anch-dT.83 | ACGACGCTCTTCCGATCTNNNNNNNNAACCGCTGGTTTTTTTTTTTTTTTTTTTTTTTGC | 83 |
| D12 | Anch-dT.84 | ACGACGCTCTTCCGATCTNNNNNNNNGATCGCTTCTTTTTTTTTTTTTTTTTTTTTTTGC | 84 |
| D13 | Anch-dT.85 | ACGACGCTCTTCCGATCTNNNNNNNNCTAACTAGATTTTTTTTTTTTTTTTTTTTTTTGC | 85 |
| D14 | Anch-dT.86 | ACGACGCTCTTCCGATCTNNNNNNNNGCTGGAACTTTTTTTTTTTTTTTTTTTTTTTTGC | 86 |
| D15 | Anch-dT.87 | ACGACGCTCTTCCGATCTNNNNNNNNAGGTTAGTTCTTTTTTTTTTTTTTTTTTTTTTGC | 87 |
| D16 | Anch-dT.88 | ACGACGCTCTTCCGATCTNNNNNNNNCATTCGACGGTTTTTTTTTTTTTTTTTTTTTTGC | 88 |
| D17 | Anch-dT.89 | ACGACGCTCTTCCGATCTNNNNNNNNCATTCAATCATTTTTTTTTTTTTTTTTTTTTTGC | 89 |
| D18 | Anch-dT.90 | ACGACGCTCTTCCGATCTNNNNNNNNCGGATTAGAATTTTTTTTTTTTTTTTTTTTTTGC | 90 |
| D19 | Anch-dT.91 | ACGACGCTCTTCCGATCTNNNNNNNNATCGGCTATCTTTTTTTTTTTTTTTTTTTTTTGC | 91 |
| D20 | Anch-dT.92 | ACGACGCTCTTCCGATCTNNNNNNNNCCTTGATCGTTTTTTTTTTTTTTTTTTTTTTTGC | 92 |
| D21 | Anch-dT.93 | ACGACGCTCTTCCGATCTNNNNNNNNACGAAGTCAATTTTTTTTTTTTTTTTTTTTTTGC | 93 |
| D22 | Anch-dT.94 | ACGACGCTCTTCCGATCTNNNNNNNNTTACCTCGACTTTTTTTTTTTTTTTTTTTTTTGC | 94 |
| D23 | Anch-dT.95 | ACGACGCTCTTCCGATCTNNNNNNNNGGAGGATAGCTTTTTTTTTTTTTTTTTTTTTTGC | 95 |
| D24 | Anch-dT.96 | ACGACGCTCTTCCGATCTNNNNNNNNGGCTCTCTATTTTTTTTTTTTTTTTTTTTTTTGC | 96 |
| E1 | Anch-dT.97 | ACGACGCTCTTCCGATCTNNNNNNNNCGGTCAAGAATTTTTTTTTTTTTTTTTTTTTTGC | 97 |
| E2 | Anch-dT.98 | ACGACGCTCTTCCGATCTNNNNNNNNCGCTCCTAACTTTTTTTTTTTTTTTTTTTTTTGC | 98 |
| E3 | Anch-dT.99 | ACGACGCTCTTCCGATCTNNNNNNNNATCCATGACTTTTTTTTTTTTTTTTTTTTTTTGC | 99 |
| E4 | Anch-dT.100 | ACGACGCTCTTCCGATCTNNNNNNNNAACCTGGTCTTTTTTTTTTTTTTTTTTTTTTTGC | 100 |
| E5 | Anch-dT.101 | ACGACGCTCTTCCGATCTNNNNNNNNACCGAAGACCTTTTTTTTTTTTTTTTTTTTTTGC | 101 |
| E6 | Anch-dT.102 | ACGACGCTCTTCCGATCTNNNNNNNNGGTACCGGCATTTTTTTTTTTTTTTTTTTTTTGC | 102 |
| E7 | Anch-dT.103 | ACGACGCTCTTCCGATCTNNNNNNNNAAGCCAGTTATTTTTTTTTTTTTTTTTTTTTTGC | 103 |
| E8 | Anch-dT.104 | ACGACGCTCTTCCGATCTNNNNNNNNTCTTGCCGACTTTTTTTTTTTTTTTTTTTTTTGC | 104 |
| E9 | Anch-dT.105 | ACGACGCTCTTCCGATCTNNNNNNNNAAGACCGTTGTTTTTTTTTTTTTTTTTTTTTTGC | 105 |
| E10 | Anch-dT.106 | ACGACGCTCTTCCGATCTNNNNNNNNAGGTTAGCATTTTTTTTTTTTTTTTTTTTTTTGC | 106 |
| E11 | Anch-dT.107 | ACGACGCTCTTCCGATCTNNNNNNNNTTCGCCTCCATTTTTTTTTTTTTTTTTTTTTTGC | 107 |
| E12 | Anch-dT.108 | ACGACGCTCTTCCGATCTNNNNNNNNAGAGCCAAGGTTTTTTTTTTTTTTTTTTTTTTGC | 108 |
| E13 | Anch-dT.109 | ACGACGCTCTTCCGATCTNNNNNNNNAATACCATCCTTTTTTTTTTTTTTTTTTTTTTGC | 109 |
| E14 | Anch-dT.110 | ACGACGCTCTTCCGATCTNNNNNNNNAGCTCTCCTCTTTTTTTTTTTTTTTTTTTTTTGC | 110 |
| E15 | Anch-dT.111 | ACGACGCTCTTCCGATCTNNNNNNNNCTTGATTGCCTTTTTTTTTTTTTTTTTTTTTTGC | 111 |
| E16 | Anch-dT.112 | ACGACGCTCTTCCGATCTNNNNNNNNAGCTTATCCGTTTTTTTTTTTTTTTTTTTTTTGC | 112 |
| E17 | Anch-dT.113 | ACGACGCTCTTCCGATCTNNNNNNNNAAGAATCTGATTTTTTTTTTTTTTTTTTTTTTGC | 113 |
| E18 | Anch-dT.114 | ACGACGCTCTTCCGATCTNNNNNNNNCATCTCTGCATTTTTTTTTTTTTTTTTTTTTTGC | 114 |
| E19 | Anch-dT.115 | ACGACGCTCTTCCGATCTNNNNNNNNACCTGGCCAATTTTTTTTTTTTTTTTTTTTTTGC | 115 |
| E20 | Anch-dT.116 | ACGACGCTCTTCCGATCTNNNNNNNNTAACTGGTTATTTTTTTTTTTTTTTTTTTTTTGC | 116 |
| E21 | Anch-dT.117 | ACGACGCTCTTCCGATCTNNNNNNNNTTGCTAACGGTTTTTTTTTTTTTTTTTTTTTTGC | 117 |
| E22 | Anch-dT.118 | ACGACGCTCTTCCGATCTNNNNNNNNACTAGAGAGTTTTTTTTTTTTTTTTTTTTTTTGC | 118 |
| E23 | Anch-dT.119 | ACGACGCTCTTCCGATCTNNNNNNNNAATGCCGCTTTTTTTTTTTTTTTTTTTTTTTTGC | 119 |
| E24 | Anch-dT.120 | ACGACGCTCTTCCGATCTNNNNNNNNTATAGACGCATTTTTTTTTTTTTTTTTTTTTTGC | 120 |
| F1 | Anch-dT.121 | ACGACGCTCTTCCGATCTNNNNNNNNTCAATCGCATTTTTTTTTTTTTTTTTTTTTTTGC | 121 |
| F2 | Anch-dT.122 | ACGACGCTCTTCCGATCTNNNNNNNNTTCTTAATAATTTTTTTTTTTTTTTTTTTTTTGC | 122 |
| F3 | Anch-dT.123 | ACGACGCTCTTCCGATCTNNNNNNNNGTCCTAGAGGTTTTTTTTTTTTTTTTTTTTTTGC | 123 |
| F4 | Anch-dT.124 | ACGACGCTCTTCCGATCTNNNNNNNNATATTGATACTTTTTTTTTTTTTTTTTTTTTTGC | 124 |
| F5 | Anch-dT.125 | ACGACGCTCTTCCGATCTNNNNNNNNCCGCTGCCAGTTTTTTTTTTTTTTTTTTTTTTGC | 125 |
| F6 | Anch-dT.126 | ACGACGCTCTTCCGATCTNNNNNNNNCCTAGTACGTTTTTTTTTTTTTTTTTTTTTTTGC | 126 |
| F7 | Anch-dT.127 | ACGACGCTCTTCCGATCTNNNNNNNNCAATTACCGTTTTTTTTTTTTTTTTTTTTTTTGC | 127 |
| F8 | Anch-dT.128 | ACGACGCTCTTCCGATCTNNNNNNNNGGCCGTAGTCTTTTTTTTTTTTTTTTTTTTTTGC | 128 |
| F9 | Anch-dT.129 | ACGACGCTCTTCCGATCTNNNNNNNNCGATTACGGCTTTTTTTTTTTTTTTTTTTTTTGC | 129 |
| F10 | Anch-dT.130 | ACGACGCTCTTCCGATCTNNNNNNNNTAATGAACGATTTTTTTTTTTTTTTTTTTTTTGC | 130 |
| F11 | Anch-dT.131 | ACGACGCTCTTCCGATCTNNNNNNNNCCGTTCCTTATTTTTTTTTTTTTTTTTTTTTTGC | 131 |
| F12 | Anch-dT.132 | ACGACGCTCTTCCGATCTNNNNNNNNGGTACCATATTTTTTTTTTTTTTTTTTTTTTTGC | 132 |
| F13 | Anch-dT.133 | ACGACGCTCTTCCGATCTNNNNNNNNCCGATTCGCATTTTTTTTTTTTTTTTTTTTTTGC | 133 |
| F14 | Anch-dT.134 | ACGACGCTCTTCCGATCTNNNNNNNNATGGCTCTGCTTTTTTTTTTTTTTTTTTTTTTGC | 134 |
| F15 | Anch-dT.135 | ACGACGCTCTTCCGATCTNNNNNNNNGTATAATACGTTTTTTTTTTTTTTTTTTTTTTGC | 135 |
| F16 | Anch-dT.136 | ACGACGCTCTTCCGATCTNNNNNNNNATCAGCAAGTTTTTTTTTTTTTTTTTTTTTTTGC | 136 |
| F17 | Anch-dT.137 | ACGACGCTCTTCCGATCTNNNNNNNNGGCGAACTCGTTTTTTTTTTTTTTTTTTTTTTGC | 137 |
| F18 | Anch-dT.138 | ACGACGCTCTTCCGATCTNNNNNNNNTTAATTGAATTTTTTTTTTTTTTTTTTTTTTTGC | 138 |
| F19 | Anch-dT.139 | ACGACGCTCTTCCGATCTNNNNNNNNTTAGGACCGGTTTTTTTTTTTTTTTTTTTTTTGC | 139 |
| F20 | Anch-dT.140 | ACGACGCTCTTCCGATCTNNNNNNNNAAGTAAGAGCTTTTTTTTTTTTTTTTTTTTTTGC | 140 |
| F21 | Anch-dT.141 | ACGACGCTCTTCCGATCTNNNNNNNNCCTTGGTCCATTTTTTTTTTTTTTTTTTTTTTGC | 141 |
| F22 | Anch-dT.142 | ACGACGCTCTTCCGATCTNNNNNNNNCATCAGAATGTTTTTTTTTTTTTTTTTTTTTTGC | 142 |
| F23 | Anch-dT.143 | ACGACGCTCTTCCGATCTNNNNNNNNTTATAGCAGATTTTTTTTTTTTTTTTTTTTTTGC | 143 |
| F24 | Anch-dT.144 | ACGACGCTCTTCCGATCTNNNNNNNNTTACTTGGAATTTTTTTTTTTTTTTTTTTTTTGC | 144 |
| G1 | Anch-dT.145 | ACGACGCTCTTCCGATCTNNNNNNNNGCTCAGCCGGTTTTTTTTTTTTTTTTTTTTTTGC | 145 |
| G2 | Anch-dT.146 | ACGACGCTCTTCCGATCTNNNNNNNNACGTCCGCAGTTTTTTTTTTTTTTTTTTTTTTGC | 146 |
| G3 | Anch-dT.147 | ACGACGCTCTTCCGATCTNNNNNNNNTTGACTGACGTTTTTTTTTTTTTTTTTTTTTTGC | 147 |
| G4 | Anch-dT.148 | ACGACGCTCTTCCGATCTNNNNNNNNTTGCGAGGCATTTTTTTTTTTTTTTTTTTTTTGC | 148 |
| G5 | Anch-dT.149 | ACGACGCTCTTCCGATCTNNNNNNNNTTCCAACCGCTTTTTTTTTTTTTTTTTTTTTTGC | 149 |
| GE | Anch-dT.150 | ACGACGCTCTTCCGATCTNNNNNNNNTAACCTTCGGTTTTTTTTTTTTTTTTTTTTTTGC | 150 |
| G7 | Anch-dT.151 | ACGACGCTCTTCCGATCTNNNNNNNNTCAAGCCGATTTTTTTTTTTTTTTTTTTTTTTGC | 151 |
| G8 | Anch-dT.152 | ACGACGCTCTTCCGATCTNNNNNNNNCTTGCAACCTTTTTTTTTTTTTTTTTTTTTTTGC | 152 |
| G9 | Anch-dT.153 | ACGACGCTCTTCCGATCTNNNNNNNNCCATCGCGAATTTTTTTTTTTTTTTTTTTTTTGC | 153 |
| G10 | Anch-dT.154 | ACGACGCTCTTCCGATCTNNNNNNNNTAGACTTCTTTTTTTTTTTTTTTTTTTTTTTTGC | 154 |
| G11 | Anch-dT.155 | ACGACGCTCTTCCGATCTNNNNNNNNGTCCTTAAGATTTTTTTTTTTTTTTTTTTTTTGC | 155 |
| G12 | Anch-dT.156 | ACGACGCTCTTCCGATCTNNNNNNNNAGTAACGGTCTTTTTTTTTTTTTTTTTTTTTTGC | 156 |
| G13 | Anch-dT.157 | ACGACGCTCTTCCGATCTNNNNNNNNGTTCGTCAGATTTTTTTTTTTTTTTTTTTTTTGC | 157 |
| G14 | Anch-dT.158 | ACGACGCTCTTCCGATCTNNNNNNNNCGCCTAATGCTTTTTTTTTTTTTTTTTTTTTTGC | 158 |
| G15 | Anch-dT.159 | ACGACGCTCTTCCGATCTNNNNNNNNACCGGAATTATTTTTTTTTTTTTTTTTTTTTTGC | 159 |
| G16 | Anch-dT.160 | ACGACGCTCTTCCGATCTNNNNNNNNTAGGCCATAGTTTTTTTTTTTTTTTTTTTTTTGC | 160 |
| G17 | Anch-dT.161 | ACGACGCTCTTCCGATCTNNNNNNNNTAACTCTTAGTTTTTTTTTTTTTTTTTTTTTTGC | 161 |
| G18 | Anch-dT.162 | ACGACGCTCTTCCGATCTNNNNNNNNTATGAGTTAATTTTTTTTTTTTTTTTTTTTTTGC | 162 |
| G19 | Anch-dT.163 | ACGACGCTCTTCCGATCTNNNNNNNNTATCATGATCTTTTTTTTTTTTTTTTTTTTTTGC | 163 |
| G20 | Anch-dT.164 | ACGACGCTCTTCCGATCTNNNNNNNNGAGCATATGGTTTTTTTTTTTTTTTTTTTTTTGC | 164 |
| G21 | Anch-dT.165 | ACGACGCTCTTCCGATCTNNNNNNNNTAACGATCCATTTTTTTTTTTTTTTTTTTTTTGC | 165 |
| G22 | Anch-dT.166 | ACGACGCTCTTCCGATCTNNNNNNNNCGGCGTAACTTTTTTTTTTTTTTTTTTTTTTTGC | 166 |
| G23 | Anch-dT.167 | ACGACGCTCTTCCGATCTNNNNNNNNCGTCGCAGCCTTTTTTTTTTTTTTTTTTTTTTGC | 167 |
| G24 | Anch-dT.168 | ACGACGCTCTTCCGATCTNNNNNNNNGTAGCTCCATTTTTTTTTTTTTTTTTTTTTTTGC | 168 |
| H1 | Anch-dT.169 | ACGACGCTCTTCCGATCTNNNNNNNNTTGCCTTGGCTTTTTTTTTTTTTTTTTTTTTTGC | 169 |
| H2 | Anch-dT.170 | ACGACGCTCTTCCGATCTNNNNNNNNTGCTAATTCTTTTTTTTTTTTTTTTTTTTTTTGC | 170 |
| H3 | Anch-dT.171 | ACGACGCTCTTCCGATCTNNNNNNNNGTCCTACTTGTTTTTTTTTTTTTTTTTTTTTTGC | 171 |
| H4 | Anch-dT.172 | ACGACGCTCTTCCGATCTNNNNNNNNGGTAGGTTAGTTTTTTTTTTTTTTTTTTTTTTGC | 172 |
| H5 | Anch-dT.173 | ACGACGCTCTTCCGATCTNNNNNNNNGAGCATCATTTTTTTTTTTTTTTTTTTTTTTTGC | 173 |
| H6 | Anch-dT.174 | ACGACGCTCTTCCGATCTNNNNNNNNCCGCTCCGGCTTTTTTTTTTTTTTTTTTTTTTGC | 174 |
| H7 | Anch-dT.175 | ACGACGCTCTTCCGATCTNNNNNNNNTTCTTCCGGTTTTTTTTTTTTTTTTTTTTTTTGC | 175 |
| H8 | Anch-dT.176 | ACGACGCTCTTCCGATCTNNNNNNNNAGGAGAGAACTTTTTTTTTTTTTTTTTTTTTTGC | 176 |
| H9 | Anch-dT.177 | ACGACGCTCTTCCGATCTNNNNNNNNTAACTCAATTTTTTTTTTTTTTTTTTTTTTTTGC | 177 |
| H10 | Anch-dT.178 | ACGACGCTCTTCCGATCTNNNNNNNNACTATAGGTTTTTTTTTTTTTTTTTTTTTTTTGC | 178 |
| H11 | Anch-dT.179 | ACGACGCTCTTCCGATCTNNNNNNNNCAAGATGCCGTTTTTTTTTTTTTTTTTTTTTTGC | 179 |
| H12 | Anch-dT.180 | ACGACGCTCTTCCGATCTNNNNNNNNAACGTCTAGTTTTTTTTTTTTTTTTTTTTTTTGC | 180 |
| H13 | Anch-dT.181 | ACGACGCTCTTCCGATCTNNNNNNNNAGGTATACTCTTTTTTTTTTTTTTTTTTTTTTGC | 181 |
| H14 | Anch-dT.182 | ACGACGCTCTTCCGATCTNNNNNNNNTTCATAGGACTTTTTTTTTTTTTTTTTTTTTTGC | 182 |
| H15 | Anch-dT.183 | ACGACGCTCTTCCGATCTNNNNNNNNGGAGGCCTCCTTTTTTTTTTTTTTTTTTTTTTGC | 183 |
| H16 | Anch-dT.184 | ACGACGCTCTTCCGATCTNNNNNNNNTTCAATATAATTTTTTTTTTTTTTTTTTTTTTGC | 184 |
| H17 | Anch-dT.185 | ACGACGCTCTTCCGATCTNNNNNNNNACGTCATATATTTTTTTTTTTTTTTTTTTTTTGC | 185 |
| H18 | Anch-dT.186 | ACGACGCTCTTCCGATCTNNNNNNNNTTGACCAGGATTTTTTTTTTTTTTTTTTTTTTGC | 186 |
| H19 | Anch-dT.187 | ACGACGCTCTTCCGATCTNNNNNNNNCGGTTGCGCGTTTTTTTTTTTTTTTTTTTTTTGC | 187 |
| H20 | Anch-dT.188 | ACGACGCTCTTCCGATCTNNNNNNNNCAAGGAGGTCTTTTTTTTTTTTTTTTTTTTTTGC | 188 |
| H21 | Anch-dT.189 | ACGACGCTCTTCCGATCTNNNNNNNNTTACGATGAATTTTTTTTTTTTTTTTTTTTTTGC | 189 |
| H22 | Anch-dT.190 | ACGACGCTCTTCCGATCTNNNNNNNNTTGCTGGCATTTTTTTTTTTTTTTTTTTTTTTGC | 190 |
| H23 | Anch-dT.191 | ACGACGCTCTTCCGATCTNNNNNNNNGAGGCATCAATTTTTTTTTTTTTTTTTTTTTTGC | 191 |
| H24 | Anch-dT.192 | ACGACGCTCTTCCGATCTNNNNNNNNATTCGACCAATTTTTTTTTTTTTTTTTTTTTTGC | 192 |
| I1 | Anch-dT.193 | ACGACGCTCTTCCGATCTNNNNNNNNCCGCGGCTCATTTTTTTTTTTTTTTTTTTTTTGC | 193 |
| I2 | Anch-dT.194 | ACGACGCTCTTCCGATCTNNNNNNNNGGCTCCTCGTTTTTTTTTTTTTTTTTTTTTTTGC | 194 |
| I3 | Anch-dT.195 | ACGACGCTCTTCCGATCTNNNNNNNNGTTACGCAAGTTTTTTTTTTTTTTTTTTTTTTGC | 195 |
| I4 | Anch-dT.196 | ACGACGCTCTTCCGATCTNNNNNNNNAGCCGGTACCTTTTTTTTTTTTTTTTTTTTTTGC | 196 |
| I5 | Anch-dT.197 | ACGACGCTCTTCCGATCTNNNNNNNNACCTCTATCTTTTTTTTTTTTTTTTTTTTTTTGC | 197 |
| I6 | Anch-dT.198 | ACGACGCTCTTCCGATCTNNNNNNNNGGACTACTACTTTTTTTTTTTTTTTTTTTTTTGC | 198 |
| I7 | Anch-dT.199 | ACGACGCTCTTCCGATCTNNNNNNNNGTATCATCGATTTTTTTTTTTTTTTTTTTTTTGC | 199 |
| I8 | Anch-dT.200 | ACGACGCTCTTCCGATCTNNNNNNNNCCGCGATTATTTTTTTTTTTTTTTTTTTTTTTGC | 200 |
| I9 | Anch-dT.201 | ACGACGCTCTTCCGATCTNNNNNNNNATTCAGGTACTTTTTTTTTTTTTTTTTTTTTTGC | 201 |
| I10 | Anch-dT.202 | ACGACGCTCTTCCGATCTNNNNNNNNATGGAATTGGTTTTTTTTTTTTTTTTTTTTTTGC | 202 |
| I11 | Anch-dT.203 | ACGACGCTCTTCCGATCTNNNNNNNNGACGAAGCGTTTTTTTTTTTTTTTTTTTTTTTGC | 203 |
| I12 | Anch-dT.204 | ACGACGCTCTTCCGATCTNNNNNNNNCTTGCAGTAGTTTTTTTTTTTTTTTTTTTTTTGC | 204 |
| I13 | Anch-dT.205 | ACGACGCTCTTCCGATCTNNNNNNNNCTTGGTAATGTTTTTTTTTTTTTTTTTTTTTTGC | 205 |
| I14 | Anch-dT.206 | ACGACGCTCTTCCGATCTNNNNNNNNCAAGTCGACCTTTTTTTTTTTTTTTTTTTTTTGC | 206 |
| I15 | Anch-dT.207 | ACGACGCTCTTCCGATCTNNNNNNNNTAACGAATTGTTTTTTTTTTTTTTTTTTTTTTGC | 207 |
| I16 | Anch-dT.208 | ACGACGCTCTTCCGATCTNNNNNNNNTGAGAACCAATTTTTTTTTTTTTTTTTTTTTTGC | 208 |
| I17 | Anch-dT.209 | ACGACGCTCTTCCGATCTNNNNNNNNTTATTCTGAGTTTTTTTTTTTTTTTTTTTTTTGC | 209 |
| I18 | Anch-dT.210 | ACGACGCTCTTCCGATCTNNNNNNNNTTATTATGGTTTTTTTTTTTTTTTTTTTTTTTGC | 210 |
| I19 | Anch-dT.211 | ACGACGCTCTTCCGATCTNNNNNNNNATATGAGCCATTTTTTTTTTTTTTTTTTTTTTGC | 211 |
| I20 | Anch-dT.212 | ACGACGCTCTTCCGATCTNNNNNNNNCAACCAGTACTTTTTTTTTTTTTTTTTTTTTTGC | 212 |
| I21 | Anch-dT.213 | ACGACGCTCTTCCGATCTNNNNNNNNCATCCGACTATTTTTTTTTTTTTTTTTTTTTTGC | 213 |
| I22 | Anch-dT.214 | ACGACGCTCTTCCGATCTNNNNNNNNATCATGGCTGTTTTTTTTTTTTTTTTTTTTTTGC | 214 |
| I23 | Anch-dT.215 | ACGACGCTCTTCCGATCTNNNNNNNNCCGCAAGTTCTTTTTTTTTTTTTTTTTTTTTTGC | 215 |
| I24 | Anch-dT.216 | ACGACGCTCTTCCGATCTNNNNNNNNCTTCTCATTGTTTTTTTTTTTTTTTTTTTTTTGC | 216 |
| J1 | Anch-dT.217 | ACGACGCTCTTCCGATCTNNNNNNNNCAGGAGGAGATTTTTTTTTTTTTTTTTTTTTTGC | 217 |
| J2 | Anch-dT.218 | ACGACGCTCTTCCGATCTNNNNNNNNGATATCGGCGTTTTTTTTTTTTTTTTTTTTTTGC | 218 |
| J3 | Anch-dT.219 | ACGACGCTCTTCCGATCTNNNNNNNNCCAGTCCTCTTTTTTTTTTTTTTTTTTTTTTTGC | 219 |
| J4 | Anch-dT.220 | ACGACGCTCTTCCGATCTNNNNNNNNCATAGTTCGGTTTTTTTTTTTTTTTTTTTTTTGC | 220 |
| J5 | Anch-dT.221 | ACGACGCTCTTCCGATCTNNNNNNNNCGTAATGCAGTTTTTTTTTTTTTTTTTTTTTTGC | 221 |
| J6 | Anch-dT.222 | ACGACGCTCTTCCGATCTNNNNNNNNCCGTTCGGATTTTTTTTTTTTTTTTTTTTTTTGC | 222 |
| J7 | Anch-dT.223 | ACGACGCTCTTCCGATCTNNNNNNNNCCATAAGTCCTTTTTTTTTTTTTTTTTTTTTTGC | 223 |
| J8 | Anch-dT.224 | ACGACGCTCTTCCGATCTNNNNNNNNGGCAATGAGATTTTTTTTTTTTTTTTTTTTTTGC | 224 |
| J9 | Anch-dT.225 | ACGACGCTCTTCCGATCTNNNNNNNNCGGTTATGCCTTTTTTTTTTTTTTTTTTTTTTGC | 225 |
| J10 | Anch-dT.226 | ACGACGCTCTTCCGATCTNNNNNNNNTGGCCGGCCTTTTTTTTTTTTTTTTTTTTTTTGC | 226 |
| J11 | Anch-dT.227 | ACGACGCTCTTCCGATCTNNNNNNNNAGCTGCAATATTTTTTTTTTTTTTTTTTTTTTGC | 227 |
| J12 | Anch-dT.228 | ACGACGCTCTTCCGATCTNNNNNNNNTGGCCATGCATTTTTTTTTTTTTTTTTTTTTTGC | 228 |
| J13 | Anch-dT.229 | ACGACGCTCTTCCGATCTNNNNNNNNTGACGCTCCGTTTTTTTTTTTTTTTTTTTTTTGC | 229 |
| J14 | Anch-dT.230 | ACGACGCTCTTCCGATCTNNNNNNNNAACTGCTGCCTTTTTTTTTTTTTTTTTTTTTTGC | 230 |
| J15 | Anch-dT.231 | ACGACGCTCTTCCGATCTNNNNNNNNTGCGCGATGCTTTTTTTTTTTTTTTTTTTTTTGC | 231 |
| J16 | Anch-dT.232 | ACGACGCTCTTCCGATCTNNNNNNNNATTGAGATTGTTTTTTTTTTTTTTTTTTTTTTGC | 232 |
| J17 | Anch-dT.233 | ACGACGCTCTTCCGATCTNNNNNNNNTTGATATATTTTTTTTTTTTTTTTTTTTTTTTGC | 233 |
| J18 | Anch-dT.234 | ACGACGCTCTTCCGATCTNNNNNNNNCGGTAGGAATTTTTTTTTTTTTTTTTTTTTTTGC | 234 |
| J19 | Anch-dT.235 | ACGACGCTCTTCCGATCTNNNNNNNNACCAGCGCAGTTTTTTTTTTTTTTTTTTTTTTGC | 235 |
| J20 | Anch-dT.236 | ACGACGCTCTTCCGATCTNNNNNNNNCGAATGAGCTTTTTTTTTTTTTTTTTTTTTTTGC | 236 |
| J21 | Anch-dT.237 | ACGACGCTCTTCCGATCTNNNNNNNNAGTTCGAGTATTTTTTTTTTTTTTTTTTTTTTGC | 237 |
| J22 | Anch-dT.238 | ACGACGCTCTTCCGATCTNNNNNNNNTTGGACGCTGTTTTTTTTTTTTTTTTTTTTTTGC | 238 |
| J23 | Anch-dT.239 | ACGACGCTCTTCCGATCTNNNNNNNNATAGACTAGGTTTTTTTTTTTTTTTTTTTTTTGC | 239 |
| J24 | Anch-dT.240 | ACGACGCTCTTCCGATCTNNNNNNNNTATAGTAAGCTTTTTTTTTTTTTTTTTTTTTTGC | 240 |
| K1 | Anch-dT.241 | ACGACGCTCTTCCGATCTNNNNNNNNCGGTCGTTAATTTTTTTTTTTTTTTTTTTTTTGC | 241 |
| K2 | Anch-dT.242 | ACGACGCTCTTCCGATCTNNNNNNNNATGGCGGATCTTTTTTTTTTTTTTTTTTTTTTGC | 242 |
| K3 | Anch-dT.243 | ACGACGCTCTTCCGATCTNNNNNNNNCTCTGATCAGTTTTTTTTTTTTTTTTTTTTTTGC | 243 |
| K4 | Anch-dT.244 | ACGACGCTCTTCCGATCTNNNNNNNNGGCCAGTCCGTTTTTTTTTTTTTTTTTTTTTTGC | 244 |
| K5 | Anch-dT.245 | ACGACGCTCTTCCGATCTNNNNNNNNCGGAAGATATTTTTTTTTTTTTTTTTTTTTTTGC | 245 |
| K6 | Anch-dT.246 | ACGACGCTCTTCCGATCTNNNNNNNNTGGCTGATGATTTTTTTTTTTTTTTTTTTTTTGC | 246 |
| K7 | Anch-dT.247 | ACGACGCTCTTCCGATCTNNNNNNNNGAAGGTTGCCTTTTTTTTTTTTTTTTTTTTTTGC | 247 |
| K8 | Anch-dT.248 | ACGACGCTCTTCCGATCTNNNNNNNNGTTGAAGGATTTTTTTTTTTTTTTTTTTTTTTGC | 248 |
| K9 | Anch-dT.249 | ACGACGCTCTTCCGATCTNNNNNNNNCCATTCGTAATTTTTTTTTTTTTTTTTTTTTTGC | 249 |
| K10 | Anch-dT.250 | ACGACGCTCTTCCGATCTNNNNNNNNTGCGCCAGAATTTTTTTTTTTTTTTTTTTTTTGC | 250 |
| K11 | Anch-dT.251 | ACGACGCTCTTCCGATCTNNNNNNNNCGAATAATTCTTTTTTTTTTTTTTTTTTTTTTGC | 251 |
| K12 | Anch-dT.252 | ACGACGCTCTTCCGATCTNNNNNNNNGCGACGCCTTTTTTTTTTTTTTTTTTTTTTTTGC | 252 |
| K13 | Anch-dT.253 | ACGACGCTCTTCCGATCTNNNNNNNNATCAACGATTTTTTTTTTTTTTTTTTTTTTTTGC | 253 |
| K14 | Anch-dT.254 | ACGACGCTCTTCCGATCTNNNNNNNNGTTCTGAATTTTTTTTTTTTTTTTTTTTTTTTGC | 254 |
| K15 | Anch-dT.255 | ACGACGCTCTTCCGATCTNNNNNNNNGCTAACCTCATTTTTTTTTTTTTTTTTTTTTTGC | 255 |
| K16 | Anch-dT.256 | ACGACGCTCTTCCGATCTNNNNNNNNCAAGCAACTGTTTTTTTTTTTTTTTTTTTTTTGC | 256 |
| K17 | Anch-dT.257 | ACGACGCTCTTCCGATCTNNNNNNNNGGAGCGGCCGTTTTTTTTTTTTTTTTTTTTTTGC | 257 |
| K18 | Anch-dT.258 | ACGACGCTCTTCCGATCTNNNNNNNNCGCGTACGACTTTTTTTTTTTTTTTTTTTTTTGC | 258 |
| K19 | Anch-dT.259 | ACGACGCTCTTCCGATCTNNNNNNNNCGATGGCGCCTTTTTTTTTTTTTTTTTTTTTTGC | 259 |
| K20 | Anch-dT.260 | ACGACGCTCTTCCGATCTNNNNNNNNTGGTATTCATTTTTTTTTTTTTTTTTTTTTTTGC | 260 |
| K21 | Anch-dT.261 | ACGACGCTCTTCCGATCTNNNNNNNNGATAAGGCAATTTTTTTTTTTTTTTTTTTTTTGC | 261 |
| K22 | Anch-dT.262 | ACGACGCTCTTCCGATCTNNNNNNNNGCCGGTCGAGTTTTTTTTTTTTTTTTTTTTTTGC | 262 |
| K23 | Anch-dT.263 | ACGACGCTCTTCCGATCTNNNNNNNNTGCGCCATCTTTTTTTTTTTTTTTTTTTTTTTGC | 263 |
| K24 | Anch-dT.264 | ACGACGCTCTTCCGATCTNNNNNNNNAAGTCTTCCGTTTTTTTTTTTTTTTTTTTTTTGC | 264 |
| L1 | Anch-dT.265 | ACGACGCTCTTCCGATCTNNNNNNNNAGACTCAAGCTTTTTTTTTTTTTTTTTTTTTTGC | 265 |
| L2 | Anch-dT.266 | ACGACGCTCTTCCGATCTNNNNNNNNGCAGGCGACGTTTTTTTTTTTTTTTTTTTTTTGC | 266 |
| L3 | Anch-dT.267 | ACGACGCTCTTCCGATCTNNNNNNNNAATACTCTTCTTTTTTTTTTTTTTTTTTTTTTGC | 267 |
| L4 | Anch-dT.268 | ACGACGCTCTTCCGATCTNNNNNNNNCCAACTAACCTTTTTTTTTTTTTTTTTTTTTTGC | 268 |
| L5 | Anch-dT.269 | ACGACGCTCTTCCGATCTNNNNNNNNTATCCTCAATTTTTTTTTTTTTTTTTTTTTTTGC | 269 |
| L6 | Anch-dT.270 | ACGACGCTCTTCCGATCTNNNNNNNNGCCGTCGCGTTTTTTTTTTTTTTTTTTTTTTTGC | 270 |
| L7 | Anch-dT.271 | ACGACGCTCTTCCGATCTNNNNNNNNCCGCTGCTTCTTTTTTTTTTTTTTTTTTTTTTGC | 271 |
| L8 | Anch-dT.272 | ACGACGCTCTTCCGATCTNNNNNNNNTGACCGAATCTTTTTTTTTTTTTTTTTTTTTTGC | 272 |
| L9 | Anch-dT.273 | ACGACGCTCTTCCGATCTNNNNNNNNGTCTCCAGAGTTTTTTTTTTTTTTTTTTTTTTGC | 273 |
| L10 | Anch-dT.274 | ACGACGCTCTTCCGATCTNNNNNNNNAATGCTAGTCTTTTTTTTTTTTTTTTTTTTTTGC | 274 |
| L11 | Anch-dT.275 | ACGACGCTCTTCCGATCTNNNNNNNNGACGACCTGCTTTTTTTTTTTTTTTTTTTTTTGC | 275 |
| L12 | Anch-dT.276 | ACGACGCTCTTCCGATCTNNNNNNNNAGAGCCAGCCTTTTTTTTTTTTTTTTTTTTTTGC | 276 |
| L13 | Anch-dT.277 | ACGACGCTCTTCCGATCTNNNNNNNNCCAGGCCGCATTTTTTTTTTTTTTTTTTTTTTGC | 277 |
| L14 | Anch-dT.278 | ACGACGCTCTTCCGATCTNNNNNNNNCAGGTATGGATTTTTTTTTTTTTTTTTTTTTTGC | 278 |
| L15 | Anch-dT.279 | ACGACGCTCTTCCGATCTNNNNNNNNCCGGAGTTGCTTTTTTTTTTTTTTTTTTTTTTGC | 279 |
| L16 | Anch-dT.280 | ACGACGCTCTTCCGATCTNNNNNNNNTTAATTATTGTTTTTTTTTTTTTTTTTTTTTTGC | 280 |
| L17 | Anch-dT.281 | ACGACGCTCTTCCGATCTNNNNNNNNAATCAGCTGCTTTTTTTTTTTTTTTTTTTTTTGC | 281 |
| L18 | Anch-dT.282 | ACGACGCTCTTCCGATCTNNNNNNNNCCGTTGACTTTTTTTTTTTTTTTTTTTTTTTTGC | 282 |
| L19 | Anch-dT.283 | ACGACGCTCTTCCGATCTNNNNNNNNGCCAGGATCATTTTTTTTTTTTTTTTTTTTTTGC | 283 |
| L20 | Anch-dT.284 | ACGACGCTCTTCCGATCTNNNNNNNNCTTCGGCGCATTTTTTTTTTTTTTTTTTTTTTGC | 284 |
| L21 | Anch-dT.285 | ACGACGCTCTTCCGATCTNNNNNNNNCAAGGCATTCTTTTTTTTTTTTTTTTTTTTTTGC | 285 |
| L22 | Anch-dT.286 | ACGACGCTCTTCCGATCTNNNNNNNNAAGAATGGAATTTTTTTTTTTTTTTTTTTTTTGC | 286 |
| L23 | Anch-dT.287 | ACGACGCTCTTCCGATCTNNNNNNNNCGGATGAAGGTTTTTTTTTTTTTTTTTTTTTTGC | 287 |
| L24 | Anch-dT.288 | ACGACGCTCTTCCGATCTNNNNNNNNTATCGTCGGCTTTTTTTTTTTTTTTTTTTTTTGC | 288 |
| M1 | Anch-dT.289 | ACGACGCTCTTCCGATCTNNNNNNNNGCCGTATGCTTTTTTTTTTTTTTTTTTTTTTTGC | 289 |
| M2 | Anch-dT.290 | ACGACGCTCTTCCGATCTNNNNNNNNCTGAACTGGTTTTTTTTTTTTTTTTTTTTTTTGC | 290 |
| M3 | Anch-dT.291 | ACGACGCTCTTCCGATCTNNNNNNNNCATAACCAGCTTTTTTTTTTTTTTTTTTTTTTGC | 291 |
| M4 | Anch-dT.292 | ACGACGCTCTTCCGATCTNNNNNNNNAAGTTGCCATTTTTTTTTTTTTTTTTTTTTTTGC | 292 |
| M5 | Anch-dT.293 | ACGACGCTCTTCCGATCTNNNNNNNNAGGCCGCTCGTTTTTTTTTTTTTTTTTTTTTTGC | 293 |
| M6 | Anch-dT.294 | ACGACGCTCTTCCGATCTNNNNNNNNAGGTAATAGGTTTTTTTTTTTTTTTTTTTTTTGC | 294 |
| M7 | Anch-dT.295 | ACGACGCTCTTCCGATCTNNNNNNNNGTACTAGTAATTTTTTTTTTTTTTTTTTTTTTGC | 295 |
| M8 | Anch-dT.296 | ACGACGCTCTTCCGATCTNNNNNNNNGCGCGGTAGTTTTTTTTTTTTTTTTTTTTTTTGC | 296 |
| M9 | Anch-dT.297 | ACGACGCTCTTCCGATCTNNNNNNNNCTGGATTAGTTTTTTTTTTTTTTTTTTTTTTTGC | 297 |
| M10 | Anch-dT.298 | ACGACGCTCTTCCGATCTNNNNNNNNTTGGATCCTTTTTTTTTTTTTTTTTTTTTTTTGC | 298 |
| M11 | Anch-dT.299 | ACGACGCTCTTCCGATCTNNNNNNNNTTGGAATCTCTTTTTTTTTTTTTTTTTTTTTTGC | 299 |
| M12 | Anch-dT.300 | ACGACGCTCTTCCGATCTNNNNNNNNACCTGGACGCTTTTTTTTTTTTTTTTTTTTTTGC | 300 |
| M13 | Anch-dT.301 | ACGACGCTCTTCCGATCTNNNNNNNNCCTGACGTTCTTTTTTTTTTTTTTTTTTTTTTGC | 301 |
| M14 | Anch-dT.302 | ACGACGCTCTTCCGATCTNNNNNNNNGCGTTCAGCTTTTTTTTTTTTTTTTTTTTTTTGC | 302 |
| M15 | Anch-dT.303 | ACGACGCTCTTCCGATCTNNNNNNNNTTAGCAATAATTTTTTTTTTTTTTTTTTTTTTGC | 303 |
| M16 | Anch-dT.304 | ACGACGCTCTTCCGATCTNNNNNNNNTTGATGCTATTTTTTTTTTTTTTTTTTTTTTTGC | 304 |
| M17 | Anch-dT.305 | ACGACGCTCTTCCGATCTNNNNNNNNCTCTGCGGCATTTTTTTTTTTTTTTTTTTTTTGC | 305 |
| M18 | Anch-dT.306 | ACGACGCTCTTCCGATCTNNNNNNNNAATAATACCATTTTTTTTTTTTTTTTTTTTTTGC | 306 |
| M19 | Anch-dT.307 | ACGACGCTCTTCCGATCTNNNNNNNNACGCCGTTCATTTTTTTTTTTTTTTTTTTTTTGC | 307 |
| M20 | Anch-dT.308 | ACGACGCTCTTCCGATCTNNNNNNNNTTCGCTTACGTTTTTTTTTTTTTTTTTTTTTTGC | 308 |
| M21 | Anch-dT.309 | ACGACGCTCTTCCGATCTNNNNNNNNTACGGCTACGTTTTTTTTTTTTTTTTTTTTTTGC | 309 |
| M22 | Anch-dT.310 | ACGACGCTCTTCCGATCTNNNNNNNNTTCTTATCGATTTTTTTTTTTTTTTTTTTTTTGC | 310 |
| M23 | Anch-dT.311 | ACGACGCTCTTCCGATCTNNNNNNNNTTCCATGGCATTTTTTTTTTTTTTTTTTTTTTGC | 311 |
| M24 | Anch-dT.312 | ACGACGCTCTTCCGATCTNNNNNNNNAAGTAGTCAGTTTTTTTTTTTTTTTTTTTTTTGC | 312 |
| N1 | Anch-dT.313 | ACGACGCTCTTCCGATCTNNNNNNNNTCAGCTCTAATTTTTTTTTTTTTTTTTTTTTTGC | 313 |
| N2 | Anch-dT.314 | ACGACGCTCTTCCGATCTNNNNNNNNCGAATAGATGTTTTTTTTTTTTTTTTTTTTTTGC | 314 |
| N3 | Anch-dT.315 | ACGACGCTCTTCCGATCTNNNNNNNNCGGAGATCCGTTTTTTTTTTTTTTTTTTTTTTGC | 315 |
| N4 | Anch-dT.316 | ACGACGCTCTTCCGATCTNNNNNNNNACCGCAGAATTTTTTTTTTTTTTTTTTTTTTTGC | 316 |
| N5 | Anch-dT.317 | ACGACGCTCTTCCGATCTNNNNNNNNTCTCCTATAATTTTTTTTTTTTTTTTTTTTTTGC | 317 |
| N6 | Anch-dT.318 | ACGACGCTCTTCCGATCTNNNNNNNNCAACCTATATTTTTTTTTTTTTTTTTTTTTTTGC | 318 |
| N7 | Anch-dT.319 | ACGACGCTCTTCCGATCTNNNNNNNNAGTCGAGAAGTTTTTTTTTTTTTTTTTTTTTTGC | 319 |
| N8 | Anch-dT.320 | ACGACGCTCTTCCGATCTNNNNNNNNAAGACGGCCATTTTTTTTTTTTTTTTTTTTTTGC | 320 |
| N9 | Anch-dT.321 | ACGACGCTCTTCCGATCTNNNNNNNNGCCAACGCCATTTTTTTTTTTTTTTTTTTTTTGC | 321 |
| N10 | Anch-dT.322 | ACGACGCTCTTCCGATCTNNNNNNNNTCTACCATTATTTTTTTTTTTTTTTTTTTTTTGC | 322 |
| N11 | Anch-dT.323 | ACGACGCTCTTCCGATCTNNNNNNNNCTTGCGGTCTTTTTTTTTTTTTTTTTTTTTTTGC | 323 |
| N12 | Anch-dT.324 | ACGACGCTCTTCCGATCTNNNNNNNNTTACGTATACTTTTTTTTTTTTTTTTTTTTTTGC | 324 |
| N13 | Anch-dT.325 | ACGACGCTCTTCCGATCTNNNNNNNNCGATTGGTTATTTTTTTTTTTTTTTTTTTTTTGC | 325 |
| N14 | Anch-dT.326 | ACGACGCTCTTCCGATCTNNNNNNNNACTTAACTAGTTTTTTTTTTTTTTTTTTTTTTGC | 326 |
| N15 | Anch-dT.327 | ACGACGCTCTTCCGATCTNNNNNNNNGCAGACCGGTTTTTTTTTTTTTTTTTTTTTTTGC | 327 |
| N16 | Anch-dT.328 | ACGACGCTCTTCCGATCTNNNNNNNNTGAGTCCAGATTTTTTTTTTTTTTTTTTTTTTGC | 328 |
| N17 | Anch-dT.329 | ACGACGCTCTTCCGATCTNNNNNNNNTGGAGAATTCTTTTTTTTTTTTTTTTTTTTTTGC | 329 |
| N18 | Anch-dT.330 | ACGACGCTCTTCCGATCTNNNNNNNNACCAGCCTTATTTTTTTTTTTTTTTTTTTTTTGC | 330 |
| N19 | Anch-dT.331 | ACGACGCTCTTCCGATCTNNNNNNNNGGCGAGCTTATTTTTTTTTTTTTTTTTTTTTTGC | 331 |
| N20 | Anch-dT.332 | ACGACGCTCTTCCGATCTNNNNNNNNTCGAGGAGTATTTTTTTTTTTTTTTTTTTTTTGC | 332 |
| N21 | Anch-dT.333 | ACGACGCTCTTCCGATCTNNNNNNNNCCTTACTCCTTTTTTTTTTTTTTTTTTTTTTTGC | 333 |
| N22 | Anch-dT.334 | ACGACGCTCTTCCGATCTNNNNNNNNTCAGACGAACTTTTTTTTTTTTTTTTTTTTTTGC | 334 |
| N23 | Anch-dT.335 | ACGACGCTCTTCCGATCTNNNNNNNNCCGTCCAGTATTTTTTTTTTTTTTTTTTTTTTGC | 335 |
| N24 | Anch-dT.336 | ACGACGCTCTTCCGATCTNNNNNNNNGTTCCGCTAATTTTTTTTTTTTTTTTTTTTTTGC | 336 |
| O1 | Anch-dT.337 | ACGACGCTCTTCCGATCTNNNNNNNNCAGATTCGATTTTTTTTTTTTTTTTTTTTTTTGC | 337 |
| O2 | Anch-dT.338 | ACGACGCTCTTCCGATCTNNNNNNNNTGCATATAACTTTTTTTTTTTTTTTTTTTTTTGC | 338 |
| O3 | Anch-dT.339 | ACGACGCTCTTCCGATCTNNNNNNNNTAGGCAGATATTTTTTTTTTTTTTTTTTTTTTGC | 339 |
| O4 | Anch-dT.340 | ACGACGCTCTTCCGATCTNNNNNNNNTATGCCGAGTTTTTTTTTTTTTTTTTTTTTTTGC | 340 |
| O5 | Anch-dT.341 | ACGACGCTCTTCCGATCTNNNNNNNNATAGTCGTAGTTTTTTTTTTTTTTTTTTTTTTGC | 341 |
| O6 | Anch-dT.342 | ACGACGCTCTTCCGATCTNNNNNNNNGGATGCAGCATTTTTTTTTTTTTTTTTTTTTTGC | 342 |
| O7 | Anch-dT.343 | ACGACGCTCTTCCGATCTNNNNNNNNCCGCTATATTTTTTTTTTTTTTTTTTTTTTTTGC | 343 |
| O8 | Anch-dT.344 | ACGACGCTCTTCCGATCTNNNNNNNNATCGAGTCGCTTTTTTTTTTTTTTTTTTTTTTGC | 344 |
| O9 | Anch-dT.345 | ACGACGCTCTTCCGATCTNNNNNNNNGCGACGCAGATTTTTTTTTTTTTTTTTTTTTTGC | 345 |
| O10 | Anch-dT.346 | ACGACGCTCTTCCGATCTNNNNNNNNAATGGTCGACTTTTTTTTTTTTTTTTTTTTTTGC | 346 |
| O11 | Anch-dT.347 | ACGACGCTCTTCCGATCTNNNNNNNNTGGAACTAGATTTTTTTTTTTTTTTTTTTTTTGC | 347 |
| O12 | Anch-dT.348 | ACGACGCTCTTCCGATCTNNNNNNNNGTCCAACTCATTTTTTTTTTTTTTTTTTTTTTGC | 348 |
| O13 | Anch-dT.349 | ACGACGCTCTTCCGATCTNNNNNNNNGTTATGGATCTTTTTTTTTTTTTTTTTTTTTTGC | 349 |
| O14 | Anch-dT.350 | ACGACGCTCTTCCGATCTNNNNNNNNTTATAAGAACTTTTTTTTTTTTTTTTTTTTTTGC | 350 |
| O15 | Anch-dT.351 | ACGACGCTCTTCCGATCTNNNNNNNNCAAGCTTCATTTTTTTTTTTTTTTTTTTTTTTGC | 351 |
| O16 | Anch-dT.352 | ACGACGCTCTTCCGATCTNNNNNNNNCTGATTAAGATTTTTTTTTTTTTTTTTTTTTTGC | 352 |
| O17 | Anch-dT.353 | ACGACGCTCTTCCGATCTNNNNNNNNTACTTACTTATTTTTTTTTTTTTTTTTTTTTTGC | 353 |
| O18 | Anch-dT.354 | ACGACGCTCTTCCGATCTNNNNNNNNGGATCTGCAGTTTTTTTTTTTTTTTTTTTTTTGC | 354 |
| O19 | Anch-dT.355 | ACGACGCTCTTCCGATCTNNNNNNNNATGCAATATGTTTTTTTTTTTTTTTTTTTTTTGC | 355 |
| O20 | Anch-dT.356 | ACGACGCTCTTCCGATCTNNNNNNNNTTCCTAGACCTTTTTTTTTTTTTTTTTTTTTTGC | 356 |
| O21 | Anch-dT.357 | ACGACGCTCTTCCGATCTNNNNNNNNACTGCCGATATTTTTTTTTTTTTTTTTTTTTTGC | 357 |
| O22 | Anch-dT.358 | ACGACGCTCTTCCGATCTNNNNNNNNTCCAGAAGGTTTTTTTTTTTTTTTTTTTTTTTGC | 358 |
| O23 | Anch-dT.359 | ACGACGCTCTTCCGATCTNNNNNNNNTTCAAGACCATTTTTTTTTTTTTTTTTTTTTTGC | 359 |
| O24 | Anch-dT.360 | ACGACGCTCTTCCGATCTNNNNNNNNTATTACTCATTTTTTTTTTTTTTTTTTTTTTTGC | 360 |
| P1 | Anch-dT.361 | ACGACGCTCTTCCGATCTNNNNNNNNAACTGATCTTTTTTTTTTTTTTTTTTTTTTTTGC | 361 |
| P2 | Anch-dT.362 | ACGACGCTCTTCCGATCTNNNNNNNNCCGCGGACCGTTTTTTTTTTTTTTTTTTTTTTGC | 362 |
| P3 | Anch-dT.363 | ACGACGCTCTTCCGATCTNNNNNNNNAATACGCAGGTTTTTTTTTTTTTTTTTTTTTTGC | 363 |
| P4 | Anch-dT.364 | ACGACGCTCTTCCGATCTNNNNNNNNGGTCGCGTCATTTTTTTTTTTTTTTTTTTTTTGC | 364 |
| P5 | Anch-dT.365 | ACGACGCTCTTCCGATCTNNNNNNNNAATTATCAGCTTTTTTTTTTTTTTTTTTTTTTGC | 365 |
| P6 | Anch-dT.366 | ACGACGCTCTTCCGATCTNNNNNNNNCAGCTATCGTTTTTTTTTTTTTTTTTTTTTTTGC | 366 |
| P7 | Anch-dT.367 | ACGACGCTCTTCCGATCTNNNNNNNNATTGCGCTGATTTTTTTTTTTTTTTTTTTTTTGC | 367 |
| P8 | Anch-dT.368 | ACGACGCTCTTCCGATCTNNNNNNNNTTGGTAGGCGTTTTTTTTTTTTTTTTTTTTTTGC | 368 |
| P9 | Anch-dT.369 | ACGACGCTCTTCCGATCTNNNNNNNNAGCTAAGGTATTTTTTTTTTTTTTTTTTTTTTGC | 369 |
| P10 | Anch-dT.370 | ACGACGCTCTTCCGATCTNNNNNNNNTCGTAGAGAATTTTTTTTTTTTTTTTTTTTTTGC | 370 |
| P11 | Anch-dT.371 | ACGACGCTCTTCCGATCTNNNNNNNNTGATGGCCTTTTTTTTTTTTTTTTTTTTTTTTGC | 371 |
| P12 | Anch-dT.372 | ACGACGCTCTTCCGATCTNNNNNNNNTGGAAGTACCTTTTTTTTTTTTTTTTTTTTTTGC | 372 |
| P13 | Anch-dT.373 | ACGACGCTCTTCCGATCTNNNNNNNNCTCCAAGGATTTTTTTTTTTTTTTTTTTTTTTGC | 373 |
| P14 | Anch-dT.374 | ACGACGCTCTTCCGATCTNNNNNNNNAGATATATCGTTTTTTTTTTTTTTTTTTTTTTGC | 374 |
| P15 | Anch-dT.375 | ACGACGCTCTTCCGATCTNNNNNNNNCATGCTGGTTTTTTTTTTTTTTTTTTTTTTTTGC | 375 |
| P16 | Anch-dT.376 | ACGACGCTCTTCCGATCTNNNNNNNNTCCTCGAGTCTTTTTTTTTTTTTTTTTTTTTTGC | 376 |
| P17 | Anch-dT.377 | ACGACGCTCTTCCGATCTNNNNNNNNGCAAGGAATATTTTTTTTTTTTTTTTTTTTTTGC | 377 |
| P18 | Anch-dT.378 | ACGACGCTCTTCCGATCTNNNNNNNNGGCATAGCTTTTTTTTTTTTTTTTTTTTTTTTGC | 378 |
| P19 | Anch-dT.379 | ACGACGCTCTTCCGATCTNNNNNNNNCTACGGTAGCTTTTTTTTTTTTTTTTTTTTTTGC | 379 |
| P20 | Anch-dT.380 | ACGACGCTCTTCCGATCTNNNNNNNNAGTAAGCATATTTTTTTTTTTTTTTTTTTTTTGC | 380 |
| P21 | Anch-dT.381 | ACGACGCTCTTCCGATCTNNNNNNNNCGCCTCGAACTTTTTTTTTTTTTTTTTTTTTTGC | 381 |
| P22 | Anch-dT.382 | ACGACGCTCTTCCGATCTNNNNNNNNTTAGGATCTATTTTTTTTTTTTTTTTTTTTTTGC | 382 |
| P23 | Anch-dT.383 | ACGACGCTCTTCCGATCTNNNNNNNNACTACTGAAGTTTTTTTTTTTTTTTTTTTTTTGC | 383 |
| P24 | Anch-dT.384 | ACGACGCTCTTCCGATCTNNNNNNNNAATCTGGAGTTTTTTTTTTTTTTTTTTTTTTTGC | 384 |

TABLE 3
SARS-COV-2_Targeted_RT_Primers

| Name | Sequence |
|---|---|
| SARS-COV-2 TRS-TSO | /5Biosg/TAAACGAACWWC |
| | GCAGAGTGAATrGrGrG |
| | (SEQ ID NO: 385) |
| Tailed SARS-Cov-2 Mod | /5Biosg/ACACTCTTTCCC |
| | TACACGACGCTCTTCCGATC |
| | TNNNNNNNNNKSWTCTT*W* |
| | K |
| | (SEQ ID NO : 386) |
| Tailed SARS-COV-2 TRS | /5Biosg/GTCTCGTGGGCT |
| | CGGAGATGTGTATAAGAGAC |
| | AGNNNNNNTAAACGAAC*W* |
| | W |
| | (SEQ ID NO: 387) |
| Tailed CDC-N1-F | /5Biosg/GTCTCGTGGGCT |
| | CGGAGATGTGTATAAGAGAC |
| | AGNNNNNNGACCCCAAA*A* |
| | T |
| | (SEQ ID NO: 388) |
| Tailed CDC-N1-R | /5Biosg/ACACTCTTTCCC |
| | TACACGACGCTCTTCCGATC |
| | TNNNNNNNNNTCTGGTT*A* |
| | C |
| | (SEQ ID NO: 389) |
| Tailed CDC-N2-F | /5Biosg/GTCTCGTGGGCT |
| | CGGAGATGTGTATAAGAGAC |
| | AGNNNNNNTTACAAACA*T* |
| | T |
| | (SEQ ID NO: 390) |
| Tailed CDC-N2-R | /5Biosg/ACACTCTTTCCC |
| | TACACGACGCTCTTCCGATC |
| | TNNNNNNNNNGCGCGAC*A* |
| | T |
| | (SEQ ID NO: 391) |
| Tailed CDC-RP-F | /5Biosg/GTCTCGTGGGCT |
| | CGGAGATGTGTATAAGAGAC |
| | AGNNNNNNAGATTTGGA*C* |
| | C |
| | (SEQ ID NO: 392) |
| Tailed CDC-RP-R | /5Biosg/ACACTCTTTCCC |
| | TACACGACGCTCTTCCGATC |
| | TNNNNNNNNNGAGCGGC*T* |
| | G |
| | (SEQ ID NO: 393) |

TABLE 4
cDNA_Preamp_Coupling_Primers

| Name | Sequence |
|---|---|
| Univ_cDNA-Coupler_ | ACA CTC TTT CCC TAC ACG |
| Forward | ACG CTC TTC CGA* T*C*T |
| | (SEQ ID NO: 394) |
| Univ cDNA-Coupler_ | AAG CAG TGG TAT CAA CGC |
| Reverse | AG*A* G*T |
| | (SEQ ID NO: 395) |
| 3′ seq_Univ_RT-TSO | AAG CAG TGG TAT CAA CGC |
| | AGA GTG AAT rGrGrG |
| | (SEQ ID NO: 396) |
| 5′ seq_Univ Anch-dT | AAG CAG TGG TAT CAA CGC |
| | AGA GT TTTTTTTTTTTTTTTT |
| | TTTTTTVN |
| | (SEQ ID NO: 397) |

TABLE 5
rDNA Blocking Duplex (HMR)

| Name | Sequence |
|---|---|
| Sequence 1 | TTA GAG GGA CAA GTG GCG TTC |
| | AGC CAC CCG AGA TTG /3C6/ |
| | (SEQ ID NO: 398) |
| Complement 1 | CAA TCT CGG GTG GCT GAA CGC |
| | CAC TTG TCC CTC TAA /3C6/ |
| | (SEQ ID NO: 399) |

TABLE 6
Illumina_Custom_3′_sci5_96

| | | | | | Index |
|---|---|---|---|---|---|
| | | | SEQ | | SEQ |
| | | | ID | | ID |
| Well | Name | Sequence | NO. | Index | NO. |
| A1 | sci5.1 | AATGATACGGCGACCACCGAGATCTACACCTCCATC | 400 | CTCGATGGAG | 496 |
| | | GAGACACTCTTTCCCTACACGACGCTCTTCCGATCT | | | |
| B1 | sci5.2 | AATGATACGGCGACCACCGAGATCTACACTTGGTAG | 401 | CGACTACCAA | 497 |
| | | TCGACACTCTTTCCCTACACGACGCTCTTCCGATCT | | | |
| C1 | sci5.3 | AATGATACGGCGACCACCGAGATCTACACGGCCGTC | 402 | GTTGACGGCC | 498 |
| | | AACACACTCTTTCCCTACACGACGCTCTTCCGATCT | | | |
| D1 | sci5.4 | AATGATACGGCGACCACCGAGATCTACACCCTAGAC | 403 | CTCGTCTAGG | 499 |
| | | GAGACACTCTTTCCCTACACGACGCTCTTCCGATCT | | | |
| E1 | sci5.5 | AATGATACGGCGACCACCGAGATCTACACTCGTTAG | 404 | GCTCTAACGA | 500 |
| | | AGCACACTCTTTCCCTACACGACGCTCTTCCGATCT | | | |
| F1 | sci5.6 | AATGATACGGCGACCACCGAGATCTACACCGTTCTA | 405 | TGATAGAACG | 501 |
| | | TCAACACTCTTTCCCTACACGACGCTCTTCCGATCT | | | |
| G1 | sci5.7 | AATGATACGGCGACCACCGAGATCTACACCGGAATC | 406 | TTAGATTCCG | 502 |
| | | TAAACACTCTTTCCCTACACGACGCTCTTCCGATCT | | | |
| H1 | sci5.8 | AATGATACGGCGACCACCGAGATCTACACATGACTG | 407 | GATCAGTCAT | 503 |
| | | ATCACACTCTTTCCCTACACGACGCTCTTCCGATCT | | | |
| A2 | sci5.9 | AATGATACGGCGACCACCGAGATCTACACTCAATAT | 408 | TCGATATTGA | 504 |
| | | CGAACACTCTTTCCCTACACGACGCTCTTCCGATCT | | | |
| B2 | sci5.10 | AATGATACGGCGACCACCGAGATCTACACGTAGACC | 409 | CCAGGTCTAC | 505 |
| | | TGGACACTCTTTCCCTACACGACGCTCTTCCGATCT | | | |
| C2 | sci5.11 | AATGATACGGCGACCACCGAGATCTACACTTATGAC | 410 | TTGGTCATAA | 506 |
| | | CAAACACTCTTTCCCTACACGACGCTCTTCCGATCT | | | |
| D2 | sci5.12 | AATGATACGGCGACCACCGAGATCTACACTTGGTCC | 411 | AACGGACCAA | 507 |
| | | GTTACACTCTTTCCCTACACGACGCTCTTCCGATCT | | | |
| E2 | sci5.13 | AATGATACGGCGACCACCGAGATCTACACGGTACGT | 412 | TTAACGTACC | 508 |
| | | TAAACACTCTTTCCCTACACGACGCTCTTCCGATCT | | | |
| F2 | sci5.14 | AATGATACGGCGACCACCGAGATCTACACCAATGAG | 413 | GGACTCATTG | 509 |
| | | TCCACACTCTTTCCCTACACGACGCTCTTCCGATCT | | | |
| G2 | sci5.15 | AATGATACGGCGACCACCGAGATCTACACGATGCAG | 414 | GAACTGCATC | 510 |
| | | TTCACACTCTTTCCCTACACGACGCTCTTCCGATCT | | | |
| H2 | sci5.16 | AATGATACGGCGACCACCGAGATCTACACCCATCGT | 415 | GGAACGATGG | 511 |
| | | TCCACACTCTTTCCCTACACGACGCTCTTCCGATCT | | | |
| A3 | sci5.17 | AATGATACGGCGACCACCGAGATCTACACTTGAGAG | 416 | ACTCTCTCAA | 512 |
| | | AGTACACTCTTTCCCTACACGACGCTCTTCCGATCT | | | |
| B3 | sci5.18 | AATGATACGGCGACCACCGAGATCTACACACTGAGC | 417 | GTCGCTCAGT | 513 |
| | | GACACACTCTTTCCCTACACGACGCTCTTCCGATCT | | | |
| C3 | sci5.19 | AATGATACGGCGACCACCGAGATCTACACTGAGGAA | 418 | TGATTCCTCA | 514 |
| | | TCAACACTCTTTCCCTACACGACGCTCTTCCGATCT | | | |
| D3 | sci5.20 | AATGATACGGCGACCACCGAGATCTACACCCTCCGA | 419 | CCGTCGGAGG | 515 |
| | | CGGACACTCTTTCCCTACACGACGCTCTTCCGATCT | | | |
| E3 | sci5.21 | AATGATACGGCGACCACCGAGATCTACACCATTGAC | 420 | AGCGTCAATG | 516 |
| | | GCTACACTCTTTCCCTACACGACGCTCTTCCGATCT | | | |
| F3 | sci5.22 | AATGATACGGCGACCACCGAGATCTACACTCGTCCT | 421 | CGAAGGACGA | 517 |
| | | TCGACACTCTTTCCCTACACGACGCTCTTCCGATCT | | | |
| G3 | sci5.23 | AATGATACGGCGACCACCGAGATCTACACTGATACT | 422 | TTGAGTATCA | 518 |
| | | CAAACACTCTTTCCCTACACGACGCTCTTCCGATCT | | | |
| H3 | sci5.24 | AATGATACGGCGACCACCGAGATCTACACTTCTACC | 423 | TGAGGTAGAA | 519 |
| | | TCAACACTCTTTCCCTACACGACGCTCTTCCGATCT | | | |
| A4 | sci5.25 | AATGATACGGCGACCACCGAGATCTACACTCGTCGG | 424 | GTTCCGACGA | 520 |
| | | AACACACTCTTTCCCTACACGACGCTCTTCCGATCT | | | |
| B4 | sci5.26 | AATGATACGGCGACCACCGAGATCTACACATCGAGA | 425 | TCATCTCGAT | 521 |
| | | TGAACACTCTTTCCCTACACGACGCTCTTCCGATCT | | | |
| C4 | sci5.27 | AATGATACGGCGACCACCGAGATCTACACTAGACTA | 426 | GACTAGTCTA | 522 |
| | | GTCACACTCTTTCCCTACACGACGCTCTTCCGATCT | | | |
| D4 | sci5.28 | AATGATACGGCGACCACCGAGATCTACACGTCGAAG | 427 | CTGCTTCGAC | 523 |
| | | CAGACACTCTTTCCCTACACGACGCTCTTCCGATCT | | | |
| | sci5.29 | AATGATACGGCGACCACCGAGATCTACACAGGCGCT | 428 | CCTAGCGCCT | 524 |
| E4 | | AGGACACTCTTTCCCTACACGACGCTCTTCCGATCT | | | |
| F4 | sci5.30 | AATGATACGGCGACCACCGAGATCTACACAGATGCA | 429 | AGTTGCATCT | 525 |
| | | ACTACACTCTTTCCCTACACGACGCTCTTCCGATCT | | | |
| G4 | sci5.31 | AATGATACGGCGACCACCGAGATCTACACAAGCCTA | 430 | TCGTAGGCTT | 526 |
| | | CGAACACTCTTTCCCTACACGACGCTCTTCCGATCT | | | |
| H4 | sci5.32 | AATGATACGGCGACCACCGAGATCTACACGTAGGCA | 431 | AATTGCCTAC | 527 |
| | | ATTACACTCTTTCCCTACACGACGCTCTTCCGATCT | | | |
| A5 | sci5.33 | AATGATACGGCGACCACCGAGATCTACACTGCCAGT | 432 | GCAACTGGCA | 528 |
| | | TGCACACTCTTTCCCTACACGACGCTCTTCCGATCT | | | |
| B5 | sci5.34 | AATGATACGGCGACCACCGAGATCTACACCTTAGGT | 433 | GATACCTAAG | 529 |
| | | ATCACACTCTTTCCCTACACGACGCTCTTCCGATCT | | | |
| C5 | sci5.35 | AATGATACGGCGACCACCGAGATCTACACGAGACCT | 434 | GGTAGGTCTC | 530 |
| | | ACCACACTCTTTCCCTACACGACGCTCTTCCGATCT | | | |
| D5 | sci5.36 | AATGATACGGCGACCACCGAGATCTACACATTGACC | 435 | CTCGGTCAAT | 531 |
| | | GAGACACTCTTTCCCTACACGACGCTCTTCCGATCT | | | |
| E5 | sci5.37 | AATGATACGGCGACCACCGAGATCTACACGGAGGCG | 436 | CGCCGCCTCC | 532 |
| | | GCGACACTCTTTCCCTACACGACGCTCTTCCGATCT | | | |
| F5 | sci5.38 | AATGATACGGCGACCACCGAGATCTACACCCAGTAC | 437 | CAAGTACTGG | 533 |
| | | TTGACACTCTTTCCCTACACGACGCTCTTCCGATCT | | | |
| G5 | sci5.39 | AATGATACGGCGACCACCGAGATCTACACGGTCTCG | 438 | CGGCGAGACC | 534 |
| | | CCGACACTCTTTCCCTACACGACGCTCTTCCGATCT | | | |
| H5 | sci5.40 | AATGATACGGCGACCACCGAGATCTACACGGCGGAG | 439 | GACCTCCGCC | 535 |
| | | GTCACACTCTTTCCCTACACGACGCTCTTCCGATCT | | | |
| A | sci5.41 | AATGATACGGCGACCACCGAGATCTACACTAGTTCT | 440 | TCTAGAACTA | 536 |
| | | AGAACACTCTTTCCCTACACGACGCTCTTCCGATCT | | | |
| B6 | sci5.42 | AATGATACGGCGACCACCGAGATCTACACTTGGAGT | 441 | CTAACTCCAA | 537 |
| | | TAGACACTCTTTCCCTACACGACGCTCTTCCGATCT | | | |
| C6 | sci5.43 | AATGATACGGCGACCACCGAGATCTACACAGATCTT | 442 | ACCAAGATCT | 538 |
| | | GGTACACTCTTTCCCTACACGACGCTCTTCCGATCT | | | |
| D6 | sci5.44 | AATGATACGGCGACCACCGAGATCTACACGTAATGA | 443 | CGATCATTAC | 539 |
| | | TCGACACTCTTTCCCTACACGACGCTCTTCCGATCT | | | |
| E6 | sci5.45 | AATGATACGGCGACCACCGAGATCTACACCAGAGAG | 444 | GACCTCTCTG | 540 |
| | | GTCACACTCTTTCCCTACACGACGCTCTTCCGATCT | | | |
| F6 | sci5.46 | AATGATACGGCGACCACCGAGATCTACACTTAATTA | 445 | GGCTAATTAA | 541 |
| | | GCCACACTCTTTCCCTACACGACGCTCTTCCGATCT | | | |
| G6 | sci5.47 | AATGATACGGCGACCACCGAGATCTACACCTCTAAC | 446 | CGAGTTAGAG | 542 |
| | | TCGACACTCTTTCCCTACACGACGCTCTTCCGATCT | | | |
| H6 | sci5.48 | AATGATACGGCGACCACCGAGATCTACACTACGATC | 447 | GATGATCGTA | 543 |
| | | ATCACACTCTTTCCCTACACGACGCTCTTCCGATCT | | | |
| A7 | sci5.49 | AATGATACGGCGACCACCGAGATCTACACAGGCGAG | 448 | GCTCTCGCCT | 544 |
| | | AGCACACTCTTTCCCTACACGACGCTCTTCCGATCT | | | |
| B7 | sci5.50 | AATGATACGGCGACCACCGAGATCTACACTCAAGAT | 449 | ACTATCTTGA | 545 |
| | | AGTACACTCTTTCCCTACACGACGCTCTTCCGATCT | | | |
| C7 | sci5.51 | AATGATACGGCGACCACCGAGATCTACACTAATTGA | 450 | AGGTCAATTA | 546 |
| | | CCTACACTCTTTCCCTACACGACGCTCTTCCGATCT | | | |
| D7 | sci5.52 | AATGATACGGCGACCACCGAGATCTACACCAGCCGG | 451 | AAGCCGGCTG | 547 |
| | | CTTACACTCTTTCCCTACACGACGCTCTTCCGATCT | | | |
| E7 | sci5.53 | AATGATACGGCGACCACCGAGATCTACACAGAACCG | 452 | CTCCGGTTCT | 548 |
| | | GAGACACTCTTTCCCTACACGACGCTCTTCCGATCT | | | |
| F7 | sci5.54 | AATGATACGGCGACCACCGAGATCTACACGAGATGC | 453 | CATGCATCTC | 549 |
| | | ATGACACTCTTTCCCTACACGACGCTCTTCCGATCT | | | |
| GT | sci5.55 | AATGATACGGCGACCACCGAGATCTACACGATTACC | 454 | TCCGGTAATC | 550 |
| | | GGAACACTCTTTCCCTACACGACGCTCTTCCGATCT | | | |
| H7 | sci5.56 | AATGATACGGCGACCACCGAGATCTACACTCGTAAC | 455 | ACCGTTACGA | 551 |
| | | GGTACACTCTTTCCCTACACGACGCTCTTCCGATCT | | | |
| A8 | sci5.57 | AATGATACGGCGACCACCGAGATCTACACTGGCGAC | 456 | TCCGTCGCCA | 552 |
| | | GGAACACTCTTTCCCTACACGACGCTCTTCCGATCT | | | |
| B8 | sci5.58 | AATGATACGGCGACCACCGAGATCTACACAGTCATA | 457 | GGCTATGACT | 553 |
| | | GCCACACTCTTTCCCTACACGACGCTCTTCCGATCT | | | |
| C8 | sci5.59 | AATGATACGGCGACCACCGAGATCTACACGTCAAGT | 458 | TGGACTTGAC | 554 |
| | | CCAACACTCTTTCCCTACACGACGCTCTTCCGATCT | | | |
| D8 | sci5.60 | AATGATACGGCGACCACCGAGATCTACACATTCGGA | 459 | ACTTCCGAAT | 555 |
| | | AGTACACTCTTTCCCTACACGACGCTCTTCCGATCT | | | |
| E8 | sci5.61 | AATGATACGGCGACCACCGAGATCTACACGTCGGTA | 460 | AACTACCGAC | 556 |
| | | GTTACACTCTTTCCCTACACGACGCTCTTCCGATCT | | | |
| F8 | sci5.62 | AATGATACGGCGACCACCGAGATCTACACAGGACGG | 461 | CGTCCGTCCT | 557 |
| | | ACGACACTCTTTCCCTACACGACGCTCTTCCGATCT | | | |
| G8 | sci5.63 | AATGATACGGCGACCACCGAGATCTACACCTCCTGG | 462 | GGTCCAGGAG | 558 |
| | | ACCACACTCTTTCCCTACACGACGCTCTTCCGATCT | | | |
| H8 | sci5.64 | AATGATACGGCGACCACCGAGATCTACACTAGCCTC | 463 | AACGAGGCTA | 559 |
| | | GTTACACTCTTTCCCTACACGACGCTCTTCCGATCT | | | |
| A9 | sci5.65 | AATGATACGGCGACCACCGAGATCTACACGGTTGAA | 464 | ACGTTCAACC | 560 |
| | | CGTACACTCTTTCCCTACACGACGCTCTTCCGATCT | | | |
| B9 | sci5.66 | AATGATACGGCGACCACCGAGATCTACACAGGTCCT | 465 | ACGAGGACCT | 561 |
| | | CGTACACTCTTTCCCTACACGACGCTCTTCCGATCT | | | |
| C9 | sci5.67 | AATGATACGGCGACCACCGAGATCTACACGGAAGTT | 466 | TATAACTTCC | 562 |
| | | ATAACACTCTTTCCCTACACGACGCTCTTCCGATCT | | | |
| D9 | sci5.68 | AATGATACGGCGACCACCGAGATCTACACTGGTAAT | 467 | AGGATTACCA | 563 |
| | | CCTACACTCTTTCCCTACACGACGCTCTTCCGATCT | | | |
| E9 | sci5.69 | AATGATACGGCGACCACCGAGATCTACACAAGCTAG | 468 | AACCTAGCTT | 564 |
| | | GTTACACTCTTTCCCTACACGACGCTCTTCCGATCT | | | |
| F9 | sci5.70 | AATGATACGGCGACCACCGAGATCTACACTCCGCGG | 469 | AGTCCGCGGA | 565 |
| | | ACTACACTCTTTCCCTACACGACGCTCTTCCGATCT | | | |
| G9 | sci5.71 | AATGATACGGCGACCACCGAGATCTACACTGCGGAT | 470 | ACTATCCGCA | 566 |
| | | AGTACACTCTTTCCCTACACGACGCTCTTCCGATCT | | | |
| H9 | sci5.72 | AATGATACGGCGACCACCGAGATCTACACTGGCAGC | 471 | CGAGCTGCCA | 567 |
| | | TCGACACTCTTTCCCTACACGACGCTCTTCCGATCT | | | |
| A10 | sci5.73 | AATGATACGGCGACCACCGAGATCTACACTGCTACG | 472 | GACCGTAGCA | 568 |
| | | GTCACACTCTTTCCCTACACGACGCTCTTCCGATCT | | | |
| B10 | sci5.74 | AATGATACGGCGACCACCGAGATCTACACGCGCAAT | 473 | GTCATTGCGC | 569 |
| | | GACACACTCTTTCCCTACACGACGCTCTTCCGATCT | | | |
| C10 | sci5.75 | AATGATACGGCGACCACCGAGATCTACACCTTAATC | 474 | CAAGATTAAG | 570 |
| | | TTGACACTCTTTCCCTACACGACGCTCTTCCGATCT | | | |
| D10 | sci5.76 | AATGATACGGCGACCACCGAGATCTACACGGAGTTG | 475 | ACGCAACTCC | 571 |
| | | CGTACACTCTTTCCCTACACGACGCTCTTCCGATCT | | | |
| E10 | sci5.77 | AATGATACGGCGACCACCGAGATCTACACACTCGTA | 476 | TGATACGAGT | 572 |
| | | TCAACACTCTTTCCCTACACGACGCTCTTCCGATCT | | | |
| F10 | sci5.78 | AATGATACGGCGACCACCGAGATCTACACGGTAATA | 477 | CATTATTACC | 573 |
| | | ATGACACTCTTTCCCTACACGACGCTCTTCCGATCT | | | |
| G10 | sci5.79 | AATGATACGGCGACCACCGAGATCTACACTCCTTAT | 478 | TCTATAAGGA | 574 |
| | | AGAACACTCTTTCCCTACACGACGCTCTTCCGATCT | | | |
| H10 | sci5.80 | AATGATACGGCGACCACCGAGATCTACACCCGACTC | 479 | TTGGAGTCGG | 575 |
| | | CAAACACTCTTTCCCTACACGACGCTCTTCCGATCT | | | |
| A11 | sci5.81 | AATGATACGGCGACCACCGAGATCTACACGCCAAGC | 480 | CAAGCTTGGC | 576 |
| | | TTGACACTCTTTCCCTACACGACGCTCTTCCGATCT | | | |
| B11 | sci5.82 | AATGATACGGCGACCACCGAGATCTACACCATATCC | 481 | ATAGGATATG | 577 |
| | | TATACACTCTTTCCCTACACGACGCTCTTCCGATCT | | | |
| C11 | sci5.83 | AATGATACGGCGACCACCGAGATCTACACACCTACG | 482 | TGGCGTAGGT | 578 |
| | | CCAACACTCTTTCCCTACACGACGCTCTTCCGATCT | | | |
| D11 | sci5.84 | AATGATACGGCGACCACCGAGATCTACACGGAATTC | 483 | ACTGAATTCC | 579 |
| | | AGTACACTCTTTCCCTACACGACGCTCTTCCGATCT | | | |
| E11 | sci5.85 | AATGATACGGCGACCACCGAGATCTACACTGGCGTA | 484 | TTCTACGCCA | 580 |
| | | GAAACACTCTTTCCCTACACGACGCTCTTCCGATCT | | | |
| F11 | sci5.86 | AATGATACGGCGACCACCGAGATCTACACATTGCGG | 485 | TGGCCGCAAT | 581 |
| | | CCAACACTCTTTCCCTACACGACGCTCTTCCGATCT | | | |
| G11 | sci5.87 | AATGATACGGCGACCACCGAGATCTACACTTCAGCT | 486 | CCAAGCTGAA | 582 |
| | | TGGACACTCTTTCCCTACACGACGCTCTTCCGATCT | | | |
| H11 | sci5.88 | AATGATACGGCGACCACCGAGATCTACACCCATCTG | 487 | TGCCAGATGG | 583 |
| | | GCAACACTCTTTCCCTACACGACGCTCTTCCGATCT | | | |
| A12 | sci5.89 | AATGATACGGCGACCACCGAGATCTACACCTTATAA | 488 | AACTTATAAG | 584 |
| | | GTTACACTCTTTCCCTACACGACGCTCTTCCGATCT | | | |
| B12 | sci5.90 | AATGATACGGCGACCACCGAGATCTACACGATTAGA | 489 | TCATCTAATC | 585 |
| | | TGAACACTCTTTCCCTACACGACGCTCTTCCGATCT | | | |
| C12 | sci5.91 | AATGATACGGCGACCACCGAGATCTACACTATAGGA | 490 | AGATCCTATA | 586 |
| | | TCTACACTCTTTCCCTACACGACGCTCTTCCGATCT | | | |
| D12 | sci5.92 | AATGATACGGCGACCACCGAGATCTACACAGCTTAT | 491 | CCTATAAGCT | 587 |
| | | AGGACACTCTTTCCCTACACGACGCTCTTCCGATCT | | | |
| E12 | sci5.93 | AATGATACGGCGACCACCGAGATCTACACGTCTGCA | 492 | GATTGCAGAC | 588 |
| | | ATCACACTCTTTCCCTACACGACGCTCTTCCGATCT | | | |
| F12 | sci5.94 | AATGATACGGCGACCACCGAGATCTACACCGCCTCT | 493 | ATAAGAGGCG | 589 |
| | | TATACACTCTTTCCCTACACGACGCTCTTCCGATCT | | | |
| G12 | sci5.95 | AATGATACGGCGACCACCGAGATCTACACGTTGGAT | 494 | AAGATCCAAC | 590 |
| | | CTTACACTCTTTCCCTACACGACGCTCTTCCGATCT | | | |
| H12 | sci5.96 | AATGATACGGCGACCACCGAGATCTACACGCGATTG | 495 | CTGCAATCGC | 591 |
| | | CAGACACTCTTTCCCTACACGACGCTCTTCCGATCT | | | |

TABLE 7
Illumina_Custom_5′_sci7_96

| | | | | | Index |
|---|---|---|---|---|---|
| | | | SEQ ID | | SEQ ID |
| Well | Name | Sequence | NO. | Index | NO. |
| A1 | sci7.1 | CAAGCAGAAGACGGCATACGAGATccgaatccgaGTCTCGTGGGCTCGG | 592 | ccgaatccga | 688 |
| B1 | sci7.2 | CAAGCAGAAGACGGCATACGAGATataagccggaGTCTCGTGGGCTCGG | 593 | ataagccgga | 689 |
| C1 | sci7.3 | CAAGCAGAAGACGGCATACGAGATccggcggcgaGTCTCGTGGGCTCGG | 594 | ccggcggcga | 690 |
| D1 | sci7.4 | CAAGCAGAAGACGGCATACGAGATggcttgccaaGTCTCGTGGGCTCGG | 595 | ggcttgccaa | 691 |
| E1 | sci7.5 | CAAGCAGAAGACGGCATACGAGATccgctagctgGTCTCGTGGGCTCGG | 596 | ccgctagctg | 692 |
| F1 | sci7.6 | CAAGCAGAAGACGGCATACGAGATcttatcctacGTCTCGTGGGCTCGG | 597 | cttatcctac | 693 |
| G1 | sci7.7 | CAAGCAGAAGACGGCATACGAGATtgagctacttGTCTCGTGGGCTCGG | 598 | tgagctactt | 694 |
| H1 | sci7.8 | CAAGCAGAAGACGGCATACGAGATtcaggacttaGTCTCGTGGGCTCGG | 599 | tcaggactta | 695 |
| A2 | sci7.9 | CAAGCAGAAGACGGCATACGAGATccgcagccgcGTCTCGTGGGCTCGG | 600 | ccgcagccgc | 696 |
| B2 | sci7.10 | CAAGCAGAAGACGGCATACGAGATtgcgcctggtGTCTCGTGGGCTCGG | 601 | tgcgcctggt | 697 |
| C2 | sci7.11 | CAAGCAGAAGACGGCATACGAGATaatcatacggGTCTCGTGGGCTCGG | 602 | aatcatacgg | 698 |
| D2 | sci7.12 | CAAGCAGAAGACGGCATACGAGATcgccaatcaaGTCTCGTGGGCTCGG | 603 | cgccaatcaa | 699 |
| E2 | sci7.13 | CAAGCAGAAGACGGCATACGAGATcaaggcttagGTCTCGTGGGCTCGG | 604 | caaggcttag | 700 |
| F2 | sci7.14 | CAAGCAGAAGACGGCATACGAGATgcgctcgacgGTCTCGTGGGCTCGG | 605 | gcgctcgacg | 701 |
| G2 | sci7.15 | CAAGCAGAAGACGGCATACGAGATtccagcaataGTCTCGTGGGCTCGG | 606 | tccagcaata | 702 |
| H2 | sci7.16 | CAAGCAGAAGACGGCATACGAGATcatgagaactGTCTCGTGGGCTCGG | 607 | catgagaact | 703 |
| A3 | sci7.17 | CAAGCAGAAGACGGCATACGAGATaacgtaatctGTCTCGTGGGCTCGG | 608 | aacgtaatct | 704 |
| B3 | sci7.18 | CAAGCAGAAGACGGCATACGAGATattctcctctGTCTCGTGGGCTCGG | 609 | attctcctct | 705 |
| C3 | sci7.19 | CAAGCAGAAGACGGCATACGAGATtctgcgcgttGTCTCGTGGGCTCGG | 610 | tctgcgcgtt | 706 |
| D3 | sci7.20 | CAAGCAGAAGACGGCATACGAGATgctcatatgcGTCTCGTGGGCTCGG | 611 | getcatatgc | 707 |
| E3 | sci7.21 | CAAGCAGAAGACGGCATACGAGATagcggtaacgGTCTCGTGGGCTCGG | 612 | agcggtaacg | 708 |
| F3 | sci7.22 | CAAGCAGAAGACGGCATACGAGATaatgaatagtGTCTCGTGGGCTCGG | 613 | aatgaatagt | 709 |
| G3 | sci7.23 | CAAGCAGAAGACGGCATACGAGATccgtatctggGTCTCGTGGGCTCGG | 614 | ccgtatctgg | 710 |
| H3 | sci7.24 | CAAGCAGAAGACGGCATACGAGATccttagtctgGTCTCGTGGGCTCGG | 615 | ccttagtctg | 711 |
| A4 | sci7.25 | CAAGCAGAAGACGGCATACGAGATacctagttagGTCTCGTGGGCTCGG | 616 | acctagttag | 712 |
| B4 | sci7.26 | CAAGCAGAAGACGGCATACGAGATataggagtacGTCTCGTGGGCTCGG | 617 | ataggagtac | 713 |
| C4 | sci7.27 | CAAGCAGAAGACGGCATACGAGATctacgacgagGTCTCGTGGGCTCGG | 618 | ctacgacgag | 714 |
| D4 | sci7.28 | CAAGCAGAAGACGGCATACGAGATagtcgagttcGTCTCGTGGGCTCGG | 619 | agtcgagttc | 715 |
| E4 | sci7.29 | CAAGCAGAAGACGGCATACGAGATtggtccagtcGTCTCGTGGGCTCGG | 620 | tggtccagtc | 716 |
| F4 | sci7.30 | CAAGCAGAAGACGGCATACGAGATatctaagcaaGTCTCGTGGGCTCGG | 621 | atctaagcaa | 717 |
| G4 | sci7.31 | CAAGCAGAAGACGGCATACGAGATcgaattcgttGTCTCGTGGGCTCGG | 622 | cgaattogtt | 718 |
| H4 | sci7.32 | CAAGCAGAAGACGGCATACGAGATcagcgatagaGTCTCGTGGGCTCGG | 623 | cagcgataga | 719 |
| A5 | sci7.33 | CAAGCAGAAGACGGCATACGAGATggtcgctatgGTCTCGTGGGCTCGG | 624 | ggtcgctatg | 720 |
| B5 | sci7.34 | CAAGCAGAAGACGGCATACGAGATatccgttagcGTCTCGTGGGCTCGG | 625 | atccgttagc | 721 |
| C5 | sci7.35 | CAAGCAGAAGACGGCATACGAGATtcgcaattagGTCTCGTGGGCTCGG | 626 | tcgcaattag | 722 |
| D5 | sci7.36 | CAAGCAGAAGACGGCATACGAGATggctggctagGTCTCGTGGGCTCGG | 627 | ggctggctag | 723 |
| E5 | sci7.37 | CAAGCAGAAGACGGCATACGAGATacggtcttgcGTCTCGTGGGCTCGG | 628 | acggtcttgc | 724 |
| F5 | sci7.38 | CAAGCAGAAGACGGCATACGAGATgctccattcgGTCTCGTGGGCTCGG | 629 | gctccattcg | 725 |
| G5 | sci7.39 | CAAGCAGAAGACGGCATACGAGATacgataagcgGTCTCGTGGGCTCGG | 630 | acgataagcg | 726 |
| H5 | sci7.40 | CAAGCAGAAGACGGCATACGAGATaccatagcgcGTCTCGTGGGCTCGG | 631 | accatagcgc | 727 |
| A6 | sci7.41 | CAAGCAGAAGACGGCATACGAGATctcttagcggGTCTCGTGGGCTCGG | 632 | ctcttagcgg | 728 |
| B6 | sci7.42 | CAAGCAGAAGACGGCATACGAGATtgattcaactGTCTCGTGGGCTCGG | 633 | tgattcaact | 729 |
| C6 | sci7.43 | CAAGCAGAAGACGGCATACGAGATtatggccgcgGTCTCGTGGGCTCGG | 634 | tatggccgcg | 730 |
| D6 | sci7.44 | CAAGCAGAAGACGGCATACGAGATagaggtcgcaGTCTCGTGGGCTCGG | 635 | agaggtcgca | 731 |
| E6 | sci7.45 | CAAGCAGAAGACGGCATACGAGATaggagattgaGTCTCGTGGGCTCGG | 636 | aggagattga | 732 |
| F6 | sci7.46 | CAAGCAGAAGACGGCATACGAGATggctatatagGTCTCGTGGGCTCGG | 637 | ggctatatag | 733 |
| G6 | sci7.47 | CAAGCAGAAGACGGCATACGAGATtcgcgtacttGTCTCGTGGGCTCGG | 638 | tcgcgtactt | 734 |
| H6 | sci7.48 | CAAGCAGAAGACGGCATACGAGATaataataatgGTCTCGTGGGCTCGG | 639 | aataataatg | 735 |
| A7 | sci7.49 | CAAGCAGAAGACGGCATACGAGATttcgttccatGTCTCGTGGGCTCGG | 640 | ttcgttccat | 736 |
| B7 | sci7.50 | CAAGCAGAAGACGGCATACGAGATtacctaatcaGTCTCGTGGGCTCGG | 641 | tacctaatca | 737 |
| C7 | sci7.51 | CAAGCAGAAGACGGCATACGAGATaagtaatattGTCTCGTGGGCTCGG | 642 | aagtaatatt | 738 |
| D7 | sci7.52 | CAAGCAGAAGACGGCATACGAGATagctaagaatGTCTCGTGGGCTCGG | 643 | agctaagaat | 739 |
| E7 | sci7.53 | CAAGCAGAAGACGGCATACGAGATgtcgaggtatGTCTCGTGGGCTCGG | 644 | gtcgaggtat | 740 |
| F7 | sci7.54 | CAAGCAGAAGACGGCATACGAGATttattagtagGTCTCGTGGGCTCGG | 645 | ttattagtag | 741 |
| G7 | sci7.55 | CAAGCAGAAGACGGCATACGAGATtgcgaagatcGTCTCGTGGGCTCGG | 646 | tgcgaagatc | 742 |
| H7 | sci7.56 | CAAGCAGAAGACGGCATACGAGATaactacggctGTCTCGTGGGCTCGG | 647 | aactacggct | 743 |
| A8 | sci7.57 | CAAGCAGAAGACGGCATACGAGATaacggaacgcGTCTCGTGGGCTCGG | 648 | aacggaacgc | 744 |
| B8 | sci7.58 | CAAGCAGAAGACGGCATACGAGATgatgctacgaGTCTCGTGGGCTCGG | 649 | gatgctacga | 745 |
| C8 | sci7.59 | CAAGCAGAAGACGGCATACGAGATatctgccaatGTCTCGTGGGCTCGG | 650 | atctgccaat | 746 |
| D8 | sci7.60 | CAAGCAGAAGACGGCATACGAGATatcgtatcaaGTCTCGTGGGCTCGG | 651 | atcgtatcaa | 747 |
| E8 | sci7.61 | CAAGCAGAAGACGGCATACGAGATaacgcctctaGTCTCGTGGGCTCGG | 652 | aacgcctcta | 748 |
| F8 | sci7.62 | CAAGCAGAAGACGGCATACGAGATacggcaaccaGTCTCGTGGGCTCGG | 653 | acggcaacca | 749 |
| G8 | sci7.63 | CAAGCAGAAGACGGCATACGAGATcaggctaagaGTCTCGTGGGCTCGG | 654 | caggctaaga | 750 |
| H8 | sci7.64 | CAAGCAGAAGACGGCATACGAGATcgcaatatcaGTCTCGTGGGCTCGG | 655 | cocaatatca | 751 |
| A9 | sci7.65 | CAAGCAGAAGACGGCATACGAGATttcgataaccGTCTCGTGGGCTCGG | 656 | ttcgataacc | 752 |
| B9 | sci7.66 | CAAGCAGAAGACGGCATACGAGATaacctcaagaGTCTCGTGGGCTCGG | 657 | aacctcaaga | 753 |
| C9 | sci7.67 | CAAGCAGAAGACGGCATACGAGATcaggcgccatGTCTCGTGGGCTCGG | 658 | caggcgccat | 754 |
| D9 | sci7.68 | CAAGCAGAAGACGGCATACGAGATaactattataGTCTCGTGGGCTCGG | 659 | aactattata | 755 |
| E9 | sci7.69 | CAAGCAGAAGACGGCATACGAGATaagttacctaGTCTCGTGGGCTCGG | 660 | aagttaccta | 756 |
| F9 | sci7.70 | CAAGCAGAAGACGGCATACGAGATcggcagaggaGTCTCGTGGGCTCGG | 661 | cggcagagga | 757 |
| G9 | sci7.71 | CAAGCAGAAGACGGCATACGAGATgcctcaataaGTCTCGTGGGCTCGG | 662 | gcctcaataa | 758 |
| H9 | sci7.72 | CAAGCAGAAGACGGCATACGAGATttaacgccgtGTCTCGTGGGCTCGG | 663 | ttaacgccgt | 759 |
| A10 | sci7.73 | CAAGCAGAAGACGGCATACGAGATcatacgatgcGTCTCGTGGGCTCGG | 664 | catacgatgc | 760 |
| B10 | sci7.74 | CAAGCAGAAGACGGCATACGAGATaagctgacctGTCTCGTGGGCTCGG | 665 | aagctgacct | 761 |
| C10 | sci7.75 | CAAGCAGAAGACGGCATACGAGATgagtccttatGTCTCGTGGGCTCGG | 666 | gagtccttat | 762 |
| D10 | sci7.76 | CAAGCAGAAGACGGCATACGAGATcctacggcaaGTCTCGTGGGCTCGG | 667 | cctacggcaa | 763 |
| E10 | sci7.77 | CAAGCAGAAGACGGCATACGAGATaatattcgaaGTCTCGTGGGCTCGG | 668 | aatattcgaa | 764 |
| F10 | sci7.78 | CAAGCAGAAGACGGCATACGAGATttcaagaatcGTCTCGTGGGCTCGG | 669 | ttcaagaatc | 765 |
| G10 | sci7.79 | CAAGCAGAAGACGGCATACGAGATatgctcgcaaGTCTCGTGGGCTCGG | 670 | atgctcgcaa | 766 |
| H10 | sci7.80 | CAAGCAGAAGACGGCATACGAGATggagtaagccGTCTCGTGGGCTCGG | 671 | ggagtaagcc | 767 |
| A11 | sci7.81 | CAAGCAGAAGACGGCATACGAGATttatcgtattGTCTCGTGGGCTCGG | 672 | ttatcgtatt | 768 |
| B11 | sci7.82 | CAAGCAGAAGACGGCATACGAGATaagtctaataGTCTCGTGGGCTCGG | 673 | aagtctaata | 769 |
| C11 | sci7.83 | CAAGCAGAAGACGGCATACGAGATcggcttactaGTCTCGTGGGCTCGG | 674 | cggcttacta | 770 |
| D11 | sci7.84 | CAAGCAGAAGACGGCATACGAGATgatatggtctGTCTCGTGGGCTCGG | 675 | gatatggtct | 771 |
| E11 | sci7.85 | CAAGCAGAAGACGGCATACGAGATtagtcgtccaGTCTCGTGGGCTCGG | 676 | tagtcgtcca | 772 |
| F11 | sci7.86 | CAAGCAGAAGACGGCATACGAGATtagctgctacGTCTCGTGGGCTCGG | 677 | tagctgctac | 773 |
| G11 | sci7.87 | CAAGCAGAAGACGGCATACGAGATctcttcaagcGTCTCGTGGGCTCGG | 678 | ctcttcaagc | 774 |
| H11 | sci7.88 | CAAGCAGAAGACGGCATACGAGATatgaacgcgcGTCTCGTGGGCTCGG | 679 | atgaacgcgc | 775 |
| A12 | sci7.89 | CAAGCAGAAGACGGCATACGAGATgtcgacggaaGTCTCGTGGGCTCGG | 680 | gtcgacggaa | 776 |
| B12 | sci7.90 | CAAGCAGAAGACGGCATACGAGATactaattgagGTCTCGTGGGCTCGG | 681 | actaattgag | 777 |
| C12 | sci7.91 | CAAGCAGAAGACGGCATACGAGATcttgcataatGTCTCGTGGGCTCGG | 682 | cttgcataat | 778 |
| D12 | sci7.92 | CAAGCAGAAGACGGCATACGAGATtccttaccaaGTCTCGTGGGCTCGG | 683 | tccttaccaa | 779 |
| E12 | sci7.93 | CAAGCAGAAGACGGCATACGAGATtgcagcctacGTCTCGTGGGCTCGG | 684 | tgcagcctac | 780 |
| F12 | sci7.94 | CAAGCAGAAGACGGCATACGAGATggagctgaggGTCTCGTGGGCTCGG | 685 | ggagctgagg | 781 |
| G12 | sci7.95 | CAAGCAGAAGACGGCATACGAGATgcagcggactGTCTCGTGGGCTCGG | 686 | gcagcggact | 782 |
| H12 | sci7.96 | CAAGCAGAAGACGGCATACGAGATcatcgcgctcGTCTCGTGGGCTCGG | 687 | catcgcgctc | 783 |

TABLE 8
IonTorrent_Custom_3′_OuterA_96

| | | | | | Index |
|---|---|---|---|---|---|
| | | | SEQ ID | | SEQ ID |
| Well | Name | Sequence | NO. | Index | NO. |
| A1 | OuterA.1 | CCATCTCATCCCTGCGTGTCTCCGACTCAG | 784 | CTCCATCGAG | 881 |
| | | CTCCATCGAGGATACGACGCTCTTCCGAT* | | | |
| | | C*T | | | |
| B1 | OuterA.2 | CCATCTCATCCCTGCGTGTCTCCGACTCAG | 785 | TTGGTAGTCG | 882 |
| | | TTGGTAGTCGGATACGACGCTCTTCCGAT* | | | |
| | | C*T | | | |
| C1 | OuterA.3 | CCATCTCATCCCTGCGTGTCTCCGACTCAG | 786 | GGCCGTCAAC | 883 |
| | | GGCCGTCAACGATACGACGCTCTTCCGAT* | | | |
| | | C*T | | | |
| D1 | OuterA.4 | CCATCTCATCCCTGCGTGTCTCCGACTCAG | 787 | CCTAGACGAG | 884 |
| | | CCTAGACGAGGATACGACGCTCTTCCGAT* | | | |
| | | C*T | | | |
| E1 | OuterA.5 | CCATCTCATCCCTGCGTGTCTCCGACTCAG | 788 | TCGTTAGAGC | 885 |
| | | TCGTTAGAGCGATACGACGCTCTTCCGAT* | | | |
| | | C*T | | | |
| F1 | OuterA.6 | CCATCTCATCCCTGCGTGTCTCCGACTCAG | 789 | CGTTCTATCA | 886 |
| | | CGTTCTATCAGATACGACGCTCTTCCGAT* | | | |
| | | C*T | | | |
| G1 | OuterA.7 | CCATCTCATCCCTGCGTGTCTCCGACTCAG | 790 | CGGAATCTAA | 887 |
| | | CGGAATCTAAGATACGACGCTCTTCCGAT* | | | |
| | | C*T | | | |
| H1 | OuterA.8 | CCATCTCATCCCTGCGTGTCTCCGACTCAG | 791 | ATGACTGATC | 888 |
| | | ATGACTGATCGATACGACGCTCTTCCGAT* | | | |
| | | C*T | | | |
| A2 | OuterA.9 | CCATCTCATCCCTGCGTGTCTCCGACTCAG | 792 | TCAATATCGA | 889 |
| | | TCAATATCGAGATACGACGCTCTTCCGAT* | | | |
| | | C*T | | | |
| B2 | OuterA.10 | CCATCTCATCCCTGCGTGTCTCCGACTCAG | 793 | GTAGACCTGG | 890 |
| | | GTAGACCTGGGATACGACGCTCTTCCGAT* | | | |
| | | C*T | | | |
| C2 | OuterA.11 | CCATCTCATCCCTGCGTGTCTCCGACTCAG | 794 | TTATGACCAA | 891 |
| | | TTATGACCAAGATACGACGCTCTTCCGAT* | | | |
| | | C*T | | | |
| D2 | OuterA.12 | CCATCTCATCCCTGCGTGTCTCCGACTCAG | 795 | TTGGTCCGTT | 892 |
| | | TTGGTCCGTTGATACGACGCTCTTCCGAT* | | | |
| | | C*T | | | |
| E2 | OuterA.13 | CCATCTCATCCCTGCGTGTCTCCGACTCAG | 796 | GGTACGTTAA | 893 |
| | | GGTACGTTAAGATACGACGCTCTTCCGAT* | | | |
| | | C*T | | | |
| F2 | OuterA.14 | CCATCTCATCCCTGCGTGTCTCCGACTCAG | 797 | CAATGAGTCC | 894 |
| | | CAATGAGTCCGATACGACGCTCTTCCGAT* | | | |
| | | C*T | | | |
| G2 | OuterA.15 | CCATCTCATCCCTGCGTGTCTCCGACTCAG | 798 | GATGCAGTTC | 895 |
| | | GATGCAGTTCGATACGACGCTCTTCCGAT* | | | |
| | | C*T | | | |
| H2 | OuterA.16 | CCATCTCATCCCTGCGTGTCTCCGACTCAG | 799 | CCATCGTTCC | 896 |
| | | CCATCGTTCCGATACGACGCTCTTCCGAT* | | | |
| | | C*T | | | |
| A3 | OuterA.17 | CCATCTCATCCCTGCGTGTCTCCGACTCAG | 800 | TTGAGAGAGT | 897 |
| | | TTGAGAGAGTGATACGACGCTCTTCCGAT* | | | |
| | | C*T | | | |
| B3 | OuterA.18 | CCATCTCATCCCTGCGTGTCTCCGACTCAG | 801 | ACTGAGCGAC | 898 |
| | | ACTGAGCGACGATACGACGCTCTTCCGAT* | | | |
| | | C*T | | | |
| C3 | OuterA.19 | CCATCTCATCCCTGCGTGTCTCCGACTCAG | 802 | TGAGGAATCA | 899 |
| | | TGAGGAATCAGATACGACGCTCTTCCGAT* | | | |
| | | C*T | | | |
| D3 | OuterA.20 | CCATCTCATCCCTGCGTGTCTCCGACTCAG | 803 | CCTCCGACGG | 900 |
| | | CCTCCGACGGGATACGACGCTCTTCCGAT* | | | |
| | | C*T | | | |
| E3 | OuterA.21 | CCATCTCATCCCTGCGTGTCTCCGACTCAG | 804 | CATTGACGCT | 901 |
| | | CATTGACGCTGATACGACGCTCTTCCGAT* | | | |
| | | C*T | | | |
| F3 | OuterA.22 | CCATCTCATCCCTGCGTGTCTCCGACTCAG | 805 | TCGTCCTTCG | 902 |
| | | TCGTCCTTCGGATACGACGCTCTTCCGAT* | | | |
| | | C*T | | | |
| G3 | OuterA.23 | CCATCTCATCCCTGCGTGTCTCCGACTCAG | 806 | TGATACTCAA | 903 |
| | | TGATACTCAAGATACGACGCTCTTCCGAT* | | | |
| | | C*T | | | |
| H3 | OuterA.24 | CCATCTCATCCCTGCGTGTCTCCGACTCAG | 807 | TTCTACCTCA | 904 |
| | | TTCTACCTCAGATACGACGCTCTTCCGAT* | | | |
| | | C*T | | | |
| A4 | OuterA.25 | CCATCTCATCCCTGCGTGTCTCCGACTCAG | 808 | TCGTCGGAAC | 905 |
| | | TCGTCGGAACGATACGACGCTCTTCCGAT* | | | |
| | | C*T | | | |
| B4 | OuterA.26 | CCATCTCATCCCTGCGTGTCTCCGACTCAG | 809 | ATCGAGATGA | 906 |
| | | ATCGAGATGAGATACGACGCTCTTCCGAT* | | | |
| | | C*T | | | |
| C4 | OuterA.27 | CCATCTCATCCCTGCGTGTCTCCGACTCAG | 810 | TAGACTAGTC | 907 |
| | | TAGACTAGTCGATACGACGCTCTTCCGAT* | | | |
| | | C*T | | | |
| D4 | OuterA.28 | CCATCTCATCCCTGCGTGTCTCCGACTCAG | 811 | GTCGAAGCAG | 908 |
| | | GTCGAAGCAGGATACGACGCTCTTCCGAT* | | | |
| | | C*T | | | |
| E4 | OuterA.29 | CCATCTCATCCCTGCGTGTCTCCGACTCAG | 812 | AGGCGCTAGG | 909 |
| | | AGGCGCTAGGGATACGACGCTCTTCCGAT* | | | |
| | | C*T | | | |
| F4 | OuterA.30 | CCATCTCATCCCTGCGTGTCTCCGACTCAG | 813 | AGATGCAACT | 910 |
| | | AGATGCAACTGATACGACGCTCTTCCGAT* | | | |
| | | C*T | | | |
| G4 | OuterA.31 | CCATCTCATCCCTGCGTGTCTCCGACTCAG | 814 | AAGCCTACGA | 911 |
| | | AAGCCTACGAGATACGACGCTCTTCCGAT* | | | |
| | | C*T | | | |
| H4 | OuterA.32 | CCATCTCATCCCTGCGTGTCTCCGACTCAG | 815 | GTAGGCAATT | 912 |
| | | GTAGGCAATTGATACGACGCTCTTCCGAT* | | | |
| | | C*T | | | |
| A5 | OuterA.33 | CCATCTCATCCCTGCGTGTCTCCGACTCAG | 816 | TGCCAGTTGC | 913 |
| | | TGCCAGTTGCGATACGACGCTCTTCCGAT* | | | |
| | | C*T | | | |
| B5 | OuterA.34 | CCATCTCATCCCTGCGTGTCTCCGACTCAG | 817 | CTTAGGTATC | 914 |
| | | CTTAGGTATCGATACGACGCTCTTCCGAT* | | | |
| | | C*T | | | |
| C5 | OuterA.35 | CCATCTCATCCCTGCGTGTCTCCGACTCAG | 818 | GAGACCTACC | 915 |
| | | GAGACCTACCGATACGACGCTCTTCCGAT* | | | |
| | | C*T | | | |
| D5 | OuterA.36 | CCATCTCATCCCTGCGTGTCTCCGACTCAG | 819 | ATTGACCGAG | 916 |
| | | ATTGACCGAGGATACGACGCTCTTCCGAT* | | | |
| | | C*T | | | |
| E5 | OuterA.37 | CCATCTCATCCCTGCGTGTCTCCGACTCAG | 820 | GGAGGCGGCG | 917 |
| | | GGAGGCGGCGGATACGACGCTCTTCCGAT* | | | |
| | | C*T | | | |
| F5 | OuterA.38 | CCATCTCATCCCTGCGTGTCTCCGACTCAG | 821 | CCAGTACTTG | 918 |
| | | CCAGTACTTGGATACGACGCTCTTCCGAT* | | | |
| | | C*T | | | |
| G5 | OuterA.39 | CCATCTCATCCCTGCGTGTCTCCGACTCAG | 822 | GGTCTCGCCG | 919 |
| | | GGTCTCGCCGGATACGACGCTCTTCCGAT* | | | |
| | | C*T | | | |
| H5 | OuterA.40 | CCATCTCATCCCTGCGTGTCTCCGACTCAG | 823 | GGCGGAGGTC | 920 |
| | | GGCGGAGGTCGATACGACGCTCTTCCGAT* | | | |
| | | C*T | | | |
| A6 | OuterA.41 | CCATCTCATCCCTGCGTGTCTCCGACTCAG | 824 | TAGTTCTAGA | 921 |
| | | TAGTTCTAGAGATACGACGCTCTTCCGAT* | | | |
| | | C*T | | | |
| B6 | OuterA.42 | CCATCTCATCCCTGCGTGTCTCCGACTCAG | 825 | TTGGAGTTAG | 922 |
| | | TTGGAGTTAGGATACGACGCTCTTCCGAT* | | | |
| | | C*T | | | |
| C6 | OuterA.43 | CCATCTCATCCCTGCGTGTCTCCGACTCAG | 826 | AGATCTTGGT | 923 |
| | | AGATCTTGGTGATACGACGCTCTTCCGAT* | | | |
| | | C*T | | | |
| D6 | OuterA.44 | CCATCTCATCCCTGCGTGTCTCCGACTCAG | 827 | GTAATGATCG | 924 |
| | | GTAATGATCGGATACGACGCTCTTCCGAT* | | | |
| | | C*T | | | |
| E6 | OuterA.45 | CCATCTCATCCCTGCGTGTCTCCGACTCAG | 828 | CAGAGAGGTC | 925 |
| | | CAGAGAGGTCGATACGACGCTCTTCCGAT* | | | |
| | | C*T | | | |
| F6 | OuterA.46 | CCATCTCATCCCTGCGTGTCTCCGACTCAG | 829 | TTAATTAGCC | 926 |
| | | TTAATTAGCCGATACGACGCTCTTCCGAT* | | | |
| | | C*T | | | |
| G6 | OuterA.47 | CCATCTCATCCCTGCGTGTCTCCGACTCAG | 830 | CTCTAACTCG | 927 |
| | | CTCTAACTCGGATACGACGCTCTTCCGAT* | | | |
| | | C*T | | | |
| H6 | OuterA.48 | CCATCTCATCCCTGCGTGTCTCCGACTCAG | 831 | TACGATCATC | 928 |
| | | TACGATCATCGATACGACGCTCTTCCGAT* | | | |
| | | C*T | | | |
| A7 | OuterA.49 | CCATCTCATCCCTGCGTGTCTCCGACTCAG | 832 | AGGCGAGAGC | 929 |
| | | AGGCGAGAGCGATACGACGCTCTTCCGAT* | | | |
| | | C*T | | | |
| B7 | OuterA.50 | CCATCTCATCCCTGCGTGTCTCCGACTCAG | 833 | TCAAGATAGT | 930 |
| | | TCAAGATAGTGATACGACGCTCTTCCGAT* | | | |
| | | C*T | | | |
| C7 | OuterA.51 | CCATCTCATCCCTGCGTGTCTCCGACTCAG | 834 | TAATTGACCT | 931 |
| | | TAATTGACCTGATACGACGCTCTTCCGAT* | | | |
| | | C*T | | | |
| D7 | OuterA.52 | CCATCTCATCCCTGCGTGTCTCCGACTCAG | 835 | CAGCCGGCTT | 932 |
| | | CAGCCGGCTTGATACGACGCTCTTCCGAT* | | | |
| | | C*T | | | |
| E7 | OuterA.53 | CCATCTCATCCCTGCGTGTCTCCGACTCAG | 836 | AGAACCGGAG | 933 |
| | | AGAACCGGAGGATACGACGCTCTTCCGAT* | | | |
| | | C*T | | | |
| F7 | OuterA.54 | CCATCTCATCCCTGCGTGTCTCCGACTCAG | 837 | GAGATGCATG | 934 |
| | | GAGATGCATGGATACGACGCTCTTCCGAT* | | | |
| | | C*T | | | |
| G7 | OuterA.55 | CCATCTCATCCCTGCGTGTCTCCGACTCAG | 838 | GATTACCGGA | 935 |
| | | GATTACCGGAGATACGACGCTCTTCCGAT* | | | |
| | | C*T | | | |
| H7 | OuterA.56 | CCATCTCATCCCTGCGTGTCTCCGACTCAG | 839 | TCGTAACGGT | 936 |
| | | TCGTAACGGTGATACGACGCTCTTCCGAT* | | | |
| | | C*T | | | |
| A8 | OuterA.57 | CCATCTCATCCCTGCGTGTCTCCGACTCAG | 840 | TGGCGACGGA | 937 |
| | | TGGCGACGGAGATACGACGCTCTTCCGAT* | | | |
| | | C*T | | | |
| B8 | OuterA.58 | CCATCTCATCCCTGCGTGTCTCCGACTCAG | 841 | AGTCATAGCC | 938 |
| | | AGTCATAGCCGATACGACGCTCTTCCGAT* | | | |
| | | C*T | | | |
| C8 | OuterA.59 | CCATCTCATCCCTGCGTGTCTCCGACTCAG | 842 | GTCAAGTCCA | 939 |
| | | GTCAAGTCCAGATACGACGCTCTTCCGAT* | | | |
| | | C*T | | | |
| D8 | OuterA.60 | CCATCTCATCCCTGCGTGTCTCCGACTCAG | 843 | ATTCGGAAGT | 940 |
| | | ATTCGGAAGTGATACGACGCTCTTCCGAT* | | | |
| | | C*T | | | |
| E8 | OuterA.61 | CCATCTCATCCCTGCGTGTCTCCGACTCAG | 844 | GTCGGTAGTT | 941 |
| | | GTCGGTAGTTGATACGACGCTCTTCCGAT* | | | |
| | | C*T | | | |
| F8 | OuterA.62 | CCATCTCATCCCTGCGTGTCTCCGACTCAG | 845 | AGGACGGACG | 942 |
| | | AGGACGGACGGATACGACGCTCTTCCGAT* | | | |
| | | C*T | | | |
| G8 | OuterA.63 | CCATCTCATCCCTGCGTGTCTCCGACTCAG | 846 | CTCCTGGACC | 943 |
| | | CTCCTGGACCGATACGACGCTCTTCCGAT* | | | |
| | | C*T | | | |
| H8 | OuterA.64 | CCATCTCATCCCTGCGTGTCTCCGACTCAG | 847 | TAGCCTCGTT | 944 |
| | | TAGCCTCGTTGATACGACGCTCTTCCGAT* | | | |
| | | C*T | | | |
| A9 | OuterA.65 | CCATCTCATCCCTGCGTGTCTCCGACTCAG | 848 | GGTTGAACGT | 945 |
| | | GGTTGAACGTGATACGACGCTCTTCCGAT* | | | |
| | | C*T | | | |
| B9 | OuterA.66 | CCATCTCATCCCTGCGTGTCTCCGACTCAG | 849 | AGGTCCTCGT | 946 |
| | | AGGTCCTCGTGATACGACGCTCTTCCGAT* | | | |
| | | C*T | | | |
| C9 | OuterA.67 | CCATCTCATCCCTGCGTGTCTCCGACTCAG | 850 | GGAAGTTATA | 947 |
| | | GGAAGTTATAGATACGACGCTCTTCCGAT* | | | |
| | | C*T | | | |
| D9 | OuterA.68 | CCATCTCATCCCTGCGTGTCTCCGACTCAG | 851 | TGGTAATCCT | 948 |
| | | TGGTAATCCTGATACGACGCTCTTCCGAT* | | | |
| | | C*T | | | |
| E9 | OuterA.69 | CCATCTCATCCCTGCGTGTCTCCGACTCAG | 852 | AAGCTAGGTT | 949 |
| | | AAGCTAGGTTGATACGACGCTCTTCCGAT* | | | |
| | | C*T | | | |
| F9 | OuterA.70 | CCATCTCATCCCTGCGTGTCTCCGACTCAG | 853 | TCCGCGGACT | 950 |
| | | TCCGCGGACTGATACGACGCTCTTCCGAT* | | | |
| | | C*T | | | |
| G9 | OuterA.71 | CCATCTCATCCCTGCGTGTCTCCGACTCAG | 854 | TGCGGATAGT | 951 |
| | | TGCGGATAGTGATACGACGCTCTTCCGAT* | | | |
| | | C*T | | | |
| H9 | OuterA.72 | CCATCTCATCCCTGCGTGTCTCCGACTCAG | 855 | TGGCAGCTCG | 952 |
| | | TGGCAGCTCGGATACGACGCTCTTCCGAT* | | | |
| | | C*T | | | |
| A10 | OuterA.73 | CCATCTCATCCCTGCGTGTCTCCGACTCAG | 856 | TGCTACGGTC | 953 |
| | | TGCTACGGTCGATACGACGCTCTTCCGAT* | | | |
| | | C*T | | | |
| B10 | OuterA.74 | CCATCTCATCCCTGCGTGTCTCCGACTCAG | 857 | GCGCAATGAC | 954 |
| | | GCGCAATGACGATACGACGCTCTTCCGAT* | | | |
| | | C*T | | | |
| C10 | OuterA.75 | CCATCTCATCCCTGCGTGTCTCCGACTCAG | 858 | CTTAATCTTG | 955 |
| | | CTTAATCTTGGATACGACGCTCTTCCGAT* | | | |
| | | C*T | | | |
| D10 | OuterA.76 | CCATCTCATCCCTGCGTGTCTCCGACTCAG | 859 | GGAGTTGCGT | 956 |
| | | GGAGTTGCGTGATACGACGCTCTTCCGAT* | | | |
| | | C*T | | | |
| E10 | OuterA.77 | CCATCTCATCCCTGCGTGTCTCCGACTCAG | 860 | ACTCGTATCA | 957 |
| | | ACTCGTATCAGATACGACGCTCTTCCGAT* | | | |
| | | C*T | | | |
| F10 | OuterA.78 | CCATCTCATCCCTGCGTGTCTCCGACTCAG | 861 | GGTAATAATG | 958 |
| | | GGTAATAATGGATACGACGCTCTTCCGAT* | | | |
| | | C*T | | | |
| G10 | OuterA.79 | CCATCTCATCCCTGCGTGTCTCCGACTCAG | 862 | TCCTTATAGA | 959 |
| | | TCCTTATAGAGATACGACGCTCTTCCGAT* | | | |
| | | C*T | | | |
| H10 | OuterA.80 | CCATCTCATCCCTGCGTGTCTCCGACTCAG | 863 | CCGACTCCAA | 960 |
| | | CCGACTCCAAGATACGACGCTCTTCCGAT* | | | |
| | | C*T | | | |
| A11 | OuterA.81 | CCATCTCATCCCTGCGTGTCTCCGACTCAG | 864 | GCCAAGCTTG | 961 |
| | | GCCAAGCTTGGATACGACGCTCTTCCGAT* | | | |
| | | C*T | | | |
| B11 | OuterA.82 | CCATCTCATCCCTGCGTGTCTCCGACTCAG | 865 | CATATCCTAT | 962 |
| | | CATATCCTATGATACGACGCTCTTCCGAT* | | | |
| | | C*T | | | |
| C11 | OuterA.83 | CCATCTCATCCCTGCGTGTCTCCGACTCAG | 866 | ACCTACGCCA | 963 |
| | | ACCTACGCCAGATACGACGCTCTTCCGAT* | | | |
| | | C*T | | | |
| D11 | OuterA.84 | CCATCTCATCCCTGCGTGTCTCCGACTCAG | 867 | GGAATTCAGT | 964 |
| | | GGAATTCAGTGATACGACGCTCTTCCGAT* | | | |
| | | C*T | | | |
| E11 | OuterA.85 | CCATCTCATCCCTGCGTGTCTCCGACTCAG | 868 | TGGCGTAGAA | 965 |
| | | TGGCGTAGAAGATACGACGCTCTTCCGAT* | | | |
| | | C*T | | | |
| F11 | OuterA.86 | CCATCTCATCCCTGCGTGTCTCCGACTCAG | 869 | ATTGCGGCCA | 966 |
| | | ATTGCGGCCAGATACGACGCTCTTCCGAT* | | | |
| | | C*T | | | |
| G11 | OuterA.87 | CCATCTCATCCCTGCGTGTCTCCGACTCAG | 870 | TTCAGCTTGG | 967 |
| | | TTCAGCTTGGGATACGACGCTCTTCCGAT* | | | |
| | | C*T | | | |
| H11 | OuterA.88 | CCATCTCATCCCTGCGTGTCTCCGACTCAG | 871 | CCATCTGGCA | 968 |
| | | CCATCTGGCAGATACGACGCTCTTCCGAT* | | | |
| | | C*T | | | |
| A12 | OuterA.89 | CCATCTCATCCCTGCGTGTCTCCGACTCAG | 872 | CTTATAAGTT | 969 |
| | | CTTATAAGTTGATACGACGCTCTTCCGAT* | | | |
| | | C*T | | | |
| B12 | OuterA.90 | CCATCTCATCCCTGCGTGTCTCCGACTCAG | 873 | GATTAGATGA | 970 |
| | | GATTAGATGAGATACGACGCTCTTCCGAT* | | | |
| | | C*T | | | |
| C12 | OuterA.91 | CCATCTCATCCCTGCGTGTCTCCGACTCAG | 874 | TATAGGATCT | 971 |
| | | TATAGGATCTGATACGACGCTCTTCCGAT* | | | |
| | | C*T | | | |
| D12 | OuterA.92 | CCATCTCATCCCTGCGTGTCTCCGACTCAG | 875 | AGCTTATAGG | 972 |
| | | AGCTTATAGGGATACGACGCTCTTCCGAT* | | | |
| | | C*T | | | |
| E12 | OuterA.93 | CCATCTCATCCCTGCGTGTCTCCGACTCAG | 876 | GTCTGCAATC | 973 |
| | | GTCTGCAATCGATACGACGCTCTTCCGAT* | | | |
| | | C*T | | | |
| F12 | OuterA.94 | CCATCTCATCCCTGCGTGTCTCCGACTCAG | 877 | CGCCTCTTAT | 974 |
| | | CGCCTCTTATGATACGACGCTCTTCCGAT* | | | |
| | | C*T | | | |
| G12 | OuterA.95 | CCATCTCATCCCTGCGTGTCTCCGACTCAG | 878 | GTTGGATCTT | 975 |
| | | GTTGGATCTTGATACGACGCTCTTCCGAT* | | | |
| | | C*T | | | |
| H12 | OuterA.96 | CCATCTCATCCCTGCGTGTCTCCGACTCAG | 879 | GCGATTGCAG | 976 |
| | | GCGATTGCAGGATACGACGCTCTTCCGAT* | | | |
| | | C*T | | | |

| | |
|---|---|
| Backbone | CCATCTCATCCCTGCGTGTCTCCGACTCAG (SEQ ID NO: 880) |
| (adapterA) | |
| Splint | GATACGACGCTCTTCCGAT*C*T (SEQ ID NO: 977) |

TABLE 9
IonTorrent_Custom_5′_InnerP_96

| | | | | | Index |
|---|---|---|---|---|---|
| | | | SEQ ID | | SEQ ID |
| Well | Name | Sequence | NO. | Index | NO. |
| A1 | InnerP.1 | CCTCTCTATGGGCAGTCGGTGATCCGAATC | 978 | CCGAATCCGA | 1075 |
| | | CGAGTCTCGTGGGCTC*G*G | | | |
| B1 | InnerP.2 | CCTCTCTATGGGCAGTCGGTGATATAAGCC | 979 | ATAAGCCGGA | 1076 |
| | | GGAGTCTCGTGGGCTC*G*G | | | |
| C1 | InnerP.3 | CCTCTCTATGGGCAGTCGGTGATCCGGCGG | 980 | CCGGCGGCGA | 1077 |
| | | CGAGTCTCGTGGGCTC*G*G | | | |
| D1 | InnerP.4 | CCTCTCTATGGGCAGTCGGTGATGGCTTGC | 981 | GGCTTGCCAA | 1078 |
| | | CAAGTCTCGTGGGCTC*G*G | | | |
| E1 | InnerP.5 | CCTCTCTATGGGCAGTCGGTGATCCGCTAG | 982 | CCGCTAGCTG | 1079 |
| | | CTGGTCTCGTGGGCTC*G*G | | | |
| F1 | InnerP.6 | CCTCTCTATGGGCAGTCGGTGATCTTATCC | 983 | CTTATCCTAC | 1080 |
| | | TACGTCTCGTGGGCTC*G*G | | | |
| G1 | InnerP.7 | CCTCTCTATGGGCAGTCGGTGATTGAGCTA | 984 | TGAGCTACTT | 1081 |
| | | CTTGTCTCGTGGGCTC*G*G | | | |
| H1 | InnerP.8 | CCTCTCTATGGGCAGTCGGTGATTCAGGAC | 985 | TCAGGACTTA | 1082 |
| | | TTAGTCTCGTGGGCTC*G*G | | | |
| A2 | InnerP.9 | CCTCTCTATGGGCAGTCGGTGATCCGCAGC | 986 | CCGCAGCCGC | 1083 |
| | | CGCGTCTCGTGGGCTC*G*G | | | |
| B2 | InnerP.10 | CCTCTCTATGGGCAGTCGGTGATTGCGCCT | 987 | TGCGCCTGGT | 1084 |
| | | GGTGTCTCGTGGGCTC*G*G | | | |
| C2 | InnerP.11 | CCTCTCTATGGGCAGTCGGTGATAATCATA | 988 | AATCATACGG | 1085 |
| | | CGGGTCTCGTGGGCTC*G*G | | | |
| D2 | InnerP.12 | CCTCTCTATGGGCAGTCGGTGATCGCCAAT | 989 | CGCCAATCAA | 1086 |
| | | CAAGTCTCGTGGGCTC*G*G | | | |
| E2 | InnerP.13 | CCTCTCTATGGGCAGTCGGTGATCAAGGCT | 990 | CAAGGCTTAG | 1087 |
| | | TAGGTCTCGTGGGCTC*G*G | | | |
| F2 | InnerP.14 | CCTCTCTATGGGCAGTCGGTGATGCGCTCG | 991 | GCGCTCGACG | 1088 |
| | | ACGGTCTCGTGGGCTC*G*G | | | |
| G2 | InnerP.15 | CCTCTCTATGGGCAGTCGGTGATTCCAGCA | 992 | TCCAGCAATA | 1089 |
| | | ATAGTCTCGTGGGCTC*G*G | | | |
| H2 | InnerP.16 | CCTCTCTATGGGCAGTCGGTGATCATGAGA | 993 | CATGAGAACT | 1090 |
| | | ACTGTCTCGTGGGCTC*G*G | | | |
| A3 | InnerP.17 | CCTCTCTATGGGCAGTCGGTGATAACGTAA | 994 | AACGTAATCT | 1091 |
| | | TCTGTCTCGTGGGCTC*G*G | | | |
| B3 | InnerP.18 | CCTCTCTATGGGCAGTCGGTGATATTCTCC | 995 | ATTCTCCTCT | 1092 |
| | | TCTGTCTCGTGGGCTC*G*G | | | |
| C3 | InnerP.19 | CCTCTCTATGGGCAGTCGGTGATTCTGCGC | 996 | TCTGCGCGTT | 1093 |
| | | GTTGTCTCGTGGGCTC*G*G | | | |
| D3 | InnerP.20 | CCTCTCTATGGGCAGTCGGTGATGCTCATA | 997 | GCTCATATGC | 1094 |
| | | TGCGTCTCGTGGGCTC*G*G | | | |
| E3 | InnerP.21 | CCTCTCTATGGGCAGTCGGTGATAGCGGTA | 998 | AGCGGTAACG | 1095 |
| | | ACGGTCTCGTGGGCTC*G*G | | | |
| F3 | InnerP.22 | CCTCTCTATGGGCAGTCGGTGATAATGAAT | 999 | AATGAATAGT | 1096 |
| | | AGTGTCTCGTGGGCTC*G*G | | | |
| G3 | InnerP.23 | CCTCTCTATGGGCAGTCGGTGATCCGTATC | 1000 | CCGTATCTGG | 1097 |
| | | TGGGTCTCGTGGGCTC*G*G | | | |
| H3 | InnerP.24 | CCTCTCTATGGGCAGTCGGTGATCCTTAGT | 1001 | CCTTAGTCTG | 1098 |
| | | CTGGTCTCGTGGGCTC*G*G | | | |
| A4 | InnerP.25 | CCTCTCTATGGGCAGTCGGTGATACCTAGT | 1002 | ACCTAGTTAG | 1099 |
| | | TAGGTCTCGTGGGCTC*G*G | | | |
| B4 | InnerP.26 | CCTCTCTATGGGCAGTCGGTGATATAGGAG | 1003 | ATAGGAGTAC | 1100 |
| | | TACGTCTCGTGGGCTC*G*G | | | |
| C4 | InnerP.27 | CCTCTCTATGGGCAGTCGGTGATCTACGAC | 1004 | CTACGACGAG | 1101 |
| | | GAGGTCTCGTGGGCTC*G*G | | | |
| D4 | InnerP.28 | CCTCTCTATGGGCAGTCGGTGATAGTCGAG | 1005 | AGTCGAGTTC | 1102 |
| | | TTCGTCTCGTGGGCTC*G*G | | | |
| E4 | InnerP.29 | CCTCTCTATGGGCAGTCGGTGATTGGTCCA | 1006 | TGGTCCAGTC | 1103 |
| | | GTCGTCTCGTGGGCTC*G*G | | | |
| F4 | InnerP.30 | CCTCTCTATGGGCAGTCGGTGATATCTAAG | 1007 | ATCTAAGCAA | 1104 |
| | | CAAGTCTCGTGGGCTC*G*G | | | |
| G4 | InnerP .31 | CCTCTCTATGGGCAGTCGGTGATCGAATTC | 1008 | CGAATTCGTT | 1105 |
| | | GTTGTCTCGTGGGCTC*G*G | | | |
| H4 | InnerP.32 | CCTCTCTATGGGCAGTCGGTGATCAGCGAT | 1009 | CAGCGATAGA | 1106 |
| | | AGAGTCTCGTGGGCTC*G*G | | | |
| A5 | InnerP.33 | CCTCTCTATGGGCAGTCGGTGATGGTCGCT | 1010 | GGTCGCTATG | 1107 |
| | | ATGGTCTCGTGGGCTC*G*G | | | |
| B5 | InnerP.34 | CCTCTCTATGGGCAGTCGGTGATATCCGTT | 1011 | ATCCGTTAGC | 1108 |
| | | AGCGTCTCGTGGGCTC*G*G | | | |
| C5 | InnerP.35 | CCTCTCTATGGGCAGTCGGTGATTCGCAAT | 1012 | TCGCAATTAG | 1109 |
| | | TAGGTCTCGTGGGCTC*G*G | | | |
| D5 | InnerP.36 | CCTCTCTATGGGCAGTCGGTGATGGCTGGC | 1013 | GGCTGGCTAG | 1110 |
| | | TAGGTCTCGTGGGCTC*G*G | | | |
| E5 | InnerP.37 | CCTCTCTATGGGCAGTCGGTGATACGGTCT | 1014 | ACGGTCTTGC | 1111 |
| | | TGCGTCTCGTGGGCTC*G*G | | | |
| F5 | InnerP.38 | CCTCTCTATGGGCAGTCGGTGATGCTCCAT | 1015 | GCTCCATTCG | 1112 |
| | | TCGGTCTCGTGGGCTC*G*G | | | |
| G5 | InnerP.39 | CCTCTCTATGGGCAGTCGGTGATACGATAA | 1016 | ACGATAAGCG | 1113 |
| | | GCGGTCTCGTGGGCTC*G*G | | | |
| H5 | InnerP.40 | CCTCTCTATGGGCAGTCGGTGATACCATAG | 1017 | ACCATAGCGC | 1114 |
| | | CGCGTCTCGTGGGCTC*G*G | | | |
| A6 | InnerP.41 | CCTCTCTATGGGCAGTCGGTGATCTCTTAG | 1018 | CTCTTAGCGG | 1115 |
| | | CGGGTCTCGTGGGCTC*G*G | | | |
| B6 | InnerP.42 | CCTCTCTATGGGCAGTCGGTGATTGATTCA | 1019 | TGATTCAACT | 1116 |
| | | ACTGTCTCGTGGGCTC*G*G | | | |
| C6 | InnerP.43 | CCTCTCTATGGGCAGTCGGTGATTATGGCC | 1020 | TATGGCCGCG | 1117 |
| | | GCGGTCTCGTGGGCTC*G*G | | | |
| D6 | InnerP.44 | CCTCTCTATGGGCAGTCGGTGATAGAGGTC | 1021 | AGAGGTCGCA | 1118 |
| | | GCAGTCTCGTGGGCTC*G*G | | | |
| E6 | InnerP.45 | CCTCTCTATGGGCAGTCGGTGATAGGAGAT | 1022 | AGGAGATTGA | 1119 |
| | | TGAGTCTCGTGGGCTC*G*G | | | |
| F6 | InnerP.46 | CCTCTCTATGGGCAGTCGGTGATGGCTATA | 1023 | GGCTATATAG | 1120 |
| | | TAGGTCTCGTGGGCTC*G*G | | | |
| G6 | InnerP .47 | CCTCTCTATGGGCAGTCGGTGATTCGCGTA | 1024 | TCGCGTACTT | 1121 |
| | | CTTGTCTCGTGGGCTC*G*G | | | |
| H6 | InnerP.48 | CCTCTCTATGGGCAGTCGGTGATAATAATA | 1025 | AATAATAATG | 1122 |
| | | ATGGTCTCGTGGGCTC*G*G | | | |
| A7 | InnerP .49 | CCTCTCTATGGGCAGTCGGTGATTTCGTTC | 1026 | TTCGTTCCAT | 1123 |
| | | CATGTCTCGTGGGCTC*G*G | | | |
| B7 | InnerP.50 | CCTCTCTATGGGCAGTCGGTGATTACCTAA | 1027 | TACCTAATCA | 1124 |
| | | TCAGTCTCGTGGGCTC*G*G | | | |
| C7 | InnerP.51 | CCTCTCTATGGGCAGTCGGTGATAAGTAAT | 1028 | AAGTAATATT | 1125 |
| | | ATTGTCTCGTGGGCTC*G*G | | | |
| D7 | InnerP.52 | CCTCTCTATGGGCAGTCGGTGATAGCTAAG | 1029 | AGCTAAGAAT | 1126 |
| | | AATGTCTCGTGGGCTC*G*G | | | |
| E7 | InnerP.53 | CCTCTCTATGGGCAGTCGGTGATGTCGAGG | 1030 | GTCGAGGTAT | 1127 |
| | | TATGTCTCGTGGGCTC*G*G | | | |
| F7 | InnerP.54 | CCTCTCTATGGGCAGTCGGTGATTTATTAG | 1031 | TTATTAGTAG | 1128 |
| | | TAGGTCTCGTGGGCTC*G*G | | | |
| G7 | InnerP.55 | CCTCTCTATGGGCAGTCGGTGATTGCGAAG | 1032 | TGCGAAGATC | 1129 |
| | | ATCGTCTCGTGGGCTC*G*G | | | |
| H7 | InnerP.56 | CCTCTCTATGGGCAGTCGGTGATAACTACG | 1033 | AACTACGGCT | 1130 |
| | | GCTGTCTCGTGGGCTC*G*G | | | |
| A8 | InnerP.57 | CCTCTCTATGGGCAGTCGGTGATAACGGAA | 1034 | AACGGAACGC | 1131 |
| | | CGCGTCTCGTGGGCTC*G*G | | | |
| B8 | InnerP.58 | CCTCTCTATGGGCAGTCGGTGATGATGCTA | 1035 | GATGCTACGA | 1132 |
| | | CGAGTCTCGTGGGCTC*G*G | | | |
| C8 | InnerP.59 | CCTCTCTATGGGCAGTCGGTGATATCTGCC | 1036 | ATCTGCCAAT | 1133 |
| | | AATGTCTCGTGGGCTC*G*G | | | |
| D8 | InnerP.60 | CCTCTCTATGGGCAGTCGGTGATATCGTAT | 1037 | ATCGTATCAA | 1134 |
| | | CAAGTCTCGTGGGCTC*G*G | | | |
| E8 | InnerP.61 | CCTCTCTATGGGCAGTCGGTGATAACGCCT | 1038 | AACGCCTCTA | 1135 |
| | | CTAGTCTCGTGGGCTC*G*G | | | |
| F8 | InnerP.62 | CCTCTCTATGGGCAGTCGGTGATACGGCAA | 1039 | ACGGCAACCA | 1136 |
| | | CCAGTCTCGTGGGCTC*G*G | | | |
| G8 | InnerP.63 | CCTCTCTATGGGCAGTCGGTGATCAGGCTA | 1040 | CAGGCTAAGA | 1137 |
| | | AGAGTCTCGTGGGCTC*G*G | | | |
| H8 | InnerP.64 | CCTCTCTATGGGCAGTCGGTGATCGCAATA | 1041 | CGCAATATCA | 1138 |
| | | TCAGTCTCGTGGGCTC*G*G | | | |
| A9 | InnerP.65 | CCTCTCTATGGGCAGTCGGTGATTTCGATA | 1042 | TTCGATAACC | 1139 |
| | | ACCGTCTCGTGGGCTC*G*G | | | |
| B9 | InnerP.66 | CCTCTCTATGGGCAGTCGGTGATAACCTCA | 1043 | AACCTCAAGA | 1140 |
| | | AGAGTCTCGTGGGCTC*G*G | | | |
| C9 | InnerP.67 | CCTCTCTATGGGCAGTCGGTGATCAGGCGC | 1044 | CAGGCGCCAT | 1141 |
| | | CATGTCTCGTGGGCTC*G*G | | | |
| D9 | InnerP.68 | CCTCTCTATGGGCAGTCGGTGATAACTATT | 1045 | AACTATTATA | 1142 |
| | | ATAGTCTCGTGGGCTC*G*G | | | |
| E9 | InnerP.69 | CCTCTCTATGGGCAGTCGGTGATAAGTTAC | 1046 | AAGTTACCTA | 1143 |
| | | CTAGTCTCGTGGGCTC*G*G | | | |
| F9 | InnerP.70 | CCTCTCTATGGGCAGTCGGTGATCGGCAGA | 1047 | CGGCAGAGGA | 1144 |
| | | GGAGTCTCGTGGGCTC*G*G | | | |
| G9 | InnerP.71 | CCTCTCTATGGGCAGTCGGTGATGCCTCAA | 1048 | GCCTCAATAA | 1145 |
| | | TAAGTCTCGTGGGCTC*G*G | | | |
| H9 | InnerP.72 | CCTCTCTATGGGCAGTCGGTGATTTAACGC | 1049 | TTAACGCCGT | 1146 |
| | | CGTGTCTCGTGGGCTC*G*G | | | |
| A10 | InnerP.73 | CCTCTCTATGGGCAGTCGGTGATCATACGA | 1050 | CATACGATGC | 1147 |
| | | TGCGTCTCGTGGGCTC*G*G | | | |
| B10 | InnerP.74 | CCTCTCTATGGGCAGTCGGTGATAAGCTGA | 1051 | AAGCTGACCT | 1148 |
| | | CCTGTCTCGTGGGCTC*G*G | | | |
| C10 | InnerP.75 | CCTCTCTATGGGCAGTCGGTGATGAGTCCT | 1052 | GAGTCCTTAT | 1149 |
| | | TATGTCTCGTGGGCTC*G*G | | | |
| D10 | InnerP.76 | CCTCTCTATGGGCAGTCGGTGATCCTACGG | 1053 | CCTACGGCAA | 1150 |
| | | CAAGTCTCGTGGGCTC*G*G | | | |
| E10 | InnerP.77 | CCTCTCTATGGGCAGTCGGTGATAATATTC | 1054 | AATATTCGAA | 1151 |
| | | GAAGTCTCGTGGGCTC*G*G | | | |
| F10 | InnerP.78 | CCTCTCTATGGGCAGTCGGTGATTTCAAGA | 1055 | TTCAAGAATC | 1152 |
| | | ATCGTCTCGTGGGCTC*G*G | | | |
| G10 | InnerP.79 | CCTCTCTATGGGCAGTCGGTGATATGCTCG | 1056 | ATGCTCGCAA | 1153 |
| | | CAAGTCTCGTGGGCTC*G*G | | | |
| H10 | InnerP.80 | CCTCTCTATGGGCAGTCGGTGATGGAGTAA | 1057 | GGAGTAAGCC | 1154 |
| | | GCCGTCTCGTGGGCTC*G*G | | | |
| A11 | InnerP.81 | CCTCTCTATGGGCAGTCGGTGATTTATCGT | 1058 | TTATCGTATT | 1155 |
| | | ATTGTCTCGTGGGCTC*G*G | | | |
| B11 | InnerP.82 | CCTCTCTATGGGCAGTCGGTGATAAGTCTA | 1059 | AAGTCTAATA | 1156 |
| | | ATAGTCTCGTGGGCTC*G*G | | | |
| C11 | InnerP.83 | CCTCTCTATGGGCAGTCGGTGATCGGCTTA | 1060 | CGGCTTACTA | 1157 |
| | | CTAGTCTCGTGGGCTC*G*G | | | |
| D11 | InnerP.84 | CCTCTCTATGGGCAGTCGGTGATGATATGG | 1061 | GATATGGTCT | 1158 |
| | | TCTGTCTCGTGGGCTC*G*G | | | |
| E11 | InnerP.85 | CCTCTCTATGGGCAGTCGGTGATTAGTCGT | 1062 | TAGTCGTCCA | 1159 |
| | | CCAGTCTCGTGGGCTC*G*G | | | |
| F11 | InnerP.86 | CCTCTCTATGGGCAGTCGGTGATTAGCTGC | 1063 | TAGCTGCTAC | 1160 |
| | | TACGTCTCGTGGGCTC*G*G | | | |
| G11 | InnerP.87 | CCTCTCTATGGGCAGTCGGTGATCTCTTCA | 1064 | CTCTTCAAGC | 1161 |
| | | AGCGTCTCGTGGGCTC*G*G | | | |
| H11 | InnerP.88 | CCTCTCTATGGGCAGTCGGTGATATGAACG | 1065 | ATGAACGCGC | 1162 |
| | | CGCGTCTCGTGGGCTC*G*G | | | |
| A12 | InnerP.89 | CCTCTCTATGGGCAGTCGGTGATGTCGACG | 1066 | GTCGACGGAA | 1163 |
| | | GAAGTCTCGTGGGCTC*G*G | | | |
| B12 | InnerP.90 | CCTCTCTATGGGCAGTCGGTGATACTAATT | 1067 | ACTAATTGAG | 1164 |
| | | GAGGTCTCGTGGGCTC*G*G | | | |
| C12 | InnerP.91 | CCTCTCTATGGGCAGTCGGTGATCTTGCAT | 1068 | CTTGCATAAT | 1165 |
| | | AATGTCTCGTGGGCTC*G*G | | | |
| D12 | InnerP.92 | CCTCTCTATGGGCAGTCGGTGATTCCTTAC | 1069 | TCCTTACCAA | 1166 |
| | | CAAGTCTCGTGGGCTC*G*G | | | |
| E12 | InnerP.93 | CCTCTCTATGGGCAGTCGGTGATTGCAGCC | 1070 | TGCAGCCTAC | 1167 |
| | | TACGTCTCGTGGGCTC*G*G | | | |
| F12 | InnerP.94 | CCTCTCTATGGGCAGTCGGTGATGGAGCTG | 1071 | GGAGCTGAGG | 1168 |
| | | AGGGTCTCGTGGGCTC*G*G | | | |
| G12 | InnerP.95 | CCTCTCTATGGGCAGTCGGTGATGCAGCGG | 1072 | GCAGCGGACT | 1169 |
| | | ACTGTCTCGTGGGCTC*G*G | | | |
| H12 | InnerP.96 | CCTCTCTATGGGCAGTCGGTGATCATCGCG | 1073 | CATCGCGCTC | 1170 |
| | | CTCGTCTCGTGGGCTC*G*G | | | |

| | |
|---|---|
| Backbone | CCTCTCTATGGGCAGTCGGTGAT (SEQ ID NO: 1074) |
| (adapterP1) | |
| Splint To | GTCTCGTGGGCTC*G*G (SEQ ID NO: 1171) |
| TRS coupler | |

In some embodiments, method is performed in a single-pot, closed tube chemistry. For example, see Example 1 below.

In some embodiments, the method is performed in a single-pot, open tube chemistry. For example, see Example 2 below.

In some embodiments, the method is performed in a split-pot, multi-tube chemistry using PCR pre-amplification. For example, see Example 3 below.

In some embodiments, the method is performed in a split-pot, multi-tube chemistry using MDA pre-amplification. For example, see Example 4 below.

In certain embodiments, the method comprises not only detecting nucleic acid from a pathogen in the sample, but also determining the subject's gene expression patterns in response to the pathogen. The host subject's gene expression profile (GEP) patterns can be analyzed to identify gene signatures that correlate with a high or low risk of disease severity depending the associated pathogen(s). In certain embodiments, the host subject's GEP could indicate a viral versus non-viral infection, or the presence of a bacterial infection, or an acute non-infectious illness. The host GEP could discriminate non-infectious from infectious illness and bacterial from viral causes. In certain embodiments, the host subject's GEP could indicate a high viral load. In certain embodiments, the host subject's GEP could indicate the risk for severity of disease and/or infection (e.g., low risk, intermediate risk or high risk). Additionally, host response GEP biomarkers) offer an additional diagnostic that will decrease inappropriate treatments, and help triage patients predicted to be in the most need of urgent care and aggressive treatment (in particular during a global viral pandemic). Furthermore, a host GEP could allow pre-symptomatic detection of infection in humans exposed to a pathogen (or, for example, in asymptomatic patients) before typical clinical symptoms are apparent.

In certain embodiments, a gene-expression profile is comprised of the gene-expression levels of at least 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 discriminant gene(s). In one embodiment, the gene-expression profile is comprised of about 50 discriminant genes. In another embodiment, the gene-expression profile is comprised of about 40 discriminant genes. In another embodiment, the gene-expression profile is comprised of about 30 discriminant genes. In another embodiment, the gene-expression profile is comprised of about 20 discriminant genes. In another embodiment, the gene-expression profile is comprised of about 10 discriminant genes. In certain embodiments, the discriminant genes are selected from one or more genes from Tables 14 and/or 16. In certain embodiments, the discriminant genes are selected from: ARFIP2, ARMC10, ATG4C, BBX, CAMKK2, CNKSR3, DNAJC22, EFNB1, FLJ42627, HOXB7, INE2, INTS13, KDM4B, MAFF, MEAK7, NME8, NWD1, PPA2, PRKN, RBM27, SAA2, SGSM2, SYCP2, TNFAIP8L3, TNFRSF9, TNRC6A, and ZNF292. In certain embodiments, the discriminant genes are selected from: AHI1, ANXA4, ATXN1, BRAT1, CAMTA1, CCDC32, CD84, CES3, CLDN16, CLUAP1, DDHD1, ECE1, EYA4, FAM111B, FAM169A, GNAL, KLHL5, LRCH1, MAN1B1-DT, MCTS1, NM_014933, NR_027180, NRARP, OXTR, PKHD1, PNPLA6, PRDM16, PROCR, RBFOX3, RBM5, RDM1P5, RINL, RNF41, SCPEP1, SNAP29, TRIP10, TTC39A, ZBTB16, ZDHHC3, and ZNF445.

As used herein, the terms “differentially expressed” or “differential expression” refer to a difference in the level of expression of the genes that can be assayed by measuring the level of expression of the products of the genes, such as the difference in level of messenger RNA transcript expressed (or converted cDNA) or proteins expressed of the genes. In one embodiment, the difference can be statistically significant. The term “difference in the level of expression” refers to an increase or decrease in the measurable expression level of a given gene as measured by the amount of messenger RNA transcript (or converted cDNA) and/or the amount of protein in a sample as compared with the measurable expression level of a given gene in a control, or control gene or genes in the same sample (for example, a non-recurrence sample). In another embodiment, the differential expression can be compared using the ratio of the level of expression of a given gene or genes as compared with the expression level of the given gene or genes of a control, wherein the ratio is not equal to 1.0. For example, an RNA, cDNA, or protein is differentially expressed if the ratio of the level of expression in a first sample as compared with a second sample is greater than or less than 1.0. For example, a ratio of greater than 1, 1.2, 1.5, 1.7, 2, 3, 3, 5, 10, 15, 20, or more than 20, or a ratio less than 1, 0.8, 0.6, 0.4, 0.2, 0.1, 0.05, 0.001, or less than 0.0001. In yet another embodiment, the differential expression is measured using p-value. For instance, when using p-value, a biomarker is identified as being differentially expressed as between a first sample and a second sample when the p-value is less than 0.1, less than 0.05, less than 0.01, less than 0.005, or less than 0.001.

As used herein, the term “altered in a predictive manner” refers to changes in genetic expression profile that identifies or determines a subject has an infection from a pathogen, or has an increased risk of severe disease caused by a pathogen. Predictive modeling can be measured as: 1) identifies or determines severity of disease from an infection by a pathogen as low severity, intermediate severity, or high severity; and/or 2) a linear outcome based upon a probability score from 0 to 1 that reflects the correlation of the genetic expression profile of an infection from a pathogen of the samples that comprise the training set used to identify or determine an infection from a pathogen. The increasing probability score from 0 to 1 reflects incrementally increasing accuracy of an infection and/or severity of infection. For example, within the probability score range from 0 to 1, a probability score, for example, of less than about 0.33 reflects a sample with a low risk of an infection and/or severe infection, while a probability score, for example, of between about 0.33 and 0.66 reflects a sample with an intermediate risk of an infection and/or severe infection, and probability score of greater than about 0.66 reflects a sample with a high risk of an infection and/or severe infection.

As used herein, the terms “control” and “standard” refer to a specific value that one can use to determine the value obtained from the sample. In one embodiment, a dataset may be obtained from samples from a group of subjects known to have an infection from a pathogen. In one embodiment, a dataset may be obtained from samples from a group of subjects known to have an infection from SARS-CoV-2 (COVID-19). The expression data of the genes in the dataset can be used to create a control (standard) value that is used in testing samples from new subjects. In such an embodiment, the “control” or “standard” is a predetermined value for each gene or set of genes obtained from subjects with an infection from a pathogen (e.g., SARS-CoV-2 (COVID-19)) whose gene expression values and severity of disease are known.

As used herein, the terms “treatment,” “treat,” or “treating” refer to a method of reducing the effects of a disease or condition or symptom of the disease or condition. Thus, in the methods disclosed herein, treatment can refer to a 5%, 10%, 20%, 30%, 40%, 50%, 60%, 70%, 80%, 90%, or 100% reduction in the severity of an established disease or condition or symptom of the disease or condition. For example, a method of treating a disease is considered to be a treatment if there is a 5% reduction in one or more symptoms of the disease in a subject as compared to a control. Thus, the reduction can be a 5%, 10%, 20%, 30%, 40%, 50%, 60%, 70%, 80%, 90%, 100% or any percent reduction between 5% and 100% as compared to native or control levels. It is understood that treatment does not necessarily refer to a cure or complete ablation of the disease, condition, or symptoms of the disease or condition. In some embodiments, treatments can comprise one or more of convalescent plasma or other antibody therapies (for example, bamlanivimab and etesevimab, casirivimab and imdevimab, and sotrovimab, and tocilizumab), anti-viral therapies (e.g., remdesiver), corticosteroids,

The methods disclosed herein generate massive amounts of quantitative and sequencing data generated by high-throughput sequencers (NGS can generate several million to billion short-read sequences of the DNA and RNA isolated from a sample), thus, in certain embodiments, the methods disclosed herein also use data processing pipelines to analyze sequencing data. A “pipeline” as used herein refers to the algorithm(s) executed in a predefined sequence to process NGS data. For example, all the reads from a sample are received (for example, reads comprise sequence data from both the host subject and any pathogen(s) in the host sample), the reads are processed and aligned to one or more reference genomes or reference sequences or transcriptomes. In some embodiments, the pipeline performs deduplication, quality control, decontamination, assembly, and taxonomy classification of the reads in the sample.

Also disclosed herein are kits for preparing a sequencing library comprising any combination of the oligonucleotides disclosed herein. A “kit” is any article of manufacture (e.g., a package or container) comprising at least one reagent, e.g., an oligonucleotide or primer set, for specifically detecting a pathogen consensus sequence used in the methods as disclosed herein. The article of manufacture may be promoted, distributed, sold, or offered for sale as a unit for performing the methods disclosed herein. Kits can include any combination of components that facilitates the performance of the methods as disclosed herein. A kit that facilitates assessing the presence of a pathogen in a sample in conjunction with the expression of host genes may also include suitable nucleic acid-based reagents as well as suitable buffers, control reagents, and printed protocols. The kit may comprise PCR primers capable of amplifying a nucleic acid complementary to a pathogen consensus sequence as defined above. The kits may comprise 384-well and/or 96-well plates pre-loaded with any of the oligonucleotides disclosed herein. In some embodiments, the kit may be used to prepare RNA sequencing libraries. The kit may further comprise reagents, enzymes and/or buffers required to perform reactions such as ligations, reverse transcription, nucleic acid amplification (e.g., PCR), and/or sequencing. The kit may comprise one or more of forward primer and reverse primers. In addition, the kits disclosed herein may preferably contain instructions which describe a suitable detection, diagnostic and/or prognostic assay. Such kits can be conveniently used, e.g., in clinical settings, to diagnose and evaluate patients exhibiting symptoms of an infection by a pathogen. Such kits can also be conveniently used in clinical settings, to monitor a large population of subject at risk of an infection by a pathogen.

Without limiting the disclosure, a number of embodiments of the disclosure are described below for purpose of illustration.

The invention will be further described in the following examples, which do not limit the scope of the invention described in the claims.

### EXAMPLES

The Examples that follow are illustrative of specific embodiments of the disclosure, and various uses thereof. They are set forth for explanatory purposes only, and should not be construed as limiting the scope of the invention in any way.

### Materials and Methods

### (1) RNA Elution/Resuspension Buffer

Prepare, filter (0.22-μm mesh) and store at room temperature a large volume of the stock below (e.g., 50 mL) for continued use:

| | |
|---|---|
| a) 10 mM Tris-HCl pH~7.4 | [1:100 of 1M stock] |
| b) 0.1 mM EDTA | [1:1,000 of 100 mM stock] |

### (2) DNA Elution/Resuspension Buffer

Prepare, filter (0.22-μm mesh) and store at room temperature a large volume of the stock below (e.g., 50 mL) for continued use:

| | |
|---|---|
| a) 10 mM Tris-HCl pH~8.5 | [1:100 of 1M stock] |
| b) 0.1 mM EDTA | [1:1,000 of 100 mM stock] |

Optional:

| | |
|---|---|
| 0.01% (v/v) Tween-20 | [1:1,000 of 10% (v/v) stock] |

### (3) DNA SPRI Solution

Carrier buffer in solid-phase reverse immobilization (SPRI) beads. Allows reusing initial pool of beads for multiple nucleic acid purification rounds throughout protocol. Prepare, filter (0.22-μm mesh) and store at 4° C. a large volume of the stock below (e.g., 50 mL) for continued use:

| | |
|---|---|
| a) 20%(w/v) PEG-8000 | [1:2 of 40% (w/v) stock in H₂O; very viscous] |
| b) 2.5M NaCl | [1:2 of 5M stock] |

Optional:
   Supplement (3) DNA SPRI Solution right before use with each of the following components to protect nucleic acid integrity in 4° C. storage, particularly if expected DNA yields are low, or for frozen banking of plates with mid-protocol DNA templates:

| | |
|---|---|
| 10 mM Tris-HCl PH~8.5 | [1:100 of 1M stock] |
| 1 mM EDTA | [1:100 of 100 mM stock] |
| 0.05% (v/v) Tween-20 | [1:200 of 10% (v/v) stock] |

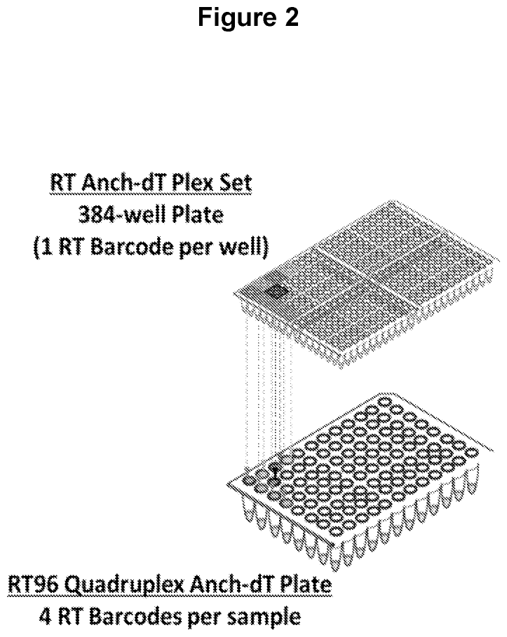

(4) RT96 Quadruplex Anch-dT Plate (96-well)
Starting from the RT Anch-dT Plex Set prepare a specimen-specific 4-way anchored oligo(dT) multiplexing 96-well plate as follows:
   Mix equal volumes from 4 distinctly barcoded reverse transcription tailing primers of the 384-well RT Anch-dT Plex Set at equimolar concentrations into a single well of a 96-well plate. Repeat for every well in the plate, making sure all barcoded primers are distinct between wells (i.e., each of the barcoded reverse transcription tailing primers is used only once, into a single 4-plex well mix; see FIG. 2)
   Dilute the resulting (4) RT96 Quadruplex Anch-dT Plate down to a 2.5 μM per RT barcode ready-to-use stock (i.e., 10 μM net) per well with (2) DNA Elution/Resuspension Buffer
   Cover plate with low-adhesion plastic film and spin briefly to collect aliquoted volumes to the bottom of wells (use a swinging bucket centrifuge or benchtop plate spinner)
   Replace low-adhesion plastic film with foil adhesive cover; roll-press thoroughly to seal
   Keep at 4° C. if using immediately, or store long-term at −20° C. frozen storage until use
   If starting with a pre-assembled (4) RT96 Quadruplex Anch-dT Plate from frozen storage:
      a) Allow plate to thaw on benchtop for 5-10 min before starting any of the protocols
      b) Spin plate to collect primer mixes to the bottom of their wells
      c) Place on bench, hold steady, and remove foil
      d) Quick chill on wet ice (or keep at 4° C.) until use, and proceed with protocol of choice

### (5) Quick RT Mix Plate (96-Well)

Starting from (4) RT96 Quadruplex Anch-dT Plate, prepare a specimen-specific 4-way generic reverse transcription multiplexing 96-well plate as follows:
   Add a matching volume of Generic Tailing RT-TSO Primer at 10 μM (in (2) DNA Elution/Resuspension Buffer) in all the wells of a (4) RT96 Quadruplex Anch-dT Plate to obtain a pooled equimolar mix of specimen-specific barcoded anchored oligo(dT) primers (5 μM net) and template-switching oligonucleotides (5 μM net).
   Cover plate with low-adhesion plastic film and spin briefly to collect aliquoted volumes to the bottom of wells (use a swinging bucket centrifuge or benchtop plate spinner).
   Replace low-adhesion plastic film with foil adhesive cover; roll-press thoroughly to seal.
   Keep at 4° C. if using immediately, or store long-term at −20° C. frozen storage until use.
   If starting with a pre-assembled (5) Quick RT Mix Plate from frozen storage:
      a) Allow plate to thaw on benchtop for 5-10 min before starting any of the protocols
      b) Spin plate to collect primer mixes to the bottom of their wells
      c) Place on bench, hold steady, and remove foil
      d) Quick chill on wet ice (or keep at 4° C.) until use, and proceed with protocol of choice

### (6) LeaSH RT Mix Plate (96-Well)

Starting from (4) RT96 Quadruplex Anch-dT Plate, prepare a specimen-specific 4-way targeted enrichment reverse transcription multiplexing 96-well plate as follows:
   Add matching volumes of standalone primers (3) listed below at 10 μM (in (2) DNA Elution/Resuspension Buffer) in all the wells of a (4) RT96 Quadruplex Anch-dT Plate to obtain pooled equimolar mixes of specimen-specific barcoded primer stocks as follows:
      a) 3′ reverse transcription primers (5 μM net; 2.5 μM each):
         (4) RT96 Quadruplex Anch-dT (in-plate, 4-plex barcoding, well-specific)
         RT SARS-CoV-2_Mod Primer (added)
      b) 5′ targeted enrichment primers (5 μM net; 2.5 μM each):
         SARS-CoV-2 TRS Tailing RT-TSO Primer (added)
         SARS-CoV-2 TRS Enrichment Coupler Reverse Primer (added)
   Cover plate with low-adhesion plastic film and spin briefly to collect aliquoted volumes to the bottom of wells (use a swinging bucket centrifuge or benchtop plate spinner).
   Replace low-adhesion plastic film with foil adhesive cover; roll-press thoroughly to seal.
   Keep at 4° C. if using immediately, or store long-term at −20° C. frozen storage until use.
   If starting with a pre-assembled (6) LeaSH RT Mix Plate from frozen storage:
      a) Allow plate to thaw on benchtop for 5-10 min before starting any of the protocols
      b) Spin plate to collect primer mixes to the bottom of their wells
      c) Place on bench, hold steady, and remove foil
      d) Quick chill on wet ice (or keep at 4° C.) until use, and proceed with protocol of choice.

### (7) CombIndex Adapter Plates (96 Unique Sets, One 96-Well Plate Each)

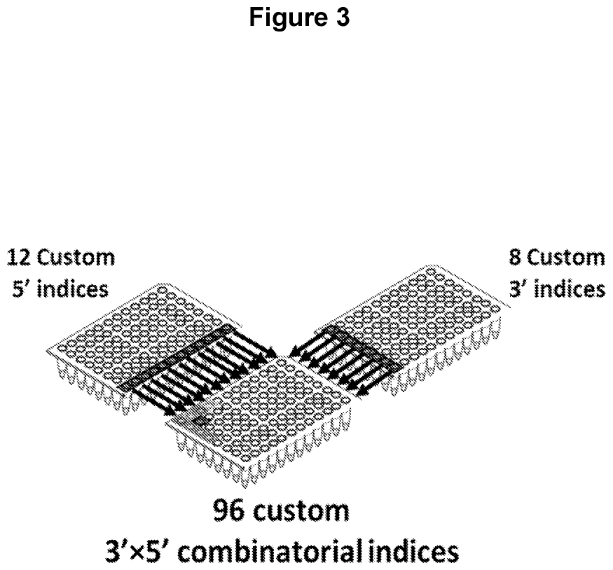

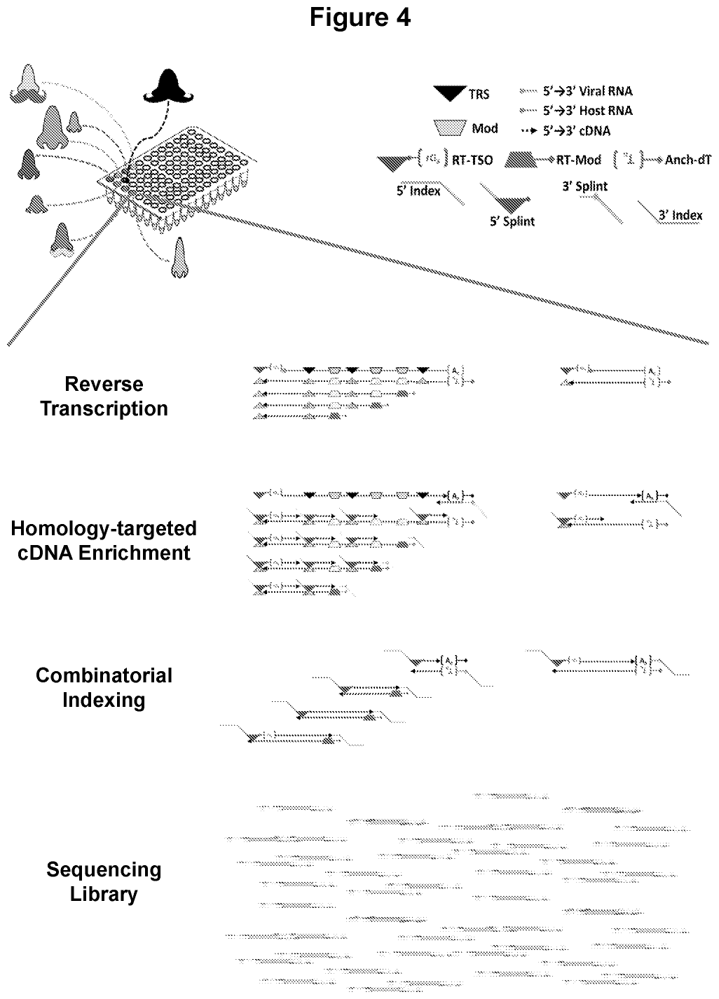

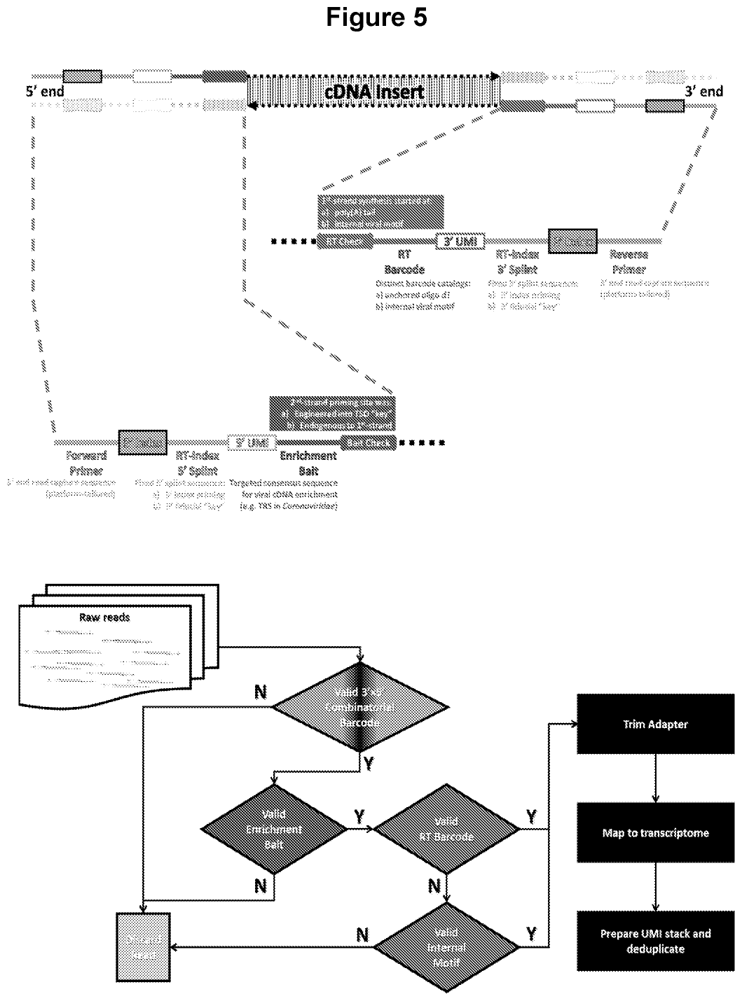

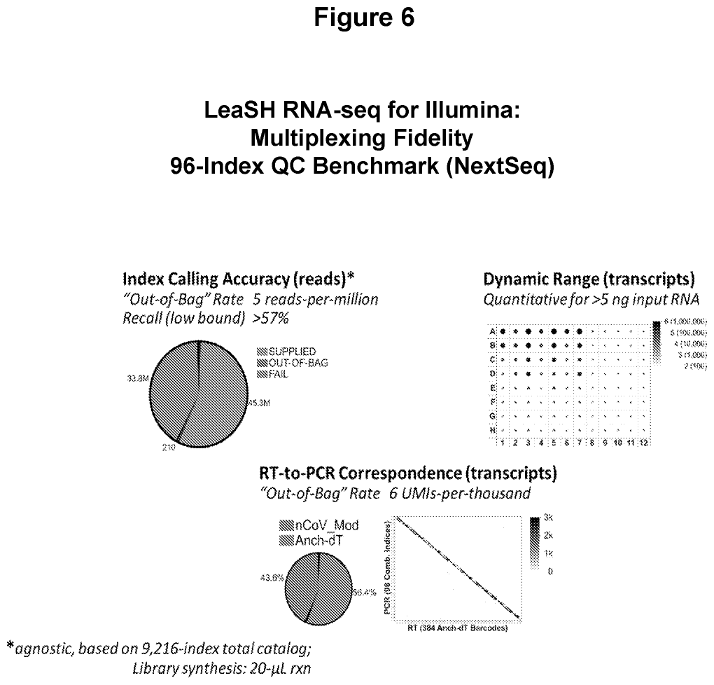

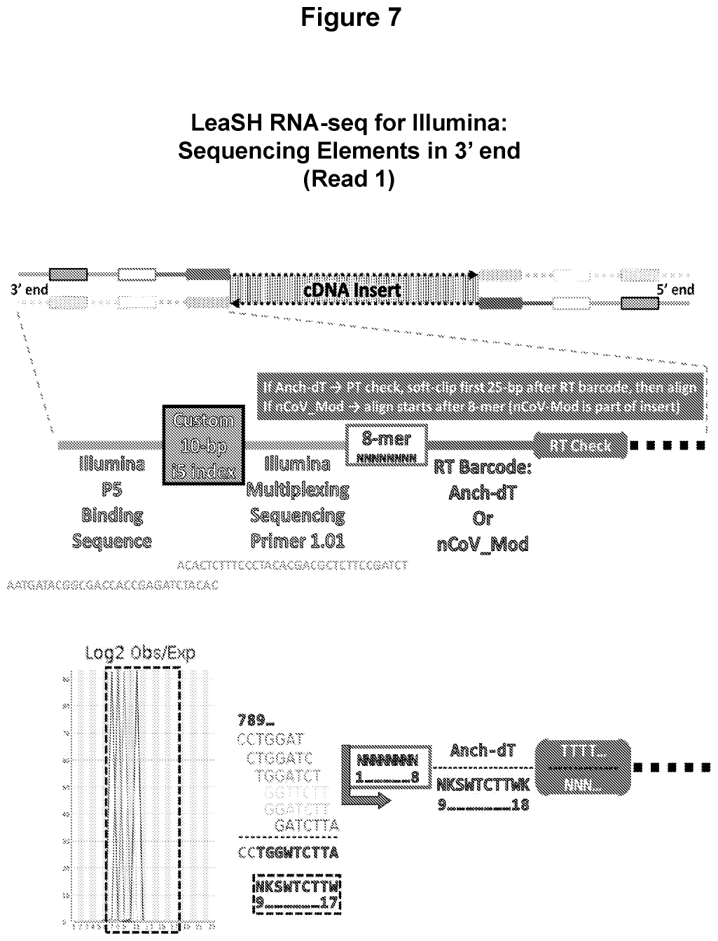

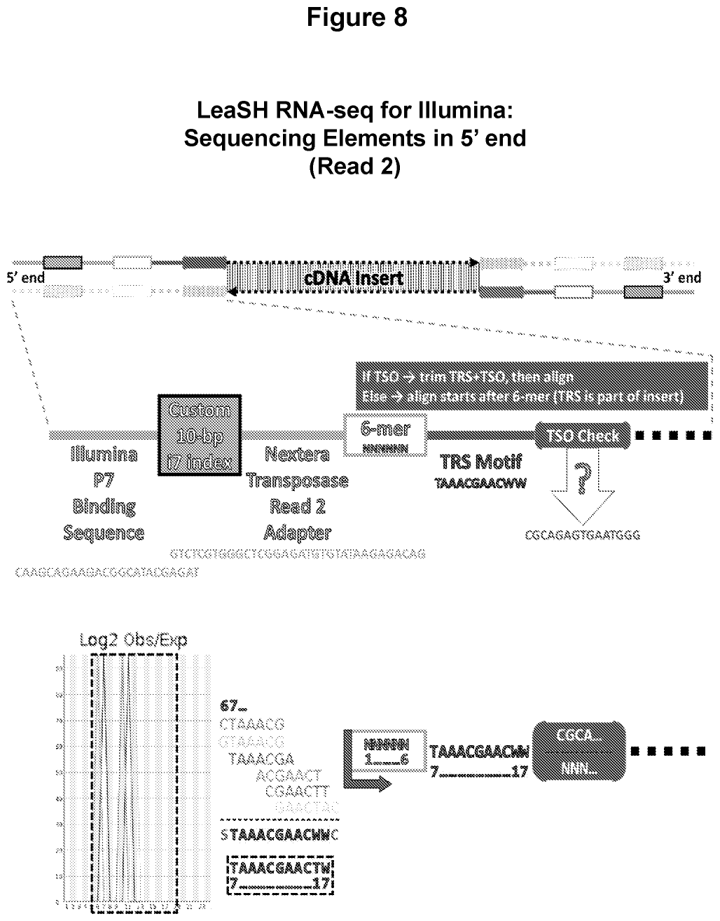

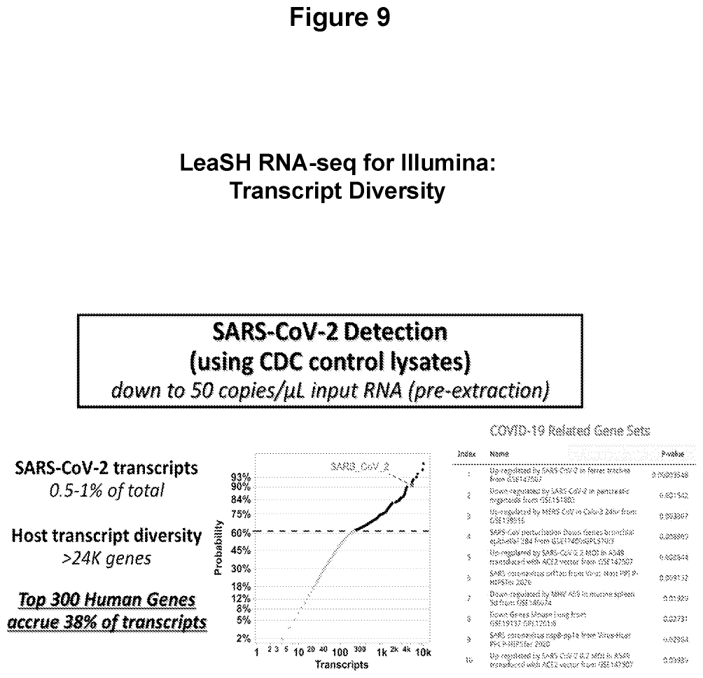

Starting from the 3′ Indexed Adapter Set and 5′ Indexed Adapter Set prepare combinatorial dual-indexing 96-plex adapter sets as follows:
   Dilute the contents of all wells in both 3′ Indexed Adapter Set and 5′ Indexed Adapter Set down to 10 μM (in (2) DNA Elution/Resuspension Buffer).
   Define a 1×reference dispense volume for 96-plex adapter set assembly by using the well with the lowest volume of 10-μM diluted stock in either plate as reference, and assuming a 120×dispense total
      e.g., if least-volume well contains 600 μL adapter at 10 μM, then: 1× dispense=(600 μL÷120)=5 μL.
   Select a column from the 3′ Indexed Adapter Set (1-12) and label 8 empty 96-well plates with the chosen column number as a prefix.
      e.g., if choosing column 5, then label 8 plates as “5 . . . ”
   Dispense 1× volumes from each of the wells in the chosen 3′ Indexed Adapter Set column into each of the wells with matching row letter (i.e., sweeping sideways, 12 wells per row) across all 8 prefix plates, for a total of 96 wells per dispensed 3′ index from the selected 3′ Indexed Adapter Set column.
      Select a row from the 5′ Indexed Adapter Set (A-H) and complete the labeling for 1 of the prefixed 3′-dispensed 96-well plates by adding the chosen row letter as its suffix.
         e.g., if choosing column A, take one of the “5 . . . ” plates and name it “5A”
      Dispense 1× volumes from each of the wells in the chosen 5′ Indexed Adapter Set row into each of the wells with matching column number (i.e., sweeping top-to-bottom, 8 wells per row) of the fully labeled plate, for a total of 8 wells per dispensed 5′ index from the selected 5′ Indexed Adapter Set row.
      Repeat the same suffix labeling and 5′-dispensing approach by matching each of the 7 other prefixed 3′-dispensed 96-well plates from the selected 3′ Indexed Adapter Set column with each of the 7 remaining rows in the 5′ Indexed Adapter Set.
         e.g., complete the “5 . . . ” set by adding plates 58 through 5H, assembled from the remaining 5′ adapter columns 8 through H.
            Repeat the entire 3′×5′ dispensing approach to account for all the remaining columns in the 3′ Indexed Adapter Set. After doing all 12 columns, each 5′ index will have been dispensed into 96 different wells across 12 different 96 well-plates (each with a different prefix number); conversely, each 3′ index will have been dispensed into 96 different wells across 8 different 96-well plates (each with a different suffix letter).
            This approach will produce 96 individual (7) CombIndex Adapter Plates, (see FIG. 3) each with a subset of 96 unique, compounded, and non-repeated 3′×5′ dual indices as 5 μM ready-to-use stocks per well, for a total catalog of 9,216 distinct and sample-specific combinatorial indices that can be organized as follows:
a) 3′ Number Set Plates (e.g., 1A, 1B . . . 1H) accounting for each of the 3′ indices from a single numbered column of the 3′ Indexed Adapter Set combined once with each of the 96 indices from the entire 5′ Indexed Adapter Set separately.
b) 5′ Letter Set Plates (e.g., 1A, 2A . . . 12A) accounting for each of the 5′ indices from a single lettered row of the 5′ Indexed Adapter Set combined once with each of the 96 indices from the entire 3′ Indexed Adapter Set separately.
Cover all plates with low-adhesion plastic film and spin briefly to collect aliquoted volumes to the bottom of wells (use a swinging bucket centrifuge or benchtop plate spinner).
Replace low-adhesion plastic film with foil adhesive cover; roll-press thoroughly to seal.
Keep at 4° C. if using immediately, or store long-term at −20° C. frozen storage until use.
If starting with a pre-assembled (7) CombIndex Adapter Plates from frozen storage:
   a) Allow plate to thaw on benchtop for 5-10 min before starting any of the protocols
   b) Spin plate to collect primer mixes to the bottom of their wells
   c) Place on bench, hold steady, and remove foil
   d) Quick chill on wet ice (or keep at 4° C.) until use, and proceed with protocol of choice.

### Incubation Settings for Reactions Used in Implemented LeaSH Chemistries

| | |
|---|---|
| Incubation Protocol (A) | rRT-qPCR |

Based on Applied Biosystems TaqPath ™ 1-step
RT-qPCR Master Mix, GC [Cat. Nos. A15299, A15300]
Reaction Volume: 20 μL/well [nominal]

| | | |
|---|---|---|
| RT Priming | 25° C. \| 2 min | |
| cDNA Synthesis | 50° C. \| 15 min | |
| RTase Denaturation & | 95° C. \| 2 min | |
| DNA Pol Activation | | |
| “Cold” | 95° C. \| 3 sec | (Fragment Denaturation) |
| Amplification | 20° C. \| 15 sec | (Primer Annealing) |
| (3 cycles) | 60° C. \| 15 sec | (Template Extension) |
| “Warm” | 98° C. \| 15 sec | (Fragment Denaturation) |
| Amplification | 55° C. \| 15 sec | (Primer Annealing) |
| (6 cycles) | 60° C. \| 15 sec | (Template Extension) |
| “Hot” | 98° C. \| 15 sec | (Fragment Denaturation) |
| Amplification | 60° C. \| 15 sec | (Primer Annealing) |
| (9 cycles) | 60° C. \| 15 sec | (Template Extension) |
| Final Extension | 72° C. \| 5 min | |
| HOLD | 4° C. \| ∞ | |

| Incubation Protocol (B) | cDNA Synthesis |
|---|---|
| Based on Thermo Scientific Maxima H Minus Reverse | |
| Transcriptase [Cat. Nos. EP0751, EP0752, EP0753] | |
| Reaction Volume: 20 μL/well [nominal] | |
| RT Priming | 25° C. \| 2 min |
| cDNA Synthesis | 55° C. \| 30 min |
| RTase Denaturation | 85° C. \| 5 min |
| HOLD | 4° C. \| ∞ |

| | |
|---|---|
| Incubation Protocol (C) | cDNA PCR Pre-Amplification |

Based on Roche KAPA HiFi HotStart ReadyMix
[Cat. No. 7958935001 (formerly KAPA Biosystems KK2602)]
Reaction Volume: 50 μL/well [nominal]

| | | |
|---|---|---|
| DNA Pol Activation | 95° C. \| 3 min | |
| PCR-based | 98° C. \| 20 sec | (Fragment Denaturation) |
| Amplification | 63° C. \| 45 sec | (Primer Annealing) |
| (18 cycles) | 72° C. \| 3 min | (Template Extension) |
| Final Extension | 72° C. \| 5 min | |

| | |
|---|---|
| HOLD | 4° C. \| ∞ |

| Incubation Protocol (D) | cDNA MDA Pre-Amplification |
|---|---|
| Based on Lucigen NxGen ® phi29 DNA Polymerase | |
| [Cat. Nos. 30221-1, 30221-2] | |
| Reaction Volume: 50 μL/well [nominal] | |
| cDNA priming | 25° C. \| 2 min |
| φ₂₉-based Amplification | 30° C. \| 16 hr |
| φ₂₉ Denaturation | 65° C. \| 10 min |
| HOLD | 4° C. \| ∞ |

| | |
|---|---|
| Incubation Protocol (E) | Targeted Library PCR Indexing |

Based on Roche KAPA HiFi HotStart ReadyMix
[Cat. No. 7958935001 (formerly KAPA Biosystems KK2602)]
Reaction Volume: 50 μL/well [nominal]

| | | |
|---|---|---|
| DNA Pol Activation | 98° C. \| 45 sec | |
| “Cold” | 98° C. \| 15 sec | (Fragment Denaturation) |
| Amplification | 20° C. \| 30 sec | (Primer Annealing) |
| (3 cycles) | 72° C. \| 30 sec | (Template Extension) |
| “Warm” | 98° C. \| 15 sec | (Fragment Denaturation) |
| Amplification | 55° C. \| 30 sec | (Primer Annealing) |
| (6 cycles) | 72° C. \| 30 sec | (Template Extension) |
| “Hot” | 98° C. \| 15 sec | (Fragment Denaturation) |
| Amplification | 63° C. \| 30 sec | (Primer Annealing) |
| (9 cycles) | 72° C. \| 30 sec | (Template Extension) |
| Final Extension | 72° C. \| 5 min | |
| HOLD | 4° C. \| ∞ | |

### Reactions to Implement LeaSH Chemistry

| | |
|---|---|
| LeaSH 1-step | rRT-qPCR |
| (single-pot/closed-tube chemistry) | |
| LeaSH 2-step | cDNA Synthesis |
| (single-pot/open-tube chemistry) | Targeted Library PCR Indexing |
| Nested PCR LeaSH | cDNA Synthesis |
| (split-pot/multi-tube chemistry) | cDNA PCR Pre-Amplification |
| | Targeted Library PCR Indexing |
| Nested MDA LeaSH | cDNA Synthesis |
| (split-pot/multi-tube chemistry) | cDNA MDA Pre-Amplification |
| | Targeted Library PCR Indexing |

Normalization of RNA-Derived, Targeted Amplicon-Enriched Sequencing Libraries with Duplex-Specific Nuclease (DSN)

After DSN treatment and purification, total library mass yield may be 10% less (or even lower) than the original template, and often undetectable by Qubit; 12-18 additional PCR amplification cycles using platform-specific library re-amplification primers may be needed.

DSN Enzyme (Evrogen Cat. Nos. EA001, EA002, EA003, or EA002) must be reconstituted at 1-2 U/μL from lyophilized storage ahead of time and following the manufacturer's instructions (stability in solution: −20° C. for at least 1 year).
1. Take 12 μL volume of library (or QS with water) preferably with 50 ng-500 ng DNA mass
2. Mix with 4 μL of 4× hybridization buffer (200 mM HEPES pH 7.5+2 M NaCl)
3. Using a thermal cycler, denature at 98° C. for 2 min, re-anneal for 30 min at 68° C.
4. Once re-annealing temperature has been reached, open thermal cycler and heat an aliquot of 10×DSN Master Buffer for 30-60 seconds in a separate PCR microtube on the thermal cycler
5. While on thermal cycler, open samples and add 2 μL of pre-heated 10×DSN Master Buffer each
6. While on thermal cycler, add 2 μL of DSN Enzyme per sample directly from storage (i.e., do NOT pre-heat enzyme)
7. While on thermal cycler, mix contents of all sample tubes by gently pipetting the whole reaction volume up and down 10 times (set pipette to 16 μL to compensate for evaporation, pipetting losses, and prevent foaming)
8. Re-cap samples, close thermal cycler, and let incubation at 68° C. continue for the remainder of the 30-min period to allow digestion to proceed
9. While on thermal cycler, add 20 μL of 2×DSN Stop Buffer (equivalent to 10 mM EDTA; does not need to be pre-heated) and mix by gently pipetting the whole reaction volume up and down 10 times (set pipette to 35 μL to compensate for evaporation, pipetting losses, and avoid foaming)
10. Re-cap tubes, close thermal cycler, and re-incubate for 5 min at 68° C.
11. Retrieve samples from thermal cycler, place on wet ice for 2-5 min, vortex briefly, and spin down contents in tabletop microcentrifuge
12. Perform library purification to size for >200-bp dsDNA (preferably by 0.8×SPRI with AMPureXP or SPRIselect purification beads or similar) and elute in at least 13 μL final volume with 10 mM Tris-HCl pH 8.0-8.5+0.01% Tween-20

| | | |
|---|---|---|
| ≥13 μL= | 2 μL | Qubit quantification |
| | 1 μL | BioAnalyzer profiling |
| | ≥10 μL | Sequencing OR additional PCR enrichment |

### Example 1: LeaSH 1-Step (Single-Pot/Closed-Tube Chemistry)

1. Organize RNA templates from specimens into 96-plex sample sets, i.e., groups of 96 input RNA samples per library preparation round (e.g., 96 specimens without replication, 48 specimens in technical duplicates, etc.).
   For each 96-plex sample set, retrieve: a) one (6) LeaSH RT Mix Plate; and b) one individual (7) CombIndex Adapter Plate. Place both on the bench at room temperature unopened
      Important: do not put back at 4° C.; condensation due to air humidity may throw off stoichiometry at these small reaction volumes
2. Moving one 96-plex sample set at a time, prepare one stock of LeaSH 1-Step Pre-Mix on ice by adding the following components in order:
   600 μL 4× TaqPath™ 1-step RT-qPCR Master Mix [Applied Biosystems]
   375 μL (1) RNA Elution/Resuspension Buffer
   125 μL rDNA Blocking Duplex @ 10 μM
   Total: 1.1 mL LeaSH 1-Step Pre-Mix (i.e., 120×9 μL reagent volumes)
   Important: make fresh, keep on ice, and use one stock at a time within 30 minutes
3. Empty one stock of LeaSH 1-Step Pre-Mix into a pipetting trough and load an empty 96-well PCR reaction plate with 9 μL per well, using a high-resolution positive displacement multichannel repeating pipettor (e.g., INTEGRA 125-μL VIAFLO).
4. Select one 96-plex sample set to work with, and remove foil covers off both one (6) LeaSH RT Mix Plate and its pre-assigned (7) CombIndex Adapter Plate.
5. Complete a LeaSH 1-step reaction plate by loading into a dispensed LeaSH 1-Step Pre-Mix plate (from step 4): one 4-plex unique RT barcode set from the (6) LeaSH RT Mix Plate, one 3′×5′ unique dual index set from the (7) CombIndex Adapter Plate, and one input RNA sample from the 96-plex sample set per well. The final reaction should be:
   LeaSH 1-step, 20 μL/well [nominal]
      9 μL LeaSH 1-Step Pre-Mix (pre-dispensed, step 4)
      5 μL (6) LeaSH RT Mix Plate (well-specific)
      2 μL (7) CombIndex Adapter Plate (well-specific)
      5 μL input RNA sample (well-specific).
6. Mix reactions gently inside every well by pipetting full volume 10-20 times, cover with clear adhesive film the LeaSH 1-step reaction plate, and spin briefly to collect contents.
7. Perform library amplification on a PCR heat block for each LeaSH 1-step reaction plate within the 30-min window from step 3 using the rRT-qPCR incubation protocol (Incubation Protocol A).
8. Repeat steps 3-8 as needed for every 96-plex sample set, moving one at a time.
9. After reactions are completed, carefully remove PCR film off LeaSH 1-step reaction plates by holding them down onto bench, and combine all reaction wells without repeating 3′×5′ unique dual indices into a single LeaSH 1-step multiplexed library.
10. Perform 1×SPRI bead cleanup on the LeaSH 1-step multiplexed library with 10-fold diluted beads in (3) DNA SPRI Solution to remove enzymatic reagent buffers and elute with 800 μL of (2) DNA Elution/Resuspension Buffer.
11. Perform one-sided size selection with SPRI beads at stock concentration on the purified library (step 11) and elute in 100 μL of (2) DNA Elution/Resuspension Buffer to retain>200-bp library templates, using anywhere between:
   0.5×SPRI (e.g., longer fragments from high-quality or full-length input RNA); and
   0.8×SPRI (e.g., shorter fragments from highly fragmented input RNA).
12. Verify the size of the LeaSH 1-step multiplexed library by gel or capillary electrophoresis, and quantify by intercalating dye fluorometry or qPCR. Store the size-selected LeaSH 1-step multiplexed library at 4° C. (up to 6 months) or −20° C. (indefinitely) until sequencing.

### Example 2: LeaSH 2-Step (Single-Pot/Open-Tube Chemistry)

1. Organize RNA templates from specimens into 96-plex sample sets, i.e., groups of 96 input RNA samples per library preparation round (e.g., 96 specimens without replication, 48 specimens in technical duplicates, etc.).
2. For each 96-plex sample set, retrieve: a) one (6) LeaSH RT Mix Plate; and b) one individual (7) CombIndex Adapter Plate. Place both on the bench at room temperature unopened
   CRITICAL: do not put back at 4° C.; condensation due to air humidity may throw off stoichiometry at these small reaction volumes
3. Moving one 96-plex sample set at a time, prepare one stock of LeaSH RT Pre-Mix on ice by adding the following components in order:
   500 μL 5× RT Buffer [Thermo Scientific]
   250 μL 10 mM dNTP Mix
   250 μL (1) RNA Elution/Resuspension Buffer
   125 μL rDNA Blocking Duplex @ 10 μM
   125 μL NxGen® RNAse Inhibitor @ 40 U/μL [Lucigen]
   0.5 μL Maxima H Minus Reverse Transcriptase @ 200 U/μL [Thermo Scientific]
   Total: 1.3 mL LeaSH RT Pre-Mix (i.e., 130×10 μL reagent volumes)
   CRITICAL: make fresh, keep on ice, and use one stock at a time within 30 minutes.
4. Empty one stock of LeaSH RT Pre-Mix into a pipetting trough and load an empty 96-well PCR reaction plate with 10 μL per well, using a high-resolution positive displacement multichannel repeating pipettor (e.g., INTEGRA 125-μL VIAFLO).
5. Select one 96-plex sample set to work with, and remove foil covers off both one LeaSH RT Mix Plate and its pre-assigned (7) CombIndex Adapter Plate.
6. Complete a LeaSH 2-step RT plate by loading into a dispensed LeaSH RT Pre-Mix plate (from step 4): one 4-plex unique RT barcode set from the (6) LeaSH RT Mix Plate, and one input RNA sample from the 96-plex sample set per well. The final reaction should be:
   LeaSH 2-step RT, 20 μL/well [nominal]
      10 μL LeaSH RT Pre-Mix (pre-dispensed, step 4)
      0.5 μL (6) LeaSH RT Mix Plate (well-specific)
      0.5 μL input RNA sample (well-specific).
7. Mix reactions gently inside every well by pipetting full volume 10-20 times, cover with clear adhesive film the LeaSH 2-step RT plate, and spin briefly to collect contents.
8. Perform RNA conversion on a PCR heat block for each LeaSH 2-step RT plate within the 30-min window from step 3 using the cDNA Synthesis incubation protocol (Incubation Protocol B).
9. After the reverse transcription reactions are completed, carefully remove heat-sealed PCR film off each LeaSH 2-step RT plate by holding them down onto bench, and convert each one into a LeaSH 2-step Indexing plate by supplementing with PCR reactants, and one 3′×5′ unique dual index set per well from the (7) CombIndex Adapter Plate. The final reaction should be:
   LeaSH 2-step Indexing, 50 μL/well [nominal]
      20 μL LeaSH 2-step RT (product, step 8)
      25 μL 2×KAPA HiFi HotStart ReadyMix [Roche]
      0.5 μL CombIndex Adapter Plate (well-specific).
10. Mix reactions gently inside every well by pipetting full volume 10-20 times, cover with clear adhesive film the LeaSH 2-step Indexing plate, and spin briefly to collect contents.
11. Perform library amplification on a PCR heat block for each LeaSH 2-step Indexing plate using the Targeted Library PCR Indexing incubation protocol (Incubation Protocol E).
12. Repeat steps 3-11 as needed for every 96-plex sample set, moving one at a time.
13. After the library amplification reactions are completed, carefully remove heat-sealed PCR film off every LeaSH 2-step Indexing plate by holding them down onto bench, and combine all reaction wells without repeating 3′×5′ unique dual indices into a single LeaSH 2-step multiplexed library.
14. Perform 1×SPRI bead cleanup on the LeaSH 2-step multiplexed library with 10-fold diluted beads in (3) DNA SPRI Solution to remove enzymatic reagent buffers and elute with 800 μL of (2) DNA Elution/Resuspension Buffer.
15. Perform one-sided size selection with SPRI beads at stock concentration on the purified library (step 11) and elute in 100 μL of (2) DNA Elution/Resuspension Buffer to retain>200-bp library templates, using anywhere between:
   0.5×SPRI (e.g., longer fragments from high-quality or full-length input RNA); and
   0.8×SPRI (e.g., shorter fragments from highly fragmented input RNA).
16. Verify the size of the LeaSH 2-step multiplexed library by gel or capillary electrophoresis, and quantify by intercalating dye fluorometry or qPCR. Store the size-selected LeaSH 2-step multiplexed library at 4° C. (up to 6 months) or −20° C. (indefinitely) until sequencing.

### Example 3: Nested PCR LeaSH (Split-Pot/Multi-Tube Chemistry)

1. Organize RNA templates from specimens into 96-plex sample sets, i.e., groups of 96 input RNA samples per library preparation round (e.g., 96 specimens without replication, 48 specimens in technical duplicates, etc.).
2. For each 96-plex sample set, retrieve one (5) Quick RT Mix Plate. Place on the bench at room temperature unopened
   CRITICAL: do not put back at 4° C.; condensation due to air humidity may throw off stoichiometry at these small reaction volumes.
3. Moving one 96-plex sample set at a time, prepare one stock of LeaSH RT Pre-Mix on ice by adding the following components in order:
   500 μL 5× RT Buffer [Thermo Scientific]
   250 μL 10 mM dNTP Mix
   250 μL (1) RNA Elution/Resuspension Buffer
   125 μL rDNA Blocking Duplex @ 10 μM
   125 μL NxGen® RNAse Inhibitor @ 40 U/μL [Lucigen]
   0.5 μL Maxima H Minus Reverse Transcriptase @ 200 U/μL [Thermo Scientific]
   Total: 1.3 mL LeaSH RT Pre-Mix (i.e., 130×10 μL reagent volumes)
   CRITICAL: make fresh, keep on ice, and use one stock at a time within 30 minutes.
4. Empty one stock of LeaSH RT Pre-Mix into a pipetting trough and load an empty 96-well PCR reaction plate with 10 μL per well, using a high-resolution positive displacement multichannel repeating pipettor (e.g., INTEGRA 125-μL VIAFLO).
5. Select one 96-plex sample set to work with, and remove cover off one (5) Quick RT Mix Plate.
6. Complete a Nested RT plate by loading into a dispensed LeaSH RT Pre-Mix plate (from step 4): one 4-plex unique RT barcode set from the (5) Quick RT Mix Plate, and one input RNA sample from the 96-plex sample set per well. The final reaction should be:
   Nested RT, 20 μL/well [nominal]
      10 μL LeaSH RT Pre-Mix (pre-dispensed, step 4)
      0.5 μL (5) Quick RT Mix Plate (well-specific)
      0.5 μL input RNA sample (well-specific).
7. Mix reactions gently inside every well by pipetting full volume 10-20 times, cover with clear adhesive film the Nested RT plate, and spin briefly to collect contents.
8. Perform RNA conversion on a PCR heat block for each Nested RT plate within the 30-min window from step 3 using the cDNA Synthesis incubation protocol (Incubation Protocol B)
9. After reverse transcription reactions are completed, prepare one stock of Nested PCR Pre-Mix on ice per each Nested RT plate by adding the following components in order:
   3000 μL 2×KAPA HiFi HotStart ReadyMix [Roche]
   0.3 μL Universal cDNA Coupler Forward Primer @ 10 μM
   0.3 μL Generic cDNA Coupler Reverse Primer @ 10 μM
   Total: 3.6 mL Nested PCR Pre-Mix (i.e., 120×30 μL reagent volumes)
   CRITICAL: make fresh, keep on ice, and use one stock at a time within 30 minutes.
10. Carefully remove heat-sealed PCR film off Nested RT plate by holding it down onto bench, empty one stock of Nested PCR Pre-Mix into a pipetting trough, and convert the Nested RT plate into a Nested PCR plate by supplementing with 30 μL of Nested PCR Pre-Mix per well. The final reaction should be:
   Nested PCR, 50 μL/well [nominal]
      20 μL Nested RT (product, step 8)
      30 μL Nested PCR Pre-Mix.
11. Mix reactions gently inside every well of the assembled Nested PCR plate by pipetting full volume 10-20 times, cover with clear adhesive film, and spin briefly to collect contents.
12. Perform cDNA pre-amplification of the Nested PCR plate on a PCR heat block using the cDNA PCR Pre-Amplification incubation protocol (Incubation Protocol C).
13. After cDNA pre-amplification reactions are completed, carefully remove heat-sealed PCR film off Nested PCR plate by holding it down onto bench, then perform in-plate 1× SPRI bead cleanup with SPRI beads at stock concentration. After 80% ethanol clearing, resuspend SPRI beads inside their wells with 15 μL of (2) DNA Elution/Resuspension Buffer.
14. Cover the Nested PCR plate with clear adhesive film, and spin briefly to collect contents.
15. Repeat steps 3-14 as needed for each 96-plex sample set, moving one at a time. Keep resulting Nested PCR plates on ice or stored (at 4° C. overnight or −20° C. indefinitely) until use.
16. For each Nested PCR plate, retrieve one individual (7) CombIndex Adapter Plate. Place on the bench at room temperature unopened
   CRITICAL: do not put back at 4° C.; condensation due to air humidity may throw off stoichiometry at these small reaction volumes.
17. Select one Nested PCR plate to work with, and remove cover off its pre-assigned (7) CombIndex Adapter Plate.
18. Prepare one stock of LeaSH Enrichment Pre-Mix on ice per each Nested PCR plate by adding the following components in order:
   3000 μL 2×KAPA HiFi HotStart ReadyMix [Roche]
   0.3 μL RT SARS-CoV-2_Mod Primer @ 10 μM
   0.3 μL SARS-CoV-2 TRS Enrichment Coupler Reverse Primer @ 10 μM
   Total: 3.6 mL LeaSH Enrichment Pre-Mix (i.e., 120×30 μL reagent volumes)
   CRITICAL: make fresh, keep on ice, and use one stock at a time within 30 minutes.
19. Carefully remove heat-sealed PCR film off Nested PCR plate by holding it down onto bench, empty one stock of LeaSH Enrichment Pre-Mix into a pipetting trough, and convert the Nested PCR plate into a Nested Indexing plate by supplementing with 30 μL of LeaSH Enrichment Pre-Mix, and 5 μL of one 3′×5′ unique dual index set per well from the (7) CombIndex Adapter Plate. The final reaction should be:
   Nested Indexing, 50 μL/well [nominal]
      15 μL Nested PCR (product, step 8)
      30 μL LeaSH Enrichment Pre-Mix
      0.5 μL (7) CombIndex Adapter Plate (well-specific).
20. Mix reactions gently inside every well by pipetting full volume 10-20 times, cover with clear adhesive film the Nested Indexing plate, and spin briefly to collect contents.
21. Perform library amplification on a PCR heat block for each Nested Indexing plate using the Targeted Library PCR Indexing incubation protocol (Incubation Protocol E).
22. Repeat steps 16-21 as needed for every Nested PCR plate, moving one at a time.
23. After the library amplification reactions are completed, carefully remove heat-sealed PCR film off every Nested Indexing plate by holding them down onto bench, and pool all wells without repeating 3′×5′ unique dual indices into a single Nested PCR LeaSH multiplexed library.
24. Perform 1×SPRI bead cleanup on the Nested PCR LeaSH multiplexed library with 10-fold diluted beads in (3) DNA SPRI Solution to remove enzymatic reagent buffers and elute with 800 μL of (2) DNA Elution/Resuspension Buffer.
25. Perform one-sided size selection with SPRI beads at stock concentration on the purified library (step 11) and elute in 100 μL of (2) DNA Elution/Resuspension Buffer to retain>200-bp library templates, using anywhere between:
   0.5×SPRI (e.g., longer fragments from high-quality or full-length input RNA); and
   0.8×SPRI (e.g., shorter fragments from highly fragmented input RNA).
26. Verify the size of the Nested PCR LeaSH multiplexed library by gel or capillary electrophoresis, and quantify by intercalating dye fluorometry or qPCR. Store the size-selected Nested PCR LeaSH multiplexed library at 4° C. (up to 6 months) or −20° C. (indefinitely) until sequencing.

### Example 4: Nested MDA LeaSH (Split-Pot/Multi-Tube Chemistry)

1. Organize RNA templates from specimens into 96-plex sample sets, i.e., groups of 96 input RNA samples per library preparation round (e.g., 96 specimens without replication, 48 specimens in technical duplicates, etc.).
2. For each 96-plex sample set, retrieve one (5) Quick RT Mix Plate. Place on the bench at room temperature unopened
   CRITICAL: do not put back at 4° C.; condensation due to air humidity may throw off stoichiometry at these small reaction volumes.
3. Moving one 96-plex sample set at a time, prepare one stock of LeaSH RT Pre-Mix on ice by adding the following components in order:
   500 μL 5× RT Buffer [Thermo Scientific]
   250 μL 10 mM dNTP Mix
   250 μL (1) RNA Elution/Resuspension Buffer
   125 μL rDNA Blocking Duplex @ 10 μM
   125 μL NxGen® RNAse Inhibitor @ 40 U/μL [Lucigen]
   0.5 μL Maxima H Minus Reverse Transcriptase @ 200 U/μL [Thermo Scientific]
   Total: 1.3 mL LeaSH RT Pre-Mix (i.e., 130×10 μL reagent volumes)
   CRITICAL: make fresh, keep on ice, and use one stock at a time within 30 minutes.
4. Empty one stock of LeaSH RT Pre-Mix into a pipetting trough and load an empty 96-well PCR reaction plate with 10 μL per well, using a high-resolution positive displacement multichannel repeating pipettor (e.g., INTEGRA 125-μL VIAFLO).
5. Select one 96-plex sample set to work with, and remove cover off one (5) Quick RT Mix Plate.
6. Complete a Nested RT plate by loading into a dispensed LeaSH RT Pre-Mix plate (from step 4): one 4-plex unique RT barcode set from the (5) Quick RT Mix Plate, and one input RNA sample from the 96-plex sample set per well. The final reaction should be:
   Nested RT, 20 μL/well [nominal]
      10 μL LeaSH RT Pre-Mix (pre-dispensed, step 4)
      05 μL (5) Quick RT Mix Plate (well-specific)
      0.5 μL input RNA sample (well-specific).
7. Mix reactions gently inside every well by pipetting full volume 10-20 times, cover with clear adhesive film the Nested RT plate, and spin briefly to collect contents.
8. Perform RNA conversion on a PCR heat block for each Nested RT plate within the 30-min window from step 3 using the cDNA Synthesis incubation protocol (Incubation Protocol B).
9. After reverse transcription reactions are completed, prepare one stock of Nested MDA Pre-Mix on ice per each Nested RT plate by adding the following components in order:
   1000 μL Exo-Resistant Random Primers @ 50 μM
   0.6 μL 10× phi29 DNA Polymerase Buffer [Lucigen]
   0.6 μL 10 mM dNTP Mix
   0.6 μL Universal cDNA Coupler Forward Primer @ 10 μM
   0.6 μL Generic cDNA Coupler Reverse Primer @ 10 μM
   0.2 μL NxGen® phi29 DNA Polymerase @ 10 U/μL [Lucigen]
   Total: 3.6 mL Nested MDA Pre-Mix (i.e., 120×30 μL reagent volumes)
   CRITICAL: make fresh, keep on ice, and use one stock at a time within 30 minutes.
10. Carefully remove heat-sealed PCR film off Nested RT plate by holding it down onto bench, empty one stock of Nested MDA Pre-Mix into a pipetting trough, and convert the Nested RT plate into a Nested MDA plate by supplementing with 30 μL of Nested MDA Pre-Mix per well. The final reaction should be:
   Nested MDA, 50 μL/well [nominal]
      20 μL Nested RT (product, step 8)
      30 μL Nested MDA Pre-Mix.
11. Mix reactions gently inside every well of the assembled Nested MDA plate by pipetting full volume 10-20 times, cover with clear adhesive film, and spin briefly to collect contents.
12. Perform cDNA pre-amplification of the Nested MDA plate on a PCR heat block using the cDNA MDA Pre-Amplification incubation protocol (Incubation Protocol D).
13. After cDNA pre-amplification reactions are completed, carefully remove heat-sealed PCR film off Nested MDA plate by holding it down onto bench, then perform in-plate 1× SPRI bead cleanup with SPRI beads at stock concentration. After 80% ethanol clearing, resuspend SPRI beads inside their wells with 15 μL of (2) DNA Elution/Resuspension Buffer.
14. Cover the Nested MDA plate with clear adhesive film, and spin briefly to collect contents.
15. Repeat steps 3-14 as needed for each 96-plex sample set, moving one at a time. Keep resulting Nested MDA plates on ice or stored (at 4° C. overnight or −20° C. indefinitely) until use.
16. For each Nested MDA plate, retrieve one individual (7) CombIndex Adapter Plate. Place on the bench at room temperature unopened
   CRITICAL: do not put back at 4° C.; condensation due to air humidity may throw off stoichiometry at these small reaction volumes.
17. Select one Nested MDA plate to work with, and remove cover off its pre-assigned (7) CombIndex Adapter Plate.
18. Prepare one stock of LeaSH Enrichment Pre-Mix on ice per each Nested MDA plate by adding the following components in order:
   3000 μL 2×KAPA HiFi HotStart ReadyMix [Roche]
   0.3 μL RT SARS-CoV-2_Mod Primer @ 10 μM
   0.3 μL SARS-CoV-2 TRS Enrichment Coupler Reverse Primer @ 10 μM
   Total: 3.6 mL LeaSH Enrichment Pre-Mix (i.e., 120×30 μL reagent volumes)
   CRITICAL: make fresh, keep on ice, and use one stock at a time within 30 minutes.
19. Carefully remove heat-sealed PCR film off Nested MDA plate by holding it down onto bench, empty one stock of LeaSH Enrichment Pre-Mix into a pipetting trough, and convert the Nested MDA plate into a Nested Indexing plate by supplementing with 30 μL of LeaSH Enrichment Pre-Mix, and 5 μL of one 3′×5′ unique dual index set per well from the (7) CombIndex Adapter Plate. The final reaction should be:
   Nested Indexing, 50 μL/well [nominal]
      15 μL Nested MDA (product, step 8)
      30 μL LeaSH Enrichment Pre-Mix
      0.5 μL (7) CombIndex Adapter Plate (well-specific).
20. Mix reactions gently inside every well by pipetting full volume 10-20 times, cover with clear adhesive film the Nested Indexing plate, and spin briefly to collect contents.
21. Perform library amplification on a PCR heat block for each Nested Indexing plate using the Targeted Library PCR Indexing incubation protocol (Incubation Protocol E).
22. Repeat steps 16-21 as needed for every Nested MDA plate, moving one at a time.
23. After the library amplification reactions are completed, carefully remove heat-sealed PCR film off every Nested Indexing plate by holding them down onto bench, and pool all wells without repeating 3′×5′ unique dual indices into a single Nested MDA LeaSH multiplexed library.
24. Perform 1×SPRI bead cleanup on the Nested MDA LeaSH multiplexed library with 10-fold diluted beads in (3) DNA SPRI Solution to remove enzymatic reagent buffers and elute with 800 μL of (2) DNA Elution/Resuspension Buffer.
25. Perform one-sided size selection with SPRI beads at stock concentration on the purified library (step 11) and elute in 100 μL of (2) DNA Elution/Resuspension Buffer to retain>200-bp library templates, using anywhere between:
   0.5×SPRI (e.g., longer fragments from high-quality or full-length input RNA); and
   0.8×SPRI (e.g., shorter fragments from highly fragmented input RNA).
      Verify the size of the Nested MDA LeaSH multiplexed library by gel or capillary electrophoresis, and quantify by intercalating dye fluorometry or qPCR. Store the size-selected Nested MDA LeaSH multiplexed library at 4° C. (up to 6 months) or −20° C. (indefinitely) until sequencing.

### Example 5: Hyperplexed Sample Barcoded Screening for SARS-CoV-2 by Next Generation Sequencing

Infectious disease outbreaks have the potential to overwhelm healthcare systems when screening tools are lacking or scarce. This backdrop is a recurring theme in surveillance and management of emerging zoonotic pathogens, particularly when human-to-human transmission is relatively new, genomic features of infectious strains are evolving rapidly, or understanding of molecular machineries that govern viral-host interactions is still incomplete. On occasion, these conditions prevail during outbreaks of infectious strains for which vaccines, prophylactic treatments or effective drugs are unavailable or inexistent. In cases when the infectious strain is non-lethal it can spread unchecked among humans and become endemic; in other cases, the strain is life-threatening, reaches pandemic scales and puts the general population at risk. A prime example of the latter is the severe acute respiratory syndrome coronavirus 2 (SARS-CoV-2), the viral pathogen responsible for Coronavirus Disease 2019 (COVID-19).

Without available vaccines or proven disease management drugs against SARS-CoV-2, healthcare systems must rely on infection screening to manage patients effectively. With that purpose, the United States Centers for Disease Control (CDC), with endorsement by the World Health Organization (WHO) and institutional commitments to confer emergency use authorizations (EUA) by the U.S. Federal Drug Administration (FDA), issued specific “gold standard” guidelines and lists of pre-approved synthetic and enzymatic reagents in fast-tracked development of clinical diagnostic tests on human specimens for SARS-CoV-2 viral genomic RNA (gRNA) titers via one-step rapid reverse transcription and quantitative polymerase chain reaction (rRT-qPCR) using probe-based (i.e., TaqMan) fluorometric readouts. In its current configuration, CDC-compliant PCR-based COVID-19 screens must rely on targeted amplification of 3 separate strain-specific templates within the structural nucleocapsid-encoding gene (N) of SARS-CoV-2 (GenBank: NC_045512.2) plus a minimum of 1 human RNase P template (as housekeeping normalization control) per technical replicate.

In practice, diagnostic-level sensitivity with PCR-based fluorometric detection is only guaranteed for single-amplicon reactions, which effectively discourages the implementation of multiplexed(color) qPCR fluorometry for SARS-CoV-2 detection in clinical-grade tests. With multiplexed qPCR fluorometry rendered inadequate for diagnostic purposes in medical care, and in the face of an ongoing COVID-19 pandemic, the effective throughput of SARS-CoV-2 multi-patient assays is currently outpaced by the demand for SARS-CoV-2 tests. These limitations have resulted in longer testing queue times for SARS-CoV-2 than other viral load tests, backlogs in COVID-19 diagnoses, and delayed access to specialized treatment for COVID-19 patients. The goal of this project is to implement an easily scalable and massively paralleled multiplexed transcriptional screening for SARS-COV-2 viral gRNA titers using next generation sequencing (NGS), both as an alternative to current qPCR fluorometry tests and as an easily retrofittable protocol requiring minimal retooling at CLIA-compliant testing laboratories with access to NGS and currently certified for PCR-based SARS-CoV-2 screening.

From an assay design perspective, the SARS-CoV-2 gRNA displays genomic features amenable to screening by poly(A) RNA sequencing: it is a 30-kb, 5′-capped and 3′-poly(A) tailed single-stranded viral transcript, starting with a ˜70-nt leader sequence acting as promoter and carrying a consensus 12-nt transcription-regulatory sequence motif (TRS-L; HUAAACGAACWW; SEQ ID NO:1174), followed by 2 polycistronic open-reading frames (ORF1a and ORF1b, 13.2-kb and 8.1-kb long) that give rise to over 37 non-structural proteins, and ending with 7 non-overlapping subgenomic RNAs (sgRNA) encoding structural and accessory proteins necessary to assemble virion progeny. Each sgRNA in the genome body is flanked by spacer sequences that also carry the transcription-regulatory motif (TRS-B), which is used during negative-strand synthesis to produce leader-to-body fusion sgRNA transcripts via canonical TRS-mediated polymerase jumping. SARS-CoV-2 mRNAs exhibit tightly controlled poly(A) tail lengths (between 30-45 nt) and accrue up to 65% of the total mRNA load from infected mammalian continuous cell lines (Vero cells, MOI=0.05, 4th passage). Both the TRS-B motifs that flank SARS-CoV-2 sgRNAs and their poly(A) tailing are candidate priming sites for combinatorially indexed multi-patient NGS library assembly, which can be exploited to devise massively paralleled screening tests for both viral infection and host transcriptional response simultaneously using sgRNA-enriched poly(A) RNA-seq technology. Principles used in Illumina-based dual-indexed sequencing, i.e., assemble catalogs of pre-determined sequencing index combinations, can be used in conjunction with oligo(dT) primed reverse transcription and targeted amplification of SARS-CoV-2 sgRNAs, to produce “patient barcoded” poly(A) RNA-seq libraries in tandem, scalable by the thousands with automated sample processing equipment, sequenced simultaneously using high-output NGS technology, and decoded bioinformatically into patient-specific data to calculate SARS-CoV-2 viral loads and “snapshots” of infected host transcriptomes from the same assay.

Anchored Oligo(dT) Primers are Compatible with Single-Pot cDNA Library Synthesis from Quantitative Mixtures of Host Total RNA and SARS-CoV-2 Viral Transcripts.

Poly(A) tails of host mRNA and SARS-CoV-2 sgRNA molecules are both useful oligo(dT) priming templates for cDNA synthesis in vitro. To show this, full-length cDNA synthesis reactions are performed, followed by capillary electrophoresis and qPCR with CDC-compliant forward PCR primers, for different mixtures of three RNA templates in various stoichiometries: total RNA from a human continuous cell line as host RNA surrogate; independently isolated SARS-CoV-2 gRNA as viral proband (BEI Resources, NIAID, NIH; Cat. No. NR-52285; Biosafety Level: 2); and a quantitative synthetic RNA control to score assay sensitivity (BEI Resources, NIAID, NIH; Cat. No. NR-52358; Biosafety Level: 1).

Sequencing Adapter Primers Appended with the Consensus SARS-CoV-2 TRS Motif Sequence Allow Targeted Enrichment of sgRNA-Derived Templates in Single-Pot Host-Viral cDNA Libraries.

Detection sensitivity for multiple SARS-CoV-2 sgRNA transcripts from a single sequencing assay are boosted using a single splint sequencing primer carrying the consensus TRS sequence of SARS-CoV-2. Therefore, full-length mixed cDNA stocks are prepared, and targeted amplification of sgRNA templates by PCR is performed with sequencing adapters carrying the TRS motif, and assemble mixed poly(A) RNA-seq libraries to be profiled by capillary electrophoresis and quantified by NGS.

Combinatorial Indexing with Barcoded Sequencing Primers Allows Tractable, Automated and Massively Paralleled SARS-CoV-2 Screening of Single-Pot Host-Viral cDNA Libraries by RNA-Seq.

Combinatorial indexing allows for high-throughput “hyper-plexed” parallel screening of thousands of separate poly(A) RNA-seq libraries at once without incurring bioinformatic data “bleed-through” between libraries due to index miscalls. To test this concept, a large catalog of uniquely barcoded combinatorial index poly(A) RNA-seq libraries are sequenced altogether to determine the relation between multiplexed barcode throughput and out-of-bag barcode information rate, i.e., the relative volume of data bioinformatically assigned in an unsupervised manner to dual-index barcodes in use vs. dual-index barcodes absent from the sequencing assay.

To curtail the COVID-19 pandemic, infection screening must keep up with the pace of disease transmission and be readily scalable to meet demands for growing numbers of incoming patients. Right now, the pandemic-level demand for clinical-grade COVID-19 diagnostics, combined with the technical limitations of qCPR-based fluorometric tests for SARS-CoV-2, amount to insufficient multi-patient parallelized screening throughput. In effect, this situation has contributed to bottlenecks in COVID-19 diagnosis, playing against COVID-19 patients who could otherwise be managed earlier during the course of infection and treated accordingly. Just as dire, slow diagnostic times also increase the occupational hazard among healthcare workers for SARS-CoV-2 transmission, who are faced with the real threat of contracting COVID-19 from undiagnosed patients while waiting for test results.

The proposed approach would allow CLIA-compliant entities to reclaim accurate and fast-turnaround SARS-CoV-2 testing capacity—and give healthcare systems the ability to monitor at-risk individuals in periodic fashion, project administrative burden with minimal delay, and triage palliative care towards patients with poor COVID-19 prognosis as quickly as possible. Looking ahead, the technical improvements embodied by a successful NGS-based viral gRNA screening platform would also highlight new means to establish strategic preparedness roadmaps for future pandemics—one in which development of new infectious disease screening platforms can be jump-started to exploit increased volume, throughput, and versatility benefits that next-generation sequencing technologies already offer.

The methods disclosed here have resulted in about 99% reads from the assembled libraries with nucleic acid extracts from SARS-CoV-2 positive nasopharyngeal human specimens (or pools of them) align to the human genome, with 1% or less aligning to the SARS-CoV-2 genome. Furthermore, a number of prevalent host genes detected are concordant with the expression patterns reported in the literature for experimental infection models of SARS-CoV-2 in mammalian systems.

### Example 6: LeaSH RNA-seq: Screening Performance

To create a high-complexity viral infection assay, it is necessary to confirm detection capabilities based on a standard method. To benchmark the ability to detect SARS-CoV-2 viral infection status in true specimens, confirmatory RNA extraction and rRT-qPCR testing was performed on a diverse array of true human specimens following CDC guidelines. The experimental samples consisted of aliquots from banked nasopharyngeal (NP) swabs, oropharyngeal (OP) swabs, or saliva raw specimens originally used for SARS-CoV-2 viral load screening via rRT-qPCR testing (Ct scoring). The raw specimens were originally procured by different U.S. and Canada organizations from donors residing in the continental United States (U.S.), Caribbean, Italy, or Ecuador. Specimen collections were performed following guidelines issued by the U.S. Centers for Disease Control and Prevention (CDC) and the World Health Organization (WHO) (see Table 10). Specimens were screened for SARS-CoV-2 viral load with rRT-qPCR-based diagnostic assays using reagents and methods with either Emergency Use Authorization (EUA) status from the U.S. Food and Drug Administration (FDA) or in adherence with guidance from the WHO. Initial diagnostic testing of all original specimens was performed at either qualified diagnostic laboratories in the U.S. with certification from the College of American Pathologists under Clinical Laboratory Improvement Amendments regulations (CAP/CLIA), or at qualified diagnostic laboratories in Canada with certification from the International Organization for Standardization for Standards 9001:2015 and 13485:2016 (ISO9001/ISO13485).

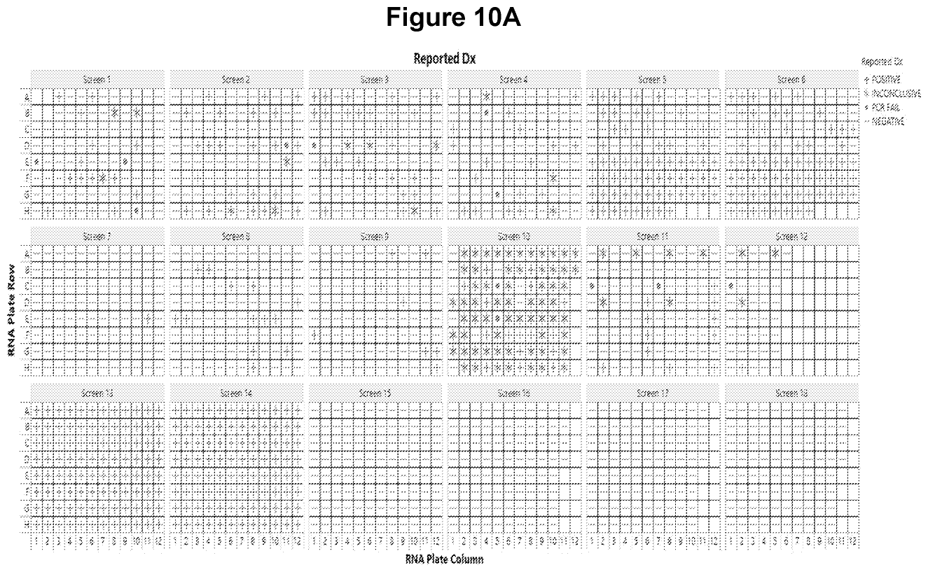

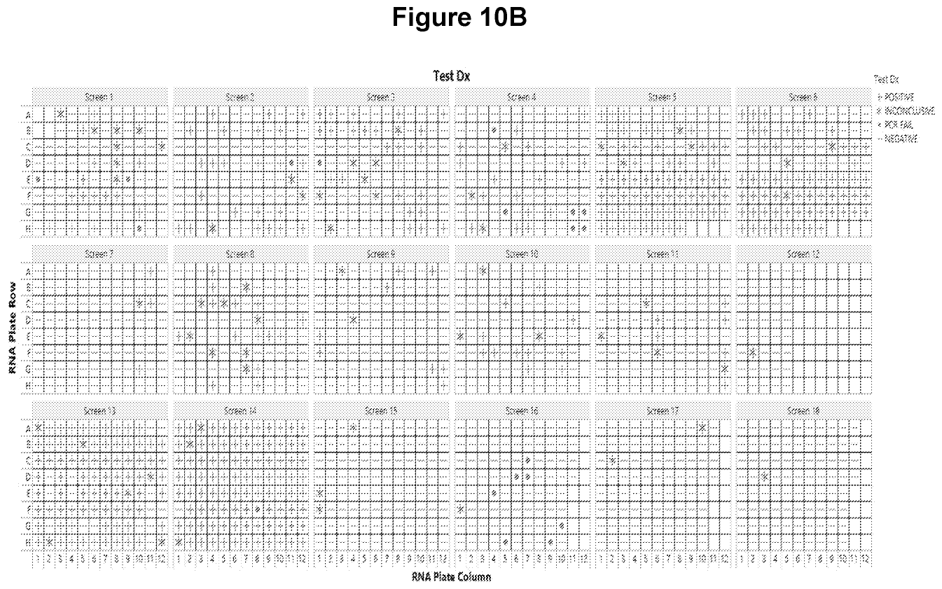

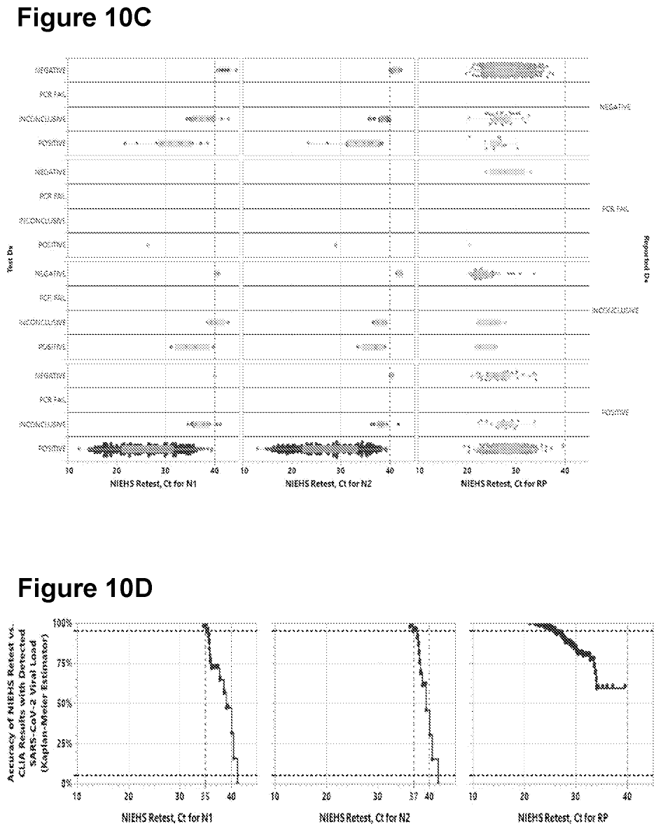

A total of 1,620 individual rRT-qPCR confirmatory retests were performed upon receipt and re-processing of specimen remnants from long-term storage conditions (−80° C.), comprising 1,184 remnants assayed once and 218 remnants assayed in duplicate collected from 1,234 total independent donors overall (see Table 10, and FIGS. 10A and 10B). Positive, negative, contrived, and no-template controls, as well as synthetic RNA standard curves, were included in each reaction plate of NIEHS retests for quality assurance of the CDC EUA rRT-qPCR assay (SARS-CoV-2 detection primer/probe sets: N1, N2; internal control primer/probe set: RP). Detection status from NIEHS retests were compared to their true reference condition, referred hereafter as CLIA Result and assumed as the SARS-CoV-2 infection status of freshly collected specimens determined by their original rRT-qPCR diagnostic testing at qualified laboratories.

The expected prevalence among CLIA Results for SARS-CoV-2 infection was 27.90% (452 detected among all 1,620 assays). Upon retest at NIEHS, an accuracy rate of 95.12% was observed with respect to CLIA Results (1,541 matching vs. 1,620 total scores); a>95% probability of NIEHS retests confirming SARS-CoV-2 detection based on a single target when either Ct<35 cycles for the N1 target or Ct<37 cycles for the N2 target, respectively; and <50% probability of NIEHS retests confirming SARS-CoV-2 detection based on a single target when Ct<39 cycles for either N1 or N2 target alone (see Tables 11-13, and FIGS. 10C and 10D).

At this point, it was taken into consideration that LeaSH RNA-seq relies on targeted priming for combinations of phylogeny-specific consensus sequences, pathogen-related structural motifs, and polyadenylated single-stranded RNA. In general, pathogen-specific consensus sequences and structural motifs have small sizes (<12 bp in length), which renders them useful as hybridization targets for primer-guided reverse transcription, but inadequate for highly specific nucleic acid amplification by thermal cycling. It was reasoned that a more adept comparison of performance to rRT-qPCR diagnostics should include, in addition to LeaSH RNA-seq as the test scenario, a fit-for-purpose chemistry equivalent that relies on rRT-qPCR primer designs to perform sequencing-based diagnostics.

TABLE 10
Summary of confirmatory screening samples and their sources used for validation and performance benchmarking of
SARS-COV-2 detection by repeated rRT-qPCR testing at NIEHS.

| | | Specimen | Number | Number | | | | NIEHS |
|---|---|---|---|---|---|---|---|---|
| | | Sharing | of | of | | Place | CLIA Testing | Retest |
| Task | Source | Mechanism | Donors | Specimens | Specimen Type(s) | of Collection | Provider | Method |
| Validation, | Integrated DNA | Commercial | n/a | 4 | Synthetic RNA | n/a | n/a | CDC EUA |
| QA/QC | Technologies¹ | | | | | | | rRT-qPCR |
| | NIEHS Department | Internal (human- | n/a | 1 | Total RNA | n/a | n/a | (One-Step |
| | of Intramural | derived cell line) | | | | | | TaqPath) |
| | Research² | | | | | | | |
| | BEI Resources^{3#} | Material Transfer | n/a | 6 | Viral culture | n/a | U.S. Centers for | |
| | | Agreement | | | supernatant (3); | | Disease Control | |
| | | | | | viral culture | | and Prevention | |
| | | | | | gRNA (2); | | (CDC) | |
| | | | | | Synthetic RNA (1) | | | |
| Bench- | Boca Biolistics^{4#} | Commercial | 104 | 104 | Donor NP swab | U.S. (continental); | Boca Biolistics | |
| marking | | | | | | Caribbean; Italy | | |
| | ReproCell^{5#} | Commercial | 48 | 48 | Donor NP swab | Beltsville, | ReproCell | |
| | | | | | | Maryland, U.S. | | |
| | University of Texas | Research | 50 | 50 | OP swab | El Paso, Texas, | University of | |
| | at El Paso | Collaboration | | | | U.S. | Texas at El Paso | |
| | (UTEP)^{5#} | Agreement | | | | | (UTEP) | |
| | Helix OpCo^{5†} | Material Transfer | 380 | 380 | Pre-extracted RNA | U.S. (continental) | Helix OpCo | |
| | | Agreement | | | (from NP swabs) | | | |
| | NIEHS Clinical | Internal | 84 | 252 | Donor sets: buccal | Durham, North | Quest | |
| | Research Unit^{6#} | (IRB-approved) | | | swab, NP swab, | Carolina, U.S. | Diagnostics | |
| | | | | | saliva (Norgen kit) | | | |
| | Norgen BioTek^{7#} | Research | 376 | 376 | Saliva | Guayaquil, | Norgen BioTek | |
| | | Collaboration | | | (Norgen kit) | Guayas, | | |
| | | Agreement | | | | Ecuador | | |
| | Emory | Service Contract | 192 | 192 | NP swab | Atlanta, Georgia, | Emory | |
| | University^{8#} | | | | | U.S. | University | |

¹Control templates and standard curves, QA/QC in every plate; used as reaction templates per manufacturer's instructions
²Negative contrived human template, QA/QC in every plate; used as reaction template, diluted <100 ng/μL
³Used at NIEHS for validation of CDC EUA rRT-qPCR (One-Step TaqPath) assay
⁴Samples 1-42 & 61-104: repeated tests, users A and B (one test each); samples 49-60: one test, user A
⁵One test, user A
⁶Samples 1-42 × 3: repeated tests, users A and B (one test each); samples 43-84 × 3: one test, user B
⁷Samples 1-286: one test, user A; samples 287-376: one test, user B
⁸One test, user B
^{#}Used 100 μL remnant specimens for RNA extraction; used RNA elutions as reaction templates directly
^{†}Used RNA elutions as reaction templates directly

TABLE 11
Confusion matrix of screening score results by rRT-qPCR
from initial testing in CLIA-certified facilities vs. repeated
testing at NIEHS for confirmatory screening samples

| | | Reported Dx | | | | |
|---|---|---|---|---|---|---|
| | | | PCR | Incon- | | Total |
| Screening Result | | Negative | Fail | clusive | Positive | (Row) |
| Test | Negative | 1011 | 4 | 74 | 40 | 1129 |
| Dx | PCR Fail | 11 | 7 | 0 | 1 | 19 |
| | Inconclusive | 36 | 0 | 7 | 20 | 63 |
| | Positive | 13 | 1 | 4 | 391 | 409 |

| | | | | | |
|---|---|---|---|---|---|
| Total (Column) | 1071 | 12 | 85 | 452 | 1620 |

TABLE 12
Confusion matrix of SARS-COV-2 detection by rRT-qPCR
from initial testing in CLIA-certified facilities vs. repeated
testing at NIEHS for confirmatory screening samples

| | | CLIA Result | | Total |
|---|---|---|---|---|
| SARS-COV-2 Status | | Undetected | Detected | (Row) |
| NIEHS | Undetected | 1150 | 61 | 1211 |
| Retest | Detected | 18 | 391 | 409 |
| | Total (Column) | 1168 | 452 | 1620 |

TABLE 13
Performance metrics of SARS-COV-2 detection by rRT-qPCR for confirmatory
screening samples by repeated testing at NIEHS relative to diagnosis by initial
testing in CLIA-certified facilities (“gold standard”).

| Metric | Net Counts | Rate | Interpretation |
|---|---|---|---|
| Accuracy | 1,541/1,620 | 95.12% | (rate of matching scores vs. true reference) |
| Prevalence | 452/1,620 | 27.90% | (rate of true detected scores among tests) |
| True Positive Rate | 391/452 | 86.50% | Sensitivity or Recall (negative score rules out) |
| True Negative Rate | 1,150/1,168 | 98.46% | Specificity or Selectivity (positive score rules in) |
| False Positive Rate | 18/1,168 | 1.54% | False alarm or Fallout |
| False Negative Rate | 61/452 | 13.50% | Miss rate |
| False Omission Rate | 61/1,211 | 5.04% | False Rule-Out (negative score is unreliable) |
| False Discovery Rate | 18/409 | 4.40% | False Rule-In (positive score is unreliable) |
| Positive Predictive Value | 391/409 | 95.60% | Precision (positive score is reliable) |
| Negative Predictive Value | 1,150/1,211 | 94.96% | (negative score is reliable) |

A fit-for-purpose chemistry equivalent for sequencing-based SARS-CoV-2 detection benchmarking was designed (termed IonSwab), that is based on sequences<12 bp in length for reverse transcription priming that are represented in the primer sets from the CDC EUA rRT-qPCR diagnostic assay (N1, N2, and RP targets). IonSwab represents a useful intermediate between rRT-qPCR and the proposed LeaSH RNA-seq diagnostics, since IonSwab integrates features from both LeaSH RNA-seq (i.e., a short-sequence priming approach to reverse transcription in combination with equal sequence backbones and reaction conditions for splint priming during the PCR stage of the workflow) and an alternative sequencing-based detection technique specific to SARS-CoV-2 called SwabSeq (i.e., based on primers from the CDC EUA rRT-qPCR SARS-CoV-2 diagnostic assay for single-pot sequencing library synthesis). Still, IonSwab differs from LeaSH RNA-seq in some critical ways: first, it replaces the Tailed SARS-CoV-2_Mod primer for an equimolar mix of 3 primers (Tailed CDC-N1-R, Tailed CDC-N2-R, and Tailed CDC-RP-R) each differing only by the last 9 nucleotides which correspond to the last 9 nucleotides found in the reverse primers used by the CDC EUA rRT-qPCR assay for the N1, N2 and RP targets; it replaces the Tailed SARS-CoV-2_TRS primer for an equimolar mix of 3 primers (Tailed CDC-N1-F, Tailed CDC-N2-F, and Tailed CDC-RP-F) each differing only by the last 11 nucleotides which correspond to the last 11 nucleotides found in the forward primers used by the CDC EUA rRT-qPCR assay for the N1, N2 and RP targets; third, it does not use anchored oligo dT primers; and fourth, it does not use a template-switching primer for complementary strand synthesis during reverse transcription. IonSwab also differs from SwabSeq in some key aspects: IonSwab relies on the LeaSH RNA-seq backbone instead for splint priming; and it uses partial, not full, primer sequences from the CDC EUA rRT-qPCR assay for N1, N2 and RP amplicon targeting.

To benchmark the ability of the LeaSH RNA-seq approach presented herein to capture SARS-CoV-2 viral copies relative to qPCR screening, an expectation dataset was created by IonSwab, which represents a sequencing-based chemistry targeting the same amplicons as the CDC EUA rRT-qPCR assay for SARS-CoV-2 diagnostics. In this experiment, the scope was narrowed to a single 96-sample set of independently collected SARS-CoV-2 positive remnant samples of different matrix types and obtained through different sources, namely: a) remnant OP swabs 1-48 donated by the University of Texas at El Paso; and b) all 48 remnant NP swabs purchased from ReproCell. This reference plate corresponds to “Screen 13” from the confirmatory retests performed at NIEHS (see FIGS. 10A and 10B).

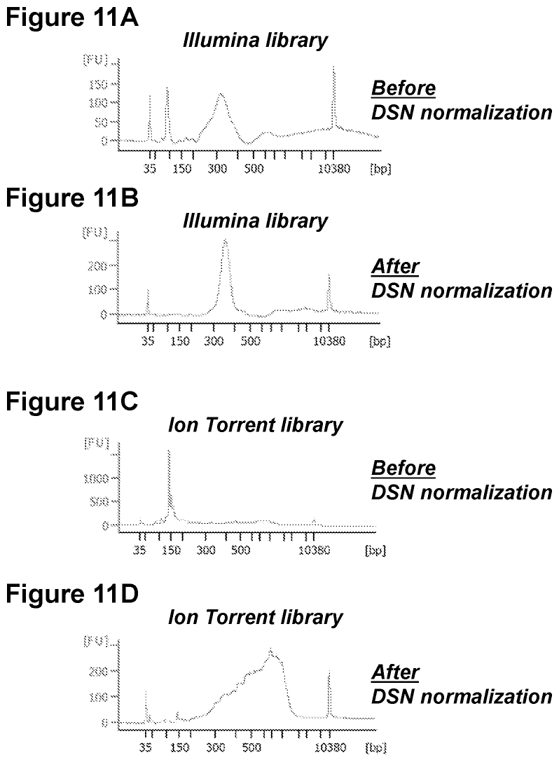

To create the IonSwab expectation dataset, the UTEP-ReproCell reference plate was used to synthesize a multiplexed IonSwab library of uniquely barcoded samples via combinatorial dual-indexing with template binding sequences for Ion Torrent sequencing platforms, enriched for the 200 bp-600 bp library fraction by 0.5×-0.7× double-sided SPRI selection, and quantified afterwards by integration of electropherogram traces from BioAnalyzer capillary electrophoresis assays. Moreover, given the proclivity to enrich for reverse-transcribed primer concatemers by the initial “cold” cycling settings in the PCR indexing portion of the LeaSH RNA-seq protocol, a 50-500 ng DNA aliquot of the size-selected IonSwab library was further subjected to duplex-specific nuclease (DSN) normalization to digest excess molar contributions from templates<200 bp that overwhelmed initial SPRI-based size selection (see FIG. 11). The DSN-treated IonSwab library was purified by 0.8× single-sided SPRI afterwards, re-amplified by PCR using Ion Torrent library amplification primers, and quantified by integration of electropherogram traces from BioAnalyzer capillary electrophoresis assays. Two aliquots from the IonSwab library stock before DSN normalization were sequenced using one Ion 520 chip and one Ion 540 chip respectively, and one aliquot of the IonSwab library stock after DSN normalization was sequenced using a separate Ion 540 chip.

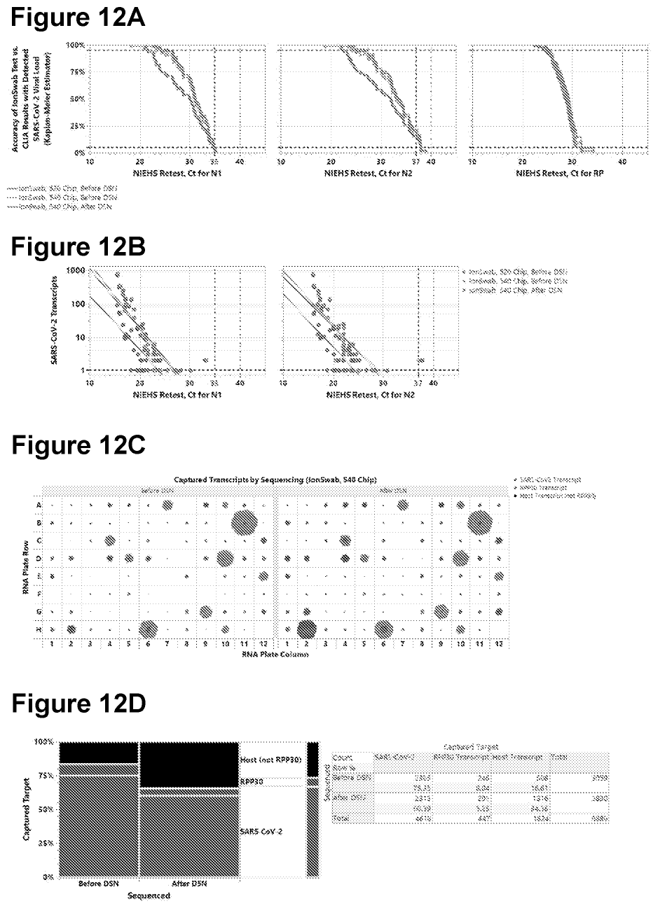

Based on IonSwab data from the UTEP-ReproCell reference plate comprising 96 independent donors and starting from a size-selected library without DSN normalization, when sequencing in a single Ion 520 Chip (3M-5M raw reads total, ˜30K-50K raw reads avg. per donor) a >95% probability of confirming SARS-CoV-2 detection was observed for samples with Ct<22 cycles for either N1 or N2 target alone and <50% probability when Ct<30 cycles for the N1 target or Ct<32 cycles for the N2 target. In contrast, when sequencing the same library in a single Ion 540 Chip (60M-80M raw reads total, ˜600K-800K raw reads avg. per donor), the >95% probability of confirmation thresholds based on CDC EUA rRT-qPCR retests at NIEHS improved to Ct<24 cycles without DSN normalization and Ct<25 cycles with DSN normalization for either N1 or N2 target alone, and Ct<31 cycles for the N1 target or Ct<33 cycles for the N2 target at the <50% probability of confirmation threshold irrespective of DSN normalization (see Table 15, and FIG. 12A).

Inspection of the IonSwab data showed that, given the same library without DSN normalization, the net difference in diagnostic performance by sequencing in Ion 540 vs. Ion 520 chips was correlated to the total read capacities (i.e., sequencing throughput) between both chips. However, that difference was not strictly proportional to the relative throughput between Ion 540 and Ion 520 chips, suggesting also that the effective difference in diagnostic performance was due to the intrinsic complexity of viral transcripts counted from the IonSwab library, only partially sampled in the Ion 520 run but sequenced beyond saturation in the Ion 540 run. This explanation is reinforced by two other observations: first, that the shift in diagnostic performance when going from Ion 520 to Ion 540 chips of the same IonSwab library without DSN normalization is directly proportional to the underlying Ct score of the specimens—as shown by semi-log regressions between SARS-CoV-2 transcript counts vs. observed Ct by rRT-qPCR retests (see FIG. 12B)—meaning it is largest for specimens with higher viral load (i.e., the >95% probability of confirmation threshold for samples with low Ct scores by IonSwab shifts from Ct<22 cycles in Ion 520 chips to Ct<24 cycles in Ion 540 chips) and null for “borderline” positive samples (i.e., no change for samples with Ct>35 cycles past the >95% probability of confirmation threshold with CDC EUA rRT-qPCR retests); and second, when sequencing in Ion 540 chips, enzymatic removal of PCR artifacts<200 bp from the IonSwab library using DSN normalization improved capture rates both for SARS-CoV-2 transcripts in confirmed positive specimens with high viral loads (i.e., for the >95% probability of confirmation threshold by IonSwab, Ct<24 cycles before vs. Ct<25 cycles after DSN library normalization) and for “off-target” host transcripts complementary to partial CDC primer sequences in samples with low viral load that failed confirmation by retest at NIEHS (see FIGS. 12C, and 12D).

It is worth noting that the comparisons in terms of net counts for SARS-CoV-2 and “off-target” host transcripts between Ion 520 and Ion 540 sequencing runs for the same UTEP-ReproCell reference plate were possible because of UMI tagging, a key element of all LeaSH RNA-seq candidate chemistries that is absent from other sequencing-based diagnostic methods available elsewhere. Because UMI tagging distinguishes between total counts of sequenced PCR clones (i.e., raw read output, in the millions overall) and total counts of native templated transcripts (i.e., library complexity, in the thousands overall), it represents a critical tool in tracking cost-benefit metrics in terms of transcript counting for experiments incurring lean vs. deep sequencing in terms of raw read throughput, as shown here when comparing IonSwab runs before DSN normalization that were sequenced to disproportionate throughputs using Ion 520 vs. Ion 540 chips (see FIGS. 12A, and 12B) or when comparing IonSwab runs before and after DSN normalization that were sequenced to similar deep throughputs using Ion 540 chips for both (see FIGS. 12C, and 12D).

Given the results observed in IonSwab sequencing experiments, the data from the IonSwab run of the UTEP-ReproCell reference plate using one Ion 540 chip after DSN normalization was defined as the IonSwab expectation dataset, and it was used as the benchmark for diagnostic performance of different LeaSH RNA-seq implementation chemistries. DSN normalization was also included as a standard final step in all LeaSH RNA-seq library synthesis assays thereafter.

To evaluate sequencing-based diagnostic performance using LeaSH RNA-seq primer sets, DSN-normalized libraries synthesized from the UTEP-ReproCell reference plate were sequenced using 3 distinct “IonPrimed” chemistries, with SARS-CoV-2 detection and host RNA capture diversity compared among them and against the IonSwab expectation dataset afterwards. Each of the “IonPrimed” chemistries used equimolar mixtures of different primer subsets represented in the overall LeaSH RNA-seq design as follows: (a) IonTSOdT, which used Anch-dT for reverse transcription, SARS-CoV-2_TRS-TSO for template switching, and Tailed SARS-CoV-2_ TRS for splinting to prioritize template-switching cDNA synthesis from 3′-polyadenylated RNA templates; (b) IonMotifs, which used Tailed SARS-CoV-2_Mod for reverse transcription, SARS-CoV-2_TRS-TSO for template switching, and Tailed SARS-CoV-2_TRS for splinting to prioritize template-switching cDNA synthesis from RNA with sequences complementary to SARS-CoV-2 TRS and structural motifs; and (c) IonRTMix, with all primers from (a) and (b) included at once in equimolar contents. In short, single multiplexed IonSwab libraries for Ion Torrent sequencing were synthesized for each IonPrimed chemistry using the UTEP-ReproCell reference plate as template, size-selected to 200 bp-600 bp size range by 0.5×-0.7× double-sided SPRI method, subjected to duplex-specific nuclease (DSN) normalization to digest excess molar contributions from templates<200 bp, purified by 0.8× single-sided SPRI afterwards, re-amplified by PCR using Ion Torrent library amplification primers, and quantified by integration of electropherogram traces from BioAnalyzer capillary electrophoresis assays. Just like the IonSwab expectation dataset, each individual IonPrimed library was sequenced independently in one Ion 540 chip (60M-80M raw reads total, ˜600K-800K raw reads avg. per donor).

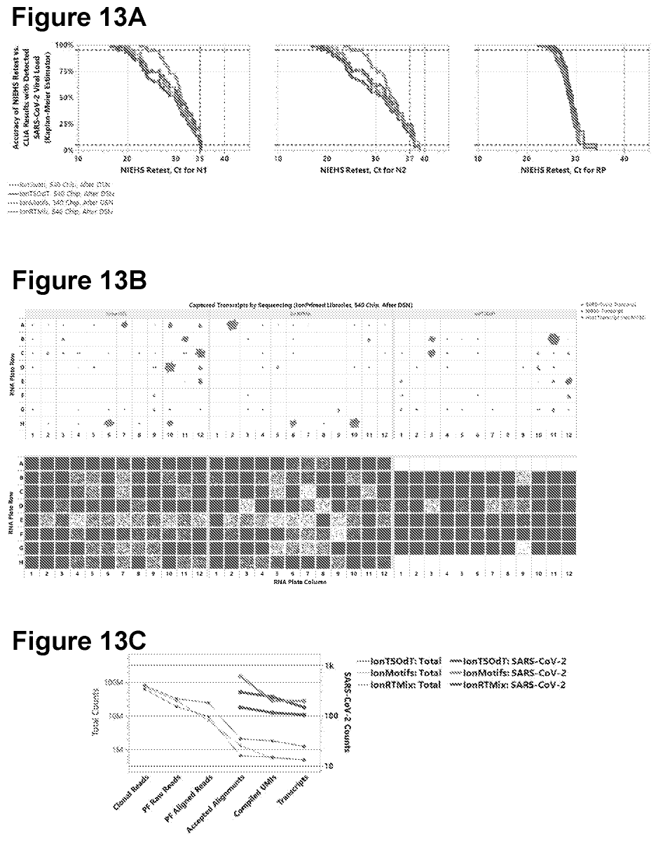

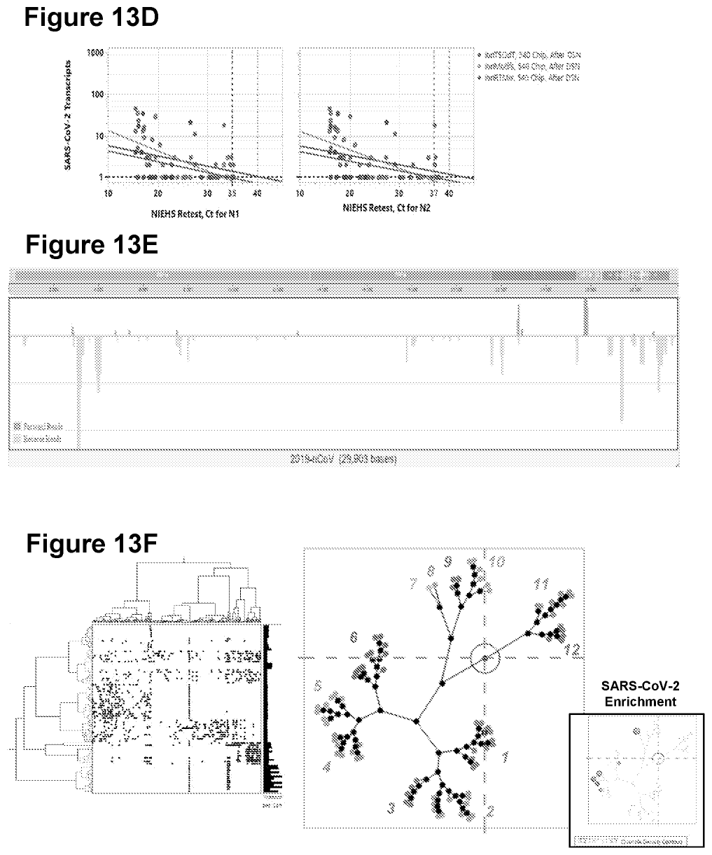

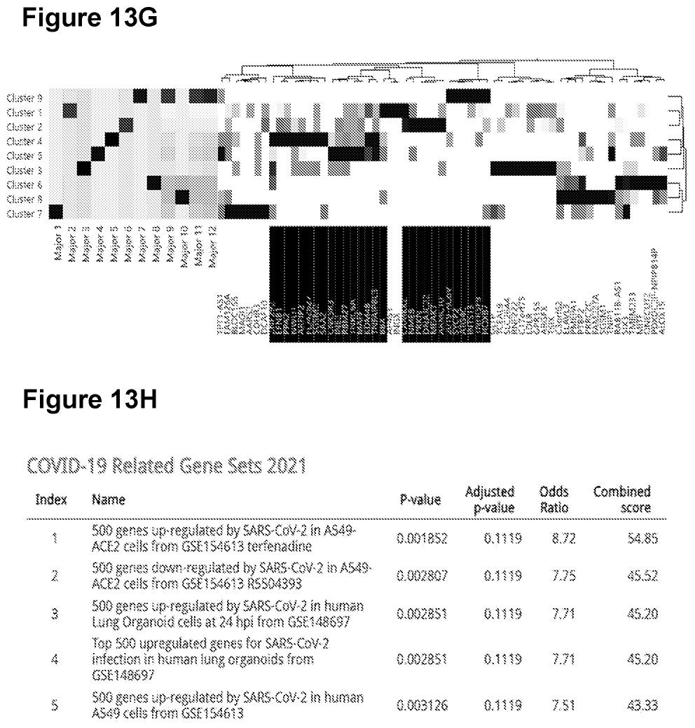

Inspection of data from IonPrimed libraries showed>95% probability of confirmation thresholds for SARS-CoV-2 detection at Ct<18, <20, or <21 cycles for the N1 target or Ct<19, <21, or <22 cycles for the N2 target by IonTSOdT, IonRTMix, or IonMotifs assays respectively. At the <50% probability of confirmation threshold, Ct<31 cycles for the N1 target or Ct<32 cycles for the N2 target by all 3 IonPrimed assays was observed (see Table 15 and FIG. 13A). Overall, these results indicated that none of the IonPrimed assays improved upon the SARS-CoV-2 diagnostic performance of the IonSwab expectation dataset (>95% probability of confirmation threshold: Ct<25 cycles) when sequenced to comparable outputs—in fact, the capability of SARS-CoV2 detection for all IonPrimed libraries was reminiscent of the IonSwab 520 Chip run before DSN normalization (˜30K-50K raw reads avg. per donor; see FIG. 12A).

However, IonSwab and IonPrimed chemistries led to differences in the constitution of transcript sources represented in their respective libraries. That difference in library complexity is so substantial that, given the same sequencing throughput by using Ion 540 chips for both, the IonSwab expectation dataset and IonPrimed libraries both detect SARS-CoV-2 transcripts at comparable net counts, yet those add up to most of the transcripts captured by IonSwab (about 60%-80% of total transcripts) but only represent a minimal contribution to the total library complexity found in IonPrimed libraries (<0.04% of total transcripts) (see FIGS. 12C, 12D, 13B, and 13C). In other words, IonPrimed libraries can probe host transcriptomes at rates far beyond the “off-target” capture rates observed in IonSwab. It also suggests that the underlying library complexity is larger in IonPrimed libraries because these allow for both SARS-CoV-2 and host RNA templates to contribute to the final tally, whereas IonSwab libraries are more restrictive and only amenable to sequencing targeted amplicons from SARS-CoV2 templates or host-derived internal controls like RPP30 (see Table 15 and FIG. 12C).

From a bioinformatics perspective, this outcome implies that, even though net SARS-CoV-2 diagnostic performance is somewhat similar at equal sequencing depths, IonPrimed libraries are far more profitable than IonSwab librariess because IonPrimed chemistries can capture large volumes of transcriptional information from the host that IonSwab designs simply do not tap into. In fact, this ability to extract host transcripts from IonPrimed libraries allowed recognizing that the IonTSOdT multiplexed library failed to include data for 24 of the 96 templates in the UTEP-ReproCell reference plate, which was later attributed to an instrumentation error during automated liquid dispensing (i.e., omission of rows A and H during reverse transcription reaction setup; see FIG. 13B).

Notably, the relationship in diagnostic performance between IonSwab and IonPrimed chemistries was no longer correlated with total read outputs—in fact, Ct values of samples confirmed by IonPrimed chemistries exhibited an extended range compared to the Ct values of samples confirmed by IonSwab (as shown by semi-log regressions between SARS-CoV-2 transcript counts vs. observed Ct by rRT-qPCR retests, see FIG. 13D). Once again, this outcome could be explained by the diversity of SARS-CoV-2 templates that IonPrimed chemistries can capture, which adopt a motif-enriched “shotgun” strategy instead of the amplicon-specific targeting used in IonSwab. This conclusion is supported by the presence of transcripts aligning to different loci across the SARS-CoV-2 genome in IonPrimed library data, as demonstrated by transcripts captured in the IonRTMix run which are consistent with Tailed SARS-CoV-2_TRS oligonucleotide priming to TRS instances in the SARS-CoV-2 genome, none of which match N1 or N2 amplicons that IonSwab chemistries target (see FIG. 13E). Therefore, IonPrimed chemistries extend the detection range of sequencing-based SARS-CoV-2 diagnostics by increasing the number of hybridization opportunities, and thus the number of possible detection events, for each of its reverse transcription primers against each individual SARS-CoV-2 RNA template in a sample.

Next, transcriptional data was analyzed to determine whether multiplexed sample-barcoded libraries synthesized using LeaSH RNA-seq chemistries allowed for segregation of samples based on latent patterns of shared gene expression from host genomes, and at sequencing depths coincident with saturated SARS-CoV-2 transcript representation. Briefly, the SALSA analytical workflow (Lozoya et al. “Patterns, Profiles, and Parsimony: Dissecting Transcriptional Signatures From Minimal Single-Cell RNA-Seq Output With SALSA” Front. Genet., Vol. 11, Article 511286, 9 Oct. 2020) was repurposed towards single-sample RNA-seq analyze data from the IonRTMix library (˜600K-800K raw reads avg. per donor) and extract major sample groupings driven by gene expression similarity of statistically sifted candidate “profiler” genes. Profilers were inspected further based on correspondence between SALSA-inferred sample groups vs. latent clusters, the former determined by a representation-weighed latent class analysis of groupxprofiler expression couplings. Subsets of candidate biomarkers, corresponding to the highest-ranking profilers based on their contribution to latent classification of samples, were determined by degree-of-correlation scores sifted through an outlier analysis of multivariate contributions to latent classification (Mahalanobis distance method). Adequacy of agnostic biomarker extraction was vetted by confirming classification correspondence levels between SALSA-inferred groups and latent clusters based on candidate biomarker data only and depicted by two-way unsupervised hierarchical clustering of contributions scores.

Bioinformatics analysis of gene expression patterns by SALSA using IonRTMix data revealed 12 major transcriptional groupings among samples in the UTEP-ReproCell reference plate, driven by differential expression of 220 profiler genes from the hosts in addition to viral SARS-CoV-2 RNA (see Table 14). The 12 major groups coincided with compartments of samples expressing similar SARS-CoV-2 transcript enrichment, particularly around majors 4-7 (see FIG. 13F). Following extraction of agnostic biomarker genes among the 220-profiler gene subset, it was observed that the transcriptional profiles of SARS-CoV-2 positive samples in majors 4, 5, and 6 corresponded with the enrichment of biomarkers represented in latent clusters 5, 4, and 2 respectively. In contrast, the transcriptional profile of the one sample in major 7, which showed the largest SARS-CoV-2 enrichment level overall, shared enrichment of biomarkers in latent cluster 9 with samples from other groupings (majors 9, 11, and 12) that, in contrast, did not exhibit SARS-CoV-2 transcript enrichment (see FIG. 13G). The latent clusters of biomarker candidates, which were best correlated with confirmed infection (majors 4-7) or suspected infection (majors 9, 11, and 12)—as determined by detection of SARS-CoV-2 transcripts in IonRTMix data—and were identified from within the 220-profiler gene set, comprised the following 27 human genes: ARFIP2, ARMC10, ATG4C, BBX, CAMKK2, CNKSR3, DNAJC22, EFNB1, FLJ42627, HOXB7, INE2, INTS13, KDM4B, MAFF, MEAK7, NME8, NWD1, PPA2, PRKN, RBM27, SAA2, SGSM2, SYCP2, TNFAIP8L3, TNFRSF9, TNRC6A, and ZNF292 (see FIG. 13G). Pathway enrichment analysis for those 27 biomarker candidates using the Enrichr engine (Xie et al. “Gene set knowledge discovery with Enrichr.” Current Protocols, 1, e90. 2021) showed highest statistically significant enrichment for 4 sets of upregulated genes (index 1, 3, 4, and 5) discovered using in vitro and ex vivo models of SARS-CoV-2 infection, and 1 set of genes (index 2) downregulated upon treatment with a candidate antiviral compound (see FIG. 13H), suggesting the subset of agnostically selected biomarkers using IonRTMix is consistent with independently reported host transcriptional response to infection with SARS-CoV-2 in mammalian cell models (Han et al. “Identification of Candidate COVID-19 Therapeutics using hPSC-derived Lung Organoids” bioRxiv preprint version posted May 5, 2020 (//doi.org/10.1101/2020.05.05.079095; Hoagland et al. “Modulating the transcriptional landscape of SARS-CoV-2 as an effective method for developing antiviral compounds” bioRxiv preprint version posted Jul. 13, 2020//doi.org/10.1101/2020.07.12.199687).

TABLE 14
List of 220 profiler host genes, identified by SALSA analysis, based on IonRTMix sequencing
data from SARS-COV-2 positive samples in the UTEP-ReproCell reference plate.
Gene Symbol

| | | | | | | | | | | |
|---|---|---|---|---|---|---|---|---|---|---|
| ARFIP2 | SAA2 | BCYRN1 | CERT1 | FECH | LDLR | MITF | PCLAF | RBBP5 | SMG7- | UBIAD1 |
| | | | | | | | | | AS1 | |
| ARMC10 | SGSM2 | BLOC1S5 | CHM | FUT2 | LIMS1 | MTG2 | PCSK7 | RBM3 | SNAP29 | UBN2 |
| ATG4C | SYCP2 | BLOC1S6 | CHST6 | GATAD1 | LINC00470 | MTMR9 | PDE4C | RINL | SOWAHC | USP1 |
| BBX | TNFAIP8L | BMS1P1 | CHURC1 | GBP4 | LINC00958 | MYO1C | PDXDC2P- | RIPOR2 | SPAG9 | VPS53 |
| | 3 | | | | | | NPIPB14P | | | |
| CAMKK2 | TNFRSF9 | BNC2 | CNTF | GGPS1 | LINC01299 | NAIF1 | PGPEP1 | RND2 | SRSF8 | VSIG1 |
| CNKSR3 | TNRC6A | C3orf62 | CRYBB2 | GLTP | LINC02381 | NCRUPAR | PHC3 | RNF14 | TACC1 | WWC1 |
| | | | P1 | | | | | | | |
| DNAJC22 | ZNF292 | C17orf75 | DAG1 | GNB4 | LINC02878 | NKIRAS2 | PIK3C2A | RNF141 | TBC1D32 | XAF1 |
| EFNB1 | AARS2 | C21orf62 | DCAF7 | GNE | LOC286437 | NLN | PLCXD1 | RNF157 | TCEAL9 | XPNPEP3 |
| FLJ42627 | ABHD11 | CADM2 | DCAF10 | GPR155 | LPP | NM_13846 | PLPP5 | RNF216 | TMC5 | ZBP1 |
| | | | | | | 4 | | | | |
| HOXB7 | AKAP5 | CARF | DMC1 | GRK3 | LRRC27 | NPHS1 | PLXDC1 | RNF222 | TMC7 | ZFP14 |
| INE2 | ALOX15 | CBFA2T2 | DNM1L | GUCA1B | LRRC74B | NR_003132 | PMEPA1 | RPS15AP | TMEM181 | ZHX3 |
| | | | | | | | | 10 | | |
| INTS13 | AMY2A | CCBE1 | ECE1 | GXYLT1 | LYRM7 | NR_026905 | PNPLA8 | RSL1D1 | TMEM184 | ZNF37A |
| | | | | | | | | | A | |
| KDM4B | ANAPC16 | CCDC114 | EID2B | HBS1L | MACROD2 | NUPR2 | POFUT1 | RTL10 | TMEM233 | ZNF114 |
| MAFF | AP1S3 | CCL28 | ELAVL3 | IDS | MAGI1 | ODF2L | PRKCB | SCAI | TNIP1 | ZNF329 |
| MEAK7 | AP4S1 | CD82 | ELMOD1 | IKZF3 | MAN1B1- | ONECUT2 | PRR11 | SCAND2P | TONSL | ZNF430 |
| | | | | | DT | | | | | |
| NME8 | APOL1 | CD109 | FAM111B | INGX | MANEAL | OSBPL11 | PRRC2C | SGSM1 | TPT1-AS1 | ZNF441 |
| NWD1 | ARGFX | CDHR3 | FAM126A | KCNJ5 | MCUR1 | P2RX7 | PTBP2 | SHROOM | TRIM65 | ZNF445 |
| | | | | | | | | 4 | | |
| PPA2 | ASB11 | CDK2 | FAM227A | KLRD1 | METTL2A | PAF1 | RAB3B | SIX3 | TSC22D1- | ZNF485 |
| | | | | | | | | | AS1 | |
| PRKN | ATG14 | CDK4 | FBXO33 | KLRG1 | METTL2B | PARD6G | RAB11B- | SLC14A2 | TSIX | ZNF594 |
| | | | | | | | AS1 | | | |
| RBM27 | B3GNT6 | CEP68 | FCF1 | LAX1 | MFSD8 | PCDH9 | RABL3 | SLC26A4 | TTPAL | ZNF793 |

Considering our prior knowledge of SARS-CoV-2 positivity in all samples based on their initial diagnostic results from CLIA-certified facilities (see Table 10) the transcriptional similarity for candidate host-derived biomarkers of infection between samples with high-grade and low-grade SARS-CoV-2 detection rates suggests a means to scoring SARS-CoV-2 infection risk based on polygenic profiling of host transcriptomes in lieu of SARS-CoV-2 detection. This dual capacity to extract host transcriptome information using LeaSH RNA-seq chemistries is also more powerful than sequencing strategies limited to SARS-CoV-2 amplicon targeting: these results show that host-derived transcripts are vastly more abundant and diversely represented than SARS-CoV-2 viral templates found in RNA extracted directly from diagnostic swabs of SARS-CoV-2 positive donors, and that host transcriptomes can be probed to higher net transcripts counts with substantially leaner sequencing runs than SARS-CoV-2 transcripts alone (see FIGS. 13B, and 13C).

In summary, we found that libraries synthesized using IonPrimed chemistries showed an enriched overall diversity of captured transcripts relative to IonSwab libraries sequenced to comparable raw read outputs, yet the number of detected SARS-CoV-2 transcripts was lower in all IonPrimed versions. Inspection of genomic annotations on sequenced transcripts confirmed that all 3 IonPrimed methods captured diverse pools of patient-derived transcripts otherwise inaccessible to traditional rRT-qPCR diagnostic assays, pathogen-specific tiling primer kits like COVIDseq or ARTIC, and targeted amplicon-specific sequencing chemistries like IonSwab or SwabSeq. These results speak to the “shotgun” strategy that LeaSH RNA-seq is aimed towards simultaneous SARS-CoV-2 and host transcriptome representation, and suggests at least two possible causes in combination: a) that sequencing saturation is achieved in IonSwab libraries with fewer raw reads than in IonPrimed libraries; and b) that SARS-CoV-2 transcript capture based on short-motif targeting competes for hybridization primers and enzymatic reactants with off-target and motif-encoding transcripts from the host. Given those potential causes, it was reasoned that net outputs of SARS-CoV-2 transcripts (which inform infection diagnoses) may saturate before net outputs of host-derived transcripts (which inform response to infection by the host), and that requisite sequencing depths for LeaSH RNAseq assays should be informed by both their diagnostic performance (based on captured SARS-CoV-2 transcripts) and their ability to dissect transcriptional correlate metrics (based on patterns of host gene expression) for SARS-CoV-2 infection. Therefore, to implement LeaSH RNA-seq for Ion Torrent-based sequencing, an all-at-once formulation similar to the IonRTMix design was adopted as the reference stoichiometry hereafter, which we termed IonLeaSH, and that included a revised stoichiometry between 3′ reverse transcription primers (1:1 in IonRTMix vs. 4:1 in IonLeaSH for [Tailed SARS-CoV-2_Mod]:[barcoded Anch-dT]) with the purpose of enhancing cDNA synthesis from templates with SARS-CoV-2 post-translational modification motifs relative to 3′-polyadenylated RNA templates. This change in 3′ reverse transcription primer apportionments was introduced in the IonLeaSH chemistry to counterbalance the outcome for SARS-CoV-2 transcripts observed in IonPrimed sequencing runs, all of which were overwhelmingly dominated by host transcripts but otherwise accrued fewer SARS-CoV-2 transcripts than the IonSwab expectation dataset that had been sequenced to equal depth (see FIGS. 12C, and 13B).

To evaluate the effect of sequencing depth on saturation of LeaSH RNA-seq library complexity, particularly among host-derived transcripts, a DSN-normalized IonLeaSH library synthesized from the UTEP-ReproCell reference plate was sequenced in technical replicates using two Ion 510, two Ion 520, two Ion 530, and two Ion 540 chips. In short, the single multiplexed IonLeaSH library for Ion Torrent sequencing was synthesized using the UTEP-ReproCell reference plate as template, size-selected to 200 bp-600 bp size range by 0.5×-0.7× double-sided SPRI method, subjected to duplex-specific nuclease (DSN) normalization to digest excess molar contributions from templates<200 bp, purified by 0.8× single-sided SPRI afterwards, re-amplified by PCR using Ion Torrent library amplification primers, and quantified by integration of electropherogram traces from BioAnalyzer capillary electrophoresis assays. For analysis, SARS-CoV-2 detection and host RNA capture diversity were compared on the basis of chip type (e.g., “2×510 Chip” refers to unique transcript data from the same library, accrued from two sequencing runs combined, and using one Ion 510 chip in each instance).

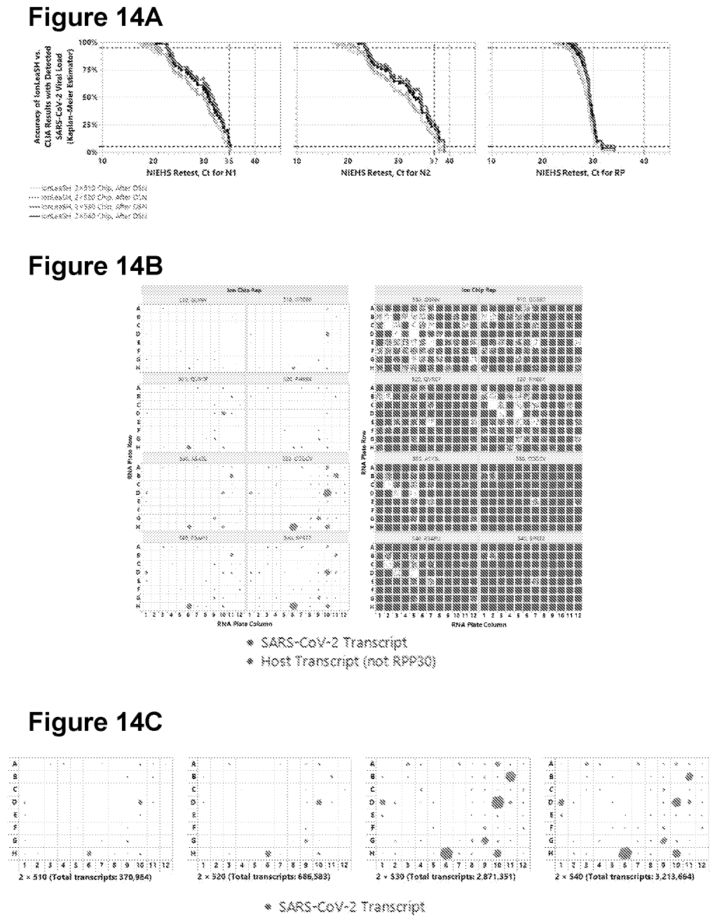

Inspection of IonLeaSH data compiled from duplicate runs in matched sequencing chips with different read outputs (2×510 Chip: 4M-6M raw reads total, ˜40K-60K raw reads avg. per donor; 2×520 Chip: 6M-10M raw reads total, ˜60K-100K raw reads avg. per donor; 2×530 Chip: 30M-40M raw reads total, ˜300K-400K raw reads avg. per donor; 2×540 Chip: 120M-160M raw reads total, ˜1.2M-1.6MK raw reads avg. per donor) showed>95% probability of confirmation thresholds based on CDC EUA rRT-qPCR retests at NIEHS for SARS-CoV-2 detection at Ct<19, <21, or <23 cycles for the N1 target or Ct<20, <22, or <24 cycles for the N2 target with combined sequenced data from 2×510 chips, from 2×520 chips, or from either 2×530 chips or 2×540 chips respectively. At the <50% probability of confirmation threshold, we observed Ct<30, <32, or <31 cycles for the N1 target or Ct<32, <34, or <33 cycles for the N2 target with combined sequenced data from either 2×510 chips or 2×520 chips, from 2×530 chips, or from 2×540 chips respectively. SARS-CoV-2 diagnostic performance by IonLeaSH improved with increasing sequencing depth, both approaching confirmation levels similar to the IonSwab expectation dataset (e.g., >95% probability of confirmation threshold for N2 target: Ct<24 vs. Ct<25 cycles for 2×530 IonLeaSH vs. 1×540 IonSwab, respectively) and reaching sequencing saturation of SARS-CoV-2 transcripts with fewer net reads overall (at ˜300K-400K vs. ˜600K-800K raw reads avg. per donor for 2×530 IonLeaSH vs. 1×540 IonSwab, respectively). Therefore, the revised stoichiometry between 3′ reverse transcription primers that was adopted for IonLeaSH implementation enhanced detection performance for SARS-CoV-2 transcripts to levels matching the IonSwab expectation dataset when run to comparable sequencing depths (see Table 15, and FIG. 14A).

Inspection of transcripts pools in IonLeash runs confirmed that the IonLeaSH chemistry was capable of probing host transcriptomes (see FIG. 14B). Notably, the net output of transcripts, dominated by host-derived transcripts, grew in proportion to Ion chip sequencing capacities with increasing sequencing depths up to the Ion 530 chip runs; also, the reproducibility of transcript apportionments among multiplexed samples improved between technical replicates with deeper sequencing (FIG. 14B). In sum, these results confirmed two critical features of IonLeaSH libraries: first, that library complexity in regard to SARS-CoV-2 transcripts is already sequenced near saturation with technical replication at an overall ˜300K-400K vs. ˜600K-800K raw reads avg. per donor; and second, that benefits on capture rates for diverse pools of host-derived transcripts in IonLeaSH libraries, which grow with additional throughput, also taper off at about the same sequencing rate that saturates SARS-CoV-2 representation (see FIG. 14C).

TABLE 15
Summary of confirmatory probability thresholds among SARS-Cov-2 positive
samples in the UTEP-ReproCell reference plate by Ion Torrent sequencing relative to repeated
rRT-qPCR testing at NIEHS.

| | | | Expected | | Thresholds of confirmation probability | |
|---|---|---|---|---|---|---|
| | | Expected | Raw | | for SARS-COV-2 positive samples | |
| | | Raw Read | Read Output | | relative to scores from retest at NIEHS | |
| | Sequencer | Output | per | DSN | by CDC EUA rRT-qPCR assay | |
| Chemistry | Loading | Overall | Sample | Normalization | >95% probability | <50% probability |
| IonSwab | 1 × Ion 520 | 3M-5M | 30K-50K | No | N1: Ct <22 cycles | N1: Ct <30 cycles |
| | | | | | N2: Ct <22 cycles | N2: Ct <32 cycles |
| | 1 × Ion 540 | 60M-80M | 600K-800K | No | N1: Ct <24 cycles | N1: Ct <31 cycles |
| | | | | | N2: Ct <24 cycles | N2: Ct <33 cycles |
| | | | | Yes | N1: Ct <25 cycles | N1: Ct <31 cycles |
| | | | | | N2: Ct <25 cycles | N2: Ct <33 cycles |
| IonTSOdT | 1 × Ion 540 | 60M-80M | 600K-800K | Yes | N1: Ct <18 cycles | N1: Ct <31 cycles |
| | | | | | N2: Ct <19 cycles | N2: Ct <32 cycles |
| IonRTMix | | | | | N1: Ct <20 cycles | N1: Ct <31 cycles |
| | | | | | N2: Ct <21 cycles | N2: Ct <32 cycles |
| IonMotifs | | | | | N1: Ct <21 cycles | N1: Ct <31 cycles |
| | | | | | N2: Ct <22 cycles | N2: Ct <32 cycles |
| IonLeaSH | 2 × Ion 510 | 4M-6M | 40K-60K | Yes | N1: Ct <19 cycles | N1: Ct <30 cycles |
| | | | | | N2: Ct <20 cycles | N2: Ct <32 cycles |
| | 2 × Ion 520 | 6M-10M | 60K-100K | | N1: Ct <21 cycles | N1: Ct <30 cycles |
| | | | | | N2: Ct <22 cycles | N2: Ct <32 cycles |
| | 2 × Ion 530 | 30M-40M | 300K-400K | | N1: Ct <23 cycles | N1: Ct <32 cycles |
| | 2 × Ion 540 | 120M-160M | 1.2M-1.6M | | N1: Ct <23 cycles | N1: Ct <31 cycles |
| | | | | | N2: Ct <24 cycles | N2: Ct <33 cycles |
| | | | | | N2: Ct <24 cycles | N2: Ct <34 cycles |

Altogether, our exploration of library complexities and saturation rates across IonSwab, IonPrimed, and IonLeaSh chemistries support the notion that SARS-CoV-2 detection by sequencing can be enhanced by increasing the overall transcript diversity represented in the library. In LeaSH RNA-seq, transcript diversity is increased relative to amplicon-targeted techniques by allowing for agnostic capture of host transcripts. In turn, access to host gene expression data within the same assay can be used independently from SARS-CoV-2 viral loads to extract gene expression signatures correlated with pathologically relevant SARS-CoV-2 infection. Paired to clinical outcomes from patient cohorts, this method can be deployed to determine biomarker-driven models that forecast COVID-19 onset or severity along the course of the disease, in the absence or independent from the life cycle of SARS-CoV-2 detection viral transmission and detection, and based on transcriptional profiles expressed by the host that can be recovered from non-invasive swabs used in routine diagnostic testing.

### Example 7: Hyperplexed Sample Barcoded Screening for SARS-CoV-2 by Next Generation Sequencing Provides Host Transciptomes and is Compared to COVID-19 Clinical Outcomes and Severity

To evaluate whether IonLeaSH detects SARS-CoV-2 infection and dissects potential transcriptional markers of COVID-19 presentation from host transcriptomes, RNA was extracted in experimental duplicates (one extraction per user, two independent users total) from 161 specimens (NP swabs and/or saliva) donated by 111 total individuals from the Dominican Republic (1 donor), Peru (29 donors), or the United States (81 donors) with or without clinically confirmed presentation of COVID-19 symptoms in the period between June 2020 and February 2021. Among the 111 total donors, 76 presented with COVID-19 symptoms (97 specimens); of those 76 COVID-19 symptomatic donors, 23 had received mechanical ventilation treatment during their hospital stay (24 specimens) and 5 were hospitalized and undergoing mechanical ventilation at the point of collection (5 specimens). Based on self-reporting, the specimens from COVID-19 symptomatic donors that were used for this experiment were collected ˜1-2 weeks after initial symptom onset and upon admission into a healthcare facility. The remainder of all donors in the test cohort were recruited through asymptomatic screening, their specimens collected upon walk-in. Both RNA extraction sets were retested at NIEHS for SARS-CoV-2 positivity by the CDC EUA rRT-qPCR method. An independent library was synthesized for each replicate RNA extraction set, resulting in two IonLeaSH multiplexed library comprising all 161 specimens, each sequenced in a separate Ion 540 chip (˜750K-1M raw reads avg. per specimen combined). Captured transcripts were compiled across runs on a per-specimen basis for secondary bioinformatics analysis using the SALSA workflow repurposed for single-sample RNA-seq (Lozoya et al. “Patterns, Profiles, and Parsimony: Dissecting Transcriptional Signatures From Minimal Single-Cell RNA-Seq Output With SALSA” Front. Genet., Vol. 11, Article 511286, 9 Oct. 2020).

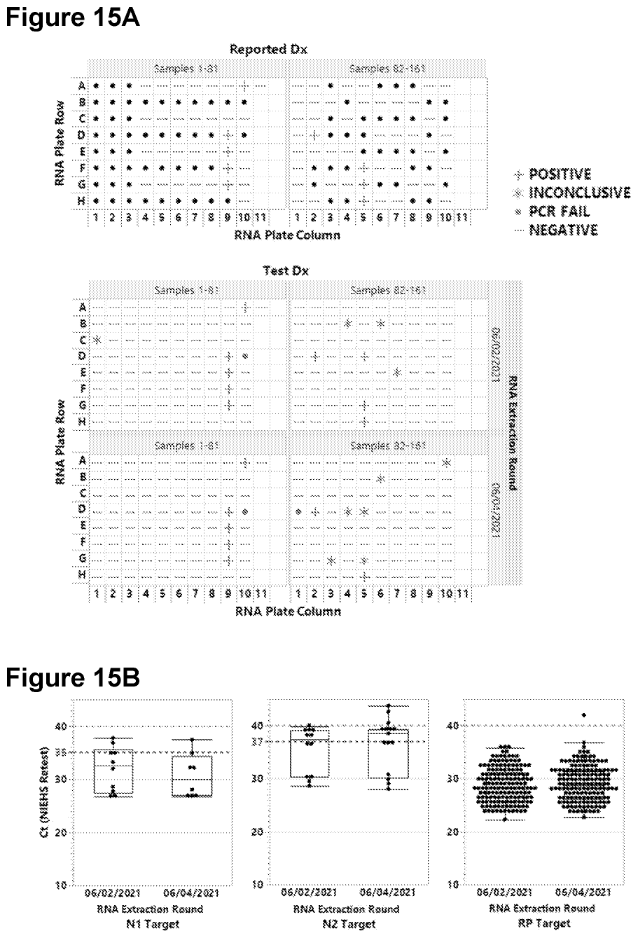

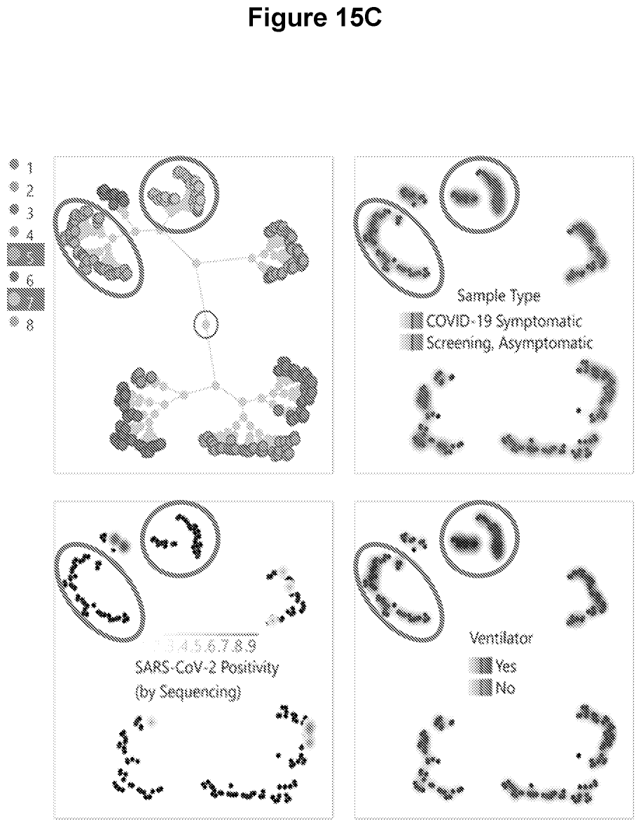

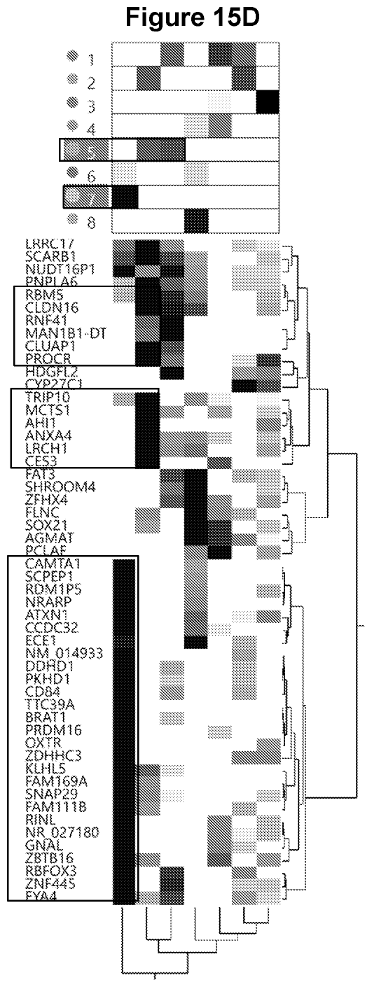

A key point of distinction for this cohort is that, in most cases, the SARS-CoV-2 infection history of each donor was determined by antibody-based assays across the board, or well into the post-symptomatic stage in the specific case of donors with COVID-19 presentation. In effect, given the timecourse observed in SARS-CoV-2 infection (predominantly asymptomatic or pre-symptomatic), this initial testing scheme is already stacked against confirmation by qPCR-based assays. The rRT-qPCR retests performed at NIEHS in experimental duplicates confirmed this roadblock: no more than 9 specimens, confirmed by rRT-qPCR in separate extractions, carried detectable SARS-CoV-2 viral loads (see FIGS. 15A, and 15B); moreover, their Ct values were often closer to the “borderline” positivity status—i.e., accesible to SARS-CoV-2 detection by rRT-qPCR retests (see FIG. 10D) by either N1 or N2 target alone, but substantially less likely to detect by IonLeaSH sequencing (see FIG. 14A). This “borderline” positivity features were confirmed by the paucity of SARS-CoV-2 transcripts in the combined IonLeaSH sequenced data overall.

Still, data from host RNA transcripts was analyzed to determine whether multiplexed sample-barcoded libraries synthesized using LeaSH RNA-seq chemistries allowed for segregation of samples based on latent patterns of shared gene expression from host genomes, independent of SARS-CoV-2 viral load, and at sequencing depths coincident with saturated SARS-CoV-2 transcript representation. Bioinformatics analysis of gene expression patterns by SALSA using IonLeaSH data revealed 8 major transcriptional groupings driven by differential expression of 374 profiler genes from the hosts in addition to viral SARS-CoV-2 RNA (see Table 16, and FIG. 15C). Reported clinical COVID-19 outcomes and therapeutic interventions were available to check for correspondence in relation to their major groupings.

Of note, major groupings 5 and 7, which were the most predominantly coincident with samples from COVID-19 symptomatic donors that required mechanical ventilation, did not show SARS-CoV-2 transcripts, suggesting once again that the timetables for SARS-CoV-2 detection and COVID-19 onset are not in phase (see FIG. 15C). Also, these results showed that the ability to distinguish between diagnosing SARS-CoV-2 infection and forecasting COVID-19 risk and severity, which is not possible based solely on SARS-CoV-2 transcript capture by IonLeaSH sequencing, is feasible based on host transcriptome data (see FIG. 15D). Following extraction of agnostic biomarker genes among the 374-profiler gene subset, the best candidate biomarkers whose expression is richest in samples from patients with severe COVID-19 presentation (majors 5, and 7)—i.e., eventual hospitalization and need for ventilator support of donor subjects—comprised the following 40 human genes: AHI1, ANXA4, ATXN1, BRAT1, CAMTA1, CCDC32, CD84, CES3, CLDN16, CLUAP1, DDHD1, ECE1, EYA4, FAM111B, FAM169A, GNAL, KLHL5, LRCH1, MAN1B1-DT, MCTS1, NM_014933, NR_027180, NRARP, OXTR, PKHD1, PNPLA6, PRDM16, PROCR, RBFOX3, RBM5, RDM1P5, RINL, RNF41, SCPEP1, SNAP29, TRIP10, TTC39A, ZBTB16, ZDHHC3, and ZNF445 (see FIG. 15D). In sum, the analysis of “borderline” SARS-CoV-2 positive specimens, combined with clinical outcome information from donors, was able to dissect transcriptional profiles concomitant with severe COVID-19 presentation, independent of SARS-CoV-2 transcript capture in sequenced data, and directly from NP swabs or saliva samples (see FIGS. 15C, and 15D).

TABLE 16
List of 374 profiler host genes, identified by SALSA analysis, based on IonLeaSH sequencing data for 161 samples from
111 donors with or without clinical COVID-19 diagnosis.
Gene Symbol

| | | | | | | |
|---|---|---|---|---|---|---|
| AHI1 | RDM1P5 | ASB11 | CHORDC | DNAL1 | GOLGA6L1 | KCMF1 |
| ANXA4 | RINL | ASGR1 | CHRNA5 | DNM1L | GORASP2 | KCNJ3 |
| ATXN1 | RNF41 | ATCAY | CHRNB1 | DNTT | GRK3 | KCNJ5 |
| BRAT1 | SCPEP1 | ATG4C | CIAO1 | DUXA | GRM3 | KCNK3 |
| CAMTA1 | SNAP29 | ATG14 | CLHC1 | EBF1 | GRSF1 | KDM4B |
| CCDC32 | TRIP10 | BAG5 | CLIP3 | EGFEM1P | GSTA2 | KDSR |
| CD84 | TTC39A | BASP1-AS1 | COA7 | EMP2 | GTF2F1 | KIAA0408 |
| CES3 | ZBTB16 | BFSP2 | CPT1A | ENAH | HDGFL2 | KIF1C |
| CLDN16 | ZDHHC3 | BMS1P1 | CRX | FA2H | HEATR5A | KIF3C |
| CLUAP1 | ZNF445 | BORCS7 | CSAD | FADS1 | HIGD1B | KIF13A |
| DDHD1 | ABI2 | BRCA2 | CSNK1G1 | FAM41C | HIP1 | KLF15 |
| ECE1 | ACBD7 | C1QTNF2 | CTRL | FAM163A | HMGN3 | KLRD1 |
| EYA4 | ACP7 | C4orf19 | CUL5 | FARP1 | HOXB7 | L1TD1 |
| FAM111B | ACTG1 | C9orf24 | CWC25 | FAT3 | HOXB13 | LDB3 |
| FAM169A | ADCY1 | CABP4 | CXorf38 | FBXO22 | HTR3B | LDLR |
| GNAL | ADCY8 | CACNG8 | CYLD | FDPSP2 | IAPP | LGALSL |
| KLHL5 | ADCY10P1 | CADM2 | CYP27C1 | FECH | ICA1L | LILRB3 |
| LRCH1 | AFF1-AS1 | CALN1 | DBT | FFAR4 | IFIT3 | LIMCH1 |
| MAN1B1-DT | AGMAT | CARF | DCAF10 | FKBP14 | IL2RA | LIN7C |
| MCTS1 | AIPL1 | CASC2 | DCLRE1C | FLNC | IL17RA | LINC00470 |
| NM_014933 | AK3 | CBX5 | DCP2 | FSCN2 | IL17RC | LINC00514 |
| NR_027180 | AKAP5 | CCBE1 | DDX51 | FUZ | IL20 | LINC00665 |
| NRARP | ALDH9A1 | CD74 | DDX55 | GALNT15 | ILF3-DT | LINC00926 |
| OXTR | AMY2A | CDH4 | DEFB118 | GFPT1 | INGX | LINC01502 |
| PKHD1P1 | ANKRD26 | CECR3 | DENND1B | GGCX | IPP | LINC01973 |
| PNPLA6 | ANKRD30BP2 | CEP85 | DENND5B | GK5 | IRGQ | LINC02878 |
| PRDM16 | AP4S1 | CEP350 | DFFA | GLIPR1 | ISY1 | LIPP |
| PROCR | AP5B1 | CERS5 | DGAT1 | GNL3L | ITGB2-AS1 | LOC283856 |
| RBFOX3 | APOL1 | CHDH | DHX30 | GNRHR2 | ITPRIPL2 | LOC374443 |
| RBM5 | APOLD1 | CHM | DIS3L | GNS | KBTBD12 | LOC100505912 |

List of 374 profiler host genes, identified by SALSA analysis, based on IonLeaSH sequencing data for 161 samples from
111 donors with or without clinical COVID-19 diagnosis.
Gene Symbol

| | | | | | |
|---|---|---|---|---|---|
| LRP3 | NME8 | PRMT3 | SHROOM4 | TENT4B | ZNF91 |
| LRRC17 | NMNAT1 | PTCD3 | SLC6A17 | TEP1 | ZNF271P |
| LRRN4CL | NOS1 | PTMA | SLC14A2 | THAP3 | ZNF302 |
| MALAT1 | NOTCH1 | PUS7L | SLC25A15 | TIMM23 | ZNF417 |
| MANEAL | NR_003666 | RAB11FIP3 | SLC25A23 | TKFC | ZNF462 |
| MAPK13 | NR_00369 | RABGEF19 | SLC26A9 | TLCD4 | ZNF526 |
| MARCHF2 | NR_024474 | RASGRP1 | SLC35E1 | TMEM47 | ZNF563 |
| MAVS | NR_027995 | RASSF4 | SLC39A7 | TMEM192 | ZNF669 |
| MCUR1 | NR_037867 | RBM43 | SMAP2 | TMEM241 | ZNF692 |
| MEAF6 | NRXN3 | RBM47 | SMG1 | TNFRSF13B | ZNF827 |
| MEAK7 | NUDT9 | RBMS3 | SMG7-AS1 | TNFRSF25 | ZNF829 |
| MED15P9 | NUDT16P1 | RFK | SMS | TNIP1 | ZNF862 |
| MEG3 | NUP214 | RGS16 | SOGA3 | TPI1 | ZPBP2 |
| METTL6 | NWD1 | RGS17 | SORL1 | TPTE2P1 | ZWILCH |
| MGAT4A | OAZ3 | RIMKLA | SOX21 | TRIM66 | |
| MORF4L1 | PABPC1P2 | RIPPLY3 | SP100 | TRMT2B | |
| MR1 | PAG1 | RNF14 | SPART | TSACC | |
| MREG | PALM2AKAP2 | RPL17 | SPATS2 | TSHZ3 | |
| MRFAP1L1 | PAPOLB | RPL35A | SPIB | UBE2G2 | |
| MSH3 | PBX4 | RPN2 | SPIRE2 | ULBP1 | |
| MTF1 | PCDH17 | RPS6KA6 | SPN | ULK4 | |
| MTMR9 | PCLAF | RPS15AP10 | SPPL3 | VPS37B | |
| MTRNR2L8 | PCSK9 | SCARB1 | SRSF8 | WAC-AS1 | |
| MYO3B | PFN4 | SDS | STX16 | WDR82 | |
| NFATC2IP | PHKA2-AS1 | SEC1P | SWSAP1 | WSB1 | |
| NIPAL3 | PLCXD3 | SELENON | SYCP2 | XAF1 | |
| NLRP12 | PLEKHA5 | SEMA5A | SYNGR4 | YWHAB | |
| NM_001123040 | PPIG | SEPTIN6 | TAF8 | ZBTB8A | |
| NM_004080 | PPP2R3C | SERTAD4 | TBC1D15 | ZC3H12B | |
| NM_019607 | PRKX | SGK3 | TCEAL9 | ZFHX4 | |

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

1. A method for detecting a plurality of nucleic acids in a sample from a subject, comprising:
(a) obtaining the sample from the subject and extracting nucleic acid from the sample to generate a nucleic acid sample;
(b) preparing a library of nucleic acid sequences from the nucleic acid sample; wherein the library of nucleic acid sequences is prepared using:
(i) an anchored oligonucleotide comprising:
(1) a 3′ splint
(2) a unique molecule identifier (UMI)
(3) a sample-specific barcode; and
(4) an oligo-dT;
(ii) a pathogen-specific oligonucleotide primer comprising:
(1) an extended 3′ end cDNA splint
(2) a minimal 3′ end cDNA splint
(3) a 3′ end cDNA UMI; and
(4) a pathogen specific consensus sequence;
(iii) a 3′ indexed adapter oligonucleotide comprising:
(1) a 3′ adapter;
(2) a 3′ barcode; and
(3) a 3′ coupling sequence; and
(iv) a 5′ indexed adapter oligonucleotide comprising:
(1) a 5′ adapter;
(2) a 5′ barcode; and
(3) a 5′ coupling sequence; and
(c) detecting the plurality of nucleic acids by sequencing the library of nucleic acid sequences to generate a plurality of nucleic acid reads.

2. The method of claim 1, wherein preparing the library further comprises using:
(v) a pathogen specific template switching oligonucleotide comprising:
(1) a pathogen specific consensus sequence; and
(2) a template switching motif
(vi) a generic template switching oligonucleotide comprising:
(1) a generic tailing motif; and
(2) a template switching motif;
(vii) a universal cDNA coupler forward primer oligonucleotide comprising:
(1) an extended 3′ end cDNA splint; and
(2) a minimal 3′ end cDNA splint
(viii) a pathogen specific enrichment coupler reverse primer oligonucleotide comprising:
(1) a minimal 5′ end cDNA splint;
(2) an extended 5′ end cDNA splint;
(3) a 5′ end cDNA UMI; and
(4) a pathogenic specific consensus sequence;
(ix) a generic cDNA coupler reverse primer oligonucleotide comprising:
(1) a generic tailing motif; and
(2) a template switching motif; and/or
(x) a rDNA blocking duplex oligonucleotide.

3-6. (canceled)

7. The method of claim 1, wherein the sample is selected from the group consisting of a nasopharyngeal swab, an oropharyngeal swab, a buccal swab, whole saliva sample, cell-free saliva sample, blood plasma, blood serum, whole blood, sputum, stool, urine, cerebral spinal fluid, synovial fluid, peritoneal fluid, pleural fluid, pericardial fluid, and bone marrow.

8. (canceled)

9. The method of claim 1, wherein the sample comprises nucleic acid from both the subject and the pathogen.

10-11. (canceled)

12. The method of claim 1, wherein the pathogen is selected from:
Acinetobacter baumannii, Actinomyces gerencseriae, Actinomyces israelii, Alphavirus species (e.g., Chikungunya virus, Eastern equine encephalitis virus, Venezuelan equine encephalitis virus, and Western equine encephalitis virus), Anaplasma species, Ancylostoma duodenale, Angiostrongylus cantonensis, Angiostrongylus costaricensis, Arcanobacterium haemolyticum, Ascaris lumbricoides, Aspergillus species, Astroviridae species, Babesia species, Bacillus anthracis, Bacillus cereus, Bacteroides species, Balantidium coli, Bartonella bacilliformis, Bartonella henselae, Bartonella, Batrachochytrium dendrabatidis, Baylisascaris species, Blastocystis species, Blastomyces dermatitidis, Bordetella pertussis, Borrelia afzelii, Borrelia burgdorferi, Borrelia garinii, Brucella species, Burkholderia mallei, Burkholderia pseudomallei, Burkholderia species, Caliciviridae species, Campylobacter species, Candida albicans, Capillaria aerophila, Capillaria philippinensis, Chlamydia trachomatis, Chlamydophila pneumoniae, Clonorchis sinensis, Clostridium botulinum, Clostridium difficile, Clostridium perfringens, Clostridium tetani, Coccidioides immitis, Coccidioides posadasii, Colorado tick fever virus (CTFV), Corynebacterium diphtheria, Crimean-Congo hemorrhagic fever virus, Cryptococcus neoformans, Cryptosporidium species, Cyclospora cayetanensis, Cytomegalovirus, Dengue viruses (DEN-1, DEN-2, DEN-3 and DEN-4), Dientamoeba fragilis, Dracunculus medinensis, Ebolavirus (EBOV), Entamoeba histolytica, Enterobius vermicularis, Enterococcus species, Epstein-Barr virus (EBV), Escherichia coli, Fasciola gigantica, Fasciola hepatica, Fasciolopsis buski, Flavivirus species, Geotrichum candidum, Giardia lamblia, Haemophilus ducreyi, Haemophilus influenza, Hantaviridae family, Helicobacter pylori, Hepatitis A virus, Hepatitis B virus, Hepatitis C virus, Hepatitis D Virus, Hepatitis E virus, Herpes simplex virus 1 (HSV-1), Herpes simplex virus 2 (HSV-2), Histoplasma capsulatum, HIV (Human immunodeficiency virus), Human herpesvirus 6 (HHV-6), Human herpesvirus 7 (HHV-7), Human papillomavirus PV), Junin virus, Klebsiella granulomatis, Lassa virus, Legionella pneumophila, Leishmania species, Leptospira species, Listeria monocytogenes, Machupo virus, Measles morbillivirus, Metagonimus yokagawai, Middle East respiratory syndrome coronavirus (MERS), Monkeypox virus, Mumps orthorubulavirus, Mycobacterium leprae, Mycobacterium lepromatosis, Mycobacterium tuberculosis, Mycobacterium ulcerans, Mycoplasma genitalium, Mycoplasma pneumoniae, Necator americanus, Neisseria gonorrhea, Neisseria meningitides, Norovirus, Orthomyxoviridae species, Parvovirus B19, Piedraia hortae, Plasmodium species, Pneumocystis jirovecii, Poliovirus, Propionibacterium propionicus, Rabies virus, Rhinovirus, Rickettsia akari, Rickettsia rickettsia, Rickettsia species, Rickettsia typhi, Rift Valley fever virus, Rotavirus, Rubella virus, Sabia virus, Salmonella species, Sarcoptes scabiei, Schistosoma species, Severe acute respiratory syndrome coronavirus 2 (SARS-CoV-2), Shigella species, Sin Nombre virus, Sporothrix schenckii, Staphylococcus aureus, Staphylococcus species, Streptococcus agalactiae, Streptococcus pneumoniae, Streptococcus pyogenes, Taenia solium, Toxoplasma gondii, Trichinella spiralis, Trichomonas vaginalis, Trichuris trichiura, Trypanosoma brucei, Trypanosoma cruzi, Varicella zoster virus (VZV), Variola major, Variola minor, Venezuelan equine encephalitis virus, Vibrio cholera, Vibrio vulnificus, West Nile virus, Yellow fever virus, Yersinia enterocolitica, Yersinia pestis, Yersinia pseudotuberculosis, Zeaspora fungus, and Zika virus.

13. (canceled)

14. The method of claim 1, wherein the subject is a vertebrate, a mammal, a mouse, a primate, a simian, or a human.

15. (canceled)

16. The method of claim 1, wherein a plurality of samples are obtained, each corresponding to a plurality of subjects, and a plurality of nucleic acid libraries are prepared simultaneously and then sequenced simultaneously.

17. The method of claim 1, wherein the method is performed:
in a single-pot, closed tube chemistry;
in a single-pot, open tube chemistry;
in a split-pot, multi-tube chemistry using PCR pre-amplification; or
in a split-pot, multi-tube chemistry using MDA pre-amplification.

18-20. (canceled)

21. The method of claim 1, wherein the method further comprises determining an infection status of the subject based on the plurality of nucleic acid reads from the subject's library.

22. A method for screening for a pathogen in a plurality of samples using next generation sequencing (NGS), the method comprising:
(a) obtaining the plurality of samples from a plurality of subjects and preparing an agnostic nucleic acid library from each sample in the plurality of samples, wherein each agnostic nucleic acid library comprises a sample specific barcode;
(b) selectively enriching each agnostic nucleic acid library for a plurality of pathogen specific consensus sequences from the pathogen to generate a plurality of enriched, barcoded nucleic acid libraries, wherein selective enrichment comprises targeted amplification of the plurality of conserved sequences in the pathogen; and
(c) sequencing the plurality of enriched, barcoded nucleic acid libraries at the same time using NGS to detect the presence of one or more of the plurality of conserved sequences in the pathogen.

23. The method of claim 22, wherein the method further comprises:
(d) determining an infection status of the subject based on the subject's library.

24. The method of claim 22, wherein the method comprises using one or more of the following oligonucleotides:
an anchored oligonucleotide comprising:
(1) a 3′ splint
(2) a unique molecule identifier (UMI)
(3) a sample-specific barcode; and
(4) an oligo-dT;
(ii) a pathogen-specific oligonucleotide primer comprising:
(1) an extended 3′ end cDNA splint
(2) a minimal 3′ end cDNA splint
(3) a 3′ end cDNA UMI; and
(4) a pathogen specific consensus sequence;
(iii) a 3′ indexed adapter oligonucleotide comprising:
(1) a 3′ adapter;
(2) a 3′ barcode; and
(3) a 3′ coupling sequence;
(iv) a 5′ indexed adapter oligonucleotide comprising:
(1) a 5′ adapter;
(2) a 5′ barcode; and
(3) a 5′ coupling sequence;
(v) a pathogen specific template switching oligonucleotide comprising:
(1) a pathogen specific consensus sequence; and
(2) a template switching motif;
(vi) a generic template switching oligonucleotide comprising:
(1) a generic tailing motif; and
(2) a template switching motif;
(vii) a universal cDNA coupler forward primer oligonucleotide comprising:
(1) an extended 3′ end cDNA splint; and
(2) a minimal 3′ end cDNA splint;
(viii) a pathogen specific enrichment coupler reverse primer oligonucleotide comprising:
(1) a minimal 5′ end cDNA splint;
(2) an extended 5′ end cDNA splint;
(3) a 5′ end cDNA UMI; and
(4) a pathogenic specific consensus sequence;
(ix) a generic cDNA coupler reverse primer oligonucleotide comprising:
(1) a generic tailing motif; and
(2) a template switching motif; or
(x) a rDNA blocking duplex oligonucleotide.

25-40. (canceled)

41. A method of diagnosing SARS-CoV-2 (COVID-19) infection in a subject, comprising:
(a) obtaining a sample from a subject suspected of suffering from SARS-CoV-2;
(b) measuring the expression of one or more genes selected from the genes listed in Tables 14 and/or 16;
(c) comparing the measured expression levels of the one or more genes selected from Tables 14 and/or 16 to the expression levels of the same one or more genes measured in a sample from an individual not suffering from SARS-CoV-2; and
(d) detecting a difference in the expression levels of the one or more genes selected from Tables 14 and/or 16 in the subject suspected of suffering from SARS-CoV-2.

42. A method of diagnosing SARS-CoV-2 (COVID-19) in a subject, comprising:
(a) obtaining a sample from a subject suspected of suffering from SARS-CoV-2;
(b) measuring the expression of one or more genes selected from the genes listed in Tables 14 and/or 16; and
(c) comparing the measured expression levels of the one or more genes selected from Tables 14 and/or 16 to a reference value,
wherein a diagnosis of SARS-CoV-2 is made if the measured gene expression differs from the reference value.

43. A method of detecting SARS-CoV-2 (COVID-19) in a subject, comprising:
(a) obtaining a sample from the subject;
(b) measuring the expression of one or more genes selected from the genes listed in Tables 14 and/or 16; and
(c) comparing the measured expression levels of the one or more genes to the expression levels of the same genes in one or more samples taken from one or more individuals without SARS-CoV-2,
wherein SARS-CoV-2 is detected if the measured gene expression level in the sample taken from the subject differs from the gene expression level measured in the sample taken from the one or more individuals without SARS-CoV-2.

44. A method of treating SARS-CoV-2 (COVID-19), comprising:
(a) obtaining a sample from a subject suspected of having SARS-CoV-2;
(b) measuring the expression of one or more genes selected from the genes listed in Tables 14 and/16;
(c) determining a difference between the expression of the one or more genes in the sample and the expression of the one or more genes in one or more reference samples; and
(d) altering the treatment of the subject based on the difference.

45. A method of diagnosing and/or treating SARS-CoV-2 (COVID-19) in a subject, comprising:
(a) obtaining a sample from a subject suspected of suffering from SARS-CoV-2;
(b) measuring the expression of one or more genes selected from the genes listed in Tables 14 and/16; and
(c) comparing the measured expression levels of the one or more genes selected from Tables 14 and/or 16 to a reference value; wherein a diagnosis of SARS-CoV-2 is made if the measured gene expression differs from the reference value; and
(d) altering the treatment of the subject based on the difference.

46. A method of screening patients for SARS-CoV-2 (COVID-19), comprising:
(a) obtaining a sample from the subject;
(b) measuring the expression of one or more genes selected from the genes listed in Tables 14 and/or 16;
(c) comparing the measured expression of the one or more genes to the expression of the same genes in a reference sample; and
(d) classifying the subject as having a low-risk, intermediate-risk, or high-risk of developing severe COVID-19.

47-55. (canceled)

56. A kit for detecting SARS-CoV-2 (COVID-19) in a subject, wherein the kit comprises reagents useful, sufficient, and/or necessary for determining the level of one or more genes in Tables 14 and/or 16.

57-61. (canceled)
